SEQUENCE LISTING (395 sequences)

SEQ ID NO: 1 - DNA, 20 bp, Synthetic construct
  misc_feature 1..20: note = PCSK9
  source 1..20: mol_type = other DNA
gcgcgtaatctgacgctgtt

SEQ ID NO: 2 - DNA, 20 bp, Synthetic construct
  misc_feature 1..20: note = PCSK9
  source 1..20: mol_type = other DNA
atcagataggatcgtccgat

SEQ ID NO: 3 - DNA, 20 bp, Synthetic construct
  misc_feature 1..20: note = PCSK9
  source 1..20: mol_type = other DNA
aggtttccgcagcgacgtcg

SEQ ID NO: 4 - DNA, 20 bp, Synthetic construct
  misc_feature 1..20: note = PCSK9
  source 1..20: mol_type = other DNA
gggcgccgccgttcagttca

SEQ ID NO: 5 - DNA, 20 bp, Synthetic construct
  misc_feature 1..20: note = PCSK9
  source 1..20: mol_type = other DNA
ggtgctagccttgcgttccg

SEQ ID NO: 6 - DNA, 20 bp, Synthetic construct
  misc_feature 1..20: note = PCSK9
  source 1..20: mol_type = other DNA
cattaacggaacccccggac

SEQ ID NO: 7 - DNA, 20 bp, Synthetic construct
  misc_feature 1..20: note = PCSK9
  source 1..20: mol_type = other DNA
aggatcgtccgatggggctc

SEQ ID NO: 8 - DNA, 20 bp, Synthetic construct
  misc_feature 1..20: note = PCSK9
  source 1..20: mol_type = other DNA
ccgttaatgtttaatcagat

SEQ ID NO: 9 - DNA, 19 bp, Synthetic construct
  misc_feature 1..19: note = PCSK9
  source 1..19: mol_type = other DNA
cgtaatctgacgctgtttg

SEQ ID NO: 10 - DNA, 20 bp, Synthetic construct
  misc_feature 1..20: note = PCSK9
  source 1..20: mol_type = other DNA
ggtgtgggtgcttgacgcct

SEQ ID NO: 11 - DNA, 20 bp, Synthetic construct
  misc_feature 1..20: note = PCSK9
  source 1..20: mol_type = other DNA
acccactgcacgctggacag

SEQ ID NO: 12 - DNA, 20 bp, Synthetic construct
  misc_feature 1..20: note = PCSK9
  source 1..20: mol_type = other DNA
gcacagtaacaacccctggt

SEQ ID NO: 13 - DNA, 20 bp, Synthetic construct
  misc_feature 1..20: note = PCSK9
  source 1..20: mol_type = other DNA
ccatccattctttctctagg

SEQ ID NO: 14 - DNA, 20 bp, Synthetic construct
  misc_feature 1..20: note = LPA
  source 1..20: mol_type = other DNA
aaggagacataaaggcaatg

SEQ ID NO: 15 - DNA, 20 bp, Synthetic construct
  misc_feature 1..20: note = LPA
  source 1..20: mol_type = other DNA
ggcaatgtggagcagctgag

SEQ ID NO: 16 - DNA, 20 bp, Synthetic construct
  misc_feature 1..20: note = LPA
  source 1..20: mol_type = other DNA
ggagcagctgaggggggaaa

SEQ ID NO: 17 - DNA, 20 bp, Synthetic construct
  misc_feature 1..20: note = LPA
  source 1..20: mol_type = other DNA
tgtcaatagatgctgggaag

SEQ ID NO: 18 - DNA, 20 bp, Synthetic construct
  misc_feature 1..20: note = LPA
  source 1..20: mol_type = other DNA
agtgcaatgtcaatagatgc

SEQ ID NO: 19 - DNA, 20 bp, Synthetic construct
  misc_feature 1..20: note = LPA
  source 1..20: mol_type = other DNA
tttataagactctatattca

SEQ ID NO: 20 - DNA, 20 bp, Synthetic construct
  misc_feature 1..20: note = LPA
  source 1..20: mol_type = other DNA
catgtaagtcaacaatgtcc

SEQ ID NO: 21 - DNA, 20 bp, Synthetic construct
  misc_feature 1..20: note = LPA
  source 1..20: mol_type = other DNA
gtcaacaatgtcctgggatt

SEQ ID NO: 22 - DNA, 20 bp, Synthetic construct
  misc_feature 1..20: note = LPA
  source 1..20: mol_type = other DNA
catatacaagattttgaact

SEQ ID NO: 23 - DNA, 20 bp, Synthetic construct
  misc_feature 1..20: note = LPA
  source 1..20: mol_type = other DNA
gcaccgtgacagtcttcacg

SEQ ID NO: 24 - DNA, 20 bp, Synthetic construct
  misc_feature 1..20: note = MYLIP
  source 1..20: mol_type = other DNA
ttggcggggacccgagctga

SEQ ID NO: 25 - DNA, 20 bp, Synthetic construct
  misc_feature 1..20: note = MYLIP
  source 1..20: mol_type = other DNA
ctgtcgcagcgcaggcagtt

SEQ ID NO: 26 - DNA, 20 bp, Synthetic construct
  misc_feature 1..20: note = MYLIP
  source 1..20: mol_type = other DNA
gctggagtgcggcgccaccg

SEQ ID NO: 27 - DNA, 20 bp, Synthetic construct
  misc_feature 1..20: note = MYLIP
  source 1..20: mol_type = other DNA
cggcgccaccgcggaggaca

SEQ ID NO: 28 - DNA, 20 bp, Synthetic construct
  misc_feature 1..20: note = MYLIP
  source 1..20: mol_type = other DNA
cagctctgcggacccttgtc

SEQ ID NO: 29 - DNA, 20 bp, Synthetic construct
  misc_feature 1..20: note = MYLIP
  source 1..20: mol_type = other DNA
ccccgcgcacaccaaagaga

SEQ ID NO: 30 - DNA, 20 bp, Synthetic construct
  misc_feature 1..20: note = MYLIP
  source 1..20: mol_type = other DNA
cctcgtcacataacacagca

SEQ ID NO: 31 - DNA, 20 bp, Synthetic construct
  misc_feature 1..20: note = MYLIP
  source 1..20: mol_type = other DNA
acctccatcagcaccgcgtc

SEQ ID NO: 32 - DNA, 20 bp, Synthetic construct
  misc_feature 1..20: note = MYLIP
  source 1..20: mol_type = other DNA
ggaggcgaaagccaacggcg

SEQ ID NO: 33 - DNA, 20 bp, Synthetic construct
  misc_feature 1..20: note = MYLIP
  source 1..20: mol_type = other DNA
gttgaggcagtcctcgccgt

SEQ ID NO: 34 - DNA, 20 bp, Synthetic construct
  misc_feature 1..20: note = ANGLPTL3
  source 1..20: mol_type = other DNA
tacattcgtgcaagttaaca

SEQ ID NO: 35 - DNA, 20 bp, Synthetic construct
  misc_feature 1..20: note = ANGLPTL3
  source 1..20: mol_type = other DNA
cctaccaaccttaccttttc

SEQ ID NO: 36 - DNA, 20 bp, Synthetic construct
  misc_feature 1..20: note = ANGLPTL3
  source 1..20: mol_type = other DNA
tatatagagttaagaagtct

SEQ ID NO: 37 - DNA, 20 bp, Synthetic construct
  misc_feature 1..20: note = ANGLPTL3
  source 1..20: mol_type = other DNA
aacgtggaactgttttcttc

SEQ ID NO: 38 - DNA, 20 bp, Synthetic construct
  misc_feature 1..20: note = ANGLPTL3
  source 1..20: mol_type = other DNA
attttcaatttcaagcaacg

SEQ ID NO: 39 - DNA, 20 bp, Synthetic construct
  misc_feature 1..20: note = ANGLPTL3
  source 1..20: mol_type = other DNA
attctggaggaaataactag

SEQ ID NO: 40 - DNA, 20 bp, Synthetic construct
  misc_feature 1..20: note = ANGLPTL3
  source 1..20: mol_type = other DNA
gcaaatcttgattttggctc

SEQ ID NO: 41 - DNA, 20 bp, Synthetic construct
  misc_feature 1..20: note = ANGLPTL3
  source 1..20: mol_type = other DNA
agccaatggcctccttcagt

SEQ ID NO: 42 - DNA, 20 bp, Synthetic construct
  misc_feature 1..20: note = ANGLPTL3
  source 1..20: mol_type = other DNA
taagaccatgtcccaactga

SEQ ID NO: 43 - DNA, 20 bp, Synthetic construct
  misc_feature 1..20: note = ANGLPTL3
  source 1..20: mol_type = other DNA
agactttgtccataagacga

SEQ ID NO: 44 - DNA, 20 bp, Synthetic construct
  misc_feature 1..20: note = APOC3
  source 1..20: mol_type = other DNA
ggggcacccgtccagctccg

SEQ ID NO: 45 - DNA, 20 bp, Synthetic construct
  misc_feature 1..20: note = APOC3
  source 1..20: mol_type = other DNA
tgacctttgcccagcgccct

SEQ ID NO: 46 - DNA, 20 bp, Synthetic construct
  misc_feature 1..20: note = APOC3
  source 1..20: mol_type = other DNA
tccagatgcagcaagcgggc

SEQ ID NO: 47 - DNA, 20 bp, Synthetic construct
  misc_feature 1..20: note = APOC3
  source 1..20: mol_type = other DNA
tagggatgaactgagcagac

SEQ ID NO: 48 - DNA, 20 bp, Synthetic construct
  misc_feature 1..20: note = APOC3
  source 1..20: mol_type = other DNA
agaagcacttgctagagcta

SEQ ID NO: 49 - DNA, 20 bp, Synthetic construct
  misc_feature 1..20: note = APOC3
  source 1..20: mol_type = other DNA
ctgctccaggtaatgccctc

SEQ ID NO: 50 - DNA, 20 bp, Synthetic construct
  misc_feature 1..20: note = APOC3
  source 1..20: mol_type = other DNA
gggagagttgggaaatccct

SEQ ID NO: 51 - DNA, 20 bp, Synthetic construct
  misc_feature 1..20: note = APOC3
  source 1..20: mol_type = other DNA
aggaagcctcggagctggac

SEQ ID NO: 52 - DNA, 20 bp, Synthetic construct
  misc_feature 1..20: note = APOC3
  source 1..20: mol_type = other DNA
ccctggagatgatataaaac

SEQ ID NO: 53 - DNA, 20 bp, Synthetic construct
  misc_feature 1..20: note = APOC3
  source 1..20: mol_type = other DNA
tcataacctgaagaacatgg

SEQ ID NO: 54 - DNA, 20 bp, Synthetic construct
  misc_feature 1..20: note = APOB
  source 1..20: mol_type = other DNA
gtccatcgccagctgcggtg

SEQ ID NO: 55 - DNA, 20 bp, Synthetic construct
  misc_feature 1..20: note = APOB
  source 1..20: mol_type = other DNA
ggcgcccgcaccccatttat

SEQ ID NO: 56 - DNA, 20 bp, Synthetic construct
  misc_feature 1..20: note = APOB
  source 1..20: mol_type = other DNA
cagagcggccgcgcactcac

SEQ ID NO: 57 - DNA, 20 bp, Synthetic construct
  misc_feature 1..20: note = APOB
  source 1..20: mol_type = other DNA
ctcagcggcagcaaccgaga

SEQ ID NO: 58 - DNA, 20 bp, Synthetic construct
  misc_feature 1..20: note = APOB
  source 1..20: mol_type = other DNA
tcccggtgggaatgcgcggc

SEQ ID NO: 59 - DNA, 20 bp, Synthetic construct
  misc_feature 1..20: note = APOB
  source 1..20: mol_type = other DNA
gcattcccaccgggacctgc

SEQ ID NO: 60 - DNA, 20 bp, Synthetic construct
  misc_feature 1..20: note = APOB
  source 1..20: mol_type = other DNA
gcctcgcggccctggctggc

SEQ ID NO: 61 - DNA, 20 bp, Synthetic construct
  misc_feature 1..20: note = APOB
  source 1..20: mol_type = other DNA
cccggccaacctcgtgccgc

SEQ ID NO: 62 - DNA, 20 bp, Synthetic construct
  misc_feature 1..20: note = APOB
  source 1..20: mol_type = other DNA
agcgccagcagcgcgggcct

SEQ ID NO: 63 - DNA, 20 bp, Synthetic construct
  misc_feature 1..20: note = APOB
  source 1..20: mol_type = other DNA
ctccctctgcgcccgcagag

SEQ ID NO: 64 - RNA, 20 nt, Synthetic construct
  misc_feature 1..20: note = PCSK9
  source 1..20: mol_type = other RNA
gcgcgtaatctgacgctgtt

SEQ ID NO: 65 - RNA, 20 nt, Synthetic construct
  misc_feature 1..20: note = PCSK9
  source 1..20: mol_type = other RNA
atcagataggatcgtccgat

SEQ ID NO: 66 - RNA, 20 nt, Synthetic construct
  misc_feature 1..20: note = PCSK9
  source 1..20: mol_type = other RNA
aggtttccgcagcgacgtcg

SEQ ID NO: 67 - RNA, 20 nt, Synthetic construct
  misc_feature 1..20: note = PCSK9
  source 1..20: mol_type = other RNA
gggcgccgccgttcagttca

SEQ ID NO: 68 - RNA, 20 nt, Synthetic construct
  misc_feature 1..20: note = PCSK9
  source 1..20: mol_type = other RNA
ggtgctagccttgcgttccg

SEQ ID NO: 69 - RNA, 20 nt, Synthetic construct
  source 1..20: mol_type = other RNA
cattaacggaacccccggac

SEQ ID NO: 70 - RNA, 20 nt, Synthetic construct
  misc_feature 1..20: note = PCSK9
  source 1..20: mol_type = other RNA
aggatcgtccgatggggctc

SEQ ID NO: 71 - RNA, 20 nt, Synthetic construct
  misc_feature 1..20: note = PCSK9
  source 1..20: mol_type = other RNA
ccgttaatgtttaatcagat

SEQ ID NO: 72 - RNA, 19 nt, Synthetic construct
  misc_feature 1..19: note = PCSK9
  source 1..19: mol_type = other RNA
cgtaatctgacgctgtttg

SEQ ID NO: 73 - RNA, 20 nt, Synthetic construct
  misc_feature 1..20: note = PCSK9
  source 1..20: mol_type = other RNA
ggtgtgggtgcttgacgcct

SEQ ID NO: 74 - RNA, 20 nt, Synthetic construct
  misc_feature 1..20: note = PCSK9
  source 1..20: mol_type = other RNA
acccactgcacgctggacag

SEQ ID NO: 75 - RNA, 20 nt, Synthetic construct
  misc_feature 1..20: note = PCSK9
  source 1..20: mol_type = other RNA
gcacagtaacaacccctggt

SEQ ID NO: 76 - RNA, 20 nt, Synthetic construct
  misc_feature 1..20: note = PCSK9
  source 1..20: mol_type = other RNA
ccatccattctttctctagg

SEQ ID NO: 77 - RNA, 20 nt, Synthetic construct
  misc_feature 1..20: note = LPA
  source 1..20: mol_type = other RNA
aaggagacataaaggcaatg

SEQ ID NO: 78 - RNA, 20 nt, Synthetic construct
  misc_feature 1..20: note = LPA
  source 1..20: mol_type = other RNA
ggcaatgtggagcagctgag

SEQ ID NO: 79 - RNA, 20 nt, Synthetic construct
  misc_feature 1..20: note = LPA
  source 1..20: mol_type = other RNA
ggagcagctgaggggggaaa

SEQ ID NO: 80 - RNA, 20 nt, Synthetic construct
  misc_feature 1..20: note = LPA
  source 1..20: mol_type = other RNA
tgtcaatagatgctgggaag

SEQ ID NO: 81 - RNA, 20 nt, Synthetic construct
  misc_feature 1..20: note = LPA
  source 1..20: mol_type = other RNA
agtgcaatgtcaatagatgc

SEQ ID NO: 82 - RNA, 20 nt, Synthetic construct
  misc_feature 1..20: note = LPA
  source 1..20: mol_type = other RNA
tttataagactctatattca

SEQ ID NO: 83 - RNA, 20 nt, Synthetic construct
  misc_feature 1..20: note = LPA
  source 1..20: mol_type = other RNA
catgtaagtcaacaatgtcc

SEQ ID NO: 84 - RNA, 20 nt, Synthetic construct
  misc_feature 1..20: note = LPA
  source 1..20: mol_type = other RNA
gtcaacaatgtcctgggatt

SEQ ID NO: 85 - RNA, 20 nt, Synthetic construct
  misc_feature 1..20: note = LPA
  source 1..20: mol_type = other RNA
catatacaagattttgaact

SEQ ID NO: 86 - RNA, 20 nt, Synthetic construct
  misc_feature 1..20: note = LPA
  source 1..20: mol_type = other RNA
gcaccgtgacagtcttcacg

SEQ ID NO: 87 - RNA, 20 nt, Synthetic construct
  misc_feature 1..20: note = MYLIP
  source 1..20: mol_type = other RNA
ttggcggggacccgagctga

SEQ ID NO: 88 - RNA, 20 nt, Synthetic construct
  misc_feature 1..20: note = MYLIP
  source 1..20: mol_type = other RNA
ctgtcgcagcgcaggcagtt

SEQ ID NO: 89 - RNA, 20 nt, Synthetic construct
  misc_feature 1..20: note = MYLIP
  source 1..20: mol_type = other RNA
gctggagtgcggcgccaccg

SEQ ID NO: 90 - RNA, 20 nt, Synthetic construct
  misc_feature 1..20: note = MYLIP
  source 1..20: mol_type = other RNA
cggcgccaccgcggaggaca

SEQ ID NO: 91 - RNA, 20 nt, Synthetic construct
  misc_feature 1..20: note = MYLIP
  source 1..20: mol_type = other RNA
cagctctgcggacccttgtc

SEQ ID NO: 92 - RNA, 20 nt, Synthetic construct
  misc_feature 1..20: note = MYLIP
  source 1..20: mol_type = other RNA
ccccgcgcacaccaaagaga

SEQ ID NO: 93 - RNA, 20 nt, Synthetic construct
  misc_feature 1..20: note = MYLIP
  source 1..20: mol_type = other RNA
cctcgtcacataacacagca

SEQ ID NO: 94 - RNA, 20 nt, Synthetic construct
  misc_feature 1..20: note = MYLIP
  source 1..20: mol_type = other RNA
acctccatcagcaccgcgtc

SEQ ID NO: 95 - RNA, 20 nt, Synthetic construct
  misc_feature 1..20: note = MYLIP
  source 1..20: mol_type = other RNA
ggaggcgaaagccaacggcg

SEQ ID NO: 96 - RNA, 20 nt, Synthetic construct
  misc_feature 1..20: note = MYLIP
  source 1..20: mol_type = other RNA
gttgaggcagtcctcgccgt

SEQ ID NO: 97 - RNA, 20 nt, Synthetic construct
  misc_feature 1..20: note = ANGLPTL3
  source 1..20: mol_type = other RNA
tacattcgtgcaagttaaca

SEQ ID NO: 98 - RNA, 20 nt, Synthetic construct
  misc_feature 1..20: note = ANGLPTL3
  source 1..20: mol_type = other RNA
cctaccaaccttaccttttc

SEQ ID NO: 99 - RNA, 20 nt, Synthetic construct
  misc_feature 1..20: note = ANGLPTL3
  source 1..20: mol_type = other RNA
tatatagagttaagaagtct

SEQ ID NO: 100 - RNA, 20 nt, Synthetic construct
  misc_feature 1..20: note = ANGLPTL3
  source 1..20: mol_type = other RNA
aacgtggaactgttttcttc

SEQ ID NO: 101 - RNA, 20 nt, Synthetic construct
  misc_feature 1..20: note = ANGLPTL3
  source 1..20: mol_type = other RNA
attttcaatttcaagcaacg

SEQ ID NO: 102 - RNA, 20 nt, Synthetic construct
  misc_feature 1..20: note = ANGLPTL3
  source 1..20: mol_type = other RNA
attctggaggaaataactag

SEQ ID NO: 103 - RNA, 20 nt, Synthetic construct
  misc_feature 1..20: note = ANGLPTL3
  source 1..20: mol_type = other RNA
gcaaatcttgattttggctc

SEQ ID NO: 104 - RNA, 20 nt, Synthetic construct
  misc_feature 1..20: note = ANGLPTL3
  source 1..20: mol_type = other RNA
agccaatggcctccttcagt

SEQ ID NO: 105 - RNA, 20 nt, Synthetic construct
  misc_feature 1..20: note = ANGLPTL3
  source 1..20: mol_type = other RNA
taagaccatgtcccaactga

SEQ ID NO: 106 - RNA, 20 nt, Synthetic construct
  misc_feature 1..20: note = ANGLPTL3
  source 1..20: mol_type = other RNA
agactttgtccataagacga

SEQ ID NO: 107 - RNA, 20 nt, Synthetic construct
  misc_feature 1..20: note = APOC3
  source 1..20: mol_type = other RNA
ggggcacccgtccagctccg

SEQ ID NO: 108 - RNA, 20 nt, Synthetic construct
  misc_feature 1..20: note = APOC3
  source 1..20: mol_type = other RNA
tgacctttgcccagcgccct

SEQ ID NO: 109 - RNA, 20 nt, Synthetic construct
  misc_feature 1..20: note = APOC3
  source 1..20: mol_type = other RNA
tccagatgcagcaagcgggc

SEQ ID NO: 110 - RNA, 20 nt, Synthetic construct
  misc_feature 1..20: note = APOC3
  source 1..20: mol_type = other RNA
tagggatgaactgagcagac

SEQ ID NO: 111 - RNA, 20 nt, Synthetic construct
  misc_feature 1..20: note = APOC3
  source 1..20: mol_type = other RNA
agaagcacttgctagagcta

SEQ ID NO: 112 - RNA, 20 nt, Synthetic construct
  misc_feature 1..20: note = APOC3
  source 1..20: mol_type = other RNA
ctgctccaggtaatgccctc

SEQ ID NO: 113 - RNA, 20 nt, Synthetic construct
  misc_feature 1..20: note = APOC3
  source 1..20: mol_type = other RNA
gggagagttgggaaatccct

SEQ ID NO: 114 - RNA, 20 nt, Synthetic construct
  misc_feature 1..20: note = APOC3
  source 1..20: mol_type = other RNA
aggaagcctcggagctggac

SEQ ID NO: 115 - RNA, 20 nt, Synthetic construct
  misc_feature 1..20: note = APOC3
  source 1..20: mol_type = other RNA
ccctggagatgatataaaac

SEQ ID NO: 116 - RNA, 20 nt, Synthetic construct
  misc_feature 1..20: note = APOC3
  source 1..20: mol_type = other RNA
tcataacctgaagaacatgg

SEQ ID NO: 117 - RNA, 20 nt, Synthetic construct
  misc_feature 1..20: note = APOB
  source 1..20: mol_type = other RNA
gtccatcgccagctgcggtg

SEQ ID NO: 118 - RNA, 20 nt, Synthetic construct
  misc_feature 1..20: note = APOB
  source 1..20: mol_type = other RNA
ggcgcccgcaccccatttat

SEQ ID NO: 119 - RNA, 20 nt, Synthetic construct
  misc_feature 1..20: note = APOB
  source 1..20: mol_type = other RNA
cagagcggccgcgcactcac

SEQ ID NO: 120 - RNA, 20 nt, Synthetic construct
  misc_feature 1..20: note = APOB
  source 1..20: mol_type = other RNA
ctcagcggcagcaaccgaga

SEQ ID NO: 121 - RNA, 20 nt, Synthetic construct
  misc_feature 1..20: note = APOB
  source 1..20: mol_type = other RNA
tcccggtgggaatgcgcggc

SEQ ID NO: 122 - RNA, 20 nt, Synthetic construct
  misc_feature 1..20: note = APOB
  source 1..20: mol_type = other RNA
gcattcccaccgggacctgc

SEQ ID NO: 123 - RNA, 20 nt, Synthetic construct
  misc_feature 1..20: note = APOB
  source 1..20: mol_type = other RNA
gcctcgcggccctggctggc

SEQ ID NO: 124 - RNA, 20 nt, Synthetic construct
  misc_feature 1..20: note = APOB
  source 1..20: mol_type = other RNA
cccggccaacctcgtgccgc

SEQ ID NO: 125 - RNA, 20 nt, Synthetic construct
  misc_feature 1..20: note = APOB
  source 1..20: mol_type = other RNA
agcgccagcagcgcgggcct

SEQ ID NO: 126 - RNA, 20 nt, Synthetic construct
  misc_feature 1..20: note = APOB
  source 1..20: mol_type = other RNA
ctccctctgcgcccgcagag

SEQ ID NO: 127 - RNA, 106 nt, Synthetic construct
  misc_feature 1..106: note = PCSK9
  source 1..106: mol_type = other RNA
gcgcgtaatctgacgctgttgtttaagagctatgctggaaacagcatagcaagtttaaataaggctagtccgttatcaacttgaaaaagtggcaccgagtcggtgc

SEQ ID NO: 128 - RNA, 106 nt, Synthetic construct
  misc_feature 1..106: note = PCSK9
  source 1..106: mol_type = other RNA
atcagataggatcgtccgatgtttaagagctatgctggaaacagcatagcaagtttaaataaggctagtccgttatcaacttgaaaaagtggcaccgagtcggtgc

SEQ ID NO: 129 - RNA, 106 nt, Synthetic construct
  misc_feature 1..106: note = PCSK9
  source 1..106: mol_type = other RNA
aggtttccgcagcgacgtcggtttaagagctatgctggaaacagcatagcaagtttaaataaggctagtccgttatcaacttgaaaaagtggcaccgagtcggtgc

SEQ ID NO: 130 - RNA, 106 nt, Synthetic construct
  misc_feature 1..106: note = PCSK9
  source 1..106: mol_type = other RNA
gggcgccgccgttcagttcagtttaagagctatgctggaaacagcatagcaagtttaaataaggctagtccgttatcaacttgaaaaagtggcaccgagtcggtgc

SEQ ID NO: 131 - RNA, 106 nt, Synthetic construct
  misc_feature 1..106: note = PCSK9
  source 1..106: mol_type = other RNA
ggtgctagccttgcgttccggtttaagagctatgctggaaacagcatagcaagtttaaataaggctagtccgttatcaacttgaaaaagtggcaccgagtcggtgc

SEQ ID NO: 132 - RNA, 106 nt, Synthetic construct
  misc_feature 1..106: note = PCSK9
  source 1..106: mol_type = other RNA
cattaacggaacccccggacgtttaagagctatgctggaaacagcatagcaagtttaaataaggctagtccgttatcaacttgaaaaagtggcaccgagtcggtgc

SEQ ID NO: 133 - RNA, 106 nt, Synthetic construct
  misc_feature 1..106: note = PCSK9
  source 1..106: mol_type = other RNA
aggatcgtccgatggggctcgtttaagagctatgctggaaacagcatagcaagtttaaataaggctagtccgttatcaacttgaaaaagtggcaccgagtcggtgc

SEQ ID NO: 134 - RNA, 106 nt, Synthetic construct
  misc_feature 1..106: note = PCSK9
  source 1..106: mol_type = other RNA
ccgttaatgtttaatcagatgtttaagagctatgctggaaacagcatagcaagtttaaataaggctagtccgttatcaacttgaaaaagtggcaccgagtcggtgc

SEQ ID NO: 135 - RNA, 105 nt, Synthetic construct
  misc_feature 1..105: note = PCSK9
  source 1..105: mol_type = other RNA
cgtaatctgacgctgtttggtttaagagctatgctggaaacagcatagcaagtttaaataaggctagtccgttatcaacttgaaaaagtggcaccgagtcggtgc

SEQ ID NO: 136 - RNA, 106 nt, Synthetic construct
  misc_feature 1..106: note = PCSK9
  source 1..106: mol_type = other RNA
ggtgtgggtgcttgacgcctgtttaagagctatgctggaaacagcatagcaagtttaaataaggctagtccgttatcaacttgaaaaagtggcaccgagtcggtgc

SEQ ID NO: 137 - RNA, 106 nt, Synthetic construct
  misc_feature 1..106: note = PCSK9
  source 1..106: mol_type = other RNA
acccactgcacgctggacaggtttaagagctatgctggaaacagcatagcaagtttaaataaggctagtccgttatcaacttgaaaaagtggcaccgagtcggtgc

SEQ ID NO: 138 - RNA, 106 nt, Synthetic construct
  misc_feature 1..106: note = PCSK9
  source 1..106: mol_type = other RNA
gcacagtaacaacccctggtgtttaagagctatgctggaaacagcatagcaagtttaaataaggctagtccgttatcaacttgaaaaagtggcaccgagtcggtgc

SEQ ID NO: 139 - RNA, 106 nt, Synthetic construct
  misc_feature 1..106: note = PCSK9
  source 1..106: mol_type = other RNA
ccatccattctttctctagggtttaagagctatgctggaaacagcatagcaagtttaaataaggctagtccgttatcaacttgaaaaagtggcaccgagtcggtgc

SEQ ID NO: 140 - RNA, 106 nt, Synthetic construct
  misc_feature 1..106: note = LPA
  source 1..106: mol_type = other RNA
aaggagacataaaggcaatggtttaagagctatgctggaaacagcatagcaagtttaaataaggctagtccgttatcaacttgaaaaagtggcaccgagtcggtgc

SEQ ID NO: 141 - RNA, 106 nt, Synthetic construct
  misc_feature 1..106: note = LPA
  source 1..106: mol_type = other RNA
ggcaatgtggagcagctgaggtttaagagctatgctggaaacagcatagcaagtttaaataaggctagtccgttatcaacttgaaaaagtggcaccgagtcggtgc

SEQ ID NO: 142 - RNA, 106 nt, Synthetic construct
  misc_feature 1..106: note = LPA
  source 1..106: mol_type = other RNA
ggagcagctgaggggggaaagtttaagagctatgctggaaacagcatagcaagtttaaataaggctagtccgttatcaacttgaaaaagtggcaccgagtcggtgc

SEQ ID NO: 143 - RNA, 106 nt, Synthetic construct
  misc_feature 1..106: note = LPA
  source 1..106: mol_type = other RNA
tgtcaatagatgctgggaaggtttaagagctatgctggaaacagcatagcaagtttaaataaggctagtccgttatcaacttgaaaaagtggcaccgagtcggtgc

SEQ ID NO: 144 - RNA, 106 nt, Synthetic construct
  misc_feature 1..106: note = LPA
  source 1..106: mol_type = other RNA
agtgcaatgtcaatagatgcgtttaagagctatgctggaaacagcatagcaagtttaaataaggctagtccgttatcaacttgaaaaagtggcaccgagtcggtgc

SEQ ID NO: 145 - RNA, 106 nt, Synthetic construct
  misc_feature 1..106: note = LPA
  source 1..106: mol_type = other RNA
tttataagactctatattcagtttaagagctatgctggaaacagcatagcaagtttaaataaggctagtccgttatcaacttgaaaaagtggcaccgagtcggtgc

SEQ ID NO: 146 - RNA, 106 nt, Synthetic construct
  misc_feature 1..106: note = LPA
  source 1..106: mol_type = other RNA
catgtaagtcaacaatgtccgtttaagagctatgctggaaacagcatagcaagtttaaataaggctagtccgttatcaacttgaaaaagtggcaccgagtcggtgc

SEQ ID NO: 147 - RNA, 106 nt, Synthetic construct
  misc_feature 1..106: note = LPA
  source 1..106: mol_type = other RNA
gtcaacaatgtcctgggattgtttaagagctatgctggaaacagcatagcaagtttaaataaggctagtccgttatcaacttgaaaaagtggcaccgagtcggtgc

SEQ ID NO: 148 - RNA, 106 nt, Synthetic construct
  misc_feature 1..106: note = LPA
  source 1..106: mol_type = other RNA
catatacaagattttgaactgtttaagagctatgctggaaacagcatagcaagtttaaataaggctagtccgttatcaacttgaaaaagtggcaccgagtcggtgc

SEQ ID NO: 149 - RNA, 106 nt, Synthetic construct
  misc_feature 1..106: note = LPA
  source 1..106: mol_type = other RNA
gcaccgtgacagtcttcacggtttaagagctatgctggaaacagcatagcaagtttaaataaggctagtccgttatcaacttgaaaaagtggcaccgagtcggtgc

SEQ ID NO: 150 - RNA, 106 nt, Synthetic construct
  misc_feature 1..106: note = MYLIP
  source 1..106: mol_type = other RNA
ttggcggggacccgagctgagtttaagagctatgctggaaacagcatagcaagtttaaataaggctagtccgttatcaacttgaaaaagtggcaccgagtcggtgc

SEQ ID NO: 151 - RNA, 106 nt, Synthetic construct
  misc_feature 1..106: note = MYLIP
  source 1..106: mol_type = other RNA
ctgtcgcagcgcaggcagttgtttaagagctatgctggaaacagcatagcaagtttaaataaggctagtccgttatcaacttgaaaaagtggcaccgagtcggtgc

SEQ ID NO: 152 - RNA, 106 nt, Synthetic construct
  misc_feature 1..106: note = MYLIP
  source 1..106: mol_type = other RNA
gctggagtgcggcgccaccggtttaagagctatgctggaaacagcatagcaagtttaaataaggctagtccgttatcaacttgaaaaagtggcaccgagtcggtgc

SEQ ID NO: 153 - RNA, 106 nt, Synthetic construct
  misc_feature 1..106: note = MYLIP
  source 1..106: mol_type = other RNA
cggcgccaccgcggaggacagtttaagagctatgctggaaacagcatagcaagtttaaataaggctagtccgttatcaacttgaaaaagtggcaccgagtcggtgc

SEQ ID NO: 154 - RNA, 106 nt, Synthetic construct
  misc_feature 1..106: note = MYLIP
  source 1..106: mol_type = other RNA
cagctctgcggacccttgtcgtttaagagctatgctggaaacagcatagcaagtttaaataaggctagtccgttatcaacttgaaaaagtggcaccgagtcggtgc

SEQ ID NO: 155 - RNA, 106 nt, Synthetic construct
  misc_feature 1..106: note = MYLIP
  source 1..106: mol_type = other RNA
ccccgcgcacaccaaagagagtttaagagctatgctggaaacagcatagcaagtttaaataaggctagtccgttatcaacttgaaaaagtggcaccgagtcggtgc

SEQ ID NO: 156 - RNA, 106 nt, Synthetic construct
  misc_feature 1..106: note = MYLIP
  source 1..106: mol_type = other RNA
cctcgtcacataacacagcagtttaagagctatgctggaaacagcatagcaagtttaaataaggctagtccgttatcaacttgaaaaagtggcaccgagtcggtgc

SEQ ID NO: 157 - RNA, 106 nt, Synthetic construct
  misc_feature 1..106: note = MYLIP
  source 1..106: mol_type = other RNA
acctccatcagcaccgcgtcgtttaagagctatgctggaaacagcatagcaagtttaaataaggctagtccgttatcaacttgaaaaagtggcaccgagtcggtgc

SEQ ID NO: 158 - RNA, 106 nt, Synthetic construct
  misc_feature 1..106: note = MYLIP
  source 1..106: mol_type = other RNA
ggaggcgaaagccaacggcggtttaagagctatgctggaaacagcatagcaagtttaaataaggctagtccgttatcaacttgaaaaagtggcaccgagtcggtgc

SEQ ID NO: 159 - RNA, 106 nt, Synthetic construct
  misc_feature 1..106: note = MYLIP
  source 1..106: mol_type = other RNA
gttgaggcagtcctcgccgtgtttaagagctatgctggaaacagcatagcaagtttaaataaggctagtccgttatcaacttgaaaaagtggcaccgagtcggtgc

SEQ ID NO: 160 - RNA, 106 nt, Synthetic construct
  misc_feature 1..106: note = ANGPTL3
  source 1..106: mol_type = other RNA
tacattcgtgcaagttaacagtttaagagctatgctggaaacagcatagcaagtttaaataaggctagtccgttatcaacttgaaaaagtggcaccgagtcggtgc

SEQ ID NO: 161 - RNA, 106 nt, Synthetic construct
  misc_feature 1..106: note = ANGPTL3
  source 1..106: mol_type = other RNA
cctaccaaccttaccttttcgtttaagagctatgctggaaacagcatagcaagtttaaataaggctagtccgttatcaacttgaaaaagtggcaccgagtcggtgc

SEQ ID NO: 162 - RNA, 106 nt, Synthetic construct
  misc_feature 1..106: note = ANGPTL3
  source 1..106: mol_type = other RNA
tatatagagttaagaagtctgtttaagagctatgctggaaacagcatagcaagtttaaataaggctagtccgttatcaacttgaaaaagtggcaccgagtcggtgc

SEQ ID NO: 163 - RNA, 106 nt, Synthetic construct
  misc_feature 1..106: note = ANGPTL3
  source 1..106: mol_type = other RNA
aacgtggaactgttttcttcgtttaagagctatgctggaaacagcatagcaagtttaaataaggctagtccgttatcaacttgaaaaagtggcaccgagtcggtgc

SEQ ID NO: 164 - RNA, 106 nt, Synthetic construct
  misc_feature 1..106: note = ANGPTL3
  source 1..106: mol_type = other RNA
attttcaatttcaagcaacggtttaagagctatgctggaaacagcatagcaagtttaaataaggctagtccgttatcaacttgaaaaagtggcaccgagtcggtgc

SEQ ID NO: 165 - RNA, 106 nt, Synthetic construct
  misc_feature 1..106: note = ANGPTL3
  source 1..106: mol_type = other RNA
attctggaggaaataactaggtttaagagctatgctggaaacagcatagcaagtttaaataaggctagtccgttatcaacttgaaaaagtggcaccgagtcggtgc

SEQ ID NO: 166 - RNA, 106 nt, Synthetic construct
  misc_feature 1..106: note = ANGPTL3
  source 1..106: mol_type = other RNA
gcaaatcttgattttggctcgtttaagagctatgctggaaacagcatagcaagtttaaataaggctagtccgttatcaacttgaaaaagtggcaccgagtcggtgc

SEQ ID NO: 167 - RNA, 106 nt, Synthetic construct
  misc_feature 1..106: note = ANGPTL3
  source 1..106: mol_type = other RNA
agccaatggcctccttcagtgtttaagagctatgctggaaacagcatagcaagtttaaataaggctagtccgttatcaacttgaaaaagtggcaccgagtcggtgc

SEQ ID NO: 168 - RNA, 106 nt, Synthetic construct
  misc_feature 1..106: note = ANGPTL3
  source 1..106: mol_type = other RNA
taagaccatgtcccaactgagtttaagagctatgctggaaacagcatagcaagtttaaataaggctagtccgttatcaacttgaaaaagtggcaccgagtcggtgc

SEQ ID NO: 169 - RNA, 106 nt, Synthetic construct
  misc_feature 1..106: note = ANGPTL3
  source 1..106: mol_type = other RNA
agactttgtccataagacgagtttaagagctatgctggaaacagcatagcaagtttaaataaggctagtccgttatcaacttgaaaaagtggcaccgagtcggtgc

SEQ ID NO: 170 - RNA, 106 nt, Synthetic construct
  misc_feature 1..106: note = APOC3
  source 1..106: mol_type = other RNA
ggggcacccgtccagctccggtttaagagctatgctggaaacagcatagcaagtttaaataaggctagtccgttatcaacttgaaaaagtggcaccgagtcggtgc

SEQ ID NO: 171 - RNA, 106 nt, Synthetic construct
  misc_feature 1..106: note = APOC3
  source 1..106: mol_type = other RNA
tgacctttgcccagcgccctgtttaagagctatgctggaaacagcatagcaagtttaaataaggctagtccgttatcaacttgaaaaagtggcaccgagtcggtgc

SEQ ID NO: 172 - RNA, 106 nt, Synthetic construct
  misc_feature 1..106: note = APOC3
  source 1..106: mol_type = other RNA
tccagatgcagcaagcgggcgtttaagagctatgctggaaacagcatagcaagtttaaataaggctagtccgttatcaacttgaaaaagtggcaccgagtcggtgc

SEQ ID NO: 173 - RNA, 106 nt, Synthetic construct
  misc_feature 1..106: note = APOC3
  source 1..106: mol_type = other RNA
tagggatgaactgagcagacgtttaagagctatgctggaaacagcatagcaagtttaaataaggctagtccgttatcaacttgaaaaagtggcaccgagtcggtgc

SEQ ID NO: 174 - RNA, 106 nt, Synthetic construct
  misc_feature 1..106: note = APOC3
  source 1..106: mol_type = other RNA
agaagcacttgctagagctagtttaagagctatgctggaaacagcatagcaagtttaaataaggctagtccgttatcaacttgaaaaagtggcaccgagtcggtgc

SEQ ID NO: 175 - RNA, 106 nt, Synthetic construct
  misc_feature 1..106: note = APOC3
  source 1..106: mol_type = other RNA
ctgctccaggtaatgccctcgtttaagagctatgctggaaacagcatagcaagtttaaataaggctagtccgttatcaacttgaaaaagtggcaccgagtcggtgc

SEQ ID NO: 176 - RNA, 106 nt, Synthetic construct
  misc_feature 1..106: note = APOC3
  source 1..106: mol_type = other RNA
gggagagttgggaaatccctgtttaagagctatgctggaaacagcatagcaagtttaaataaggctagtccgttatcaacttgaaaaagtggcaccgagtcggtgc

SEQ ID NO: 177 - RNA, 106 nt, Synthetic construct
  misc_feature 1..106: note = APOC3
  source 1..106: mol_type = other RNA
aggaagcctcggagctggacgtttaagagctatgctggaaacagcatagcaagtttaaataaggctagtccgttatcaacttgaaaaagtggcaccgagtcggtgc

SEQ ID NO: 178 - RNA, 106 nt, Synthetic construct
  misc_feature 1..106: note = APOC3
  source 1..106: mol_type = other RNA
ccctggagatgatataaaacgtttaagagctatgctggaaacagcatagcaagtttaaataaggctagtccgttatcaacttgaaaaagtggcaccgagtcggtgc

SEQ ID NO: 179 - RNA, 106 nt, Synthetic construct
  misc_feature 1..106: note = APOC3
  source 1..106: mol_type = other RNA
tcataacctgaagaacatgggtttaagagctatgctggaaacagcatagcaagtttaaataaggctagtccgttatcaacttgaaaaagtggcaccgagtcggtgc

SEQ ID NO: 180 - RNA, 106 nt, Synthetic construct
  misc_feature 1..106: note = APOB
  source 1..106: mol_type = other RNA
gtccatcgccagctgcggtggtttaagagctatgctggaaacagcatagcaagtttaaataaggctagtccgttatcaacttgaaaaagtggcaccgagtcggtgc

SEQ ID NO: 181 - RNA, 106 nt, Synthetic construct
  misc_feature 1..106: note = APOB
  source 1..106: mol_type = other RNA
ggcgcccgcaccccatttatgtttaagagctatgctggaaacagcatagcaagtttaaataaggctagtccgttatcaacttgaaaaagtggcaccgagtcggtgc

SEQ ID NO: 182 - RNA, 106 nt, Synthetic construct
  misc_feature 1..106: note = APOB
  source 1..106: mol_type = other RNA
cagagcggccgcgcactcacgtttaagagctatgctggaaacagcatagcaagtttaaataaggctagtccgttatcaacttgaaaaagtggcaccgagtcggtgc

SEQ ID NO: 183 - RNA, 106 nt, Synthetic construct
  misc_feature 1..106: note = APOB
  source 1..106: mol_type = other RNA
ctcagcggcagcaaccgagagtttaagagctatgctggaaacagcatagcaagtttaaataaggctagtccgttatcaacttgaaaaagtggcaccgagtcggtgc

SEQ ID NO: 184 - RNA, 106 nt, Synthetic construct
  misc_feature 1..106: note = APOB
  source 1..106: mol_type = other RNA
tcccggtgggaatgcgcggcgtttaagagctatgctggaaacagcatagcaagtttaaataaggctagtccgttatcaacttgaaaaagtggcaccgagtcggtgc

SEQ ID NO: 185 - RNA, 106 nt, Synthetic construct
  misc_feature 1..106: note = APOB
  source 1..106: mol_type = other RNA
gcattcccaccgggacctgcgtttaagagctatgctggaaacagcatagcaagtttaaataaggctagtccgttatcaacttgaaaaagtggcaccgagtcggtgc

SEQ ID NO: 186 - RNA, 106 nt, Synthetic construct
  misc_feature 1..106: note = APOB
  source 1..106: mol_type = other RNA
gcctcgcggccctggctggcgtttaagagctatgctggaaacagcatagcaagtttaaataaggctagtccgttatcaacttgaaaaagtggcaccgagtcggtgc

SEQ ID NO: 187 - RNA, 106 nt, Synthetic construct
  misc_feature 1..106: note = APOB
  source 1..106: mol_type = other RNA
cccggccaacctcgtgccgcgtttaagagctatgctggaaacagcatagcaagtttaaataaggctagtccgttatcaacttgaaaaagtggcaccgagtcggtgc

SEQ ID NO: 188 - RNA, 106 nt, Synthetic construct
  misc_feature 1..106: note = APOB
  source 1..106: mol_type = other RNA
agcgccagcagcgcgggcctgtttaagagctatgctggaaacagcatagcaagtttaaataaggctagtccgttatcaacttgaaaaagtggcaccgagtcggtgc

SEQ ID NO: 189 - RNA, 106 nt, Synthetic construct
  misc_feature 1..106: note = APOB
  source 1..106: mol_type = other RNA
ctccctctgcgcccgcagaggtttaagagctatgctggaaacagcatagcaagtttaaataaggctagtccgttatcaacttgaaaaagtggcaccgagtcggtgc

SEQ ID NO: 190 - DNA, 86 bp, Synthetic construct
  misc_feature 1..86: note = SpCas9 gRNA scaffold
  source 1..86: mol_type = other DNA
gtttaagagctatgctggaaacagcatagcaagtttaaataaggctagtccgttatcaacttgaaaaagtggcaccgagtcggtgc

SEQ ID NO: 191 - RNA, 86 nt, Synthetic construct
  misc_feature 1..86: note = SpCas9 gRNA scaffold
  source 1..86: mol_type = other RNA
gtttaagagctatgctggaaacagcatagcaagtttaaataaggctagtccgttatcaacttgaaaaagtggcaccgagtcggtgc

SEQ ID NO: 192 - DNA, 195 bp, Synthetic construct
  misc_feature 1..195: note = KRAB
  source 1..195: mol_type = other DNA
cggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtg

SEQ ID NO: 193 - protein, 65 aa, Synthetic construct
  REGION 1..65: note = KRAB
RTLVTFKDVFVDFTREEWKLLDTAQQILYRNVMLENYKNLVSLGYQLTKPDVILRLEKGEEPWLV

SEQ ID NO: 194 - DNA, 966 bp, Synthetic construct
  misc_feature 1..966: note = DNMT3A
  source 1..966: mol_type = other DNA
acctacgggctgctgcggcggcgagaggactggccctcccggctccagatgttcttcgctaataaccacgaccaggaatttgaccctccaaaggtttacccacctgtcccagctgagaagaggaagcccatccgggtgctgtctctctttgatggaatcgctacagggctcctggtgctgaaggacttgggcattcaggtggaccgctacattgcctcggaggtgtgtgaggactccatcacggtgggcatggtgcggcaccaggggaagatcatgtacgtcggggacgtccgcagcgtcacacagaagcatatccaggagtggggcccattcgatctggtgattgggggcagtccctgcaatgacctctccatcgtcaaccctgctcgcaagggcctctacgagggcactggccggctcttctttgagttctaccgcctcctgcatgatgcgcggcccaaggagggagatgatcgccccttcttctggctctttgagaatgtggtggccatgggcgttagtgacaagagggacatctcgcgatttctcgagtccaaccctgtgatgattgatgccaaagaagtgtcagctgcacacagggcccgctacttctggggtaaccttcccggtatgaacaggccgttggcatccactgtgaatgataagctggagctgcaggagtgtctggagcatggcaggatagccaagttcagcaaagtgaggaccattactacgaggtcaaactccataaagcagggcaaagaccagcattttcctgtcttcatgaatgagaaagaggacatcttatggtgcactgaaatggaaagggtatttggtttcccagtccactatactgacgtatccaacatgagccgcttggcgaggcagagactgctgggccggtcatggagcgtgccagtcatccgccacctcttcgctccgctgaaggagtattttgcgtgtgtg

SEQ ID NO: 195 - protein, 322 aa, Synthetic construct
  REGION 1..322: note = DNMT3A
TYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 196 - DNA, 1158 bp, Synthetic construct
  misc_feature 1..1158: note = DNMT3L
  source 1..1158: mol_type = other DNA
atggcggccatcccagccctggacccagaggccgagcccagcatggacgtgattttggtgggatccagtgagctctcaagctccgtttcacccgggacaggcagagatcttattgcatatgaagtcaaggctaaccagcgaaatatagaagacatctgcatctgctgcggaagtctccaggttcacacacagcaccctctgtttgagggagggatctgcgccccatgtaaggacaagttcctggatgccctcttcctgtacgacgatgacgggtaccaatcctactgctccatctgctgctccggagaaacgctgctcatctgcggaaaccctgattgcacccgatgctactgcttcgagtgtgtggatagcctggtcggccccgggacctcggggaaggtgcacgccatgagcaactgggtgtgctacctgtgcctgccgtcctcccgaagcgggctgctgcagcgtcggaggaagtggcgcagccagctcaaggccttctacgaccgagagtcggagaatccccttgagatgttcgaaaccgtgcctgtgtggaggagacagccagtccgggtgctgtccctttttgaagacatcaagaaagagctgacgagtttgggctttttggaaagtggttctgacccgggacaactgaagcatgtggttgatgtcacagacacagtgaggaaggatgtggaggagtggggacccttcgatcttgtgtacggcgccacacctcccctgggccacacctgtgaccgtcctcccagctggtacctgttccagttccaccggctcctgcagtacgcacggcccaagccaggcagccccaggcccttcttctggatgttcgtggacaatctggtgctgaacaaggaagacctggacgtcgcatctcgcttcctggagatggagccagtcaccatcccagatgtccacggcggatccttgcagaatgctgtccgcgtgtggagcaacatcccagccataaggagcaggcactgggctctggtttcggaagaagaattgtccctgctggcccagaacaagcagagctcgaagctcgcggccaagtggcccaccaagctggtgaagaactgctttctccccctaagagaatatttcaagtatttttcaacagaactcacttcctcttta

SEQ ID NO: 197 - protein, 386 aa, Synthetic construct
  REGION 1..386: note = DNMT3L
MAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 198 - DNA, 1629 bp, Synthetic construct
  misc_feature 1..1629: note = DNMT3A/L v1
  source 1..1629: mol_type = other DNA
aaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctctt

SEQ ID NO: 199 - protein, 543 aa, Synthetic construct
  REGION 1..543: note = DNMT3A/L v1
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 200 - DNA, 1629 bp, Synthetic construct
  misc_feature 1..1629: note = DNMT3A/L v2
  source 1..1629: mol_type = other DNA
aaccacgatcaggagtttgacccccctaaggtgtacccacccgtgccagccgagaagaggaagcccatccgcgtgctgtccctgttcgacggcatcgccacaggcctgctggtgctgaaggatctgggcatccaggtggacagatatatcgcctccgaggtgtgcgaggattctatcaccgtgggcatggtgaggcaccagggcaagatcatgtacgtgggcgacgtgcgcagcgtgacacagaagcacatccaggagtggggacccttcgacctggtcatcggaggcagcccctgtaatgacctgtccatcgtgaaccctgcaaggaagggcctgtatgagggaaccggcagactgttctttgagttctacaggctgctgcacgacgcccgccctaaggagggcgatgacaggccattcttttggctgtttgagaacgtggtggccatgggcgtgagcgacaagcgggatatctccagattcctggagtctaatcccgtgatgatcgatgcaaaggaggtgtctgccgcacacagggcaaggtacttttggggaaatctgcctggcatgaaccgcccactggccagcaccgtgaacgacaagctggagctgcaggagtgcctggagcacggaaggatcgccaagttctccaaggtgcggacaatcaccacaagatctaacagcatcaagcagggcaaggatcagcacttccccgtgttcatgaatgagaaggaggacatcctgtggtgtaccgagatggagcgcgtgttcggctttccagtgcactatacagacgtgagcaatatgagccggctggcaaggcagagactgctgggccggtcctggtctgtgccagtgatcagacacctgttcgcccccctgaaggagtactttgcctgcgtgtctagcggcaactctaatgccaacagcagaggcccttccttttcctctggcctggtgccactgtctctgaggggcagccacatgggccccatggagatctacaagaccgtgtccgcctggaagaggcagcctgtgcgcgtgctgtctctgttccgcaacatcgacaaggtgctgaagagcctgggctttctggagagcggatccggatctggaggaggcaccctgaagtatgtggaggatgtgacaaatgtggtgcggagagatgtggagaagtggggccccttcgatctggtgtacggatccacccagccactgggaagctcctgcgataggtgtccaggatggtatatgttccagtttcacagaatcctgcagtacgcactgccaaggcaggagagccagcgccctttcttttggatctttatggacaacctgctgctgacagaggatgaccaggagacaacaacccgcttcctgcagacagaggcagtgaccctgcaggatgtgaggggacgcgactatcagaatgccatgcgggtgtggtctaacatccctggcctgaagagcaagcacgcccccctgacccctaaggaggaggagtacctgcaggcccaggtgcggagcagatccaagctggatgcccctaaggtggacctgctggtgaagaattgtctgctgccactgcgggagtacttcaagtactttagtcagaatagcctgccactg

SEQ ID NO: 201 - protein, 543 aa, Synthetic construct; identical to SEQ ID NO: 199
  REGION 1..543: note = DNMT3A/L v2

SEQ ID NO: 202 - skipped

SEQ ID NO: 203 - protein, 6 aa, Synthetic construct
  REGION 1..6: note = PAM - SaCas9
NNGRRT

SEQ ID NO: 204 - protein, 1053 aa, Synthetic construct
  REGION 1..1053: note = SaCas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 205 - protein, 1052 aa, Synthetic construct
  REGION 1..1052: note = dSaCas9
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 206 - protein, 1368 aa, Synthetic construct [sequence omitted]

SEQ ID NO: 207 - protein, 1367 aa, Synthetic construct
  REGION 1..1367: note = dSpCas9
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 208 - DNA, 4509 bp, Synthetic construct
  misc_feature 1..4509: note = dSpCas9-KRAB
  source 1..4509: mol_type = other DNA
ccaaagaagaagcggaaggtcggtatccacggagtcccagcagccgacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaaggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 209 - protein, 1503 aa, Synthetic construct
  REGION 1..1503: note = dSpCas9-KRAB
PKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 210 - protein, 8 aa, Synthetic construct
  REGION 1..8: note = PAM - N. meningitidis Cas9
NNNNGATT

SEQ ID NO: 211 - protein, 8 aa, Synthetic construct
  REGION 1..8: note = PAM - N. meningitidis Cas9
NNNNRYAC

SEQ ID NO: 212 - protein, 7 aa, Synthetic construct
  REGION 1..7: note = PAM - S. thermophilus
NNAGAAW

SEQ ID NO: 213 - skipped

SEQ ID NO: 214 - protein, 6 aa, Synthetic construct
  REGION 1..6: note = PAM - T. denticola
NAAAAC

SEQ ID NO: 215 - protein, 4 aa, Synthetic construct
  REGION 1..4: note = PAM - Cas12a/Cpf1
TTTV

SEQ ID NO: 216 - protein, 4 aa, Synthetic construct
  REGION 1..4: note = Variant PAM - SpCas9 variant
NGAN

SEQ ID NO: 217 - protein, 4 aa, Synthetic construct
  REGION 1..4: note = Variant PAM - SpCas9 variant
NGNG

SEQ ID NO: 218 - protein, 4 aa, Synthetic construct
  REGION 1..4: note = Variant PAM - SpCas9 variant
NGAG

SEQ ID NO: 219 - protein, 4 aa, Synthetic construct
  REGION 1..4: note = Variant PAM - SpCas9 variant
NGCG

SEQ ID NO: 220 - protein, 548 aa, Synthetic construct
  REGION 1..548: note = ERF domain
MKTPADTGFAFPDWAYKPESSPGSRQIQLWHFILELLRKEEYQGVIAWQGDYGEFVIKDPDEVARLWGVRKCKPQMNYDKLSRALRYYYNKRILHKTKGKRFTYKFNFNKLVLVNYPFIDVGLAGGAVPQSAPPVPSGGSHFRFPPSTPSEVLSPTEDPRSPPACSSSSSSLFSAVVARRLGRGSVSDCSDGTSELEEPLGEDPRARPPGPPDLGAFRGPPLARLPHDPGVFRVYPRPRGGPEPLSPFPVSPLAGPGSLLPPQLSPALPMTPTHLAYTPSPTLSPMYPSGGGGPSGSGGGSHFSFSPEDMKRYLQAHTQSVYNYHLSPRAFLHYPGLVVPQPQRPDKCPLPPMAPETPPVPSSASSSSSSSSSPFKFKLQPPPLGRRQRAAGEKAVAGADKSGGSAGGLAEGAGALAPPPPPPQIKVEPISEGESEEVEVTDISDEDEEDGEVFKTPRAPPAPPKPEPGEAPGASQCMPLKLRFKRRWSEDCRLEGGGGPAGGFEDEGEDKKVRGEGPGEAGGPLTPRRVSSDLQHATAQLSLEHRDS

SEQ ID NO: 221 - protein, 228 aa, Synthetic construct
  REGION 1..228: note = MXI1 domain
MERVKMINVQRLLEAAEFLERRERECEHGYASSFPSMPSPRLQHSKPPRRLSRAQKHSSGSSNTSTANRSTHNELEKNRRAHLRLCLERLKVLIPLGPDCTRHTTLGLLNKAKAHIKKLEEAERKSQHQLENLEREQRFLKWRLEQLQGPQEMERIRMDSIGSTISSDRSDSEREEIEVDVESTEFSHGEVDNISTTSISDIDDHSSLPSIGSDEGYSSASVKLSFTS

SEQ ID NO: 222 - protein, 144 aa, Synthetic construct
  REGION 1..144: note = SID4X domain
ASPKKKRKVEASGSGMNIQMLLEAADYLERREREAEHGYASMLPGSGMNIQMLLEAADYLERREREAEHGYASMLPGSGMNIQMLLEAADYLERREREAEHGYASMLPGSGMNIQMLLEAADYLERREREAEHGYASMLPSRSR

SEQ ID NO: 223 - protein, 220 aa, Synthetic construct
  REGION 1..220: note = MAD-SID domain
MAAAVRMNIQMLLEAADYLERREREAEHGYASMLPYNNKDRDALKRRNKSKKNNSSSRSTHNEMEKNRRAHLRLCLEKLKGLVPLGPESSRHTTLSLLTKAKLHIKKLEDCDRKAVHQIDQLQREQRHLKRQLEKLGIERIRMDSIGSTVSSERSDSDREEIDVDVESTDYLTGDLDWSSSSVSDSDERGSMQSLGSDEGYSSTSIKRIKLQDSHKACLG

SEQ ID NO: 224 - protein, 853 aa, Synthetic construct
  REGION 1..853: note = DNMT3B
MKGDTRHLNGEEDAGGREDSILVNGACSDQSSDSPPILEAIRTPEIRGRRSSSRLSKREVSSLLSYTQDLTGDGDGEDGDGSDTPVMPKLFRETRTRSESPAVRTRNNNSVSSRERHRPSPRSTRGRQGRNHVDESPVEFPATRSLRRRATASAGTPWPSPPSSYLTIDLTDDTEDTHGTPQSSSTPYARLAQDSQQGGMESPQVEADSGDGDSSEYQDGKEFGIGDLVWGKIKGFSWWPAMVVSWKATSKRQAMSGMRWVQWFGDGKFSEVSADKLVALGLFSQHFNLATFNKLVSYRKAMYHALEKARVRAGKTFPSSPGDSLEDQLKPMLEWAHGGFKPTGIEGLKPNNTQPVVNKSKVRRAGSRKLESRKYENKTRRRTADDSATSDYCPAPKRLKTNCYNNGKDRGDEDQSREQMASDVANNKSSLEDGCLSCGRKNPVSFHPLFEGGLCQTCRDRFLELFYMYDDDGYQSYCTVCCEGRELLLCSNTSCCRCFCVECLEVLVGTGTAAEAKLQEPWSCYMCLPQRCHGVLRRRKDWNVRLQAFFTSDTGLEYEAPKLYPAIPAARRRPIRVLSLFDGIATGYLVLKELGIKVGKYVASEVCEESIAVGTVKHEGNIKYVNDVRNITKKNIEEWGPFDLVIGGSPCNDLSNVNPARKGLYEGTGRLFFEFYHLLNYSRPKEGDDRPFFWMFENVVAMKVGDKRDISRFLECNPVMIDAIKVSAAHRARYFWGNLPGMNRPVIASKNDKLELQDCLEYNRIAKLKKVQTITTKSNSIKQGKNQLFPVVMNGKEDVLWCTELERIFGFPVHYTDVSNMGRGARQKLLGRSWSVPVIRHLFAPLKDYFACE

SEQ ID NO: 225 - protein, 852 aa, Synthetic construct
  REGION 1..852: note = LSD1
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 226 - protein, 22 aa, Synthetic construct
  REGION 1..22: note = SunTag GCN4 peptide
LLPKNYHLENEVARLKKLVGER

SEQ ID NO: 227 - protein, 5 aa, Synthetic construct
  REGION 1..5: note = GGSGG linker
GGSGG

SEQ ID NO: 228 - protein, 5 aa, Synthetic construct
  REGION 1..5: note = GGGGS linker
GGGGS

SEQ ID NO: 229 - protein, 5 aa, Synthetic construct
  REGION 1..5: note = GGGGG linker
GGGGG

SEQ ID NO: 230 - protein, 5 aa, Synthetic construct
  REGION 1..5: note = GGAGG linker
GGAGG

SEQ ID NO: 231 - protein, 7 aa, Synthetic construct
  REGION 1..7: note = GGGGSSS linker
GGGGSSS

SEQ ID NO: 232 - protein, 7 aa, Synthetic construct
  REGION 1..7: note = GGGGAAA linker
GGGGAAA

SEQ ID NO: 233 - protein, 80 aa, Synthetic construct
  REGION 1..80: note = XTEN80
GGPSSGAPPPSGGSPAGSPTSTEEGTSESATPESGPGTSTEPSEGSAPGSPAGSPTSTEEGTSTEPSEGSAPGTSTEPSE

SEQ ID NO: 234 - protein, 7 aa, Synthetic construct
  REGION 1..7: note = NLS - SV40
PKKKRKV

SEQ ID NO: 235 - protein, 9 aa, Synthetic construct
  REGION 1..9: note = NLS - cMyc
PAAKRVKLD

SEQ ID NO: 236 - protein, 11 aa, Synthetic construct
  REGION 1..11: note = NLS - cMyc
RQRRNELKRSP

SEQ ID NO: 237 - protein, 38 aa, Synthetic construct
  REGION 1..38: note = NLS - hRNPA1 M9
NQSSNFGPMKGGNFGGRSSGPYGGGGQYFAKPRNQGGY

SEQ ID NO: 238 - protein, 42 aa, Synthetic construct
  REGION 1..42: note = NLS - IBB domain importin-alpha
RMRIZFKNKGKDTAELRRRRVEVSVELRKAKKDEQILKRRNV

SEQ ID NO: 239 - protein, 8 aa, Synthetic construct
  REGION 1..8: note = NLS - myoma T protein
VSRKRPRP

SEQ ID NO: 240 - protein, 8 aa, Synthetic construct
  REGION 1..8: note = NLS - myoma T protein
PPKKARED

SEQ ID NO: 241 - protein, 8 aa, Synthetic construct
  REGION 1..8: note = NLS - human p53
PQPKKKPL

SEQ ID NO: 242 - protein, 12 aa, Synthetic construct
  REGION 1..12: note = NLS - mouse c-abl IV
SALIKKKKKMAP

SEQ ID NO: 243 - protein, 5 aa, Synthetic construct
  REGION 1..5: note = NLS - influenza NS1
DRLRR

SEQ ID NO: 244 - protein, 7 aa, Synthetic construct
  REGION 1..7: note = NLS - influenza NS1
PKQKKRK

SEQ ID NO: 245 - protein, 10 aa, Synthetic construct
  REGION 1..10: note = NLS - hepatitis delta antigen
RKLKKKIKKL

SEQ ID NO: 246 - protein, 10 aa, Synthetic construct
  REGION 1..10: note = NLS - mouse Mx1
REKKKFLKRR

SEQ ID NO: 247 - protein, 20 aa, Synthetic construct
  REGION 1..20: note = NLS - human poly(ADP-ribose) polymerase
KRKGDEVDGVDEVAKKKSKK

SEQ ID NO: 248 - protein, 17 aa, Synthetic construct
  REGION 1..17: note = NLS - glucocorticoid
RKCLQAGMNLEARKTKK

SEQ ID NO: 249 - protein, 16 aa, Synthetic construct
  REGION 1..16: note = NLS - nucleoplasmin
KRPAATKKAGQAKKKK

SEQ ID NO: 250 - DNA, 20 bp, Synthetic construct
  misc_feature 1..20: note = gRNA-mousePCSK9-A spacer DNA
  source 1..20: mol_type = other DNA
gggaagggatacaggctgga

SEQ ID NO: 251 - DNA, 20 bp, Synthetic construct
  misc_feature 1..20: note = gRNA-mousePCSK9-B spacer DNA
  source 1..20: mol_type = other DNA
gtcccgtttgcagcccaatt

SEQ ID NO: 252 - DNA, 20 bp, Synthetic construct
  misc_feature 1..20: note = gRNA-mousePCSK9-C spacer DNA
  source 1..20: mol_type = other DNA
gagcgtcatttgacgctgtc

SEQ ID NO: 253 - DNA, 20 bp, Synthetic construct
  misc_feature 1..20: note = gRNA-mousePCSK9-D spacer DNA
  source 1..20: mol_type = other DNA
ggatcttccgatggggctcg

SEQ ID NO: 254 - DNA, 20 bp, Synthetic construct
  misc_feature 1..20: note = gRNA-mousePCSK9-E spacer DNA
  source 1..20: mol_type = other DNA
gtgaaggtggaagccttctg

SEQ ID NO: 255 - DNA, 20 bp, Synthetic construct
  misc_feature 1..20: note = gRNA-mousePCSK9-F spacer DNA
  source 1..20: mol_type = other DNA
gtggacgcgcaggctgccgg

SEQ ID NO: 256 - DNA, 20 bp, Synthetic construct
  misc_feature 1..20: note = gRNA-mousePCSK9-G spacer DNA
  source 1..20: mol_type = other DNA
ggggcgaggagaggtgcgcg

SEQ ID NO: 257 - DNA, 20 bp, Synthetic construct
  misc_feature 1..20: note = gRNA-mousePCSK9-H spacer DNA
  source 1..20: mol_type = other DNA
agtgggtgcccatcggggcg

SEQ ID NO: 258 - DNA, 20 bp, Synthetic construct
  misc_feature 1..20: note = gRNA-mousePCSK9-I spacer DNA
  source 1..20: mol_type = other DNA
ctactgtgccccaccggcgc

SEQ ID NO: 259 - RNA, 20 nt, Synthetic construct
  misc_feature 1..20: note = gRNA-mousePCSK9-A spacer RNA
  source 1..20: mol_type = other RNA
gggaagggatacaggctgga

SEQ ID NO: 260 - RNA, 20 nt, Synthetic construct
  misc_feature 1..20: note = gRNA-mousePCSK9-B spacer RNA
  source 1..20: mol_type = other RNA
gtcccgtttgcagcccaatt

SEQ ID NO: 261 - RNA, 20 nt, Synthetic construct
  misc_feature 1..20: note = gRNA-mousePCSK9-C spacer RNA
  source 1..20: mol_type = other RNA
gagcgtcatttgacgctgtc

SEQ ID NO: 262 - RNA, 20 nt, Synthetic construct
  misc_feature 1..20: note = gRNA-mousePCSK9-D spacer RNA
  source 1..20: mol_type = other RNA
ggatcttccgatggggctcg

SEQ ID NO: 263 - RNA, 20 nt, Synthetic construct
  misc_feature 1..20: note = gRNA-mousePCSK9-E spacer RNA
  source 1..20: mol_type = other RNA
gtgaaggtggaagccttctg

SEQ ID NO: 264 - RNA, 20 nt, Synthetic construct
  misc_feature 1..20: note = gRNA-mousePCSK9-F spacer RNA
  source 1..20: mol_type = other RNA
gtggacgcgcaggctgccgg

SEQ ID NO: 265 - RNA, 20 nt, Synthetic construct
  misc_feature 1..20: note = gRNA-mousePCSK9-G spacer RNA
  source 1..20: mol_type = other RNA
ggggcgaggagaggtgcgcg

SEQ ID NO: 266 - RNA, 20 nt, Synthetic construct
  misc_feature 1..20: note = gRNA-mousePCSK9-H spacer RNA
  source 1..20: mol_type = other RNA
agtgggtgcccatcggggcg

SEQ ID NO: 267 - RNA, 20 nt, Synthetic construct
  misc_feature 1..20: note = gRNA-mousePCSK9-I spacer RNA
  source 1..20: mol_type = other RNA
ctactgtgccccaccggcgc

SEQ ID NO: 268 - RNA, 106 nt, Synthetic construct
  misc_feature 1..106: note = gRNA-mousePCSK9-A gRNA
  source 1..106: mol_type = other RNA
gggaagggatacaggctggagtttaagagctatgctggaaacagcatagcaagtttaaataaggctagtccgttatcaacttgaaaaagtggcaccgagtcggtgc

SEQ ID NO: 269 - RNA, 106 nt, Synthetic construct
  misc_feature 1..106: note = gRNA-mousePCSK9-B gRNA
  source 1..106: mol_type = other RNA
gtcccgtttgcagcccaattgtttaagagctatgctggaaacagcatagcaagtttaaataaggctagtccgttatcaacttgaaaaagtggcaccgagtcggtgc

SEQ ID NO: 270 - RNA, 106 nt, Synthetic construct
  misc_feature 1..106: note = gRNA-mousePCSK9-C gRNA
  source 1..106: mol_type = other RNA
gagcgtcatttgacgctgtcgtttaagagctatgctggaaacagcatagcaagtttaaataaggctagtccgttatcaacttgaaaaagtggcaccgagtcggtgc

SEQ ID NO: 271 - RNA, 106 nt, Synthetic construct
  misc_feature 1..106: note = gRNA-mousePCSK9-D gRNA
  source 1..106: mol_type = other RNA
ggatcttccgatggggctcggtttaagagctatgctggaaacagcatagcaagtttaaataaggctagtccgttatcaacttgaaaaagtggcaccgagtcggtgc

SEQ ID NO: 272 - RNA, 106 nt, Synthetic construct
  misc_feature 1..106: note = gRNA-mousePCSK9-E gRNA
  source 1..106: mol_type = other RNA
gtgaaggtggaagccttctggtttaagagctatgctggaaacagcatagcaagtttaaataaggctagtccgttatcaacttgaaaaagtggcaccgagtcggtgc

SEQ ID NO: 273 - RNA, 106 nt, Synthetic construct
  misc_feature 1..106: note = gRNA-mousePCSK9-F gRNA
  source 1..106: mol_type = other RNA
gtggacgcgcaggctgccgggtttaagagctatgctggaaacagcatagcaagtttaaataaggctagtccgttatcaacttgaaaaagtggcaccgagtcggtgc

SEQ ID NO: 274 - RNA, 106 nt, Synthetic construct
  misc_feature 1..106: note = gRNA-mousePCSK9-G gRNA
  source 1..106: mol_type = other RNA
ggggcgaggagaggtgcgcggtttaagagctatgctggaaacagcatagcaagtttaaataaggctagtccgttatcaacttgaaaaagtggcaccgagtcggtgc

SEQ ID NO: 275 - RNA, 106 nt, Synthetic construct
  misc_feature 1..106: note = gRNA-mousePCSK9-H gRNA
  source 1..106: mol_type = other RNA
agtgggtgcccatcggggcggtttaagagctatgctggaaacagcatagcaagtttaaataaggctagtccgttatcaacttgaaaaagtggcaccgagtcggtgc

SEQ ID NO: 276 - RNA, 106 nt, Synthetic construct
  misc_feature 1..106: note = gRNA-mousePCSK9-I gRNA
  source 1..106: mol_type = other RNA
ctactgtgccccaccggcgcgtttaagagctatgctggaaacagcatagcaagtttaaataaggctagtccgttatcaacttgaaaaagtggcaccgagtcggtgc

SEQ ID NO: 277 - DNA, 6204 bp, Synthetic construct
  misc_feature 1..6204: note = dSpCas9-KRAB-DNTM3A/L
  source 1..6204: mol_type = other DNA
aaccacgatcaggagtttgacccccctaaggtgtacccacccgtgccagccgagaagaggaagcccatccgcgtgctgtccctgttcgacggcatcgccacaggcctgctggtgctgaaggatctgggcatccaggtggacagatatatcgcctccgaggtgtgcgaggattctatcaccgtgggcatggtgaggcaccagggcaagatcatgtacgtgggcgacgtgcgcagcgtgacacagaagcacatccaggagtggggacccttcgacctggtcatcggaggcagcccctgtaatgacctgtccatcgtgaaccctgcaaggaagggcctgtatgagggaaccggcagactgttctttgagttctacaggctgctgcacgacgcccgccctaaggagggcgatgacaggccattcttttggctgtttgagaacgtggtggccatgggcgtgagcgacaagcgggatatctccagattcctggagtctaatcccgtgatgatcgatgcaaaggaggtgtctgccgcacacagggcaaggtacttttggggaaatctgcctggcatgaaccgcccactggccagcaccgtgaacgacaagctggagctgcaggagtgcctggagcacggaaggatcgccaagttctccaaggtgcggacaatcaccacaagatctaacagcatcaagcagggcaaggatcagcacttccccgtgttcatgaatgagaaggaggacatcctgtggtgtaccgagatggagcgcgtgttcggctttccagtgcactatacagacgtgagcaatatgagccggctggcaaggcagagactgctgggccggtcctggtctgtgccagtgatcagacacctgttcgcccccctgaaggagtactttgcctgcgtgtctagcggcaactctaatgccaacagcagaggcccttccttttcctctggcctggtgccactgtctctgaggggcagccacatgggccccatggagatctacaagaccgtgtccgcctggaagaggcagcctgtgcgcgtgctgtctctgttccgcaacatcgacaaggtgctgaagagcctgggctttctggagagcggatccggatctggaggaggcaccctgaagtatgtggaggatgtgacaaatgtggtgcggagagatgtggagaagtggggccccttcgatctggtgtacggatccacccagccactgggaagctcctgcgataggtgtccaggatggtatatgttccagtttcacagaatcctgcagtacgcactgccaaggcaggagagccagcgccctttcttttggatctttatggacaacctgctgctgacagaggatgaccaggagacaacaacccgcttcctgcagacagaggcagtgaccctgcaggatgtgaggggacgcgactatcagaatgccatgcgggtgtggtctaacatccctggcctgaagagcaagcacgcccccctgacccctaaggaggaggagtacctgcaggcccaggtgcggagcagatccaagctggatgcccctaaggtggacctgctggtgaagaattgtctgctgccactgcgggagtacttcaagtactttagtcagaatagcctgccactggaggcaagcggatccggaagggcatctcctggaatcccaggaagcacccgcaaccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggacgccatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaaggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttag

SEQ ID NO: 278 - protein, 2067 aa, Synthetic construct
  REGION 1..2067: note = dSpCas9-KRAB-DNTM3A/L
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLEASGSGRASPGIPGSTRNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 279 - DNA, 6399 bp, Synthetic construct
  misc_feature 1..6399: note = DNMT3A/L-XTEN80-dSpCas9-KRAB
  source 1..6399: mol_type = other DNA
atgaaccacgatcaggagtttgacccccctaaggtgtacccacccgtgccagccgagaagaggaagcccatccgcgtgctgtccctgttcgacggcatcgccacaggcctgctggtgctgaaggatctgggcatccaggtggacagatatatcgcctccgaggtgtgcgaggattctatcaccgtgggcatggtgaggcaccagggcaagatcatgtacgtgggcgacgtgcgcagcgtgacacagaagcacatccaggagtggggacccttcgacctggtcatcggaggcagcccctgtaatgacctgtccatcgtgaaccctgcaaggaagggcctgtatgagggaaccggcagactgttctttgagttctacaggctgctgcacgacgcccgccctaaggagggcgatgacaggccattcttttggctgtttgagaacgtggtggccatgggcgtgagcgacaagcgggatatctccagattcctggagtctaatcccgtgatgatcgatgcaaaggaggtgtctgccgcacacagggcaaggtacttttggggaaatctgcctggcatgaaccgcccactggccagcaccgtgaacgacaagctggagctgcaggagtgcctggagcacggaaggatcgccaagttctccaaggtgcggacaatcaccacaagatctaacagcatcaagcagggcaaggatcagcacttccccgtgttcatgaatgagaaggaggacatcctgtggtgtaccgagatggagcgcgtgttcggctttccagtgcactatacagacgtgagcaatatgagccggctggcaaggcagagactgctgggccggtcctggtctgtgccagtgatcagacacctgttcgcccccctgaaggagtactttgcctgcgtgtctagcggcaactctaatgccaacagcagaggcccttccttttcctctggcctggtgccactgtctctgaggggcagccacatgggccccatggagatctacaagaccgtgtccgcctggaagaggcagcctgtgcgcgtgctgtctctgttccgcaacatcgacaaggtgctgaagagcctgggctttctggagagcggatccggatctggaggaggcaccctgaagtatgtggaggatgtgacaaatgtggtgcggagagatgtggagaagtggggccccttcgatctggtgtacggatccacccagccactgggaagctcctgcgataggtgtccaggatggtatatgttccagtttcacagaatcctgcagtacgcactgccaaggcaggagagccagcgccctttcttttggatctttatggacaacctgctgctgacagaggatgaccaggagacaacaacccgcttcctgcagacagaggcagtgaccctgcaggatgtgaggggacgcgactatcagaatgccatgcgggtgtggtctaacatccctggcctgaagagcaagcacgcccccctgacccctaaggaggaggagtacctgcaggcccaggtgcggagcagatccaagctggatgcccctaaggtggacctgctggtgaagaattgtctgctgccactgcgggagtacttcaagtactttagtcagaatagcctgccactgggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgaggttaaccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggacgccatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaaggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttag

SEQ ID NO: 280 - protein, 2132 aa, Synthetic construct
  REGION 1..2132: note = DNMT3A/L-XTEN80-dSpCas9-KRAB
MNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 281 - DNA, 6399 bp, Synthetic construct
  misc_feature 1..6399: note = DNMT3A/L(CRISPROFF)-XTEN80-dSpCas9-KRAB
  source 1..6399: mol_type = other DNA
agtaaccatgaccaggaatttgaccccccaaaggtttacccacctgtgccagctgagaagaggaagcccatccgcgtgctgtctctctttgatgggattgctacagggctcctggtgctgaaggacctgggcatccaagtggaccgctacattgcctccgaggtgtgtgaggactccatcacggtgggcatggtgcggcaccagggaaagatcatgtacgtcggggacgtccgcagcgtcacacagaagcatatccaggagtggggcccattcgacctggtgattggaggcagtccctgcaatgacctctccattgtcaaccctgcccgcaagggactttatgagggtactggccgcctcttctttgagttctaccgcctcctgcatgatgcgcggcccaaggagggagatgatcgccccttcttctggctctttgagaatgtggtggccatgggcgttagtgacaagagggacatctcgcgatttcttgagtctaaccccgtgatgattgacgccaaagaagtgtctgctgcacacagggcccgttacttctggggtaaccttcctggcatgaacaggcctttggcatccactgtgaatgataagctggagctgcaagagtgtctggagcacggcagaatagccaagttcagcaaagtgaggaccattaccaccaggtcaaactctataaagcagggcaaagaccagcatttccccgtcttcatgaacgagaaggaggacatcctgtggtgcactgaaatggaaagggtgtttggcttccccgtccactacacagacgtctccaacatgagccgcttggcgaggcagagactgctgggccgatcgtggagcgtgccggtcatccgccacctcttcgctccgctgaaggaatattttgcttgtgtgtctagcggcaatagtaacgctaacagccgcgggccgagcttcagcagcggcctggtgccgttaagcttgcgcggcagccatatgggccctatggagatatacaagacagtgtctgcatggaagagacagccagtgcgggtactgagcctcttcagaaacatcgacaaggtactaaagagtttgggcttcttggaaagcggttctggttctgggggaggaacgctgaagtacgtggaagatgtcacaaatgtcgtgaggagagacgtggagaaatggggcccctttgacctggtgtacggctcgacgcagcccctaggcagctcttgtgatcgctgtcccggctggtacatgttccagttccaccggatcctgcagtatgcgctgcctcgccaggagagtcagcggcccttcttctggatattcatggacaatctgctgctgactgaggatgaccaagagacaactacccgcttccttcagacagaggctgtgaccctccaggatgtccgtggcagagactaccagaatgctatgcgggtgtggagcaacattccagggctgaagagcaagcatgcgcccctgaccccaaaggaagaagagtatctgcaagcccaagtcagaagcaggagcaagctggacgccccgaaagttgacctcctggtgaagaactgccttctcccgctgagagagtacttcaagtatttttctcaaaactcacttcctcttggagggccgagctctggcgcacccccaccaagtggagggtctcctgccgggtccccaacatctactgaagaaggcaccagcgaatccgcaacgcccgagtcaggccctggtacctccacagaaccatctgaaggtagtgcgcctggttccccagctggaagccctacttccaccgaagaaggcacgtcaaccgaaccaagtgaaggatctgcccctgggaccagcactgaaccatctgaggttaaccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggacgccatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaaggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtttag

SEQ ID NO: 282 - protein, 2132 aa, Synthetic construct
  REGION 1..2132: note = DNMT3A/L(CRISPROFF)-XTEN80-dSpCas9-KRAB
SNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPLGGPSSGAPPPSGGSPAGSPTSTEEGTSESATPESGPGTSTEPSEGSAPGSPAGSPTSTEEGTSTEPSEGSAPGTSTEPSEVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 283 - protein, 746 aa, Synthetic construct
  REGION 1..746: note = EZH2
MGQTGKKSEKGPVCWRKRVKSEYMRLRQLKRFRRADEVKTMFSSNRQKILERTETLNQEWKQRRIQPVHIMTSVSSLRGTRECSVTSDLDFPAQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPDEREEKQKDLEDNRDDKETCPPRKFPADKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTISVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPVPTEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 284 - protein, 910 aa, Synthetic construct
  REGION 1..910: note = DNMT3A
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFA

SEQ ID NO: 285 - protein, 301 aa, Synthetic construct
  REGION 1..301: note = Human DNMT3A
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 286 - protein, 215 aa, Synthetic construct
  REGION 1..215: note = Murine DNMT3L
MGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 287 - protein, 214 aa, Synthetic construct
  REGION 1..214: note = C-terminal human DNM3L
MNPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 288 - protein, 27 aa, Synthetic construct
  REGION 1..27: note = Linker
SSGNSNANSRGPSFSSGLVPLSLRGSH

SEQ ID NO: 289 - protein, 421 aa, Synthetic construct
  REGION 1..421: note = Murine DNMT3L
MGSRETPSSCSKTLETLDLETSDSSSPDADSPLEEQWLKSSPALKEDSVDVVLEDCKEPLSPSSPPTGREMIRYEVKVNRRSIEDICLCCGTLQVYTRHPLFEGGLCAPCKDKFLESLFLYDDDGHQSYCTICCSGGTLFICESPDCTRCYCFECVDILVGPGTSERINAMACWVCFLCLPFSRSGLLQRRKRWRHQLKAFHDQEGAGPMEIYKTVSAWKRQPVRVLSLFRNIDKVLKSLGFLESGSGSGGGTLKYVEDVTNVVRRDVEKWGPFDLVYGSTQPLGSSCDRCPGWYMFQFHRILQYALPRQESQRPFFWIFMDNLLLTEDDQETTTRFLQTEAVTLQDVRGRDYQNAMRVWSNIPGLKSKHAPLTPKEEEYLQAQVRSRSKLDAPKVDLLVKNCLLPLREYFKYFSQNSLPL

SEQ ID NO: 290 - protein, 87 aa, Synthetic construct
  REGION 1..87: note = KRAB
RTLVTFKDVFVDFTREEWKLLDTAQQILYRNVMLENYKNLVSLGYQLTKPDVILRLEKGEEPWLVEREIHQETHPDSETAFEIKSSV

SEQ ID NO: 291 - protein, 15 aa, Synthetic construct
  REGION 1..15: note = NLS and linker
PKKKRKVGIHGVPAA

SEQ ID NO: 292 - protein, 8 aa, Synthetic construct
  REGION 1..8: note = Flag tag
DYKDDDDK

SEQ ID NO: 293 - protein, 17 aa, Synthetic construct
  REGION 1..17: note = Linker
EASGSGRASPGIPGSTR

SEQ ID NO: 294 - protein, 8 aa, Synthetic construct
  REGION 1..8: note = Linker
GIHGVPAA

SEQ ID NO: 295 - protein, 23 aa, Synthetic construct
  REGION 1..23: note = 3 x Flag peptide
MDYKDHDGDYKDHDIDYKDDDDK

SEQ ID NO: 296 - protein, 9 aa, Synthetic construct
  REGION 1..9: note = HA tag
YPYDVPDYA

SEQ ID NO: 297 - protein, 6 aa, Synthetic construct
  REGION 1..6: note = poly-histidine tag
HHHHHH

SEQ ID NO: 298 - protein, 27 aa, Synthetic construct
  REGION 1..27: note = Linker
KRPAATKKAGQAKKKKASDAKSLTAWS

SEQ ID NO: 299 - protein, 16 aa, Synthetic construct
  REGION 1..16: note = Linker
SGSETPGTSESATPES

SEQ ID NO: 300 - protein, 85 aa, Synthetic construct
  REGION 1..85: note = N term Npu Intein
CLSYETEILTVEYGLLPIGKIVEKRIECTVYSVDNNGNIYTQPVAQWHDRGEQEVFEYCLEDGSLIRATKDHKFMTVDGQMLPID

SEQ ID NO: 301 - DNA, 105 bp, Synthetic construct
  misc_feature 1..105: note = C term Npu Intein
  source 1..105: mol_type = other DNA
atcaagattgctacacggaaatacctgggaaagcagaacgtgtacgacatcggcgtggagcgggatcacaacttcgccctgaagaatggctttatcgccagcaat

SEQ ID NO: 302 - protein, 35 aa, Synthetic construct
  REGION 1..35: note = C term Npu Intein
IKIATRKYLGKQNVYDIGVERDHNFALKNGFIASN

SEQ ID NO: 303 - DNA, 2598 bp, Synthetic construct
  misc_feature 1..2598: note = dSpCas9-573-C Intein
  source 1..2598: mol_type = other DNA
atgaaacggacagccgacggaagcgagttcgagtcaccaaagaagaagcggaaagtcatcaagattgctacacggaaatacctgggaaagcagaacgtgtacgacatcggcgtggagcgggatcacaacttcgccctgaagaatggctttatcgccagcaattgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaagtga

SEQ ID NO: 304 - protein, 865 aa, Synthetic construct
  REGION 1..865: note = dSpCas9-573-C Intein
MKRTADGSEFESPKKKRKVIKIATRKYLGKQNVYDIGVERDHNFALKNGFIASNCFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 305 - DNA, 255 bp, Synthetic construct
  misc_feature 1..255: note = N term Npu Intein
  source 1..255: mol_type = other DNA
tgcctgtcctacgagacagagatcctgacagtggagtatggcctgctgccaatcggcaagatcgtggagaagaggatcgagtgtaccgtgtactctgtggataacaatggcaacatctatacacagcccgtggcacagtggcacgataggggagagcaggaggtgttcgagtattgcctggaggacggcagcctgatcagggcaaccaaggaccacaagttcatgacagtggatggccagatgctgcccatcgac

SEQ ID NO: 306 - DNA, 20 bp, Synthetic construct
  misc_feature 1..20: note = PCSK9
  source 1..20: mol_type = other DNA
gtcgaggcgctcatggttgc

SEQ ID NO: 307 - DNA, 20 bp, Synthetic construct
  misc_feature 1..20: note = PCSK9
  source 1..20: mol_type = other DNA
ttccagcccagttaggattt

SEQ ID NO: 308 - DNA, 20 bp, Synthetic construct
  misc_feature 1..20: note = PCSK9
  source 1..20: mol_type = other DNA
tcctaactgggctggaaggc

SEQ ID NO: 309 - DNA, 20 bp, Synthetic construct
  misc_feature 1..20: note = PCSK9
  source 1..20: mol_type = other DNA
tcaggagcagggcgcgtgaa

SEQ ID NO: 310 - DNA, 20 bp, Synthetic construct
  misc_feature 1..20: note = PCSK9
  source 1..20: mol_type = other DNA
cagcgacgtcgaggcgctca

SEQ ID NO: 311 - DNA, 20 bp, Synthetic construct
  misc_feature 1..20: note = PCSK9
  source 1..20: mol_type = other DNA
ccgtcagctccaggcggtcc

SEQ ID NO: 312 - DNA, 20 bp, Synthetic construct
  misc_feature 1..20: note = PCSK9
  source 1..20: mol_type = other DNA
aacctgatcctccagtccgg

SEQ ID NO: 313 - DNA, 20 bp, Synthetic construct
  misc_feature 1..20: note = PCSK9
  source 1..20: mol_type = other DNA
tcatgggcaccgtcagctcc

SEQ ID NO: 314 - DNA, 20 bp, Synthetic construct
  misc_feature 1..20: note = PCSK9
  source 1..20: mol_type = other DNA
ccgccggcgtggaccgcgca

SEQ ID NO: 315 - DNA, 20 bp, Synthetic construct
  misc_feature 1..20: note = PCSK9
  source 1..20: mol_type = other DNA
gaaggcaggccggcgcccta

SEQ ID NO: 316 - DNA, 20 bp, Synthetic construct
  misc_feature 1..20: note = PCSK9
  source 1..20: mol_type = other DNA
gcgccttgagccttgcggtg

SEQ ID NO: 317 - DNA, 20 bp, Synthetic construct
  misc_feature 1..20: note = PCSK9
  source 1..20: mol_type = other DNA
cccgcaccttggcgcagcgg

SEQ ID NO: 318 - RNA, 20 nt, Synthetic construct
  misc_feature 1..20: note = PCSK9
  source 1..20: mol_type = other RNA
gtcgaggcgctcatggttgc

SEQ ID NO: 319 - RNA, 20 nt, Synthetic construct
  misc_feature 1..20: note = PCSK9
  source 1..20: mol_type = other RNA
ttccagcccagttaggattt

SEQ ID NO: 320 - RNA, 20 nt, Synthetic construct
  misc_feature 1..20: note = PCSK9
  source 1..20: mol_type = other RNA
tcctaactgggctggaaggc

SEQ ID NO: 321 - RNA, 20 nt, Synthetic construct
  misc_feature 1..20: note = PCSK9
  source 1..20: mol_type = other RNA
tcaggagcagggcgcgtgaa

SEQ ID NO: 322 - RNA, 20 nt, Synthetic construct
  misc_feature 1..20: note = PCSK9
  source 1..20: mol_type = other RNA
cagcgacgtcgaggcgctca

SEQ ID NO: 323 - RNA, 20 nt, Synthetic construct
  misc_feature 1..20: note = PCSK9
  source 1..20: mol_type = other RNA
ccgtcagctccaggcggtcc

SEQ ID NO: 324 - RNA, 20 nt, Synthetic construct
  misc_feature 1..20: note = PCSK9
  source 1..20: mol_type = other RNA
aacctgatcctccagtccgg

SEQ ID NO: 325 - RNA, 20 nt, Synthetic construct
  misc_feature 1..20: note = PCSK9
  source 1..20: mol_type = other RNA
tcatgggcaccgtcagctcc

SEQ ID NO: 326 - RNA, 20 nt, Synthetic construct
  misc_feature 1..20: note = PCSK9
  source 1..20: mol_type = other RNA
ccgccggcgtggaccgcgca

SEQ ID NO: 327 - RNA, 20 nt, Synthetic construct
  misc_feature 1..20: note = PCSK9
  source 1..20: mol_type = other RNA
gaaggcaggccggcgcccta

SEQ ID NO: 328 - RNA, 20 nt, Synthetic construct
  misc_feature 1..20: note = PCSK9
  source 1..20: mol_type = other RNA
gcgccttgagccttgcggtg

SEQ ID NO: 329 - RNA, 20 nt, Synthetic construct
  misc_feature 1..20: note = PCSK9
  source 1..20: mol_type = other RNA
cccgcaccttggcgcagcgg

SEQ ID NO: 330 - RNA, 106 nt, Synthetic construct
  misc_feature 1..106: note = PCSK9
  source 1..106: mol_type = other RNA
gtcgaggcgctcatggttgcgtttaagagctatgctggaaacagcatagcaagtttaaataaggctagtccgttatcaacttgaaaaagtggcaccgagtcggtgc

SEQ ID NO: 331 - RNA, 106 nt, Synthetic construct
  misc_feature 1..106: note = PCSK9
  source 1..106: mol_type = other RNA
ttccagcccagttaggatttgtttaagagctatgctggaaacagcatagcaagtttaaataaggctagtccgttatcaacttgaaaaagtggcaccgagtcggtgc

SEQ ID NO: 332 - RNA, 106 nt, Synthetic construct
  misc_feature 1..106: note = PCSK9
  source 1..106: mol_type = other RNA
tcctaactgggctggaaggcgtttaagagctatgctggaaacagcatagcaagtttaaataaggctagtccgttatcaacttgaaaaagtggcaccgagtcggtgc

SEQ ID NO: 333 - RNA, 106 nt, Synthetic construct
  misc_feature 1..106: note = PCSK9
  source 1..106: mol_type = other RNA
tcaggagcagggcgcgtgaagtttaagagctatgctggaaacagcatagcaagtttaaataaggctagtccgttatcaacttgaaaaagtggcaccgagtcggtgc

SEQ ID NO: 334 - RNA, 106 nt, Synthetic construct
  misc_feature 1..106: note = PCSK9
  source 1..106: mol_type = other RNA
cagcgacgtcgaggcgctcagtttaagagctatgctggaaacagcatagcaagtttaaataaggctagtccgttatcaacttgaaaaagtggcaccgagtcggtgc

SEQ ID NO: 335 - RNA, 106 nt, Synthetic construct
  misc_feature 1..106: note = PCSK9
  source 1..106: mol_type = other RNA
ccgtcagctccaggcggtccgtttaagagctatgctggaaacagcatagcaagtttaaataaggctagtccgttatcaacttgaaaaagtggcaccgagtcggtgc

SEQ ID NO: 336 - RNA, 106 nt, Synthetic construct
  misc_feature 1..106: note = PCSK9
  source 1..106: mol_type = other RNA
aacctgatcctccagtccgggtttaagagctatgctggaaacagcatagcaagtttaaataaggctagtccgttatcaacttgaaaaagtggcaccgagtcggtgc

SEQ ID NO: 337 - RNA, 106 nt, Synthetic construct
  misc_feature 1..106: note = PCSK9
  source 1..106: mol_type = other RNA
tcatgggcaccgtcagctccgtttaagagctatgctggaaacagcatagcaagtttaaataaggctagtccgttatcaacttgaaaaagtggcaccgagtcggtgc

SEQ ID NO: 338 - RNA, 106 nt, Synthetic construct
  misc_feature 1..106: note = PCSK9
  source 1..106: mol_type = other RNA
ccgccggcgtggaccgcgcagtttaagagctatgctggaaacagcatagcaagtttaaataaggctagtccgttatcaacttgaaaaagtggcaccgagtcggtgc

SEQ ID NO: 339 - RNA, 106 nt, Synthetic construct
  misc_feature 1..106: note = PCSK9
  source 1..106: mol_type = other RNA
gaaggcaggccggcgccctagtttaagagctatgctggaaacagcatagcaagtttaaataaggctagtccgttatcaacttgaaaaagtggcaccgagtcggtgc

SEQ ID NO: 340 - RNA, 106 nt, Synthetic construct
  misc_feature 1..106: note = PCSK9
  source 1..106: mol_type = other RNA
gcgccttgagccttgcggtggtttaagagctatgctggaaacagcatagcaagtttaaataaggctagtccgttatcaacttgaaaaagtggcaccgagtcggtgc

SEQ ID NO: 341 - RNA, 106 nt, Synthetic construct
  misc_feature 1..106: note = PCSK9
  source 1..106: mol_type = other RNA
cccgcaccttggcgcagcgggtttaagagctatgctggaaacagcatagcaagtttaaataaggctagtccgttatcaacttgaaaaagtggcaccgagtcggtgc

SEQ ID NO: 342 - DNA, 20 bp, Synthetic construct
  misc_feature 1..20: note = MYLIP
  source 1..20: mol_type = other DNA
gcactgcggcggcagccggg

SEQ ID NO: 343 - DNA, 20 bp, Synthetic construct
  misc_feature 1..20: note = MYLIP
  source 1..20: mol_type = other DNA
ggcgccaccgcggaggacag

SEQ ID NO: 344 - DNA, 20 bp, Synthetic construct
  misc_feature 1..20: note = MYLIP
  source 1..20: mol_type = other DNA
atgctcataggatgtattca

SEQ ID NO: 345 - DNA, 20 bp, Synthetic construct
  misc_feature 1..20: note = MYLIP
  source 1..20: mol_type = other DNA
ccacaataaacacatggtct

SEQ ID NO: 346 - DNA, 20 bp, Synthetic construct
  misc_feature 1..20: note = MYLIP
  source 1..20: mol_type = other DNA
gggtcccaccagtgacaagg

SEQ ID NO: 347 - DNA, 20 bp, Synthetic construct
  misc_feature 1..20: note = MYLIP
  source 1..20: mol_type = other DNA
ggactgcctcaaccaggtga

SEQ ID NO: 348 - DNA, 20 bp, Synthetic construct
  misc_feature 1..20: note = MYLIP
  source 1..20: mol_type = other DNA
cagagtccctgtcgcagcgc

SEQ ID NO: 349 - DNA, 20 bp, Synthetic construct
  misc_feature 1..20: note = MYLIP
  source 1..20: mol_type = other DNA
ctcagagtgagcgatcgccc

SEQ ID NO: 350 - DNA, 20 bp, Synthetic construct
  misc_feature 1..20: note = MYLIP
  source 1..20: mol_type = other DNA
ctgagtttccctggccgccc

SEQ ID NO: 351 - DNA, 20 bp, Synthetic construct
  misc_feature 1..20: note = MYLIP
  source 1..20: mol_type = other DNA
gtttccctggccgccccggg

SEQ ID NO: 352 - RNA, 20 nt, Synthetic construct
  misc_feature 1..20: note = MYLIP
  source 1..20: mol_type = other RNA
gcactgcggcggcagccggg

SEQ ID NO: 353 - RNA, 20 nt, Synthetic construct
  misc_feature 1..20: note = MYLIP
  source 1..20: mol_type = other RNA
ggcgccaccgcggaggacag

SEQ ID NO: 354 - RNA, 20 nt, Synthetic construct
  misc_feature 1..20: note = MYLIP
  source 1..20: mol_type = other RNA
atgctcataggatgtattca

SEQ ID NO: 355 - RNA, 20 nt, Synthetic construct
  misc_feature 1..20: note = MYLIP
  source 1..20: mol_type = other RNA
ccacaataaacacatggtct

SEQ ID NO: 356 - RNA, 20 nt, Synthetic construct
  misc_feature 1..20: note = MYLIP
  source 1..20: mol_type = other RNA
gggtcccaccagtgacaagg

SEQ ID NO: 357 - RNA, 20 nt, Synthetic construct
  misc_feature 1..20: note = MYLIP
  source 1..20: mol_type = other RNA
ggactgcctcaaccaggtga

SEQ ID NO: 358 - RNA, 20 nt, Synthetic construct
  misc_feature 1..20: note = MYLIP
  source 1..20: mol_type = other RNA
cagagtccctgtcgcagcgc

SEQ ID NO: 359 - RNA, 20 nt, Synthetic construct
  misc_feature 1..20: note = MYLIP
  source 1..20: mol_type = other RNA
ctcagagtgagcgatcgccc

SEQ ID NO: 360 - RNA, 20 nt, Synthetic construct
  misc_feature 1..20: note = MYLIP
  source 1..20: mol_type = other RNA
ctgagtttccctggccgccc

SEQ ID NO: 361 - RNA, 20 nt, Synthetic construct
  misc_feature 1..20: note = MYLIP
  source 1..20: mol_type = other RNA
gtttccctggccgccccggg

SEQ ID NO: 362 - RNA, 106 nt, Synthetic construct
  misc_feature 1..106: note = MYLIP
  source 1..106: mol_type = other RNA
gcactgcggcggcagccggggtttaagagctatgctggaaacagcatagcaagtttaaataaggctagtccgttatcaacttgaaaaagtggcaccgagtcggtgc

SEQ ID NO: 363 - RNA, 106 nt, Synthetic construct
  misc_feature 1..106: note = MYLIP
  source 1..106: mol_type = other RNA
ggcgccaccgcggaggacaggtttaagagctatgctggaaacagcatagcaagtttaaataaggctagtccgttatcaacttgaaaaagtggcaccgagtcggtgc

SEQ ID NO: 364 - RNA, 106 nt, Synthetic construct
  misc_feature 1..106: note = MYLIP
  source 1..106: mol_type = other RNA
atgctcataggatgtattcagtttaagagctatgctggaaacagcatagcaagtttaaataaggctagtccgttatcaacttgaaaaagtggcaccgagtcggtgc

SEQ ID NO: 365 - RNA, 106 nt, Synthetic construct
  misc_feature 1..106: note = MYLIP
  source 1..106: mol_type = other RNA
ccacaataaacacatggtctgtttaagagctatgctggaaacagcatagcaagtttaaataaggctagtccgttatcaacttgaaaaagtggcaccgagtcggtgc

SEQ ID NO: 366 - RNA, 106 nt, Synthetic construct
  misc_feature 1..106: note = MYLIP
  source 1..106: mol_type = other RNA
gggtcccaccagtgacaagggtttaagagctatgctggaaacagcatagcaagtttaaataaggctagtccgttatcaacttgaaaaagtggcaccgagtcggtgc

SEQ ID NO: 367 - RNA, 106 nt, Synthetic construct
  misc_feature 1..106: note = MYLIP
  source 1..106: mol_type = other RNA
ggactgcctcaaccaggtgagtttaagagctatgctggaaacagcatagcaagtttaaataaggctagtccgttatcaacttgaaaaagtggcaccgagtcggtgc

SEQ ID NO: 368 - RNA, 106 nt, Synthetic construct
  misc_feature 1..106: note = MYLIP
  source 1..106: mol_type = other RNA
cagagtccctgtcgcagcgcgtttaagagctatgctggaaacagcatagcaagtttaaataaggctagtccgttatcaacttgaaaaagtggcaccgagtcggtgc

SEQ ID NO: 369 - RNA, 106 nt, Synthetic construct
  misc_feature 1..106: note = MYLIP
  source 1..106: mol_type = other RNA
ctcagagtgagcgatcgcccgtttaagagctatgctggaaacagcatagcaagtttaaataaggctagtccgttatcaacttgaaaaagtggcaccgagtcggtgc

SEQ ID NO: 370 - RNA, 106 nt, Synthetic construct
  misc_feature 1..106: note = MYLIP
  source 1..106: mol_type = other RNA
ctgagtttccctggccgcccgtttaagagctatgctggaaacagcatagcaagtttaaataaggctagtccgttatcaacttgaaaaagtggcaccgagtcggtgc

SEQ ID NO: 371 - RNA, 106 nt, Synthetic construct
  misc_feature 1..106: note = MYLIP
  source 1..106: mol_type = other RNA
gtttccctggccgccccggggtttaagagctatgctggaaacagcatagcaagtttaaataaggctagtccgttatcaacttgaaaaagtggcaccgagtcggtgc

SEQ ID NO: 372 - DNA, 20 bp, Synthetic construct
  misc_feature 1..20: note = APOB
  source 1..20: mol_type = other DNA
tgagggcctcccactctaca

SEQ ID NO: 373 - DNA, 20 bp, Synthetic construct
  misc_feature 1..20: note = APOB
  source 1..20: mol_type = other DNA
ccagagcactgaagacgctt

SEQ ID NO: 374 - DNA, 20 bp, Synthetic construct
  misc_feature 1..20: note = APOB
  source 1..20: mol_type = other DNA
actggaggaaacctagaagc

SEQ ID NO: 375 - DNA, 20 bp, Synthetic construct
  misc_feature 1..20: note = APOB
  source 1..20: mol_type = other DNA
cactgaagacgcttggggaa

SEQ ID NO: 376 - DNA, 20 bp, Synthetic construct
  misc_feature 1..20: note = APOB
  source 1..20: mol_type = other DNA
tgaagaaggcacccctggtc

SEQ ID NO: 377 - DNA, 20 bp, Synthetic construct
  misc_feature 1..20: note = APOB
  source 1..20: mol_type = other DNA
tgagtgcgcggccgctctgc

SEQ ID NO: 378 - RNA, 20 nt, Synthetic construct
  misc_feature 1..20: note = APOB
  source 1..20: mol_type = other RNA
tgagggcctcccactctaca

SEQ ID NO: 379 - RNA, 20 nt, Synthetic construct
  misc_feature 1..20: note = APOB
  source 1..20: mol_type = other RNA
ccagagcactgaagacgctt

SEQ ID NO: 380 - RNA, 20 nt, Synthetic construct
  misc_feature 1..20: note = APOB
  source 1..20: mol_type = other RNA
actggaggaaacctagaagc

SEQ ID NO: 381 - RNA, 20 nt, Synthetic construct
  misc_feature 1..20: note = APOB
  source 1..20: mol_type = other RNA
cactgaagacgcttggggaa

SEQ ID NO: 382 - RNA, 20 nt, Synthetic construct
  misc_feature 1..20: note = APOB
  source 1..20: mol_type = other RNA
tgaagaaggcacccctggtc

SEQ ID NO: 383 - RNA, 20 nt, Synthetic construct
  misc_feature 1..20: note = APOB
  source 1..20: mol_type = other RNA
tgagtgcgcggccgctctgc

SEQ ID NO: 384 - RNA, 106 nt, Synthetic construct
  misc_feature 1..106: note = APOB
  source 1..106: mol_type = other RNA
tgagggcctcccactctacagtttaagagctatgctggaaacagcatagcaagtttaaataaggctagtccgttatcaacttgaaaaagtggcaccgagtcggtgc

SEQ ID NO: 385 - RNA, 106 nt, Synthetic construct
  misc_feature 1..106: note = APOB
  source 1..106: mol_type = other RNA
ccagagcactgaagacgcttgtttaagagctatgctggaaacagcatagcaagtttaaataaggctagtccgttatcaacttgaaaaagtggcaccgagtcggtgc

SEQ ID NO: 386 - RNA, 106 nt, Synthetic construct
  misc_feature 1..106: note = APOB
  source 1..106: mol_type = other RNA
actggaggaaacctagaagcgtttaagagctatgctggaaacagcatagcaagtttaaataaggctagtccgttatcaacttgaaaaagtggcaccgagtcggtgc

SEQ ID NO: 387 - RNA, 106 nt, Synthetic construct
  misc_feature 1..106: note = APOB
  source 1..106: mol_type = other RNA
cactgaagacgcttggggaagtttaagagctatgctggaaacagcatagcaagtttaaataaggctagtccgttatcaacttgaaaaagtggcaccgagtcggtgc

SEQ ID NO: 388 - RNA, 106 nt, Synthetic construct
  misc_feature 1..106: note = APOB
  source 1..106: mol_type = other RNA
tgaagaaggcacccctggtcgtttaagagctatgctggaaacagcatagcaagtttaaataaggctagtccgttatcaacttgaaaaagtggcaccgagtcggtgc

SEQ ID NO: 389 - RNA, 106 nt, Synthetic construct
  misc_feature 1..106: note = APOB
  source 1..106: mol_type = other RNA
tgagtgcgcggccgctctgcgtttaagagctatgctggaaacagcatagcaagtttaaataaggctagtccgttatcaacttgaaaaagtggcaccgagtcggtgc

SEQ ID NO: 390 - DNA, 20 bp, Synthetic construct
  misc_feature 1..20: note = cPCSK9-C gRNA target site
  source 1..20: mol_type = other DNA
aggtttccgcagcggcgtcg

SEQ ID NO: 391 - RNA, 20 nt, Synthetic construct
  misc_feature 1..20: note = cPCSK9-C gRNA spacer
  source 1..20: mol_type = other RNA
aggtttccgcagcggcgtcg

SEQ ID NO: 392 - RNA, 100 nt, Synthetic construct
  misc_feature 1..100: note = cPCSK9-C full length gRNA
  source 1..100: mol_type = other RNA
aggtttccgcagcggcgtcggttttagagctagaaatagcaagttaaaataaggctagtccgttatcaacttgaaaaagtggcaccgagtcggtgctttt

SEQ ID NO: 393 - RNA, 63 nt, Synthetic construct
  misc_feature 1..63: note = 5' UTR (5U4)
  source 1..63: mol_type = other RNA
ggagcaattgaccgggaagctcagaataaacgctcaactttggccggatcttctagagccacc

SEQ ID NO: 394 - RNA, 46 nt, Synthetic construct
  misc_feature 1..46: note = 3' UTR (3U2)
  source 1..46: mol_type = other RNA
tagtaagaattcaaagaaagtttcttcacattctctcgagcgtacg

SEQ ID NO: 395 - RNA, 6433 nt, Synthetic construct
  misc_feature 1..6433: note = dCas9-KRAB-DNMT3A/L mRNA
  source 1..6433: mol_type = other RNA
ggagcaattgaccgggaagctcagaataaacgctcaactttggccggatcttctagagccaccatgaaccacgatcaggagtttgacccccctaaggtgtacccacccgtgccagccgagaagaggaagcccatccgcgtgctgtccctgttcgacggcatcgccacaggcctgctggtgctgaaggatctgggcatccaggtggacagatatatcgcctccgaggtgtgcgaggattctatcaccgtgggcatggtgaggcaccagggcaagatcatgtacgtgggcgacgtgcgcagcgtgacacagaagcacatccaggagtggggacccttcgacctggtcatcggaggcagcccctgtaatgacctgtccatcgtgaaccctgcaaggaagggcctgtatgagggaaccggcagactgttctttgagttctacaggctgctgcacgacgcccgccctaaggagggcgatgacaggccattcttttggctgtttgagaacgtggtggccatgggcgtgagcgacaagcgggatatctccagattcctggagtctaatcccgtgatgatcgatgcaaaggaggtgtctgccgcacacagggcaaggtacttttggggaaatctgcctggcatgaaccgcccactggccagcaccgtgaacgacaagctggagctgcaggagtgcctggagcacggaaggatcgccaagttctccaaggtgcggacaatcaccacaagatctaacagcatcaagcagggcaaggatcagcacttccccgtgttcatgaatgagaaggaggacatcctgtggtgtaccgagatggagcgcgtgttcggctttccagtgcactatacagacgtgagcaatatgagccggctggcaaggcagagactgctgggccggtcctggtctgtgccagtgatcagacacctgttcgcccccctgaaggagtactttgcctgcgtgtctagcggcaactctaatgccaacagcagaggcccttccttttcctctggcctggtgccactgtctctgaggggcagccacatgggccccatggagatctacaagaccgtgtccgcctggaagaggcagcctgtgcgcgtgctgtctctgttccgcaacatcgacaaggtgctcaagagcctgggctttctggagagcggatccggatctggaggaggcaccctgaagtatgtggaggatgtgacaaatgtggtgcggagagatgtggagaagtggggccccttcgatctggtgtacggatccacccagccactgggaagctcctgcgataggtgtccaggatggtatatgttccagtttcacagaatcctgcagtacgcactgccaaggcaggagagccagcgccctttcttttggatctttatggacaacctgctgctgacagaggatgaccaggagacaacaacccgcttcctgcagacagaggcagtgaccctgcaggatgtgaggggacgcgactatcagaatgccatgcgggtgtggtctaacatccctggcctgaaaagcaagcacgcccccctgacccctaaggaggaggagtacctgcaggcccaggtgcggagcagatccaagctggatgcccctaaggtggacctgctggtgaagaattgtctgctgccactgcgggagtacttcaagtactttagtcagaatagcctgccactggaggcaagcggatccggaagggcatctcctggaatcccaggaagcacccgcaaccccaagaagaagcggaaggtgggcatccacggcgtgcccgccgccgacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaaaagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaaaagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggacgccatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaaaagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaaaagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaaggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaggaaccctggctggtggagagagaaattcaccaagagacccatcctgattcagagactgcatttgaaatcaaatcatcagttccgaaaaagaaacgcaaagtttagtaagaattcaaagaaagtttcttcacattctctcgagcgtacgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2024/0401014 A1
(43) Pub. Date: Dec. 5, 2024

(54) COMPOSITIONS, SYSTEMS, AND METHODS FOR REDUCING LOW-DENSITY LIPOPROTEIN THROUGH TARGETED GENE REPRESSION

(71) Applicant: Tune Therapeutics, Inc., Seattle, WA (US)

(72) Inventors: Jennifer KWON, Durham, NC (US); Kendra CONGDON, Durham, NC (US)

(73) Assignee: Tune Therapeutics, Inc., Seattle, WA (US)

(21) Appl. No.: 18/799,946

(22) Filed: Aug. 9, 2024

Related U.S. Application Data
(62) Division of application No. 18/479,758, filed on Oct. 2, 2023, now Pat. No. 12,098,399.
Application No. 18/479,758 is a continuation of application No. PCT/US2023/069031, filed on Jun. 23, 2023.
(60) Provisional application No. 63/355,540, filed on Jun. 24, 2022.
(60) Provisional application No. 63/399,625, filed on Aug. 19, 2022.
(60) Provisional application No. 63/401,558, filed on Aug. 26, 2022.
(60) Provisional application No. 63/453,044, filed on Mar. 17, 2023.
(60) Provisional application No. 63/466,681, filed on May 15, 2023.
(60) Provisional application No. 63/472,224, filed on Jun. 9, 2023.

(51) Int. Cl. C12N 9/22; C12N 15/11

(52) U.S. Cl. CPC C12N 9/22; C12N 15/11; C12N 2310/20

(57) ABSTRACT

Provided in some aspects are epigenetic-modifying DNA-targeting systems, such as CRISPR-Cas/guide RNA (gRNA) systems for the transcriptional repression of genes to promote a cellular phenotype that leads to reduction of low-density lipoprotein (LDL). In some embodiments, the epigenetic-modifying DNA-targeting systems bind to or target a target site of at least one gene or regulatory element thereof that regulate LDL. In some embodiments, the systems are multiplexed systems that bind to or target a target site in at least two genes or regulatory elements thereof. Also provided herein are methods and uses related to the provided epigenetic-modifying DNA targeting systems in connection with treatments for cardiovascular disease and familial hypercholesterolemia.

### CROSS-REFERENCE TO RELATED APPLICATIONS

This application is a Divisional Application of U.S. patent application Ser. No. 18/479,758, filed Oct. 2, 2023, which is a Continuation Application of International Application No. PCT/US2023/069031, filed on Jun. 23, 2023, which claims priority from U.S. provisional application No. 63/355,540 filed Jun. 24, 2022, U.S. provisional application No. 63/399,625 filed Aug. 19, 2022, U.S. provisional application No. 63/401,558 filed Aug. 26, 2022, U.S. provisional application No. 63/453,044 filed Mar. 17, 2023, U.S. provisional application No. 63/466,681 filed May 15, 2023, and U.S. provisional application No. 63/472,224 filed Jun. 9, 2023, the contents of which are incorporated by reference in their entireties.

### INCORPORATION BY REFERENCE OF SEQUENCE LISTING

The present application is being filed along with a Sequence Listing in electronic format. The Sequence Listing is provided as a file entitled 224742001910SeqList.xml, created Aug. 8, 2024, which is 561,656 bytes in size. The information in the electronic format of the Sequence Listing is incorporated by reference in its entirety.

### FIELD

The present disclosure relates in some aspects to epigenetic-modifying DNA-targeting systems, such as CRISPR-Cas/guide RNA (gRNA) systems, for the transcriptional repression of genes to promote a cellular phenotype that leads to reduction of low-density lipoprotein (LDL). In some embodiments, the epigenetic-modifying DNA-targeting systems bind to or target a target site of at least one gene or regulatory element thereof that regulate LDL. In some embodiments, the systems are multiplexed systems that bind to or target a target site in at least two genes or regulatory elements thereof. In some aspects, the present disclosure also provides methods and uses related to the provided epigenetic-modifying DNA targeting systems in connection with treatments for cardiovascular disease and familial hypercholesterolemia.

### BACKGROUND

Familial hypercholesterolemia (FH) is a genetic condition resulting in elevated levels of LDL cholesterol in the bloodstream, resulting in increased risk of cardiovascular disease. Current treatments for FH, including administration of statins and antibody therapy, face challenges including toxicity and high cost associated with repeated drug administration. There is a need for new and improved methods to overcome these challenges. The present disclosure addresses these and other needs.

### SUMMARY

In some aspects, provided herein is an epigenetic-modifying DNA-targeting system comprising a plurality of DNA-targeting modules for repressing transcription of a plurality of genes that regulate low-density lipoprotein (LDL), wherein the plurality of DNA-targeting modules comprises a first DNA-targeting module for repressing transcription of a first gene of the plurality of genes, and a second DNA-targeting module for repressing transcription of a second gene of the plurality of genes, and wherein each DNA-targeting module comprises a fusion protein comprising: (a) a DNA-binding domain for targeting to a target site of one of the plurality of genes, and (b) at least one transcriptional repressor domain. In some of any of the provided embodiments, the DNA-targeting system does not introduce a genetic disruption or a DNA break. In some of any of the provided embodiments, the fusion protein of each DNA-targeting module comprises a DNA-binding domain selected from: a Clustered Regularly Interspaced Short Palindromic Repeats associated (Cas) protein or a variant thereof, a zinc finger protein (ZFP); a transcription activator-like effector (TALE); a meganuclease; a homing endonuclease; or an I-SceI enzyme or a variant thereof, optionally wherein the DNA-binding domain comprises a catalytically inactive variant of any of the foregoing. In some of any of the provided embodiments, the fusion protein of the first DNA-targeting module comprises a DNA-binding domain for targeting a target site of the first gene or regulatory DNA element thereof, and at least one transcriptional repressor domain; and the fusion protein of the second DNA-targeting module comprises a DNA-binding domain for targeting a target site of the second gene or regulatory DNA element thereof, and at least one transcriptional repressor domain. In some of any of the provided embodiments, any two or more of the DNA-targeting modules comprise the same fusion protein. In some of any of the provided embodiments, the first and second DNA-targeting modules comprise the same fusion protein. In some of any of the provided embodiments, any two or more of the DNA-targeting modules comprise different fusion proteins. In some of any of the provided embodiments, the first and second DNA-targeting modules comprise different fusion proteins.

In some aspects, provided herein is an epigenetic-modifying DNA-targeting system comprising a plurality of DNA-targeting modules for repressing transcription of a plurality of genes that regulate low-density lipoprotein (LDL), comprising: (1) a first DNA-targeting module that reduces transcription of a first gene that regulates low-density lipoprotein (LDL), wherein the first DNA-targeting module comprises a first fusion protein comprising (a) a DNA-binding domain for targeting a target site of the first gene or regulatory DNA element thereof, and (b) at least one transcriptional repressor domain; and (2) a second DNA-targeting module that reduces transcription of a second gene that regulates LDL, wherein the second DNA-targeting module comprises a second fusion protein comprising (a) a DNA-binding domain for targeting a target site of the second gene or regulatory DNA element thereof, and (b) at least one transcriptional repressor domain. In some of any of the provided embodiments, the first DNA-targeting module comprises a first targeting polynucleotide for targeting to the target site of the first gene and the second DNA-targeting module comprises a second targeting polynucleotide for targeting to the target site of the second gene, wherein the first and second targeting polynucleotides complex with the DNA-binding domain of the fusion protein. In some of any of the provided embodiments, the DNA-binding domain is a Clustered Regularly Interspaced Short Palindromic Repeats associated (Cas) protein or variant thereof and the first and second targeting polynucleotides comprise a first gRNA and a second gRNA, respectively.

In some aspects, provided herein is an epigenetic-modifying DNA-targeting system comprising: (a) a fusion protein comprising a Clustered Regularly Interspaced Short Palindromic Repeats associated (Cas) protein or variant thereof and at least one transcriptional repressor domain; and (b) a plurality of guide RNAs (gRNAs) comprising at least a first gRNA and a second gRNA, wherein the first gRNA targets a target site of a first gene that regulates low-density lipoprotein (LDL) and the second gRNA targets a target site of a second gene that regulates LDL.

In some of any of the provided embodiments, the system further comprises a third DNA-targeting module for repressing transcription of a third gene that regulates low-density lipoprotein (LDL). In some of any of the provided embodiments, the system further comprises a third gRNA that targets a target site of a third gene that regulates LDL, optionally wherein the system further comprises a fourth gRNA that targets a target site of a fourth gene that regulates LDL, optionally a fifth gRNA that targets a target site of a fifth gene that regulates LDL, and/or optionally a sixth gRNA that targets a target site of a sixth gene that regulates LDL.

In some of any of the provided embodiments, the first gene and the second gene are independently selected from the group consisting of PCSK9, LPA, MYLIP, ANGPTL3, APOB, and APOC3. In some of any of the provided embodiments, the first gene and the second gene are different. In some of any of the provided embodiments, the first gene and the second gene are: PCSK9 and LPA; PCSK9 and MYLIP; PCSK9 and ANGPTL3; PCSK9 and APOC3; PCSK9 and APOB; LPA and MYLIP; LPA and ANGPTL3; LPA and APOC3; LPA and APOB; MYLIP and ANGPTL3; MYLIP and APOC3; MYLIP and APOB; ANGPTL3 and APOC3; ANGPTL3 and APOB; or APOC3 and APOB. In some of any of the provided embodiments, the first gene, the second gene, and the third gene are each independently selected from the group consisting of PCSK9, LPA, MYLIP, ANGPTL3, APOC3, and APOB. In some of any of the provided embodiments, the first gene, the second gene, and the third gene are different. In some of any of the provided embodiments, the first gene, the second gene, and the third gene are: PCSK9, LPA, and MYLIP; PCSK9, LPA, and ANGPTL3; PCSK9, LPA, and APOC3; PCSK9, LPA, and APOB; PCSK9, MYLIP, and ANGPTL3; PCSK9, MYLIP, and APOC3; PCSK9, MYLIP, and APOB; PCSK9, ANGPTL3, and APOC3; PCSK9, ANGPTL3, and APOB; PCSK9, APOC3, and APOB; LPA, MYLIP, and ANGPTL3; LPA, MYLIP, and APOC3; LPA, MYLIP, and APOB; LPA, ANGPTL3, and APOC3; LPA, ANGPTL3, and APOB; LPA, APOC3, and APOB; MYLIP, ANGPTL3, and APOC3; MYLIP, ANGPTL3, and APOB; MYLIP, APOC3, and APOB; or ANGPTL3, APOC3, and APOB. In some of any of the provided embodiments, at least one gene is PCSK9. In some of any of the provided embodiments, at least two genes are PCSK9 and LPA. In some of any of the provided embodiments, at least three genes are PCSK9, LPA, and MYLIP. In some of any of the provided embodiments, at least three genes are PCSK9, MYLIP, and APOB.

In some of any of the provided embodiments, the target site of each of the plurality of genes is in the gene or a regulatory DNA element thereof. In some of any of the provided embodiments, the regulatory DNA element is an enhancer or a promoter. In some of any of the provided embodiments, the target site of the first gene and the second gene are selected from two different members of the group consisting of (a)-(f): (a) a target site for PCSK9, located within 500 bp of human genome assembly GRCh38 (hg38) genomic coordinates chr1:55,039,548; (b) a target site for LPA, located within 500 bp of the hg38 genomic coordinates chr6:160,664,275; (c) a target site for MYLIP, located within 500 bp of the hg38 genomic coordinates chr6:16,129,086; (d) a target site for ANGPTL3, located within 500 bp of the hg38 genomic coordinates chr1:62,597,520; (e) a target site for APOC3, located within 500 bp of the hg38 genomic coordinates chr11:116,829,907; and (f) a target site for APOB, located within 500 bp of the hg38 genomic coordinates chr2:21,044,073. In some of any of the provided embodiments, the target site of the first gene and the second gene are selected from two different members of the group consisting of (a)-(f): (a) a target site located within 500 bp of a transcriptional start site of PCSK9; (b) a target site located within 500 bp of a transcriptional start site of LPA; (c) a target site located within 500 bp of a transcriptional start site of MYLIP; (d) a target site located within 500 bp of a transcriptional start site of ANGPTL3; (e) a target site located within 500 bp of a transcriptional start site of APOC3; and (f) a target site located within 500 bp of a transcriptional start site of APOB. In some of any of the provided embodiments, the target site of the first gene, the second gene and the third gene are selected from three different members of the group consisting of (a)-(f): (a) a target site for PCSK9, located within 500 bp of human genome assembly GRCh38 (hg38) genomic coordinates chr1:55,039,548; (b) a target site for LPA, located within 500 bp of the hg38 genomic coordinates chr6:160,664,275; (c) a target site for MYLIP, located within 500 bp of the hg38 genomic coordinates chr6:16,129,086; (d) a target site for ANGPTL3, located within 500 bp of the hg38 genomic coordinates chr1:62,597,520; (e) a target site for APOC3, located within 500 bp of the hg38 genomic coordinates chr11:116,829,907; and (f) a target site for APOB, located within 500 bp of the hg38 genomic coordinates chr2:21,044,073. In some of any of the provided embodiments, the target site of the first gene, the second gene and the third gene are selected from three different members of the group consisting of (a)-(f): (a) a target site located within 500 bp of a transcriptional start site of PCSK9; (b) a target site located within 500 bp of a transcriptional start site of LPA; (c) a target site located within 500 bp of a transcriptional start site of MYLIP; (d) a target site located within 500 bp of a transcriptional start site of ANGPTL3; (e) a target site located within 500 bp of a transcriptional start site of APOC3; and (f) a target site located within 500 bp of a transcriptional start site of APOB. In some of any of the provided embodiments, the target site of the first gene and the second gene are selected from two different members of the group consisting of (a)-(f): (a) a target site for PCSK9 having the sequence set forth in any one of SEQ ID NOS:1-13 or 306-317, a contiguous portion thereof of at least 14 nucleotides (nt), or a complementary sequence of any of the foregoing; (b) a target site for LPA having the sequence set forth in any one of SEQ ID NOS:14-23, a contiguous portion thereof of at least 14 nucleotides (nt), or a complementary sequence of any of the foregoing; (c) a target site for MYLIP having the sequence set forth in any one of SEQ ID NOS:24-33 or 342-351, a contiguous portion thereof of at least 14 nucleotides (nt), or a complementary sequence of any of the foregoing; (d) a target site for ANGPTL3 having the sequence set forth in any one of SEQ ID NOS:34-43, a contiguous portion thereof of at least 14 nucleotides (nt), or a complementary sequence of any of the foregoing; (e) a target site for APOC3 having the sequence set forth in any one of SEQ ID NOS:44-53, a contiguous portion thereof of at least 14 nucleotides (nt), or a complementary sequence of any of the foregoing; and (f) a target site for APOB having the sequence set forth in any one of SEQ ID NOS:54-63 or 372-377, a contiguous portion thereof of at least 14 nucleotides (nt), or a complementary sequence of any of the foregoing. In some embodiments, the target site of PCSK9 is (i) set forth in SEQ ID NO:3, (ii) has a contiguous portion of SEQ ID NO:3 of at least 14 nucleotides, or (iii) is a complementary sequence of (i) or (ii). In some of any of the provided embodiments, the target site of the first gene and the second gene are selected from two different members of the group consisting of (a)-(f): (a) a target site of PCSK9 having the sequence set forth in any one of SEQ ID NOS:1-13 or 306-317; (b) a target site of LPA having the sequence set forth in any one of SEQ ID NOS:14-23; (c) a target site of MYLIP having the sequence set forth in any one of SEQ ID NOS:24-33 or 342-351; (d) a target site of ANGPTL3 having the sequence set forth in any one of SEQ ID NOS: 34-43; (e) a target site of APOC3 having the sequence set forth in any one of SEQ ID NOS:44-53; and (f) a target site of APOB having the sequence set forth in any one of SEQ ID NOS:54-63 or 372-377. In some embodiments, the target site of PCSK9 is set forth in SEQ ID NO:3. In some of any of the provided embodiments, the target site of the first gene, the second gene and the third gene are selected from three different members of the group consisting of (a)-(f): (a) a target site of PCSK9 having the sequence set forth in any one of SEQ ID NOS:1-13 or 306-317, a contiguous portion thereof of at least 14 nucleotides (nt), or a complementary sequence of any of the foregoing; (b) a target site of LPA having the sequence set forth in any one of SEQ ID NOS:14-23, a contiguous portion thereof of at least 14 nucleotides (nt), or a complementary sequence of any of the foregoing; (c) a target site of MYLIP having the sequence set forth in any one of SEQ ID NOS:24-33 or 342-351, a contiguous portion thereof of at least 14 nucleotides (nt), or a complementary sequence of any of the foregoing; (d) a target site of ANGPTL3 having the sequence set forth in any one of SEQ ID NOS:34-43, a contiguous portion thereof of at least 14 nucleotides (nt), or a complementary sequence of any of the foregoing; (e) a target site of APOC3 having the sequence set forth in any one of SEQ ID NOS:44-53, a contiguous portion thereof of at least 14 nucleotides (nt), or a complementary sequence of any of the foregoing; and (f) a target site of APOB having the sequence set forth in any one of SEQ ID NOS:54-63 or 372-377, a contiguous portion thereof of at least 14 nucleotides (nt), or a complementary sequence of any of the foregoing. In some embodiments, the target site of PCSK9 is (i) set forth in SEQ ID NO:3, (ii) has a contiguous portion of SEQ ID NO:3 of at least 14 nucleotides, or (iii) is a complementary sequence of (i) or (ii). In some of any of the provided embodiments, the target site of the first gene, the second gene or the third gene are selected from three different members of the group consisting of (a)-(f): (a) a target site of PCSK9 having the sequence set forth in any one of SEQ ID NOS:1-13 or 306-317; (b) a target site of LPA having the sequence set forth in any one of SEQ ID NOS:14-23; (c) a target site of MYLIP having the sequence set forth in any one of SEQ ID NOS:24-33 or 342-351; (d) a target site of ANGPTL3 having the sequence set forth in any one of SEQ ID NOS: 34-43; (e) a target site of APOC3 having the sequence set forth in any one of SEQ ID NOS:44-53; and (f) a target site of APOB having the sequence set forth in any one of SEQ ID NOS:54-63 or 372-377. In some embodiments, the target site of PCSK9 is set forth in SEQ ID NO:3.

In some of any of the provided embodiments, the Cas protein or variant thereof is a variant Cas protein that is a deactivated (dCas) protein. In some of any of the provided embodiments, the dCas protein lacks nuclease activity. In some of any of the provided embodiments, the dCas protein is a dCas9 protein. In some of any of the provided embodiments, the dCas protein is a dCasl2 protein.

In some of any of the provided embodiments, the dCas9 protein is a Staphylococcus aureus dCas9 (dSaCas9) protein. In some of any of the provided embodiments, the dSaCas9 comprises at least one amino acid mutation selected from D10A and N580A, with reference to numbering of positions of SEQ ID NO:204. In some of any of the provided embodiments, the dSaCas9 protein comprises the sequence set forth in SEQ ID NO:205, or an amino acid sequence that has at least 90%, 91%, 92%, 93%, 94%, 95%, 96%, 97%, 98%, or 99% sequence identity thereto. In some of any of the provided embodiments, the dSaCas9 is set forth in SEQ ID NO:205.

In some of any of the provided embodiments, the dCas9 protein is a Streptococcus pyogenes dCas9 (dSpCas9) protein. In some of any of the provided embodiments, the dSpCas9 protein comprises at least one amino acid mutation selected from D10A and H840A, with reference to numbering of positions of SEQ ID NO:206. In some of any of the provided embodiments, the dSpCas9 comprises the sequence set forth in SEQ ID NO:207, or an amino acid sequence that has at least 90%, 91%, 92%, 93%, 94%, 95%, 96%, 97%, 98%, or 99% sequence identity thereto. In some of any of the provided embodiments, the dSpCas9 is set forth in SEQ ID NO:207.

In some of any of the provided embodiments, each gRNA comprises a gRNA spacer sequence that is complementary to the target site of the respective gene. In some of any of the provided embodiments, the first gRNA and the second gRNA are selected from two different members of the group consisting of (a)-(f): (a) a gRNA targeting a target site of PCSK9 comprising a gRNA spacer sequence comprising the sequence set forth in any one of SEQ ID NOS:64-76 or 318-329, or a contiguous portion thereof of at least 14 nt; (b) a gRNA targeting a target site of LPA comprising a gRNA spacer sequence comprising the sequence set forth in any one of SEQ ID NOS:77-86, or a contiguous portion thereof of at least 14 nt; (c) a gRNA targeting a target site of MYLIP comprising a gRNA spacer sequence comprising the sequence set forth in any one of SEQ ID NOS:87-96 or 352-361, or a contiguous portion thereof of at least 14 nt; (d) a gRNA targeting a target site of ANGPTL3 comprising a gRNA spacer sequence comprising the sequence set forth in any one of SEQ ID NOS:97-106, or a contiguous portion thereof of at least 14 nt; (e) a gRNA targeting a target site of APOC3 comprising a gRNA spacer sequence comprising the sequence set forth in any one of SEQ ID NOS:107-116, or a contiguous portion thereof of at least 14 nt; and (f) a gRNA targeting a target site of APOB comprising a gRNA spacer sequence comprising the sequence set forth in any one of SEQ ID NOS:117-126 or 378-383, or a contiguous portion thereof of at least 14 nt. In some embodiments, the gRNA spacer sequence targeting a target site of PCSK9 comprises the sequence set forth in SEQ ID NO:66 or a contiguous portion of at least 14 nt. In some of any of the provided embodiments, the first gRNA, the second gRNA, and the third gRNA are selected from three different members of the group consisting of (a)-(f): (a) a gRNA targeting a target site of PCSK9 comprising a gRNA spacer sequence comprising the sequence set forth in any one of SEQ ID NOS:64-76 or 318-329, or a contiguous portion thereof of at least 14 nt; (b) a gRNA targeting a target site of LPA comprising a gRNA spacer sequence comprising the sequence set forth in any one of SEQ ID NOS:77-86, or a contiguous portion thereof of at least 14 nt; (c) a gRNA targeting a target site of MYLIP comprising a gRNA spacer sequence comprising the sequence set forth in any one of SEQ ID NOS:87-96 or 352-361, or a contiguous portion thereof of at least 14 nt; (d) a gRNA targeting a target site of ANGPTL3 comprising a gRNA spacer sequence comprising the sequence set forth in any one of SEQ ID NOS:97-106, or a contiguous portion thereof of at least 14 nt; (e) a gRNA targeting a target site of APOC3 comprising a gRNA spacer sequence comprising the sequence set forth in any one of SEQ ID NOS:107-116, or a contiguous portion thereof of at least 14 nt; and (f) a gRNA targeting a target site of APOB comprising a gRNA spacer sequence comprising the sequence set forth in any one of SEQ ID NOS:117-126 or 378-383, or a contiguous portion thereof of at least 14 nt. In some embodiments, the gRNA spacer sequence targeting a target site of PCSK9 comprises the sequence set forth in SEQ ID NO:66 or a contiguous portion of at least 14 nt. In some of any of the provided embodiments, each gRNA independently comprises a spacer sequence between 14 nt and 24 nt, or between 16 nt and 22 nt in length. In some of any of the provided embodiments, each gRNA independently comprises a spacer sequence that is 18 nt, 19 nt, 20 nt, 21 nt, or 22 nt in length. In some of any of the provided embodiments, the first gRNA and the second gRNA are selected from two different members of the group consisting of (a)-(f): (a) a gRNA targeting a target site of PCSK9 comprising the gRNA spacer sequence set forth in any one of SEQ ID NOS:64-76 or 318-329; (b) a gRNA targeting a target site in LPA comprising the gRNA spacer sequence set forth in any one of SEQ ID NOS:77-86; (c) a gRNA targeting a target site in MYLIP comprising the gRNA spacer sequence set forth in any one of SEQ ID NOS:87-96 or 352-361; (d) a gRNA targeting a target site in ANGPTL3 comprising the gRNA spacer sequence set forth in any one of SEQ ID NOS:97-106; (e) a gRNA targeting a target site in APOC3 comprising the gRNA spacer sequence set forth in any one of SEQ ID NOS:107-116; and (f) a gRNA targeting a target site in APOB comprising the gRNA spacer sequence set forth in any one of SEQ ID NOS:117-126 or 378-383. In some embodiments, the gRNA spacer sequence targeting a target site of PCSK9 comprises the sequence set forth in SEQ ID NO:66. In some of any of the provided embodiments, each gRNA further comprises a scaffold sequence set forth in SEQ ID NO:191. In some of any of the provided embodiments, the first gRNA and the second gRNA are selected from two different members of the group consisting of (a)-(f): (a) a gRNA targeting a target site in PCSK9 comprising the sequence set forth in any one of SEQ ID NOS:127-139 or 330-341; (b) a gRNA targeting a target site in LPA comprising the sequence set forth in any one of SEQ ID NOS:140-149; (c) a gRNA targeting a target site in MYLIP comprising the sequence set forth in any one of SEQ ID NOS:150-159 or 362-371; (d) a gRNA targeting a target site in ANGPTL3 comprising the sequence set forth in any one of SEQ ID NOS:160-169; (e) a gRNA targeting a target site in APOC3 comprising the sequence set forth in any one of SEQ ID NOS:170-179; and (f) a gRNA targeting a target site in APOB comprising the sequence set forth in any one of SEQ ID NOS:180-189 or 384-389. In some embodiments, the gRNA targeting a target site of PCSK9 comprises the sequence set forth in SEQ ID NO:129. In some of any of the provided embodiments, the first gRNA and the second gRNA are selected from two different members of the group consisting of (a)-(f): (a) a gRNA targeting a target site in PCSK9 set forth in any one of SEQ ID NOS:127-139 or 330-341; (b) a gRNA targeting a target site in LPA set forth in any one of SEQ ID NOS:140-149; (c) a gRNA targeting a target site in MYLIP set forth in any one of SEQ ID NOS:150-159 or 362-371; (d) a gRNA targeting a target site in ANGPTL3 set forth in any one of SEQ ID NOS:160-169; (e) a gRNA targeting a target site in APOC3 set forth in any one of SEQ ID NOS:170-179; and (f) a gRNA targeting a target site in APOB set forth in any one of SEQ ID NOS:180-189 or 384-389. In some embodiments, the gRNA targeting a target site of PCSK9 is set forth in SEQ ID NO:129. In some of any of the provided embodiments, the first gRNA, the second gRNA, and the third gRNA are selected from three different members of the group consisting of (a)-(f): (a) a gRNA targeting a target site in PCSK9 comprising the sequence set forth in any one of SEQ ID NOS:127-139 or 330-341; (b) a gRNA targeting a target site in LPA comprising the sequence set forth in any one of SEQ ID NOS:140-149; (c) a gRNA targeting a target site in MYLIP comprising the sequence set forth in any one of SEQ ID NOS:150-159 or 362-371; (d) a gRNA targeting a target site in ANGPTL3 comprising the sequence set forth in any one of SEQ ID NOS:160-169; (e) a gRNA targeting a target site in APOC3 comprising the sequence set forth in any one of SEQ ID NOS:170-179; and (f) a gRNA targeting a target site in APOB comprising the sequence set forth in any one of SEQ ID NOS:180-189 or 384-389. In some embodiments, the gRNA targeting a target site of PCSK9 comprises the sequence set forth in SEQ ID NO:129. In some of any of the provided embodiments, the first gRNA, the second gRNA, and the third gRNA are selected from three different members of the group consisting of (a)-(f): (a) a gRNA targeting a target site in PCSK9 set forth in any one of SEQ ID NOS:127-139 or 330-341; (b) a gRNA targeting a target site in LPA set forth in any one of SEQ ID NOS:140-149; (c) a gRNA targeting a target site in MYLIP set forth in any one of SEQ ID NOS:150-159 or 362-371; (d) a gRNA targeting a target site in ANGPTL3 set forth in any one of SEQ ID NOS:160-169; (e) a gRNA targeting a target site in APOC3 set forth in any one of SEQ ID NOS:170-179; and (f) a gRNA targeting a target site in APOB set forth in any one of SEQ ID NOS:180-189 or 384-389. In some embodiments, the gRNA targeting a target site of PCSK9 is set forth in SEQ ID NO:129. In some of any of the provided embodiments, at least one gRNA comprises modified nucleotides for increased stability.

In some of any of the provided embodiments, at least one transcriptional repressor domain is capable of reducing transcription of the gene. In some of any of the provided embodiments, the transcriptional repressor domain is selected from the group consisting of a KRAB domain, a DNMT3A domain, a DNMT3L domain, a DNMT3B domain, a DNMT3A-DNMT3L fusion protein domain, an ERF repressor domain, an Mxi1 repressor domain, a SID4X repressor domain, a Mad-SID repressor domain, an LSD1 repressor domain, an EZH2 repressor domain, a SunTag domain, or a variant or portion of any of the foregoing, or a combination of any of the foregoing. In some of any of the provided embodiments, the transcriptional repressor domain is a KRAB domain, a DNMT3A domain, or a DNMT3L domain, or a combination of any of the foregoing. In some of any of the provided embodiments, at least one transcriptional repressor domain comprises a sequence selected from any one of SEQ ID NOS:193, 195, 197, 199, 201, 220-226, 283, 284, 285, 286, 287, 289 and 290 or a domain thereof, a portion thereof, or an amino acid sequence that has at least 90%, 91%, 92%, 93%, 94%, 95%, 96%, 97%, 98%, or 99% sequence identity to any one of SEQ ID NOS:193, 195, 197, 199, 201, 220-226, 283, 284, 285, 286, 287, 289 and 290.

In some of any of the provided embodiments, the at least one transcriptional repressor domain comprises a KRAB domain or a variant or portion thereof that exhibits transcriptional repressor activity. In some of any of the provided embodiments, the at least one transcriptional repressor domain comprises the sequence set forth in SEQ ID NO:193, a portion thereof, or an amino acid sequence that has at least 90%, 91%, 92%, 93%, 94%, 95%, 96%, 97%, 98%, or 99% sequence identity to SEQ ID NO:193. In some of any of the provided embodiments, the at least one transcriptional repressor domain comprises the sequence set forth in SEQ ID NO:290, a portion thereof, or an amino acid sequence that has at least 90%, 91%, 92%, 93%, 94%, 95%, 96%, 97%, 98%, or 99% sequence identity to SEQ ID NO:290. In some of any above embodiments, the KRAB domain, portion or variant thereof is a sequence that exhibits transcriptional repressor activity.

In some of any of the provided embodiments, the at least one transcriptional repressor domain comprises a DNMT3A domain or a variant or portion thereof that exhibits transcriptional repressor activity. In some embodiments, the DNMT3A domain, or portion thereof, is from a human DNMT3A. In some of any of the provided embodiments, the at least one effector domain comprises the sequence set forth in SEQ ID NO:284, or is a portion thereof, or an amino acid sequence that has at least 90%, 91%, 92%, 93%, 94%, 95%, 96%, 97%, 98%, or 99% sequence identity to SEQ ID NO: 284. In some embodiments, the portion thereof is a catalytically active portion that exhibits transcription repressor activity. In some of any of the provided embodiments, the at least one transcriptional repressor domain comprises the sequence set forth in SEQ ID NO:195, a portion thereof, or an amino acid sequence that has at least 90%, 91%, 92%, 93%, 94%, 95%, 96%, 97%, 98%, or 99% sequence identity to SEQ ID NO: 195. In some embodiments, the at least one effector domain is or includes the catalytically active portion of DNMT3A set forth in SEQ ID NO: 195. In some of any of the provided embodiments, the at least one effector domain comprises the sequence set forth in SEQ ID NO:285, or is a portion thereof, or an amino acid sequence that has at least 90%, 91%, 92%, 93%, 94%, 95%, 96%, 97%, 98%, or 99% sequence identity to SEQ ID NO: 285. In some embodiments, the at least one effector domain is or includes the catalytically active protion of DNMT3A set forth in SEQ ID NO: 285.

In some of any of the provided embodiments, the at least one transcriptional repressor domain comprises a DNMT3L domain or a variant or portion thereof that exhibits transcriptional repressor activity. In some of any of the provided embodiments, the at least one transcriptional repressor domain is a fusion domain comprising a DNMT3A domain, such as any DNMT3A domain or C-terminal portion or variant described above, and a DNMT3L domain or a variant or portion thereof, in which the fusion domain exhibits transcriptional repressor activity. In some embodiments, DNMT3L domain, or the the variant or C-terminal portion of a DNMT3L domain, is one that stimulates enhanced transcription repressor activity of DNMT3A. In some embodiments, DNMT3L domain, or the the variant or C-terminal portion of a DNMT3L domain, is one that interacts with DNMT3A. In some embodiments, the DNMT3L domain, or C-terminal portion thereof, is from a murine DNMT3L. In some embodiments, the DNMT3L domain, or C-terminal portion thereof, is a human or humanized DNMT3L. In some of any of the provided embodiments, the DNTML domain comprises the sequence set forth in SEQ ID NO:289, a C-terminal portion thereof, or an amino acid sequence that has at least 90%, 91%, 92%, 93%, 94%, 95%, 96%, 97%, 98%, or 99% sequence identity to SEQ ID NO: 289. In some of any of the provided embodiments, the DNMT3L comprises the sequence set forth in SEQ ID NO:286, or an amino acid sequence that has at least 90%, 91%, 92%, 93%, 94%, 95%, 96%, 97%, 98%, or 99% sequence identity to SEQ ID NO: 286. In some embodiments, the DNMT3L domain is or includes the C-terminal portion of DNMT3L set forth in SEQ ID NO: 286. In some of any of the provided embodiments, the DNMT3L domain comprises the sequence set forth in SEQ ID NO:197, a C-terminal portion thereof, or an amino acid sequence that has at least 90%, 91%, 92%, 93%, 94%, 95%, 96%, 97%, 98%, or 99% sequence identity to SEQ ID NO: 197. In some of any of the provided embodiments, the DNMT3L comprises the sequence set forth in SEQ ID NO:287, or an amino acid sequence that has at least 90%, 91%, 92%, 93%, 94%, 95%, 96%, 97%, 98%, or 99% sequence identity to SEQ ID NO: 287. In some embodiments, the DNMT3L domain is or includes the C-terminal portion of DNMT3L set forth in SEQ ID NO: 287.

In some of any of the provided embodiments, the at least one transcriptional repressor domain is a DNMT3A-DNMT3L fusion protein domain or a variant thereof that exhibits transcriptional repressor activity. In some embodiments, the DNMT3A domain is set forth in SEQ ID NO:285 and the DNMT3L domain is set forth in SEQ ID NO: 286, which can be present in any order. In some embodiments, a linker is present between the DNMT3A domain and the DNMT3L domain. In some of any of the provided embodiments, the at least one transcriptional repressor domain comprises the sequence set forth in SEQ ID NO:199 or SEQ ID NO:201, a portion thereof, or an amino acid sequence that has at least 90%, 91%, 92%, 93%, 94%, 95%, 96%, 97%, 98%, or 99% sequence identity to SEQ ID NO: 199 or SEQ ID NO: 201.

In some of any of the provided embodiments, the at least one transcriptional repressor domain is fused to the N-terminus, the C-terminus, or both the N-terminus and the C-terminus, of the DNA-binding domain. In some of any of the provided embodiments, the fusion protein further comprises one or more nuclear localization signals (NLS). In some of any of the provided embodiments, the fusion protein further comprises one or more linkers connecting two or more of: the DNA-binding domain, the at least one transcriptional repressor domain, and the one or more nuclear localization signals.

In some embodiments, the fusion protein is a dCas9-KRAB or a variant thereof.

In some of any of the provided embodiments, the fusion protein comprises the sequence set forth in any one of SEQ ID NOS:209, or an amino acid sequence that has at least 90%, 91%, 92%, 93%, 94%, 95%, 96%, 97%, 98%, or 99% sequence identity to SEQ ID NO: 209. In some embodiments, the fusion protein has the sequence set forth in SEQ ID NO: 209.

In any of the embodiments herein, the fusion protein is a DNMT3A/3L-dCas9-KRAB domain or a variant thereof.

In any of the embodiments herein, the fusion protein comprises the sequence set forth in any one of SEQ ID NOs: 278, 280, or 282, or an amino acid sequence that has at least 90%, 91%, 92%, 93%, 94%, 95%, 96%, 97%, 98%, or 99% sequence identity to any one of SEQ ID NOS: 278, 280, or 282. In some embodiments, the fusion protein comprises the sequence set forth in SEQ ID NO:280. In some embodiments, the fusion protein has the sequence set forth in SEQ ID NO: 278. In some embodiments, the fusion protein has the sequence set forth in SEQ ID NO: 280. In some embodiments, the fusion protein has the sequence set forth in SEQ ID NO: 282.

In some of any of the provided embodiments, each of the DNA-targeting modules reduces expression of each of the respective genes by a log 2 fold-change of at or lesser than −1.0.

In some of any of the provided embodiments, repressed transcription of the plurality of genes in a cell or population of cells leads to a reduction of low-density lipoprotein (LDL). In some of any of the provided embodiments, the reduction of LDL is greater than the reduction of LDL resulting from comparable repressed transcription of any of the individual genes in the plurality of genes alone. In some of any of the provided embodiments, the reduction of LDL occurs extracellularly. In some of any of the provided embodiments, the cell or population of cells is a liver cell or comprises liver cells. In some of any of the provided embodiments, the cell or population of cells is in a subject. In some of any of the provided embodiments, the reduction of LDL occurs in the subject or a fluid, tissue, or organ thereof. In some of any of the provided embodiments, the reduction of LDL occurs in the blood of a subject.

In some aspects, provided herein is an epigenetic-modifying DNA-targeting system for repressing transcription of a gene that regulates low-density lipoprotein, comprising a fusion protein comprising: (a) a DNA-binding domain for targeting to a target site of a gene, and (b) at least one transcriptional repressor domain. In some of any of the provided embodiments, the target site of the gene is in the gene or a regulatory DNA element thereof. In some of any of the provided embodiments, the DNA-targeting system does not introduce a genetic disruption or a DNA break. In some of any of the provided embodiments, the DNA-binding domain is selected from: a Clustered Regularly Interspaced Short Palindromic Repeats associated (Cas) protein or a variant thereof; a zinc finger protein (ZFP); a transcription activator-like effector (TALE); a meganuclease; a homing endonuclease; or an I-SceI enzyme or a variant thereof, optionally wherein the DNA-binding domain comprises a catalytically inactive variant of any of the foregoing. In some of any of the provided embodiments, the DNA-binding domain is a Clustered Regularly Interspaced Short Palindromic Repeats associated (Cas) protein or variant thereof, and the system further comprises a gRNA for targeting the DNA-binding domain to the target site of the gene.

In some aspects, provided herein is an epigenetic-modifying DNA-targeting system comprising: (a) a fusion protein comprising a Clustered Regularly Interspaced Short Palindromic Repeats associated (Cas) protein or variant thereof and at least one transcriptional repressor domain; and (b) a gRNA that targets a target site of a gene that regulates low-density lipoprotein (LDL).

In some of any of the provided embodiments, the target site of the gene is in the gene and/or a regulatory DNA element thereof. In some of any of the provided embodiments, the regulatory DNA element is a promoter or enhancer. In some of any of the provided embodiments, the gene is selected from the group consisting of: PCSK9, LPA, MYLIP, ANGPTL3, APOB, and APOC3. In some of any of the provided embodiments, the target site is selected from: (a) a target site for PCSK9, located within 500 bp of human genome assembly GRCh38 (hg38) genomic coordinates chr1:55,039,548; (b) a target site for LPA, located within 500 bp of the hg38 genomic coordinates chr6:160,664,275; (c) a target site for MYLIP, located within 500 bp of the hg38 genomic coordinates chr6:16,129,086; (d) a target site for ANGPTL3, located within 500 bp of the hg38 genomic coordinates chr1:62,597,520; (e) a target site for APOC3, located within 500 bp of the hg38 genomic coordinates chr11:116,829,907; and (f) a target site for APOB, located within 500 bp of the hg38 genomic coordinates chr2:21,044,073. In some of any of the provided embodiments, the target site is selected from: (a) a target site located within 500 bp of a transcriptional start site of PCSK9; (b) a target site located within 500 bp of a transcriptional start site of LPA; (c) a target site located within 500 bp of a transcriptional start site of MYLIP; (d) a target site located within 500 bp of a transcriptional start site of ANGPTL3; (e) a target site located within 500 bp of a transcriptional start site of APOC3; and (f) a target site located within 500 bp of a transcriptional start site of APOB. In some of any of the provided embodiments, the target site is selected from: (a) a target site for PCSK9 having the sequence set forth in any one of SEQ ID NOS:1-13 or 306-317, a contiguous portion thereof of at least 14 nucleotides (nt), or a complementary sequence of any of the foregoing; (b) a target site for LPA having the sequence set forth in any one of SEQ ID NOS:14-23, a contiguous portion thereof of at least 14 nucleotides (nt), or a complementary sequence of any of the foregoing; (c) a target site for MYLIP having the sequence set forth in any one of SEQ ID NOS:24-33 or 342-351, a contiguous portion thereof of at least 14 nucleotides (nt), or a complementary sequence of any of the foregoing; (d) a target site for ANGPTL3 having the sequence set forth in any one of SEQ ID NOS:34-43, a contiguous portion thereof of at least 14 nucleotides (nt), or a complementary sequence of any of the foregoing; (e) a target site for APOC3 having the sequence set forth in any one of SEQ ID NOS:44-53, a contiguous portion thereof of at least 14 nucleotides (nt), or a complementary sequence of any of the foregoing; and (f) a target site for APOB having the sequence set forth in any one of SEQ ID NOS:54-63 or 372-377, a contiguous portion thereof of at least 14 nucleotides (nt), or a complementary sequence of any of the foregoing. In some embodiments, the target site for PCSK9 has (i) the sequence set forth in SEQ ID NO:3, (ii) a contiguous portion of at least 14 nucleotides set forth in SEQ ID NO:3, or (iii) a complementary sequence of (i) or (ii). In some of any of the provided embodiments, the target site is selected from: (a) a target site of PCSK9 having the sequence set forth in any one of SEQ ID NOS:1-13 or 306-317; (b) a target site of LPA having the sequence set forth in any one of SEQ ID NOS:14-23; (c) a target site of MYLIP having the sequence set forth in any one of SEQ ID NOS:24-33 or 342-351; (d) a target site of ANGPTL3 having the sequence set forth in any one of SEQ ID NOS: 34-43; (e) a target site of APOC3 having the sequence set forth in any one of SEQ ID NOS:44-53; and (f) a target site of APOB having the sequence set forth in any one of SEQ ID NOS:54-63 or 372-377. In some embodiments, the target site for PCSK9 has the sequence set forth in SEQ ID NO:3.

In some of any of the provided embodiments, the Cas protein or variant thereof is a variant Cas protein that is a deactivated (dCas) protein. In some of any of the provided embodiments, the dCas protein lacks nuclease activity. In some of any of the provided embodiments, the dCas protein is a dCas9 protein. In some of any of the provided embodiments, the dCas protein is a dCasl2 protein. In some of any of the provided embodiments, the dCas9 protein is a Staphylococcus aureus dCas9 (dSaCas9) protein. In some of any of the provided embodiments, the dSaCas9 comprises at least one amino acid mutation selected from D10A and N580A, with reference to numbering of positions of SEQ ID NO:204. In some of any of the provided embodiments, the dSaCas9 protein comprises the sequence set forth in SEQ ID NO:205, or an amino acid sequence that has at least 90%, 91%, 92%, 93%, 94%, 95%, 96%, 97%, 98%, or 99% sequence identity thereto. In some of any of the provided embodiments, the dSaCas9 is set forth in SEQ ID NO:205. In some of any of the provided embodiments, the dCas9 protein is a Streptococcus pyogenes dCas9 (dSpCas9) protein. In some of any of the provided embodiments, the dSpCas9 protein comprises at least one amino acid mutation selected from D10A and H840A, with reference to numbering of positions of SEQ ID NO:206. In some of any of the provided embodiments, the dSpCas9 comprises the sequence set forth in SEQ ID NO:207, or an amino acid sequence that has at least 90%, 91%, 92%, 93%, 94%, 95%, 96%, 97%, 98%, or 99% sequence identity thereto. In some of any of the provided embodiments, the dSpCas9 is set forth in SEQ ID NO:207.

In some of any of the provided embodiments, the gRNA comprises a gRNA spacer that is complementary to the target site of the gene. In some of any of the provided embodiments, the gRNA is selected from: (a) a gRNA targeting a target site of PCSK9 comprising a gRNA spacer sequence comprising the sequence set forth in any one of SEQ ID NOS:64-76 or 318-329, or a contiguous portion thereof of at least 14 nt; (b) a gRNA targeting a target site of LPA comprising a gRNA spacer sequence comprising the sequence set forth in any one of SEQ ID NOS:77-86, or a contiguous portion thereof of at least 14 nt; (c) a gRNA targeting a target site of MYLIP comprising a gRNA spacer sequence comprising the sequence set forth in any one of SEQ ID NOS:87-96 or 352-361, or a contiguous portion thereof of at least 14 nt; (d) a gRNA targeting a target site of ANGPTL3 comprising a gRNA spacer sequence comprising the sequence set forth in any one of SEQ ID NOS:97-106, or a contiguous portion thereof of at least 14 nt; (e) a gRNA targeting a target site of APOC3 comprising a gRNA spacer sequence comprising the sequence set forth in any one of SEQ ID NOS:107-116, or a contiguous portion thereof of at least 14 nt; and (f) a gRNA targeting a target site of APOB comprising a gRNA spacer sequence comprising the sequence set forth in any one of SEQ ID NOS:117-126 or 378-383, or a contiguous portion thereof of at least 14 nt. In some embodiments, the gRNA targeting a target site of PCSK9 comprises a gRNA spacer sequence set forth in SEQ ID NO:66 or a contiguous portion thereof of at least 14 nt.

In some of any of the provided embodiments, the gRNA comprises a spacer sequence between 14 nt and 24 nt, or between 16 nt and 22 nt in length. In some of any of the provided embodiments, the gRNA comprises a spacer sequence that is 18 nt, 19 nt, 20 nt, 21 nt, or 22 nt in length. In some of any of the provided embodiments, the gRNA is selected from: (a) a gRNA targeting a target site of PCSK9 comprising the gRNA spacer sequence set forth in any one of SEQ ID NOS:64-76 or 318-329; (b) a gRNA targeting a target site in LPA comprising the gRNA spacer sequence set forth in any one of SEQ ID NOS:77-86; (c) a gRNA targeting a target site in MYLIP comprising the gRNA spacer sequence set forth in any one of SEQ ID NOS:87-96 or 352-361; (d) a gRNA targeting a target site in ANGPTL3 comprising the gRNA spacer sequence set forth in any one of SEQ ID NOS:97-106; (e) a gRNA targeting a target site in APOC3 comprising the gRNA spacer sequence set forth in any one of SEQ ID NOS:107-116; and (f) a gRNA targeting a target site in APOB comprising the gRNA spacer sequence set forth in any one of SEQ ID NOS:117-126 or 378-383. In some embodiments, the gRNA targeting a target site of PCSK9 comprises a gRNA spacer sequence set forth in SEQ ID NO:66. In some of any of the provided embodiments, the gRNA further comprises a scaffold sequence set forth in SEQ ID NO:191. In some of any of the provided embodiments, the gRNA is selected from: (a) a gRNA targeting a target site in PCSK9 comprising the sequence set forth in any one of SEQ ID NOS:127-139 or 330-341; (b) a gRNA targeting a target site in LPA comprising the sequence set forth in any one of SEQ ID NOS:140-149; (c) a gRNA targeting a target site in MYLIP comprising the sequence set forth in any one of SEQ ID NOS:150-159 or 362-371; (d) a gRNA targeting a target site in ANGPTL3 comprising the sequence set forth in any one of SEQ ID NOS:160-169; (e) a gRNA targeting a target site in APOC3 comprising the sequence set forth in any one of SEQ ID NOS:170-179; and (f) a gRNA targeting a target site in APOB comprising the sequence set forth in any one of SEQ ID NOS:180-189 or 384-389. In some embodiments, the gRNA targeting a target site of PCSK9 comprises the sequence set forth in SEQ ID NO:129. In some of any of the provided embodiments, the gRNA is selected from: (a) a gRNA targeting a target site in PCSK9 set forth in any one of SEQ ID NOS:127-139 or 330-341; (b) a gRNA targeting a target site in LPA set forth in any one of SEQ ID NOS:140-149; (c) a gRNA targeting a target site in MYLIP set forth in any one of SEQ ID NOS:150-159 or 362-371; (d) a gRNA targeting a target site in ANGPTL3 set forth in any one of SEQ ID NOS:160-169; (e) a gRNA targeting a target site in APOC3 set forth in any one of SEQ ID NOS:170-179; and (f) a gRNA targeting a target site in APOB set forth in any one of SEQ ID NOS:180-189 or 384-389. In some embodiments, the gRNA targeting a target site of PCSK9 is set forth in SEQ ID NO:129. In some of any of the provided embodiments, the gRNA comprises modified nucleotides for increased stability.

In some of any of the provided embodiments, the at least one transcriptional repressor domain is capable of reducing transcription of the gene. In some of any of the provided embodiments, the transcriptional repressor domain is selected from the group consisting of a KRAB domain, a DNMT3A domain, a DNMT3L domain, a DNMT3B domain, a DNMT3A-DNMT3L fusion protein domain, an ERF repressor domain, an Mxi1 repressor domain, a SID4X repressor domain, a Mad-SID repressor domain, an LSD1 repressor domain, an EZH2 repressor domain, a SunTag domain, or a variant or portion of any of the foregoing, or a combination of any of the foregoing. In some of any of the provided embodiments, the transcriptional repressor domain is a KRAB domain, a DNMT3A domain, or a DNMT3L domain, or a combination of any of the foregoing. In some of any of the provided embodiments, at least one transcriptional repressor domain comprises a sequence selected from any one of SEQ ID NOS:193, 195, 197, 199, 201, 220-226, 283, 284, 285, 286, 287, 289 and 290 or a domain thereof, a portion thereof, or an amino acid sequence that has at least 90%, 91%, 92%, 93%, 94%, 95%, 96%, 97%, 98%, or 99% sequence identity to any one of SEQ ID NOS:193, 195, 197, 199, 201, 220-226, 283, 284, 285, 286, 287, 289 and 290.

In some of any of the provided embodiments, the at least one transcriptional repressor domain comprises a KRAB domain or a variant or portion thereof that exhibits transcriptional repressor activity. In some of any of the provided embodiments, the at least one transcriptional repressor domain comprises the sequence set forth in SEQ ID NO:193, a portion thereof, or an amino acid sequence that has at least 90%, 91%, 92%, 93%, 94%, 95%, 96%, 97%, 98%, or 99% sequence identity to SEQ ID NO: 193. In some of any of the provided embodiments, the at least one transcriptional repressor domain comprises the sequence set forth in SEQ ID NO:290, a portion thereof, or an amino acid sequence that has at least 90%, 91%, 92%, 93%, 94%, 95%, 96%, 97%, 98%, or 99% sequence identity to SEQ ID NO:290. In some of any above embodiments, the KRAB domain, portion or variant thereof is a sequence that exhibits transcriptional repressor activity.

In some of any of the provided embodiments, the at least one transcriptional repressor domain comprises a DNMT3A domain or a variant or portion thereof that exhibits transcriptional repressor activity. In some embodiments, the DNMT3A domain, or portion thereof, is from a human DNMT3A. In some of any of the provided embodiments, the at least one effector domain comprises the sequence set forth in SEQ ID NO:284, or is a portion thereof, or an amino acid sequence that has at least 90%, 91%, 92%, 93%, 94%, 95%, 96%, 97%, 98%, or 99% sequence identity to SEQ ID NO: 284. In some embodiments, the portion thereof is a catalytically active portion that exhibits transcription repressor activity. In some of any of the provided embodiments, the at least one transcriptional repressor domain comprises the sequence set forth in SEQ ID NO:195, a portion thereof, or an amino acid sequence that has at least 90%, 91%, 92%, 93%, 94%, 95%, 96%, 97%, 98%, or 99% sequence identity to SEQ ID NO: 195. In some embodiments, the at least one effector domain is or includes the catalytically active portion of DNMT3A set forth in SEQ ID NO: 195. In some of any of the provided embodiments, the at least one effector domain comprises the sequence set forth in SEQ ID NO:285, or is a portion thereof, or an amino acid sequence that has at least 90%, 91%, 92%, 93%, 94%, 95%, 96%, 97%, 98%, or 99% sequence identity to SEQ ID NO: 285. In some embodiments, the at least one effector domain is or includes the catalytically active protion of DNMT3A set forth in SEQ ID NO: 285.

In some of any of the provided embodiments, the at least one transcriptional repressor domain comprises a DNMT3L domain or a variant or portion thereof that exhibits transcriptional repressor activity. In some of any of the provided embodiments, the at least one transcriptional repressor domain is a fusion domain comprising a DNMT3A domain, such as any DNMT3A domain or C-terminal portion or variant described above, and a DNMT3L domain or a variant or portion thereof, in which the fusion domain exhibits transcriptional repressor activity. In some embodiments, DNMT3L domain, or the the variant or C-terminal portion of a DNMT3L domain, is one that stimulates enhanced transcription repressor activity of DNMT3A. In some embodiments, DNMT3L domain, or the the variant or C-terminal portion of a DNMT3L domain, is one that interacts with DNMT3A. In some embodiments, the DNMT3L domain, or C-terminal portion thereof, is from a murine DNMT3L. In some embodiments, the DNMT3L domain, or C-terminal portion thereof, is a human or humanized DNMT3L. In some of any of the provided embodiments, the DNTML domain comprises the sequence set forth in SEQ ID NO:289, a C-terminal portion thereof, or an amino acid sequence that has at least 90%, 91%, 92%, 93%, 94%, 95%, 96%, 97%, 98%, or 99% sequence identity to SEQ ID NO: 289. In some of any of the provided embodiments, the DNMT3L comprises the sequence set forth in SEQ ID NO:286, or an amino acid sequence that has at least 90%, 91%, 92%, 93%, 94%, 95%, 96%, 97%, 98%, or 99% sequence identity to SEQ ID NO: 286. In some embodiments, the DNMT3L domain is or includes the C-terminal portion of DNMT3L set forth in SEQ ID NO: 286. In some of any of the provided embodiments, the DNMT3L domain comprises the sequence set forth in SEQ ID NO:197, a C-terminal portion thereof, or an amino acid sequence that has at least 90%, 91%, 92%, 93%, 94%, 95%, 96%, 97%, 98%, or 99% sequence identity to SEQ ID NO: 197. In some of any of the provided embodiments, the DNMT3L comprises the sequence set forth in SEQ ID NO:287, or an amino acid sequence that has at least 90%, 91%, 92%, 93%, 94%, 95%, 96%, 97%, 98%, or 99% sequence identity to SEQ ID NO: 287. In some embodiments, the DNMT3L domain is or includes the C-terminal portion of DNMT3L set forth in SEQ ID NO: 287.

In some of any of the provided embodiments, the at least one transcriptional repressor domain is a DNMT3A-DNMT3L fusion protein domain or a variant thereof that exhibits transcriptional repressor activity. In some embodiments, the DNMT3A domain is set forth in SEQ ID NO:285 and the DNMT3L domain is set forth in SEQ ID NO: 286, which can be present in any order. In some embodiments, a linker is present between the DNMT3A domain and the DNMT3L domain. In some of any of the provided embodiments, the at least one transcriptional repressor domain comprises the sequence set forth in SEQ ID NO:199 or SEQ ID NO:201, a portion thereof, or an amino acid sequence that has at least 90%, 91%, 92%, 93%, 94%, 95%, 96%, 97%, 98%, or 99% sequence identity to SEQ ID NO: 199 or SEQ ID NO: 201.

In some of any of the provided embodiments, the at least one transcriptional repressor domain is fused to the N-terminus, the C-terminus, or both the N-terminus and the C-terminus, of the DNA-binding domain. In some of any of the provided embodiments, the fusion protein further comprises one or more nuclear localization signals (NLS). In some of any of the provided embodiments, the fusion protein further comprises one or more linkers connecting two or more of: the DNA-binding domain, the at least one transcriptional repressor domain, and the one or more nuclear localization signals.

In some embodiments, the fusion protein is a dCas9-KRAB or a variant thereof.

In some of any of the provided embodiments, the fusion protein comprises the sequence set forth in any one of SEQ ID NOS:209, or an amino acid sequence that has at least 90%, 91%, 92%, 93%, 94%, 95%, 96%, 97%, 98%, or 99% sequence identity to SEQ ID NO: 209. In some embodiments, the fusion protein has the sequence set forth in SEQ ID NO: 209.

In any of the embodiments herein, the fusion protein is a DNMT3A/3L-dCas9-KRAB domain or a variant thereof.

In any of the embodiments herein, the fusion protein comprises the sequence set forth in any one of SEQ ID NOs: 278, 280, or 282, or an amino acid sequence that has at least 90%, 91%, 92%, 93%, 94%, 95%, 96%, 97%, 98%, or 99% sequence identity to any one of SEQ ID NOS: 278, 280, or 282. In some embodiments, the fusion protein has the sequence set forth in SEQ ID NO: 278. In some embodiments, the fusion protein has the sequence set forth in SEQ ID NO: 280. In some embodiments, the fusion protein has the sequence set forth in SEQ ID NO: 282.

In any of the embodiments herein, the fusion protein comprises the sequence set forth in SEQ ID NO: 280, or an amino acid sequence that has at least 90%, 91%, 92%, 93%, 94%, 95%, 96%, 97%, 98%, or 99% sequence identity to SEQ ID NO: 280 and a gRNA that targets a target site of PCSK9 comprising the spacer sequence set forth in SEQ ID NO:66. In some embodiments, the gRNA has the sequence set forth in SEQ ID NO: 129.

In some of any of the provided embodiments, the epigenetic-modifying DNA-targeting system reduces expression of a gene by a log 2 fold-change of at or lesser than −1.0.

In some of any of the provided embodiments, repressed transcription of the gene in a cell or population of cells leads to a reduction of low-density lipoprotein (LDL). In some of any of the provided embodiments, the reduction of LDL occurs extracellularly. In some of any of the provided embodiments, the cell or population of cells is a liver cell or comprises liver cells.

In some of any of the provided embodiments, the cell or population of cells is in a subject. In some of any of the provided embodiments, the reduction of LDL occurs in the subject or a fluid, tissue, or organ thereof. In some of any of the provided embodiments, the reduction of LDL occurs in the blood of a subject.

In some aspects, provided herein is a combination of epigenetic-modifying DNA-targeting systems comprising at least two of the DNA-targeting systems provided herein, wherein each DNA-targeting system represses transcription of a different gene. In some of any of the provided embodiments, each DNA-targeting system represses transcription of a different gene.

In some aspects, provided herein is a guide RNA (gRNA) that targets a target site of a gene that regulates low-density lipoprotein (LDL). In some of any of the provided embodiments, the gene is selected from the group consisting of PCSK9, LPA, MYLIP, ANGPTL3, APOB, and APOC3. In some of any of the provided embodiments, the target site of the gene is in the gene or a regulatory DNA element thereof. In some of any of the provided embodiments, the regulatory DNA element is an enhancer or a promoter. In some of any of the provided embodiments, the target site is selected from the group consisting of: (a) a target site for PCSK9, located within 500 bp of human genome assembly GRCh38 (hg38) genomic coordinates chr1:55,039,548; (b) a target site for LPA, located within 500 bp of the hg38 genomic coordinates chr6:160,664,275; (c) a target site for MYLIP, located within 500 bp of the hg38 genomic coordinates chr6:16,129,086; (d) a target site for ANGPTL3, located within 500 bp of the hg38 genomic coordinates chr1:62,597,520; (e) a target site for APOC3, located within 500 bp of the hg38 genomic coordinates chr11:116,829,907; and (f) a target site for APOB, located within 500 bp of the hg38 genomic coordinates chr2:21,044,073. In some of any of the provided embodiments, the target site is selected from the group consisting of: (a) a target site located within 500 bp of a transcriptional start site of PCSK9; (b) a target site located within 500 bp of a transcriptional start site of LPA; (c) a target site located within 500 bp of a transcriptional start site of MYLIP; (d) a target site located within 500 bp of a transcriptional start site of ANGPTL3; (e) a target site located within 500 bp of a transcriptional start site of APOC3; and (f) a target site located within 500 bp of a transcriptional start site of APOB. In some of any of the provided embodiments, the target site is selected from: (a) a target site for PCSK9 having the sequence set forth in any one of SEQ ID NOS:1-13 or 306-317, a contiguous portion thereof of at least 14 nucleotides (nt), or a complementary sequence of any of the foregoing; (b) a target site for LPA having the sequence set forth in any one of SEQ ID NOS:14-23, a contiguous portion thereof of at least 14 nucleotides (nt), or a complementary sequence of any of the foregoing; (c) a target site for MYLIP having the sequence set forth in any one of SEQ ID NOS:24-33 or 342-351, a contiguous portion thereof of at least 14 nucleotides (nt), or a complementary sequence of any of the foregoing; (d) a target site for ANGPTL3 having the sequence set forth in any one of SEQ ID NOS:34-43, a contiguous portion thereof of at least 14 nucleotides (nt), or a complementary sequence of any of the foregoing; (e) a target site for APOC3 having the sequence set forth in any one of SEQ ID NOS:44-53, a contiguous portion thereof of at least 14 nucleotides (nt), or a complementary sequence of any of the foregoing; and (f) a target site for APOB having the sequence set forth in any one of SEQ ID NOS:54-63 or 372-377, a contiguous portion thereof of at least 14 nucleotides (nt), or a complementary sequence of any of the foregoing. In some embodiments, the target site has (i) the sequence set forth in SEQ ID NO: 3, (ii) a contiguous portion of SEQ ID NO:3 of at least 14 nt, or (iii) a complementary sequence of (i) or (ii). In some of any of the provided embodiments, the target site is selected from: (a) a target site of PCSK9 having the sequence set forth in any one of SEQ ID NOS:1-13 or 306-317; (b) a target site of LPA having the sequence set forth in any one of SEQ ID NOS:14-23; (c) a target site of MYLIP having the sequence set forth in any one of SEQ ID NOS:24-33 or 342-351; (d) a target site of ANGPTL3 having the sequence set forth in any one of SEQ ID NOS: 34-43; (e) a target site of APOC3 having the sequence set forth in any one of SEQ ID NOS:44-53; and (f) a target site of APOB having the sequence set forth in any one of SEQ ID NOS:54-63 or 372-377. In some embodiments, the target site has the sequence set forth in SEQ ID NO: 3.In some of any of the provided embodiments, the gRNA is selected from: (a) a gRNA targeting a target site of PCSK9 comprising a gRNA spacer sequence comprising the sequence set forth in any one of SEQ ID NOS:64-76 or 318-329, or a contiguous portion thereof of at least 14 nt; (b) a gRNA targeting a target site of LPA comprising a gRNA spacer sequence comprising the sequence set forth in any one of SEQ ID NOS:77-86, or a contiguous portion thereof of at least 14 nt; (c) a gRNA targeting a target site of MYLIP comprising a gRNA spacer sequence comprising the sequence set forth in any one of SEQ ID NOS:87-96 or 352-361, or a contiguous portion thereof of at least 14 nt; (d) a gRNA targeting a target site of ANGPTL3 comprising a gRNA spacer sequence comprising the sequence set forth in any one of SEQ ID NOS:97-106, or a contiguous portion thereof of at least 14 nt; (e) a gRNA targeting a target site of APOC3 comprising a gRNA spacer sequence comprising the sequence set forth in any one of SEQ ID NOS:107-116, or a contiguous portion thereof of at least 14 nt; and (f) a gRNA targeting a target site of APOB comprising a gRNA spacer sequence comprising the sequence set forth in any one of SEQ ID NOS:117-126 or 378-383, or a contiguous portion thereof of at least 14 nt. In some embodiments, the gRNA targeting a target site of PCSK9 comprises a gRNA spacer sequence comprising the sequence set forth in SEQ ID NO: 66 or a continuous portion thereof of at least 14 nt. In some of any of the provided embodiments, the gRNA comprises a spacer sequence between 14 nt and 24 nt, or between 16 nt and 22 nt in length. In some of any of the provided embodiments, the gRNA comprises a spacer sequence that is 18 nt, 19 nt, 20 nt, 21 nt, or 22 nt in length. In some of any of the provided embodiments, the gRNA is selected from: (a) a gRNA targeting a target site of PCSK9 comprising the gRNA spacer sequence set forth in any one of SEQ ID NOS:64-76 or 318-329; (b) a gRNA targeting a target site in LPA comprising the gRNA spacer sequence set forth in any one of SEQ ID NOS:77-86; (c) a gRNA targeting a target site in MYLIP comprising the gRNA spacer sequence set forth in any one of SEQ ID NOS:87-96 or 352-361; (d) a gRNA targeting a target site in ANGPTL3 comprising the gRNA spacer sequence set forth in any one of SEQ ID NOS:97-106; (e) a gRNA targeting a target site in APOC3 comprising the gRNA spacer sequence set forth in any one of SEQ ID NOS:107-116; and (f) a gRNA targeting a target site in APOB comprising the gRNA spacer sequence set forth in any one of SEQ ID NOS:117-126 or 378-383. In some embodiments, the gRNA targeting a target site of PCSK9 comprises a gRNA spacer sequence set forth in SEQ ID NO: 66. In some of any of the provided embodiments, the gRNA further comprises a scaffold sequence set forth in SEQ ID NO:191. In some of any of the provided embodiments, the gRNA is selected from: (a) a gRNA targeting a target site in PCSK9 comprising the sequence set forth in any one of SEQ ID NOS:127-139 or 330-341; (b) a gRNA targeting a target site in LPA comprising the sequence set forth in any one of SEQ ID NOS:140-149; (c) a gRNA targeting a target site in MYLIP comprising the sequence set forth in any one of SEQ ID NOS:150-159 or 362-371; (d) a gRNA targeting a target site in ANGPTL3 comprising the sequence set forth in any one of SEQ ID NOS:160-169; (e) a gRNA targeting a target site in APOC3 comprising the sequence set forth in any one of SEQ ID NOS:170-179; or (f) a gRNA targeting a target site in APOB comprising the sequence set forth in any one of SEQ ID NOS:180-189 or 384-389. In some embodiments, the gRNA targeting a target site in PCSK9 comprises the sequence set forth in SEQ ID NO: 129. In some of any of the provided embodiments, the gRNA is selected from: (a) a gRNA targeting a target site in PCSK9 set forth in any one of SEQ ID NOS:127-139 or 330-341; (b) a gRNA targeting a target site in LPA set forth in any one of SEQ ID NOS:140-149; (c) a gRNA targeting a target site in MYLIP set forth in any one of SEQ ID NOS:150-159 or 362-371; (d) a gRNA targeting a target site in ANGPTL3 set forth in any one of SEQ ID NOS:160-169; (e) a gRNA targeting a target site in APOC3 set forth in any one of SEQ ID NOS:170-179; or (f) a gRNA targeting a target site in APOB set forth in any one of SEQ ID NOS:180-189 or 384-389. In some embodiments, the gRNA targeting a target site in PCSK9 is set forth in SEQ ID NO: 129 In some of any of the provided embodiments, the gRNA comprises modified nucleotides for increased stability.

In some aspects, provided herein is a plurality of gRNAs comprising at least a first gRNA and a second gRNA, wherein the first gRNA targets a target site of a first gene that regulates low-density lipoprotein (LDL) and the second gRNA targets a target site of a second gene that regulates LDL. In some of any of the provided embodiments, the plurality of gRNAs comprises a third gRNA that targets a target site of a third gene that regulates LDL, and optionally the plurality of gRNAs further comprises a fourth gRNA that targets a target site of a fourth gene that regulates LDL, optionally a fifth gRNA that targets a target site of a fifth gene that regulates LDL, and/or optionally a sixth gRNA that targets a target site of a sixth gene that regulates LDL. In some of any of the provided embodiments, each gRNA is selected from any gRNA provided herein. In some of any of the provided embodiments, the first gene and the second gene are independently selected from the group consisting of PCSK9, LPA, MYLIP, ANGPTL3, APOB, and APOC3. In some of any of the provided embodiments, the first gene and the second gene are different. In some of any of the provided embodiments, the first gene and the second gene are: PCSK9 and LPA; PCSK9 and MYLIP; PCSK9 and ANGPTL3; PCSK9 and APOC3; PCSK9 and APOB; LPA and MYLIP; LPA and ANGPTL3; LPA and APOC3; LPA and APOB; MYLIP and ANGPTL3; MYLIP and APOC3; MYLIP and APOB; ANGPTL3 and APOC3; ANGPTL3 and APOB; or APOC3 and APOB. In some of any of the provided embodiments, the first gene, the second gene, and the third gene are each independently selected from the group consisting of PCSK9, LPA, MYLIP, ANGPTL3, APOC3, and APOB. In some of any of the provided embodiments, the first gene, the second gene, and the third gene are different. In some of any of the provided embodiments, the first gene, the second gene, and the third gene are: PCSK9, LPA, and MYLIP; PCSK9, LPA, and ANGPTL3; PCSK9, LPA, and APOC3; PCSK9, LPA, and APOB; PCSK9, MYLIP, and ANGPTL3; PCSK9, MYLIP, and APOC3; PCSK9, MYLIP, and APOB; PCSK9, ANGPTL3, and APOC3; PCSK9, ANGPTL3, and APOB; PCSK9, APOC3, and APOB; LPA, MYLIP, and ANGPTL3; LPA, MYLIP, and APOC3; LPA, MYLIP, and APOB; LPA, ANGPTL3, and APOC3; LPA, ANGPTL3, and APOB; LPA, APOC3, and APOB; MYLIP, ANGPTL3, and APOC3; MYLIP, ANGPTL3, and APOB; MYLIP, APOC3, and APOB; or ANGPTL3, APOC3, and APOB. In some of any of the provided embodiments, at least one gene is PCSK9. In some of any of the provided embodiments, at least two genes are PCSK9 and LPA. In some of any of the provided embodiments, at least three genes are PCSK9, LPA, and MYLIP. In some of any of the provided embodiments, the target site of each of the plurality of genes is in the gene or a regulatory DNA element thereof.

In some aspects, provided herein is a Cas-guide RNA (gRNA) combination comprising: (a) a Clustered Regularly Interspaced Short Palindromic Repeats associated (Cas) protein or variant thereof, and (b) any gRNA or plurality of gRNAs provided herein. In some of any of the provided embodiments, the Cas protein or variant thereof is a variant Cas protein that is a deactivated (dCas) protein. In some of any of the provided embodiments, the dCas protein lacks nuclease activity. In some of any of the provided embodiments, the dCas protein is a dCas9 protein. In some of any of the provided embodiments, the dCas protein is a dCasl2 protein. In some of any of the provided embodiments, the dCas9 protein is a Staphylococcus aureus dCas9 (dSaCas9) protein. In some of any of the provided embodiments, the dSaCas9 comprises at least one amino acid mutation selected from D10A and N580A, with reference to numbering of positions of SEQ ID NO:204. In some of any of the provided embodiments, the dSaCas9 protein comprises the sequence set forth in SEQ ID NO:205, or an amino acid sequence that has at least 90%, 91%, 92%, 93%, 94%, 95%, 96%, 97%, 98%, or 99% sequence identity thereto. In some of any of the provided embodiments, the dSaCas9 is set forth in SEQ ID NO:205. In some of any of the provided embodiments, the dCas9 protein is a Streptococcus pyogenes dCas9 (dSpCas9) protein. In some of any of the provided embodiments, the dSpCas9 protein comprises at least one amino acid mutation selected from D10A and H840A, with reference to numbering of positions of SEQ ID NO:206. In some of any of the provided embodiments, the dSpCas9 comprises the sequence set forth in SEQ ID NO:207, or an amino acid sequence that has at least 90%, 91%, 92%, 93%, 94%, 95%, 96%, 97%, 98%, or 99% sequence identity thereto. In some of any of the provided embodiments, the dSpCas9 is set forth in SEQ ID NO:207.

In some aspects, provided herein is a polynucleotide encoding any epigenetic-modifying DNA-targeting system, combination of epigenetic-modifying DNA-targeting systems, gRNA, plurality of gRNAs, or Cas-gRNA combination provided herein, or a portion or a component of any of the foregoing. In some aspects, provided herein is a polynucleotide encoding any epigenetic-modifying DNA-targeting system provided herein. In some aspects, provided herein is a polynucleotide encoding at least one DNA-targeting module of any epigenetic-modifying DNA-targeting system provided herein. In some aspects, provided herein is a polynucleotide encoding the fusion protein and the at least first gRNA and second gRNA of any epigenetic-modifying DNA-targeting system provided herein. In some aspects, provided herein is a polynucleotide encoding the fusion protein of any epigenetic-modifying DNA-targeting system provided herein. In some aspects, provided herein is a polynucleotide encoding the fusion protein and gRNA of any epigenetic-modifying DNA-targeting system provided herein. In some aspects, provided herein is a polynucleotide encoding any combination of epigenetic-modifying DNA-targeting systems provided herein. In some aspects, provided herein is a polynucleotide encoding any gRNA provided herein. In some aspects, provided herein is a polynucleotide encoding any plurality of gRNAs provided herein. In some aspects, provided herein is a polynucleotide encoding any Cas-gRNA combination provided herein.

In some aspects, provided herein is a plurality of polynucleotides encoding any epigenetic-modifying DNA-targeting system, combination of epigenetic-modifying DNA-targeting systems, plurality of gRNAs, or Cas-gRNA combination provided herein, or a portion or a component of any of the foregoing. In some aspects, provided herein is a plurality of polynucleotides encoding any epigenetic-modifying DNA-targeting system provided herein. In some aspects, provided herein is a plurality of polynucleotides encoding at least one DNA-targeting module of any epigenetic-modifying DNA-targeting system provided herein. In some aspects, provided herein is a plurality of polynucleotides encoding the fusion protein and the at least first gRNA and second gRNA of any epigenetic-modifying DNA-targeting system provided herein. In some aspects, provided herein is a plurality of polynucleotides encoding the fusion protein of any epigenetic-modifying DNA-targeting system provided herein. In some aspects, provided herein is a plurality of polynucleotides encoding the fusion protein and gRNA of any epigenetic-modifying DNA-targeting system provided herein. In some aspects, provided herein is a plurality of polynucleotides encoding any combination of epigenetic-modifying DNA-targeting systems provided herein. In some aspects, provided herein is a plurality of polynucleotides encoding any plurality of gRNAs provided herein. In some aspects, provided herein is a plurality of polynucleotides encoding any Cas-gRNA combination provided herein.

In some aspects, provided herein is a vector comprising any polynucleotide provided herein. In some aspects, provided herein is a vector comprising any plurality of polynucleotides provided herein. In some of any of the provided embodiments, the vector is a viral vector. In some of any of the provided embodiments, the vector is an adeno-associated virus (AAV) vector. In some of any of the provided embodiments, the vector is selected from among AAV1, AAV2, AAV3, AAV4, AAV5, AAV6, AAV7, AAV8, and AAV9. In some of any of the provided embodiments, the vector is a lentiviral vector. In some of any of the provided embodiments, the vector is a non-viral vector. In some of any of the provided embodiments, the non-viral vector is selected from: a lipid nanoparticle, a liposome, an exosome, or a cell penetrating peptide. In some of any of the provided embodiments, the non-viral vector is a lipid nanoparticle. In some of any of the provided embodiments, the vector exhibits hepatocyte tropism. In some of any of the provided embodiments, the vector comprises one vector, or two or more vectors. In some aspects, provided herein is a lipid nanoparticle comprising any polynucleotide or plurality of polynucleotides provided herein.

In some aspects, provided herein is a method of decreasing transcription of at least two genes in a cell or population of cells, the method comprising administering to a cell or population of cells any epigenetic-modifying DNA-targeting system, any combination of epigenetic-modifying DNA-targeting systems, any gRNA, any plurality of gRNAs, any Cas-gRNA combination, any polynucleotide, any plurality of polynucleotides, or any vector or lipid nanoparticle provided herein, or a portion or a component of any of the foregoing. In some of any of the provided embodiments, the at least two genes are epigenetically modified. In some of any of the provided embodiments, the transcription of each of the at least two genes is decreased in comparison to a comparable cell or population of cells not subjected to the method. In some of any of the provided embodiments, the transcription of each of the at least two genes is reduced by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold. In some of any of the provided embodiments, the reduced transcription of each of the at least two genes leads to a reduction of low-density lipoprotein (LDL). In some of any of the provided embodiments, the reduction of LDL resulting from reduced transcription each of the at least two genes is greater than the reduction of LDL resulting from comparable reduced transcription of any individual gene of the at least two genes alone.

In some aspects, provided herein is a method of reducing LDL, the method comprising introducing into a cell or population of cells any epigenetic-modifying DNA-targeting system, any combination of epigenetic-modifying DNA-targeting systems, any gRNA, any plurality of gRNAs, any Cas-gRNA combination, any polynucleotide, any plurality of polynucleotides, or any vector or lipid nanoparticle provided hereinor a portion or a component of any of the foregoing. In some of any of the provided embodiments, the cell or population of cells is a liver cell or comprises liver cells. In some of any of the provided embodiments, the cell or population of cells is in a subject and the method is carried out in vivo. In some of any of the provided embodiments, LDL is reduced in the subject or a fluid, tissue, or organ thereof. In some of any of the provided embodiments, LDL is reduced in the blood of a subject. In some of any of the provided embodiments, the subject is a human. In some of any of the provided embodiments, the subject has or is suspected of having a disease, condition, or disorder. In some embodiments, the disease, condition, or disorder is a cardiovascular disease. In some of any of the provided embodiments, the subject has or is suspected of having one or more of: elevated levels of low-density lipoprotein in the blood, increased risk of cardiovascular disease, increased risk of early-onset cardiovascular disease, a mutation affecting cholesterol biosynthesis, a loss-of-function mutation in a low-density lipoprotein receptor (LDLR) gene, a loss-of-function mutation in APOB, a gain-of-function mutation in PCSK9, and familial hypercholesterolemia. In some of any of the provided embodiments, the subject has is or is suspected of having familial hypercholesterolemia.

In some aspects, provided herein is a pharmaceutical composition comprising any epigenetic-modifying DNA-targeting system, any combination of epigenetic-modifying DNA-targeting systems, any gRNA, any plurality of gRNAs, any Cas-gRNA combination, any polynucleotide, any plurality of polynucleotides, or any vector or lipid nanoparticle provided herein, or a portion or a component of any of the foregoing. In some of any of the provided embodiments, the pharmaceutical composition is for use in treating a disease, condition, or disorder in a subject. In some embodiments, the disease, condition or disorder is a cardiovascular disease. In some of any of the provided embodiments, use of the pharmaceutical composition is for the manufacture of a medicament for treating a disease, condition, or disorder in a subject. In some embodiments, the disease, condition or disorder is a cardiovascular disease. In some of any of the provided embodiments, the subject has or is suspected of having a disease, condition, or disorder. In some embodiments, the disease, condition or disorder is a cardiovascular disease. In some of any of the provided embodiments, the subject has or is suspected of having one or more of: elevated levels of low-density lipoprotein in the blood, increased risk of cardiovascular disease, increased risk of early-onset cardiovascular disease, a mutation affecting cholesterol biosynthesis, a loss-of-function mutation in a low-density lipoprotein receptor (LDLR) gene, a loss-of-function mutation in APOB, a gain-of-function mutation in PCSK9, and familial hypercholesterolemia. In some of any of the provided embodiments, the subject has is or is suspected of having familial hypercholesterolemia. In some of any of the provided embodiments, the pharmaceutical composition is to be administered to the subject in vivo. In some of any of the provided embodiments, the pharmaceutical composition is targeted to, or is to be administered to the liver of the subject. In some of any of the provided embodiments, following administration of the pharmaceutical composition, the expression of at least two genes is reduced in cells of the subject. In some of any of the provided embodiments, following administration of the pharmaceutical composition, the expression of at least two genes is reduced in liver cells of the subject. In some of any of the provided embodiments, the at least two genes are selected from the group consisting of: PCSK9, LPA, MYLIP, ANGPTL3, APOB, and APOC3.

In some aspects, provided herein is a method for treating a disease, condition, or disorder associated with elevated low-density lipoprotein (LDL) in a subject in need thereof, comprising administering to the subject any epigenetic-modifying DNA-targeting system, any combination of epigenetic-modifying DNA-targeting systems, any gRNA, any plurality of gRNAs, any Cas-gRNA combination, any polynucleotide, any plurality of polynucleotides, any vector or lipid nanoparticle, or any pharmaceutical composition provided herein, or a portion or a component of any of the foregoing. In some of any of the provided embodiments, the subject has or is suspected of having one or more of: elevated levels of low-density lipoprotein in the blood, increased risk of cardiovascular disease, increased risk of early-onset cardiovascular disease, a mutation affecting cholesterol biosynthesis, a loss-of-function mutation in a low-density lipoprotein receptor (LDLR) gene, a loss-of-function mutation in APOB, a gain-of-function mutation in PCSK9, and familial hypercholesterolemia. In some aspects the disease, condition or disorder associated with elevated LDL is a cardiovascular disease.

In some aspects, provided herein is a method for treating a familial hypercholesterolemia in a subject, comprising administering to the subject any epigenetic-modifying DNA-targeting system, any combination of epigenetic-modifying DNA-targeting systems, any gRNA, any plurality of gRNAs, any Cas-gRNA combination, any polynucleotide, any plurality of polynucleotides, any vector or lipid nanoparticle, or any pharmaceutical composition provided herein, or a portion or a component of any of the foregoing. In some of any of the provided embodiments, the administration is a single dose infusion to the subject. In some of any of the provided embodiments, the administration is repeated at least once. In some embodiments, the administration is repeated a plurality of times at regular intervals. In some embodiments, the administration is a multiple dose administration comprising at least a first dose and a second dose. In some embodiments, the first dose and the second dose are the same. In some embodiments, the second dose is lower than the first dose. In some embodiments, the second dose is 25% to 75% of the first dose. In some embodiments, the second dose is about 30%, about 40%, about 50%, about 60% or about 70%, or a percentage between any of the foregoing, of the first dose. In some embodiments, the second dose is higher than the first dose. In some embodiments, the second dose is 150% to 500% of the first dose. In some embodiments, the second dose is about 200%, about 300%, about 400% or about 500%, or a percentage between any of the foregoing, of the first dose. In some of any of the embodiments, any lipid nanoparticle provided herein is administered to the subject.

In some of any of the embodiments, the pharmaceutical composition is for single dose infusion to the subject. In some of any of the embodiments, the pharmaceutical composition is for repeated dose administration. In some embodiments, the pharmaceutical composition is for repeated dose administration a plurality of times at regular intervals. In some embodiments, the administration is a multiple dose administration comprising at least a first dose and a second dose. In some embodiments, the first dose and the second dose are the same. In some embodiments, the second dose is lower than the first dose. In some embodiments, the second dose is 25% to 75% of the first dose. In some embodiments, the second dose is about 30%, about 40%, about 50%, about 60% or about 70%, or a percentage between any of the foregoing, of the first dose. In some embodiments, the second dose is higher than the first dose. In some embodiments, the second dose is 150% to 500% of the first dose. In some embodiments, the second dose is about 200%, about 300%, about 400% or about 500%, or a percentage between any of the foregoing, of the first dose. In some of any of the embodiments, the pharmaceutical composition comprises any lipid nanoparticle provided herein.

### BRIEF DESCRIPTION OF THE DRAWINGS

FIG. 1 shows results from qRT-PCR to assess expression of PCSK9 mRNA in murine liver (AML12) cells transfected with dSpCas9-KRAB-DNMT3A/L and a non-targeting gRNA (negative control) or PCSK9-targeting gRNAs. Dots represent expression levels for experimental replicates, bars represent mean of expression from experimental replicates. Expression is shown as fold change with respect to cells expressing the non-targeting gRNA.

FIG. 2 shows results from qRT-PCR to assess expression of genes associated with regulation of low-density lipoprotein (LDL) in human hepatocellular carcinoma (Huh7) cells transfected with dSpCas9-KRAB-DNMT3A/L and a non-targeting gRNA (NT; negative control) or a gRNA targeting human PCSK9, LPA, MYLIP, ANGPTL3, APOC3, or APOB. Results are shown for 7 days post-transfection. Dots represent expression levels for experimental replicates, bars represent mean of expression from experimental replicates. Expression is shown as fold change with respect to cells expressing the non-targeting gRNA.

FIG. 3A-3E show PCSK9 mRNA expression levels and PCSK9 CpG methylation in human hepatocellular carcinoma (Huh7) cells transfected with dSpCas9-KRAB-DNMT3A/L and a non-targeting gRNA (NT; negative control) or a PCSK9-targeting gRNA.

FIG. 3A shows results from qRT-PCR to assess expression of PCSK9 mRNA in transfected Huh cells on day 3, day 7, day 14, day 21, day 30, day 40, day 50, and day 62post-transfection.

FIG. 3B shows results from qRT-PCR to assess expression of PCSK9 mRNA in transfected Huh cells on day 120 post-transfection. Dots represent expression levels for experimental replicates, bars represent mean of expression from experimental replicates. Expression is shown as fold change with respect to cells expressing the non-targeting gRNA.

FIG. 3C shows results from qRT-PCR to assess PCSK9 mRNA expression levels in Huh cells transfected with dSpCas9-KRAB-DNMT3A/L and a non-targeting gRNA (NT) or gRNA PCSK9-C from day 3 through day 180 post-transfection. Dots represent individual time points and error bars represent mean of expression from experimental replicates. FIG. 3D shows the % methylation of CpGs in the PCSK9 promoter in Huh cells transfected with dSpCas9-KRAB-DNMT3A/L and a non-targeting gRNA (NT) or gRNA PCSK9-C. The methylation profiles are shown for cells expressing the non-targeting gRNA (NT) at day 103 post-transfection and for cells expressing PCSK9-C at day 21and day 103 post-transfection. The shaded bar indicates the location of the target site for gRNA PCSK9-C in the PCSK9 promoter. FIG. 3E shows the methylation profiles of CpG islands 1-22 at the PCSK9 promoter for Huh cells expressing the non-targeting gRNA (NT) at day 103 post-transfection and for cells expressing PCSK9-C at day 21 and day 103 post-transfection. The shaded bar indicates the location of the target site for gRNA PCSK9-C in the PCSK9 promoter.

FIG. 4A-4B show PCSK9 mRNA and protein levels in Cynomolgus macaque primary hepatocytes transfected with dSpCas9-KRAB-DNMT3A/L and a non-targeting gRNA (NT), a human PCSK9-targeting gRNA, or a cynomolgus-specific PCSK9-gRNA (cPCSK9-C). FIG. 4A shows results from qRT-PCR to assess expression of Cynomolgus PCSK9 (cPCSK9) mRNA levels in transfected hepatocytes at day 3 and day 14 post-transfection. mRNA expression is shown as fold change with respect to cells expressing the non-targeting gRNA. FIG. 4B shows PCSK9 protein levels as measured by ELISA in transfected hepatocytes at day 3 and day 14 post-transfection. Dots represent values for experimental replicates, bars represent mean values for experimental replicates.

FIG. 5 shows blood PCSK9 protein levels from three individual Cynomolgus macaques (NHP1, NHP2, and NHP3) intravenously infused with a high dose (3.0 mg/kg) of dSpCas9-KRAB-DNMT3A/L and gRNA cPCSK9-C. Protein levels are shown as % change from baseline at numerous time points spanning 49 days post-infusion for NHP1 and 28 days post-infusion for NHP2 and NHP3.

FIG. 6 shows average blood PCSK9 protein levels from Cynomolgus macaques intravenously infused with a high dose (3.0 mg/kg, n=3) of dSpCas9-KRAB-DNMT3A/L and gRNA cPCSK9-C as compared to a PBS control dose (n=2). Protein levels are shown as % change from baseline at numerous time points spanning 100 days post-infusion for the PBS treated group and 120 days post-infusion for high dose treated group. Dots represent individual time points and error bars represent mean of expression from experimental replicates.

FIG. 7 shows LDL-C measurements from three individual Cynomolgus macaques (NHP1, NHP2, and NHP3) intravenously infused with a high dose (3.0 mg/kg) of dSpCas9-KRAB-DNMT3A/L and gRNA cPCSK9-C. Blood LDL-C levels are shown as % of baseline at numerous time points spanning 49 days post-infusion for NHP1 and 28 days post-infusion for NHP2 and NHP3.

FIG. 8 shows average LDL-C levels from Cynomolgus macaques intravenously infused with a high dose (3.0 mg/kg, n=3) of dSpCas9-KRAB-DNMT3A/L and gRNA cPCSK9-C as compared to a PBS control dose (n=2). Blood LDL-C levels are shown as % change from baseline at numerous time points spanning 100 days post-infusion for the PBS treated group and 120 days post-infusion for high dose treated group. Dots represent individual time points and error bars represent mean of expression from experimental replicates.

FIG. 9A-9B show alanine transaminase (ALT) and asparate aminotransferase (AST) measurements from three individual Cynomolgus macaques (NHP1, NHP2, and NHP3) intravenously infused with a high dose (3.0 mg/kg) of dSpCas9-KRAB-DNMT3A/L and gRNA cPCSK9-C. FIG. 9A shows ALT levels for each NHP at various timepoints spanning 14 days pre-infusion through 28 days post-infusion. FIG. 9B shows AST levels for each NHP at various timepoints spanning 14 days pre-infusion through 28 days post-infusion.

FIG. 10A-10B show average alanine transaminase (ALT) and asparate aminotransferase (AST) measurements from Cynomolgus macaques intravenously infused with a high dose (3.0 mg/kg, n=3) of dSpCas9-KRAB-DNMT3A/L and gRNA cPCSK9-C. FIG. 10A shows average ALT levels at various timepoints spanning 0-120 days days post-infusion. FIG. 10B shows average AST levels at various timepoints spanning 0-120 days post-infusion.

FIG. 11 shows blood PCSK9 protein levels from three individual Cynomolgus macaques (NHP4, NHP5, and NHP6) intravenously infused with a low dose (1.0 mg/mL) of dSpCas9-KRAB-DNMT3A/L and gRNA cPCSK9-C and re-dosed at 1.0 mg/kg 21 days after the initial infusion. A dashed oval marks the re-dose time point. Protein levels are shown as % change from baseline at numerous time points spanning 28 days after the initial infusion.

FIG. 12A-C show LDL-C, alanine transaminase (ALT), and asparate aminotransferase (AST) measurements from three individual Cynomolgus macaques (NHP4, NHP5, and NHP6) intravenously infused with a low dose (1.0 mg/kg) of dSpCas9-KRAB-DNMT3A/L and gRNA cPCSK9-C and re-dosed at 1.0 mg/kg 21 days after the initial infusion. A dashed oval marks the re-dose time point. FIG. 12A shows LDL-C levels as % of baseline at numerous time points spanning 28 days after the initial infusion. FIG. 12B shows ALT levels for each NHP at various timepoints spanning 14 days pre-infusion through 28 days post-infusion. FIG. 12C shows AST levels for each NHP at various timepoints spanning 14 days pre-infusion through 28 days post-infusion.

FIG. 13 shows PCSK9 mRNA levels measured from liver biopsies of Cynomolgus macaques intravenously infused with a PBS control, a high dose (3.0 mg/kg) of dSpCas9-KRAB-DNMT3A/L and gRNA cPCSK9-C, or a low dose (1.0 mg/kg) of dSpCas9-KRAB-DNMT3A/L and gRNA cPCSK9-C. Relative mRNA levels are shown from liver biopsies performed on day 7 post-infusion for each treatment group and biopsies performed on day 28 post-infusion for the low dose (1.0 mg/kg) and PBS treatment groups. mRNA levels are shown as fold change relative to the PBS controls. Dots represent values for experimental replicates, bars represent mean values for experimental replicates.

FIG. 14. shows PCSK9 mRNA levels measured from liver biopsies of three individual Cynomolgus macaques (NHP4, NHP5, and NHP6) following a third intravenous infusion of KRAB-DNMT3A/L and gRNA cPCSK9-C at varying doses 126 days after the initial infusion compared to a PBS control (NHP7). NHP4 and NHP5 were redosed at 3.0 mg/kg and NHP6 was redosed at 1.0 mg/kg. Relative mRNA levels are shown from liver biopsies performed on day 140 post-infusion for NHP4, NHP6, and NHP7 and a biopsy performed on day 129 post-infusion for NHP5. mRNA levels are shown as fold change relative to the PBS controls.

FIG. 15 shows average blood PCSK9 protein levels from Cynomolgus macaques intravenously infused with dSpCas9-KRAB-DNMT3A/L and gRNA cPCSK9-C at a high dose (3.0 mg/kg, n=3) and a low dose (1.0 mg/kg, n=3) as compared to a PBS control dose (n=2). 126 days after the initial infusion, NHP4 and NHP5 from the low dose group were redosed at 3.0 mg/kg and NHP6 was redosed at 1.0 mg/kg. Protein levels are shown as % change from baseline at numerous time points spanning 160 days post-infusion for the high dose treated group and 140 days post-infusion for the low dose and PBS treated groups. Dots represent individual time points and error bars represent mean of expression from experimental replicates.

FIG. 16 shows average LDL-C levels from Cynomolgus macaques intravenously infused with dSpCas9-KRAB-DNMT3A/L and gRNA cPCSK9-C at a high dose (3.0 mg/kg, n=3) and a low dose (1.0 mg/kg, n=3) as compared to a PBS control dose (n=2). 126 days after the initial infusion, NHP4 and NHP5 from the low dose group were redosed at 3.0 mg/kg and NHP6 was redosed at 1.0 mg/kg. Blood LDL-C levels are shown as % change from baseline at numerous time points spanning 160 days post-infusion for the high dose treated group and 140 days post-infusion for for the low dose and PBS treated groups. Dots represent individual time points and error bars represent mean of expression from experimental replicates.

FIG. 17 shows the % methylation of CpGs in the PCSK9 promoter of DNA extracted from liver biopsies of Cynomolgus macaques intravenously infused with a PBS control (n=2) or a high dose (3.0 mg/kg, n=3) of dSpCas9-KRAB-DNMT3A/L and gRNA cPCSK9-C. Liver biopsies were performed at day 7 and day 85 post-infusion. The shaded bar indicates the location of the target site for gRNA cPCSK9-C in the cPCSK9 promoter.

FIG. 18 shows the methylation signature surrounding the CpGs in the PCSK9 promoter for Cynomolgus macaques intravenously infused with a PBS control (n=2) or a high dose (3.0 mg/kg, n=3) of dSpCas9-KRAB-DNMT3A/L and gRNA cPCSK9-C at day 85 post-infusion. The shaded bar indicates the location of the target site for gRNA cPCSK9-C in the cPCSK9 promoter.

FIG. 19 shows PCSK9 protein levels plotted against the average % CpG methylation across the 2.2 kB PCSK9 promoter region for individual Cynomolgus macaques intravenously infused with a PBS control (NHP7 and NHP8), a low dose (1.0 mg/kg) of dSpCas9-KRAB-DNMT3A/L and gRNA cPCSK9-C(NHP4, NHP5, and NHP6), or a high dose (3.0 mg/kg) of dSpCas9-KRAB-DNMT3A/L and gRNA cPCSK9-C(NHP1, NHP2, and NHP3). Measurements were taken on day 7 post-infusion.

FIG. 20 shows results from qRT-PCR to assess PCSK9 mRNA expression levels in primary human hepatocytes transfected with dSpCas9-KRAB-DNMT3A/L and a non-targeting gRNA (NT) or gRNA PCSK9-C from day 3 through day 30 post-transfection. Dots represent individual time points and error bars represent mean of expression from experimental replicates.

### DETAILED DESCRIPTION

Provided herein is an epigenetic-modifying DNA-targeting system comprising one or more, such as a plurality of, DNA-targeting modules for repressing transcription of one or more, such as a plurality of, genes that regulate low-density lipoprotein (LDL). In some embodiments, provided epigenetic-modifying DNA-targeting systems for repressing transcription of a gene that regulates low-density lipoprotein includes fusion proteins comprising: (a) a DNA-binding domain for targeting to a target site of a gene that regulates LDL, and (b) at least one transcriptional repressor domain. In some embodiments, the gene associated associated with regulation of LDL includes PCSK9, LPA, MYLIP, ANGLPTL3, APOC3, and APOB, or any combination thereof. Also provided herein are epigenetic-modifying DNA-targeting systems that are multiplexed with a plurality of targeting modules such that the system is able to target a combination of such genes. In some embodiments, each module of the DNA-targeting system represses transcription of a different gene. Also provided herein are methods of using the epigenetic-modifying DNA-targeting systems for reducing LDL in a subject. In some embodiments, the methods can be used in therapies for reducing LDL, such as for treatment of Familial Hypercholestorlemia (FH).

FH is a genetic disorder of cholesterol metabolism that is characterized by abnormally elevated blood serum levels of low-density lipoprotein (LDL) cholesterol, which can lead to early-onset cardiovascular disease and death. Unless stated otherwise, reference to LDL throughout this disclosure refers to LDL cholesterol (LDL-C) as this form is a standard measure of LDL and LDL-attributable cardiovascular disease risk. FH is a relatively common disorder, affecting approximately 1 in 250 individuals world-wide. FH can result from mutations in a number of genes, several of which play a role in LDL cholesterol metabolism (Konstantina Valanti, E. et al., Metabolism 116:154461 (2020); Defesche, J. C. et al. Nat. Rev. Dis. Primers 3:17093 (2017); Bouhairie, V. E. et al. Cardiol. Clin. 33(2):169-179 (2015)).

The most common form of FH is an autosomal dominant disorder resulting from loss-of-function mutations in the gene that encodes the LDL receptor (LDLR), accounting for 85-90% of all FH cases. LDLR is a cell-surface receptor that mediates endocytosis of LDL, including in liver cells, thus reducing the concentration of circulating LDL. Loss-of-function LDLR mutations result in a decreased capacity to clear LDL from the bloodstream, and consequently elevated levels of circulating LDL.

Loss-of-function mutations in Apolipoprotein B (ApoB) are also associated with FH, accounting for approximately 5-10% of FH cases. ApoB acts as a protein component of LDL, and binds LDLR to facilitate endocytosis of LDL. FH-causing ApoB mutations commonly disrupt the ability of ApoB to bind LDLR, thus reducing the rate of LDL clearance from the bloodstream.

Rare gain-of-function mutations in proprotein convertase subtilisin/kexin type 9 (PCSK9) are have also been associated with FH. PCSK9 negatively regulates cell surface expression of LDLR by binding to the epidermal growth factor-like repeat A (EGF-A) domain of LDLR and targeting LDLR for lysosomal degradation. Mutations resulting in increased PCSK9 activity thus decrease the ability of cells to express LDLR at the cell surface, resulting in elevated circulating LDL. Conversely, loss-of-function PCSK9 mutations are associated with lowered LDL levels and protection from cardiovascular disease (Lagace, T. A. Curr. Opin. Lipidol. 25(5):387-393 (2014); Peterson, A. S. et al. J. Lipid Res. 49(6):1152-1156 (2008); Bouhairie, V. E. et al. Cardiol. Clin. 33(2):169-179 (2015)).

Lipoprotein(a) (LPA) is an LDL-like particle identified as an independent risk factor for FH and cardiovascular disease. Elevated LPA affects approximately 20% of the general population, and is more prevalent in individuals with FH (30-50% of individuals with FH have elevated LPA), creating an added risk for cardiovascular disease.

The understanding of FH as a genetic disorder affecting cholesterol metabolism has led to a number of approaches for treating the disease (Bouhairie et al.). FH is typically treated initially with statins, a class of lipid-lowering medications also known as HMG-CoA reductase inhibitors, which reduce cholesterol synthesis. Statin therapy can reduce LDL in FH patients. However, for a number of FH patients, statins do not sufficiently reduce LDL, and/or the risk of negative cardiovascular events, including for patients with additional independent risk factors for cardiovascular disease, including elevated LPA levels. However, statins alone may be ineffective for certain FH patients, and are associated with a range of toxic side effects, including statin-associated muscle symptoms (Ward, N.C. et al. Circ. Res. 124:328-350 (2019)).

Combination therapies, typically comprising a statin and a secondary non-statin treatment, are required to sufficiently lower LDL in FH patients for whom statins alone are not effective (Bouhairie, V. E. et al. Cardiol. Clin. 33(2):169-179 (2015)). Some patients may additionally require a third treatment if statins and a secondary treatment are not effective. Secondary non-statin treatments may include ezetimibe, bile-acid sequestrants (such as colesevelam, colestipol, and cholestyramine), niacin, and fibrates. Such treatments are associated with a number of side effects, including gastrointestinal and metabolic dysregulation, as well as interference with other medications, including statins themselves.

LDL apheresis, in which LDL is removed from the blood of a patient in a clinical setting, is another potential treatment for FH patients, particularly those with homozygous loss-of-function LDLR mutations, and for whom the above-described treatments are not effective. The cost and intensive nature of LDL apheresis is disadvantageous, with LDL apheresis typically requiring a 3 hour treatment session every 1-2 weeks.

PCSK9 monoclonal antibodies have emerged as a promising secondary non-statin treatment to supplement statin treatment for FH. PCKS9 antibodies inhibit binding of PCKS9 to LDLR, thus reducing LDLR degradation and increasing LDL clearance. However, PCKS9 antibodies incur significant time commitment and cost, requiring injection once every 2 to 4 weeks.

In summary, current therapies for FH face a number of challenges. First-line treatment with statins alone can be ineffective for many patients, while combination therapies are complicated by toxic side-effects, high cost, or inconvenient treatment schedules. There is a need for alternative FH therapies that effectively reduce LDL and that exhibit reduced treatment outcomes, such as greater or more sustained efficacy.

Provided embodiments herein relate to leveraging engineered DNA-binding systems to target genetic mechanisms contributing to regulation of LDL, including for treatment of cardiovascular disease and FH. An understanding of the genetic risk factors of FH, combined with emerging technologies for targeted modulation of gene expression in vivo, present new opportunities for treating the disease. Sequence-specific DNA-binding systems found in nature, such as zinc-fingers, transcription-activator-like effectors, and CRISPR/Cas systems can now be engineered to target one or more genes for activation or repression in vivo (Adli, M. Nat. Commun. 9, 1911 (2018)). The provided embodiments also relate to engineering the DNA-binding systems to target more than one gene, thus eliminating the need for a combination therapy that would previously be required to do so, and the costs and inconvenience associated with such combination therapies.

The provided embodiments relate to targeted repression of one or more genes in liver cells to promote a phenotype that reduces LDL the blood, such as by increased expression of LDLR and/or increased LDL uptake. In aspects, the provided embodiments include introducing into a liver cell epigenetic modifications using effector domains that are repressors of transcription (i.e. transcriptional repressor domains), which can be directed to regions of a target gene (e.g. regulatory elements such as promoters or enhancers) for transcriptional repression and reduced expression of the target gene. For instance, provided herein are epigenetic-modifying DNA binding systems combining a DNA-binding domain (e.g. a dCas and gRNA combination) and an effector domain, in which the effector domain is able to target a target site of the gene or a regulatory element thereof to precisely repress or reduce transcription of the gene by epigenetic regulation. Transcriptional repression, leading to reduced gene expression, increases the capacity of the cell to clear LDL from the blood. Moreover, the epigenetic modification of the cell does not modify DNA at the sequence level, thereby avoiding safety concerns with gene editing approaches.

The ability to epigenetically control the capacity of liver cells to clear LDL provides an advantageous approach for treating FH and cardiovascular disease, eliminating the need for other therapies or combinations of therapies which can be ineffective, toxic, expensive, or inconvenient. In addition, the ability to target multiple genes for repression allows for reduction of LDL via more than one mechanism with a single treatment, thereby avoiding the need for complicated schedules and toxicity associated with some combination therapies. Epigenetic repression of genes may provide a long-lasting solution to reducing LDL, with the potential for administration schedules that are less intensive than current therapies.

In particular, among provided embodiments herein is an epigenetic-modifying DNA-targeting system for repressing transcription of a gene or a plurality of genes that regulates LDL, such as any described herein, in which the epigenetic-modifying DNA-targeting system comprises a fusion protein comprising: (a) at least one DNA-binding domain for targeting to a target site of a gene, and (b) at least one transcriptional repressor domain capable of repressing or reducing transcription of the gene. In some embodiments, the provided epigenetic-modifying DNA-targeting systems are for multiplexed targeted repression of a plurality of different genes that regulate LDL, in which the system contains a plurality of DNA-targeting modules each for repressing transcription of a different gene that regulates LDL. In some embodiments, the epigenetic-modifying DNA-targeting system comprises a plurality of DNA-targeting modules for repressing transcription of a plurality of genes that regulate LDL, wherein each DNA-targeting module comprises a fusion protein comprising: (a) a DNA-binding domain for targeting to a target site of one of the plurality of genes, and (b) at least one transcriptional repressor domain. In some embodiments, at least two different genes that regulate LDL are targeted for repression, wherein the plurality of DNA-targeting modules comprises a first DNA-targeting module for repressing transcription of a first gene of the plurality of genes, and a second DNA-targeting module for repressing transcription of a second gene of the plurality of genes. Also provided herein are polynucleotides encoding the DNA-targeting systems or fusion proteins of the DNA-targeting systems, vectors, and compositions containing the same.

In some embodiments of the provided epigenetic-modifying DNA-targeting system, the DNA binding domain is a nuclease-inactive Clustered Regularly Interspaced Short Palindromic Repeats associated (Cas) protein or variant thereof, such as a dead Cas (dCas, e.g. dCas9), and the DNA-targeting system further includes at least one gRNA that can complex with the Cas. In some embodiments, the DNA-binding domain is a nuclease-inactive Clustered Regularly Interspaced Short Palindromic Repeats associated (Cas) protein or variant thereof complexed with a guide RNA (gRNA). In such systems, the gRNA has a spacer sequence that is capable of hybridizing to the target site of the gene. Also provided herein are related gRNAs, including Cas/gRNA combinations, polynucleotides, compositions and methods involving or related to the epigenetic-modifying DNA targeting systems.

In some embodiments of the provided epigenetic-modifying DNA-targeting system, the DNA-binding domain is a protein domain that is engineered for sequence-specific binding to the target site. For example, in some embodiments, the DNA-binding domain is a zinc-finger (ZFN)-based DNA-binding domain, or transcription activator-like effector DNA-binding domain, as described herein.

Also provided herein are methods of using the epigenetic-modifying DNA-targeting system for modulating transcription or a phenotype of liver cells. Also provided herein are methods of using the epigenetic-modifying DNA-targeting systems for reducing LDL in a subject. In some embodiments, the methods can be used in therapies for reducing LDL, such as for treatment of FH.

Hence, in some embodiments, the DNA-targeting systems comprise synthetic transcription factors that are able to modulate, such as reduce or repress, transcription of a gene in a targeted manner. In provided embodiments, the provided epigenetic-modifying DNA-targeting system reduces transcription of the gene or plurality of genes, and thereby promotes an LDL-reducing phenotype. The provided embodiments can be used to target multiple genetic mechanisms to reduce LDL in FH patients or patients with cardiovascular disease, while avoiding the toxicity, cost, and inconvenience of current combination therapies. For example, artheoscherotic cardiovascular disease (ASCVD) is a chronic inflammatory disease caused by build-up of LDL plaque in the arteries. ASCVD is responsible for 1 in 4 deaths in the US, hundreds of millions globally, and is the underlying cause of ˜50% of all deaths in westernized society. In addition, 75% of acute heart attacks are due to sclerotic plaque rupture. Statins can be used to stabilize but do not reverse atherosclerotic plaques. However, the need for repeated administration and side effects of statin treatments limit uptake and compliance. Combination therapy comprising statins and PCSK9 antibodies is a promising alternative, but LDL levels remain too high in many patients. Gene editing approaches targeting PCSK9 create DNA mutations and can have off-target effects while RNAi based methods have not been proven durable. Thus, improved therapies are needed.

This approach provided herein, therefore, offers substantial clinical solutions to treating FH and cardiovascular disease, such as artheoscherotic cardiovascular disease (ASCVD), by reducing circulating LDL, while circumventing the problems associated with current therapies. In particular, provided embodiments relate to methods for targeted repression of PCSK9 by provided DNA-targeting systems. Results herein demonstrate a surprisingly high sustained level of repression of PCSK9 in serum of about 80% more than 22 weeks after a single transient dose to an animal model provided with a DNA-targeting repressor system specific to the PCSK9 promoter. Results demonstrate that silencing PCSK9 results in reduction of LDL-cholesterol and thus is a therapeutic target for the prevention or treatment of cardiovascular disease and FH. While currently approved PCSK9 inhibitors require repeat administration to maintain efficacy, the provided DNA-targeting systems for targeted repression of PCSK9 offers the potential to durably silence PCSK9 without altering the genetic sequence.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### L. DNA-Targeting Systems

In some embodiments, provided are DNA-targeting systems capable of specifically targeting a target site in at least one gene (also called a target gene herein) or DNA regulatory element thereof, and reducing transcription of the at least one gene, in which each of the at least one gene encodes a gene product that regulates LDL. In provided embodiments, for each target gene that is targeted, the DNA-targeting systems include a DNA-binding domain that binds to a target site in the gene or regulatory DNA element thereof. In some embodiments, the DNA-targeting systems additionally include at least one effector domain that is able to epigenetically modify one or more DNA bases of the gene or regulatory element thereof, in which the epigenetic modification results in a reduction in transcription of the gene (e.g. inhibits transcription or reduces transcription of the gene compared to the absence of the DNA-targeting system). Hence, the terms DNA-targeting system and epigenetic-modifying DNA-targeting system may be used herein interchangeably. In some embodiments, the DNA-targeting systems include a fusion protein comprising (a) at least one DNA-binding domain capable of being targeted to the target site; and (b) at least one effector domain capable of reducing transcription of the gene. For instance, the at least one effector domain is a transcription repressor domain.

In some embodiments, the DNA-targeting system contains at least one DNA-targeting module, where each DNA-targeting module of the system is a component of the DNA-targeting system that is independently capable of targeting one target site in a target gene as provided. In some embodiments, each DNA-targeting module includes (a) a DNA-binding domain capable of being targeted to a target site of the target gene that regulates LDL and (b) an effector domain capable of reducing transcription of the gene.

In some embodiments, the DNA-targeting system includes a single DNA-targeting module for targeting repression of a single gene. In some embodiments, the DNA-targeting module includes (a) a DNA-binding domain capable of being targeted to a target site of the target gene or regulatory element in which the target gene is encodes a gene product that regulates LDL, and (b) an effector domain capable of reducing transcription of the gene.

In some embodiments, the DNA-targeting system includes a plurality of DNA-targeting modules, in which each DNA-targeting module is for targeting repression of a different gene. In some embodiments, the DNA-targeting systems are multiplexed DNA-targeting systems, i.e. targeted to target sites in more than one gene. Hence, the terms DNA-targeting system may include a multiplexed epigenetic-modifying DNA targeting system that includes more than one DNA-targeting module. A multiplexed epigenetic-modifying DNA targeting system comprises at least 2, at least 3, at least 4, at least 5, at least 6, at least 7, at least 8, at least 9, at least 10, at least 20, at least 30 DNA-targeting modules or any value between any of the foregoing

In some embodiments, any two DNA-targeting modules of a DNA-targeting system comprise separate (i.e. non-overlapping) components. In some embodiments, each DNA-targeting modules of a DNA-targeting system comprise separate (i.e. non-overlapping) components. For example, a DNA-targeting system may comprise a first DNA-targeting module comprising a first fusion protein comprising a DNA-binding domain (e.g. a ZFN or TALE-based DNA-binding domain) that targets a first target site, and a second DNA-targeting module comprising a second fusion protein comprising a second DNA-binding domain (e.g. a ZFN or TALE-based DNA-binding domain) that targets a second target site.

In some embodiments, any two DNA-targeting modules of a DNA-targeting system may comprise shared (i.e. overlapping) components. In some embodiments, each DNA-targeting modules of a DNA-targeting system comprise shared (i.e. overlapping) components. For example, a DNA-targeting system may comprise a first DNA-targeting module comprising (a) a fusion protein comprising a Cas protein and a transcriptional repressor domain, and (b) a first gRNA that complexes with the Cas protein and targets a first target site, and a second DNA-targeting module comprising (a) the fusion protein of the first DNA-targeting module, and (b) a second gRNA that complexes with the Cas protein and targets a second target site. It will be understood that providing two or more different gRNAs for a given Cas protein allows different molecules of the same Cas protein to be targeted to the target sites of the two or more gRNAs. Conversely, different Cas protein variants (e.g. SpCas9 and SaCas9) are compatible with different gRNA scaffold sequences and PAMs, as described herein. Thus, it is possible to engineer a single DNA-targeting system comprising multiple non-overlapping CRISPR/Cas-based DNA-targeting modules.

In some aspects, provided herein is an epigenetic-modifying DNA-targeting system comprising a plurality of DNA-targeting modules for repressing transcription of a plurality of genes that regulate low-density lipoprotein (LDL). In some embodiments, the plurality of DNA-targeting modules comprises a first DNA-targeting module for repressing transcription of a first gene of the plurality of genes, and a second DNA-targeting module for repressing transcription of a second gene of the plurality of genes. In some embodiments, each DNA-targeting module comprises a fusion protein comprising: (a) a DNA-binding domain for targeting to a target site of one of the plurality of genes, and (b) at least one transcriptional repressor domain.

In some of any of the provided embodiments, the gene(s) is selected from the group consisting of PCSK9, LPA, MYLIP, ANGPTL3, APOB, and APOC3. In some embodiments, the gene is PCSK9.

In some aspects, provided herein is an epigenetic-modifying DNA-targeting system comprising a plurality of DNA-targeting modules for repressing transcription of a plurality of genes that regulate low-density lipoprotein (LDL), comprising: (1) a first DNA-targeting module that reduces transcription of a first gene that regulates low-density lipoprotein (LDL), wherein the first DNA-targeting module comprises a first fusion protein comprising (a) a DNA-binding domain for targeting a target site of the first gene or regulatory DNA element thereof, and (b) at least one transcriptional repressor domain; and (2) a second DNA-targeting module that reduces transcription of a second gene that regulates LDL, wherein the second DNA-targeting module comprises a second fusion protein comprising (a) a DNA-binding domain for targeting a target site of the second gene or regulatory DNA element thereof, and (b) at least one transcriptional repressor domain.

In some aspects, provided herein is an epigenetic-modifying DNA-targeting system comprising: (a) a fusion protein comprising a Clustered Regularly Interspaced Short Palindromic Repeats associated (Cas) protein or variant thereof and at least one transcriptional repressor domain; and (b) a plurality of guide RNAs (gRNAs) comprising at least a first gRNA and a second gRNA, wherein the first gRNA targets a target site of a first gene that regulates low-density lipoprotein (LDL) and the second gRNA targets a target site of a second gene that regulates LDL.

In some aspects, provided herein is an epigenetic-modifying DNA-targeting system for repressing transcription of a gene that regulates low-density lipoprotein, comprising a fusion protein comprising: (a) a DNA-binding domain for targeting to a target site of a gene, and (b) at least one transcriptional repressor domain.

In some aspects, provided herein is an epigenetic-modifying DNA-targeting system comprising: (a) a fusion protein comprising a Clustered Regularly Interspaced Short Palindromic Repeats associated (Cas) protein or variant thereof and at least one transcriptional repressor domain; and (b) a gRNA that targets a target site of a gene that regulates low-density lipoprotein (LDL).

In aspects of the provided embodiments, a DNA-targeting system provided herein targets a combination of genes or a regulatory elements thereof to reduce transcription of the genes in a liver cell, such as a hepatocyte, in which the reduced transcription modulates one or more activities or functions of liver cells, such as a phenotype of the liver cell. In some embodiments, reduced transcription of the genes results in a reduced expression of the genes, i.e. reduced gene expression, in the liver cell. In some embodiments, the reduced transcription of the gene, such as reduced gene expression, promotes a phenotype that leads to reduction of LDL, such as in a subject (i.e. an enhanced LDL-reducing phenotype).

In some aspects, the cell is a liver cell, such as a hepatocyte. In some aspects, the cell is a hepatocyte. For instance, provided herein is a DNA-targeting system that targets a combination of genes or a regulatory elements thereof to reduce transcription of the genes in a hepatocyte, in which the reduced transcription modulates one or more activities or functions of the hepatocyte, such as a phenotype of the hepatocyte. In some embodiments, reduced transcription of the genes results in a reduction in expression of the genes, i.e. reduced gene expression, in the hepatocyte.

In some aspects, the cell is from a human subject. In some aspects the cell is a cell in a subject (i.e. a cell in vivo).

In some embodiments, the DNA-binding domain comprises or is derived from a CRISPR associated (Cas) protein, zinc finger protein (ZFP), transcription activator-like effectors (TALE), meganuclease, homing endonuclease, I-SceI enzyme, or variants thereof. In some embodiments, the DNA-binding domain comprises a catalytically inactive (e.g. nuclease-inactive or nuclease-inactivated) variant of any of the foregoing. In some embodiments, the DNA-binding domain comprises a deactivated Cas9 (dCas9) protein or variant thereof that is a catalytically inactivated so that it is inactive for nuclease activity and is not able to cleave the DNA.

In some embodiments, the DNA-binding domain comprises or is derived from a Cas protein or variant thereof, such as a nuclease-inactive Cas or dCas (e.g. dCas9, and the DNA-targeting system comprises one or more guide RNAs (gRNAs), such as a combination of gRNAs (e.g. two gRNAs or three gRNAs). In some embodiments, the gRNA comprises a spacer sequence that is capable of targeting and/or hybridizing to the target site. In some embodiments, the gRNA is capable of complexing with the Cas protein or variant thereof. In some aspects, the gRNA directs or recruits the Cas protein or variant thereof to the target site.

In some embodiments, the effector domain comprises a transcription repressor domain, and/or is capable of reducing transcription of the gene. In some embodiments, the effector domain directly or indirectly leads to reduced transcription of the gene. In some embodiments, the effector domain induces, catalyzes or leads to transcription repression. In some embodiments, the effector domain induces transcription repression. In some aspects, the effector domain is selected from a KRAB domain, ERF repressor domain, MXI1 domain, SID4X domain, MAD-SID domain, a DNMT family protein domain (e.g. DNMT3A or DNMT3B), a fusion of one or more DNMT family proteins or domains thereof (e.g. DNMT3A/L, which comprises a fusion of DNMT3A and DNMT3L domains), LSD1, EZH2, a SunTag domain, a partially or fully functional fragment or domain of any of the foregoing, or a combination of any of the foregoing.. In some embodiments, the effector domain is KRAB. In some embodiments, the effector domain is DNMT3A/L.

In some embodiments, the fusion protein of the DNA-targeting system comprises a dCas9-KRAB fusion protein. In some embodiments, the fusion protein of the DNA-targeting system comprises a dCas9-KRAB-DNMT3A/L fusion protein. For purposes herein, unless a particular SEQ ID NO is identified or a particular order specified, the term dCas9-KRAB-DNMT3A/L may refer to any orientation of the fusion protein as described above. In some embodiments, the fusion protein of the DNA-targeting system comprises in N- to C-terminal order DNMT3A/L-dCas9-KRAB-fusion protein. In some embodiments, the fusion protein of the DNA-targeting system comprises in N- to C-terminal order KRAB-dCas9-DNMT3A/L-fusion protein.

Exemplary components and features of the DNA-targeting systems are provided below in the following subsections.

### A. Target Genes and Target Sites

In some embodiments, the target gene is a gene in which reduced expression of the gene regulates a cellular phenotype. In some embodiments, the target gene is capable of regulating a phenotype in a hepatocyte. In some embodiments, the target gene is capable of regulating the ability of a hepatocyte to reduce levels of LDL in the blood of a subject. In some embodiments, reduced transcription of the gene, such as reduced gene expression, promotes a phenotype in a hepatocyte that leads to reduction of LDL (i.e. an enhanced LDL-reducing phenotype).

In some aspects, the phenotype is one that is characterized by a cell surface phenotype of the cells. In some embodiments, the phenotype comprises increased expression of low-density lipoprotein receptor (LDL-R).

It is understood that a cell that is positive (+) for a particular cell surface marker is a cell that expresses the marker on its surface at a level that is detectable. Likewise, it is understood that a cell that is negative (−) for a particular cell surface marker is a cell that expresses the marker on its surface at a level that is not detectable. Antibodies and other binding entities can be used to detect expression levels of marker proteins to identify or detect a given cell surface marker. Suitable antibodies may include polyclonal, monoclonal, fragments (such as Fab fragments), single chain antibodies and other forms of specific binding molecules. Antibody reagents for cell surface markers above are readily known to a skilled artisan. A number of well-known methods for assessing expression level of surface markers or proteins may be used, such as detection by affinity-based methods, e.g., immunoaffinity-based methods, e.g., in the context of surface markers, such as by flow cytometry. In some embodiments, the label is a fluorophore and the method for detection or identification of cell surface markers on cells (e.g. hepatocytes) is by flow cytometry. In some embodiments, different labels are used for each of the different markers by multicolor flow cytometry. In some embodiments, surface expression can be determined by flow cytometry, for example, by staining with an antibody that specifically binds to the marker and detecting the binding of the antibody to the marker.

In some embodiments, a cell (e.g. hepatocyte) is positive (pos or +) for a particular marker if there is detectable presence on or in the cell of a particular marker, which can be an intracellular marker or a surface marker. In some embodiments, surface expression is positive if staining by flow cytometry is detectable at a level substantially above the staining detected carrying out the same procedures with an isotype-matched control under otherwise identical conditions and/or at a level substantially similar to, or in some cases higher than, a cell known to be positive for the marker and/or at a level higher than that for a cell known to be negative for the marker. In some embodiments, a cell (e.g. a hepatocyte) contacted by a DNA-targeting system described herein, has increased expression for a particular marker (e.g. LDL-R) if the staining is substantially than a similar cell that was not contacted by the DNA-targeting system.

In some embodiments, a cell (e.g. hepatocyte) is negative (neg or −) for a particular marker if there is an absence of detectable presence on or in the cell of a particular marker, which can be an intracellular marker or a surface marker. In some embodiments, surface expression is negative if staining is not detectable by flow cytometry at a level substantially above the staining detected carrying out the same procedures with an isotype-matched control under otherwise identical conditions and/or at a level substantially lower than a cell known to be positive for the marker and/or at a level substantially similar to a cell known to be negative for the marker.

In some aspects, the phenotype is one that is characterized functionally. In some aspects, the phenotype can be characterized by one or more functions of the cells. In some embodiments, the phenotype comprises increased uptake of LDL by the cell. In some embodiments, the phenotype comprises reduced expression of lipoprotein(a) (LPA), an LDL variant, by the cell. In some embodiments, the phenotype comprises reduced LDL in one or more tissues of a subject comprising the cell. In some embodiments, the phenotype comprises reduced LDL in the blood of a subject comprising the cell.

The target genes for modulation by the provided multiplexed epigenetic-modifying DNA-targeting systems herein include any whose transcription and expression are reduced in cells with a particular or desired function or activity, such as cell phenotype (e.g. a phenotype for reducing LDL). Various methods may be utilized to characterize the transcription or expression levels of a gene in a cell (e.g. hepatocyte) such as after the cell has been contacted or introduced with a provided DNA-targeting system and selected for a desired activity or function, such as cell phenotype. In some embodiments, the phenotype can be a phenotype comprising one or more cell surface markers as described above. In some embodiments, the phenotype is increased LDL-R expression. In some embodiments, analyzing the transcription activity or expression of a gene may be by RNA analysis. In some embodiments, the RNA analysis includes RNA quantification. In some embodiments, the RNA quantification occurs by reverse transcription quantitative PCR (RT-qPCR), multiplexed qRT-PCR, fluorescence in situ hybridization (FISH), FlowFISH, RNA-sequencing (RNA-seq) or combinations thereof.

In some embodiments, the gene is one in which expression of the gene in the cell (e.g. hepatocyte), is reduced after having been contacted or introduced with a provided multiplexed epigenetic DNA-targeting system. In some embodiments, the reduction in gene expression in a cell (e.g. hepatocyte) is about a log 2 fold change of less than −1.0. For instance, the log 2 fold change is lesser than at or about −1.5, at or about −2.0, at or about −2.5, at or about −3.0, at or about −4.0, at or about −5.0, at or about −6.0, at or about −7.0, at or about −8.0, at or about −9.0, at or about −10.0 or any value between any of the foregoing compared to the level of the gene in a control cell.

In some embodiments, the gene is selected from the list consisting of: PCSK9, LPA, MYLIP, ANGPTL3, APOC3, and APOB. In some embodiments, the target site comprises a sequence selected from any one of SEQ ID NOS: 1-63, 306-317, 342-351, or 372-377, a contiguous portion thereof of at least 14 nucleotides of any one of SEQ ID NOS: 1-63, 306-317, 342-351, or 372-377, or a complementary sequence of any of the foregoing. In some embodiments, the target site is a contiguous portion of any one of SEQ ID NOS: 1-63, 306-317, 342-351, or 372-377 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in any one of SEQ ID NOS: 1-63, 306-317, 342-351, or 372-377.

In some embodiments, the gene is PCSK9. PCSK9 is a gene that encodes Proprotein convertase subtilisin/kexin type 9 (also known as FH3, HCHOLA3, LDLCQ1, NARC-1, NARC1, PC9, FHCL3). PCSK9 plays a role in regulation of plasma cholesterol homeostasis, and binds low-density lipid receptor family members and promotes their degradation (Poirier et al., J. Biol. Chem. 283:2363-2372 (2008)). In some embodiments the target site for PCSK9 is located within 500 bp of human genome assembly GRCh38 (hg38) genomic coordinates chr1:55,039,548(e.g., a target site that is +500 of 55,039,548 or −500 of 55,039,548 or positions between the foregoing). In some embodiments, the target site is within 400 bp, 300 bp, 200 bp, 100 bp, 80 bp, 60 bp, 50 bp, 40 bp, 30 bp or 20 bp of genomic coordinates chr1:55,039,548. In some embodiments the target site is located within about 80 bp of the genomic coordinate chr1:55,039,548. In some embodiments, the target site in the region from −40 to +40 of the genomic coordinate chr1:55,039,548. In some embodiments the target site is located within 20 bp of the genomic coordinate chr1:55,039,548. In some embodiments, the gRNA targets a target site in the region from −10 to +10 of the genomic coordinate chr1:55,039,548. In some embodiments, any of such target sites include or span the genomic coordinate chr1:55,039,548, which is a PCSK9 transcription start site (TSS). In some embodiments, the target site is within or overlaps the coordinates chr1:55,039,538-55, 039,557. In some embodiments, the target site is or includes the coordinates chr1:55,039,538-55, 039,557. In some embodiments, the target site comprises a sequence selected from any one of SEQ ID NOS: 1-13 or 306-317, a contiguous portion thereof of at least 14 nucleotides of any one of SEQ ID NOS: 1-13 or 306-317, or a complementary sequence of any of the foregoing. In some embodiments, the target site is a contiguous portion of any one of SEQ ID NOS: 1-13 or 306-317 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in any one of SEQ ID NOS: 1-13 or 306-317. In some embodiments, the target site is the sequence set forth in SEQ ID NO:3 or a contiguous portion thereof of at least 14 nucleotides of SEQ ID NO:3. In some embodiments, the target site is the sequence set forth in SEQ ID NO:3.

In some embodiments, the gene is LPA. LPA is a gene that encodes Apolipoprotein(a) (also known as AK38, APOA, LP, Lipoprotein(a), Lp(a)). Apolipoprotein(a) is a constituent of lipoprotein(a), which has been identified as a risk factor for cardiovascular disease. In some embodiments, the target site for LPA is located within 500 bp of the hg38 genomic coordinates chr6:160,664,275. In some embodiments, the target site comprises a sequence selected from any one of SEQ ID NOS: 14-23, a contiguous portion thereof of at least 14 nucleotides of any one of SEQ ID NOS: 14-23, or a complementary sequence of any of the foregoing. In some embodiments, the target site is a contiguous portion of any one of SEQ ID NOS: 14-23 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in any one of SEQ ID NOS: 14-23.

In some embodiments, the gene is MYLIP. MYLIP is a gene that encodes myosin regulatory light chain interacting protein (also known as IDOL, MIR, myosin regulatory light chain interacting protein). MYLIP is an E3 ubiquitin-protein ligase that mediates ubiquitination and subsequent proteasomal degradation of myosin regulatory light chain (MRLC), LDLR, VLDLR and LRP8. MYLIP acts as a sterol-dependent inhibitor of cellular cholesterol uptake by mediating ubiquitination and subsequent degradation of LDLR. In some embodiments, the target site for MYLIP is located within 500 bp of the hg38 genomic coordinates chr6:16,129,086. In some embodiments, the target site comprises a sequence selected from any one of SEQ ID NOS: 24-33 or 342-351, a contiguous portion thereof of at least 14 nucleotides of any one of SEQ ID NOS: 24-33 or 342-351, or a complementary sequence of any of the foregoing. In some embodiments, the target site is a contiguous portion of any one of SEQ ID NOS: 24-33 or 342-351 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in any one of SEQ ID NOS: 24-33 or 342-351.

In some embodiments, the gene is ANGPTL3. ANGPTL3 is a gene that encodes Angiopoietin-related protein 3 (also known as ANG-5, ANGPT5, ANL3, FHBL2, angiopoietin like 3). ANGPTL3 is a member of the angiopoietin-like family of secreted factors, and is involved in regulation of lipid and glucose metabolism. In some embodiments, the target site for ANGPTL3 is located within 500 bp of the hg38 genomic coordinates chr1:62,597,520. In some embodiments, the target site comprises a sequence selected from any one of SEQ ID NOS: 34-43, a contiguous portion thereof of at least 14 nucleotides of any one of SEQ ID NOS: 34-43, or a complementary sequence of any of the foregoing. In some embodiments, the target site is a contiguous portion of any one of SEQ ID NOS: 34-43 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5% 99.9% or 100% sequence identity to all or a contiguous portion of a target site sequence described herein above. In some embodiments, the target site is the sequence set forth in any one of SEQ ID NOS: 34-43.

In some embodiments, the gene is APOC3. APOC3 is a gene that encodes Apolipoprotein C-III (also known as APOCIII, HALP2, apolipoprotein C3, Apo-C3, ApoC-3). APOC3 is a component of triglyceride-rich very low density lipoproteins (VLDL) and high density lipoproteins (HDL) in plasma, and plays a role in triglyceride homeostasis. In some embodiments, the target site for APOC3 is located within 500 bp of the hg38 genomic coordinates chr11:116,829,907. In some embodiments, the target site comprises a sequence selected from any one of SEQ ID NOS: 44-53, a contiguous portion thereof of at least 14 nucleotides of any one of SEQ ID NOS: 44-53, or a complementary sequence of any of the foregoing. In some embodiments, the target site is a contiguous portion of any one of SEQ ID NOS: 44-53 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in any one of SEQ ID NOS: 44-53.

In some embodiments, the gene is APOB. APOB is a gene that encodes Apolipoprotein B-100 (also known as FLDB, LDLCQ4, apoB-100, apoB-48, apolipoprotein B, FCHL2). APOB is a constituent of low density lipoproteins and functions as a recognition signal for the cellular binding and internalization of LDL particles by the apoB/E receptor. In some embodiments, the target site for APOB is located within 500 bp of the hg38 genomic coordinates chr2:21,044,073. In some embodiments, the target site comprises a sequence selected from any one of SEQ ID NOS: 54-63 or 372-377, a contiguous portion thereof of at least 14 nucleotides of any one of SEQ ID NOS: 54-63 or 372-377, or a complementary sequence of any of the foregoing. In some embodiments, the target site is a contiguous portion of any one of SEQ ID NOS: 54-63 or 372-377 that is 15, 16, 17, 18 or 19 nucleotides in length, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in any one of SEQ ID NOS: 54-63 or 372-377.

In some embodiments, the multiplexed epigenetic-modifying DNA-targeting system targets to or binds to a target site in the gene, such as any described above. In some embodiments, the target site is located in a regulatory DNA element of the gene in the cell (e.g. hepatocyte). In some embodiments, a regulatory DNA element is a sequence to which a gene regulatory protein may bind and affect transcription of the gene. In some embodiments, the regulatory DNA element is a cis, trans, distal, proximal, upstream, or downstream regulatory DNA element of a gene. In some embodiments, the regulatory DNA element is a promoter or enhancer of the gene. In some embodiments, the target site is located within a promoter, enhancer, exon, intron, untranslated region (UTR), 5′ UTR, or 3′ UTR of the gene. In some embodiments, a promoter is a nucleotide sequence to which RNA polymerase binds to begin transcription of the gene. In some embodiments, a promoter is a nucleotide sequence located within about 100 bp, about 500 bp, about 1000 bp, or more, of a transcriptional start site of the gene. In some embodiments the target site is located within a sequence of unknown or known function that is suspected of being able to control expression of a gene.

In some embodiments, provided herein are multiplexed epigenetic-modifying DNA-targeting systems that target a combination of at least two target genes or regulatory DNA elements thereof described herein.

In some embodiments, provided herein are multiplexed epigenetic-modifying DNA-targeting systems that target a first gene and a second gene. In some embodiments, the first gene is selected from the list consisting of PCSK9, LPA, MYLIP, ANGPTL3, APOC3, and APOB, the second gene is selected from the list consisting of PCSK9, LPA, MYLIP, ANGPTL3, APOC3, and APOB, and the first and second genes are different. In some embodiments, the first gene is PCSK9 and the second gene is selected from the list consisting of LPA, MYLIP, ANGPTL3, APOC3, and APOB. In some embodiments, the first gene is PCSK9 and the second gene is LPA.

In some embodiments, the first gene and second gene are selected from a combination listed in Table 1.

TABLE 1
Combinations of a first gene and a second gene targeted by a multiplexed
epigenetic-modifying DNA-targeting system provided herein

| First gene | Second gene |
|---|---|
| PCSK9 | LPA |
| PCSK9 | MYLIP |
| PCSK9 | ANGPTL3 |
| PCSK9 | APOC3 |
| PCSK9 | APOB |
| LPA | MYLIP |
| LPA | ANGPTL3 |
| LPA | APOC3 |
| LPA | APOB |
| MYLIP | ANGPTL3 |
| MYLIP | APOC3 |
| MYLIP | APOB |
| ANGPTL3 | APOC3 |
| ANGPTL3 | APOB |
| APOC3 | APOB |

In some embodiments, provided herein are multiplexed epigenetic-modifying DNA-targeting systems that target a first gene, a second gene, and a third gene. In some embodiments, the first gene is selected from the list consisting of PCSK9, LPA, MYLIP, ANGPTL3, APOC3, and APOB, the second gene is selected from the list consisting of PCSK9, LPA, MYLIP, ANGPTL3, APOC3, and APOB, the third gene is selected from the list consisting of PCSK9, LPA, MYLIP, ANGPTL3, APOC3, and APOB, and the first, second, and third genes are different. In some embodiments, the first gene is PCSK9, the second gene is selected from the list consisting of LPA, MYLIP, ANGPTL3, APOC3, and APOB, the third gene is selected from the list consisting of LPA, MYLIP, ANGPTL3, APOC3, and APOB, and the second gene and third gene are different. In some embodiments, the first gene is PCSK9, the second gene is LPA, and the third gene is selected from the list consisting of MYLIP, ANGPTL3, APOC3, and APOB.

In some embodiments, the first gene, second gene, and third gene are selected from a combination listed in Table 2.

TABLE 2
Combinations of a first gene, second gene, and
third gene targeted by a multiplexed epigenetic-
modifying DNA-targeting system provided herein

| First gene | Second gene | Third gene |
|---|---|---|
| PCSK9 | LPA | MYLIP |
| PCSK9 | LPA | ANGPTL3 |
| PCSK9 | LPA | APOC3 |
| PCSK9 | LPA | APOB |
| PCSK9 | MYLIP | ANGPTL3 |
| PCSK9 | MYLIP | APOC3 |
| PCSK9 | MYLIP | APOB |
| PCSK9 | ANGPTL3 | APOC3 |
| PCSK9 | ANGPTL3 | APOB |
| PCSK9 | APOC3 | APOB |
| LPA | MYLIP | ANGPTL3 |
| LPA | MYLIP | APOC3 |
| LPA | MYLIP | APOB |
| LPA | ANGPTL3 | APOC3 |
| LPA | ANGPTL3 | APOB |
| LPA | APOC3 | APOB |
| MYLIP | ANGPTL3 | APOC3 |
| MYLIP | ANGPTL3 | APOB |
| MYLIP | APOC3 | APOB |
| ANGPTL3 | APOC3 | APOB |

### B. CRISPR-Based DNA-Targeting Systems

Provided herein are multiplexed epigenetic-targeting DNA-targeting systems based on CRISPR/Cas systems, i.e. CRISPR/Cas-based DNA-targeting systems, that are able to bind to a target site in a target gene, or to target sites in a combination of target genes. In some embodiments, the CRISPR/Cas DNA-binding domain is nuclease inactive, such as includes a dCas (e.g. dCas9) so that the system binds to the target site in a target gene without mediating nucleic acid cleavage at the target site. The CRISPR/Cas-based DNA-targeting systems may be used to modulate expression of a target gene in a cell, such as a hepatocyte. In some embodiments, the target gene may include any as described herein, including any described above in Section I.A. In some embodiments, the target site of the target gene may include any as described herein, including any described above in Section I.A. In some embodiments, the CRISPR/Cas-based DNA-targeting system can include any known Cas enzyme, and generally a nuclease-inactive or dCas. In some embodiments, the CRISPR/Cas-based DNA-targeting system includes a fusion protein of a nuclease-inactive Cas protein or a variant thereof and an effector domain that reduces transcription of a gene (e.g. a transcriptional repressor), and at least one gRNA.

The CRISPR system (also known as CRISPR/Cas system, or CRISPR-Cas system) refers to a conserved microbial nuclease system, found in the genomes of bacteria and archaea, that provides a form of acquired immunity against invading phages and plasmids. Clustered Regularly Interspaced Short Palindromic Repeats (CRISPR), refers to loci containing multiple repeating DNA elements that are separated by non-repeating DNA sequences called spacers. Spacers are short sequences of foreign DNA that are incorporated into the genome between CRISPR repeats, serving as a ‘memory’ of past exposures. Spacers encode the DNA-targeting portion of RNA molecules that confer specificity for nucleic acid cleavage by the CRISPR system. CRISPR loci contain or are adjacent to one or more CRISPR-associated (Cas) genes, which can act as RNA-guided nucleases for mediating the cleavage, as well as non-protein coding DNA elements that encode RNA molecules capable of programming the specificity of the CRISPR-mediated nucleic acid cleavage.

In Type II CRISPR/Cas systems with the Cas protein Cas9, two RNA molecules and the Cas9 protein form a ribonucleoprotein (RNP) complex to direct Cas9 nuclease activity. The CRISPR RNA (crRNA) contains a spacer sequence that is complementary to a target nucleic acid sequence (target site), and that encodes the sequence specificity of the complex. The trans-activating crRNA (tracrRNA) base-pairs to a portion of the crRNA and forms a structure that complexes with the Cas9 protein, forming a Cas/RNA RNP complex.

Naturally occurring CRISPR/Cas systems, such as those with Cas9, have been engineered to allow efficient programming of Cas/RNA RNPs to target desired sequences in cells of interest, both for gene-editing and modulation of gene expression. The tracrRNA and crRNA have been engineered to form a single chimeric guide RNA molecule, commonly referred to as a guide RNA (gRNA), for example as described in WO 2013/176772, WO 2014/093661, WO 2014/093655, Jinek, M. et al. Science 337(6096):816-21 (2012), or Cong, L. et al. Science 339(6121):819-23 (2013). The spacer sequence of the gRNA can be chosen by a user to target the Cas/gRNA RNP complex to a desired locus, e.g. a desired target site in the target gene.

Cas proteins have also been engineered to be catalytically inactivated or nuclease inactive to allow targeting of Cas/gRNA RNPs without inducing cleavage at the target site. Mutations in Cas proteins can reduce or abolish nuclease activity of the Cas protein, rendering the Cas protein catalytically inactive. Cas proteins with reduced or abolished nuclease activity are referred to as deactivated Cas (dCas), or nuclease-inactive Cas (iCas) proteins, as referred to interchangeably herein. An exemplary deactivated Cas9 (dCas9) derived from S. pyogenes contains silencing mutations of the RuvC and HNH nuclease domains (D10A and H840A), for example as described in WO 2013/176772, WO 2014/093661, Jinek, M. et al. Science 337(6096):816-21 (2012), and Qi, L. et al. Cell 152(5):1173-83 (2013). Exemplary dCas variants derived from the Cas12 system (i.e. Cpf1) are described, for example in WO 2017/189308 and Zetsche, B. et al. Cell 163(3):759-71 (2015). Conserved domains that mediate nucleic acid cleavage, such as RuvC and HNH endonuclease domains, are readily identifiable in Cas orthologues, and can be mutated to produce inactive variants, for example as described in Zetsche, B. et al. Cell 163(3):759-71 (2015).

dCas-fusion proteins with transcriptional and/or epigenetic regulators have been used as a versatile platform for ectopically regulating gene expression in target cells. These include fusion of a Cas with an effector domain, such as a transcriptional activator or transcriptional repressor. For example, fusing dCas9 with a transcriptional activator such as VP64 (a polypeptide composed of four tandem copies of VP16, a 16 amino acid transactivation domain of the Herpes simplex virus) can result in robust induction of gene expression. Alternatively, fusing dCas9 with a transcriptional repressor such as KRAB (Kruppel associated box) can result in robust repression of gene expression. A variety of dCas-fusion proteins with transcriptional and epigenetic regulators can be engineered for regulation of gene expression, for example as described in WO 2014/197748, WO 2016/130600, WO 2017/180915, WO 2021/226555, WO 2013/176772, WO 2014/152432, WO 2014/093661, WO 2021/247570, Adli, M. Nat. Commun. 9, 1911 (2018), Perez-Pinera, P. et al. Nat. Methods 10, 973-976 (2013), Mali, P. et al. Nat. Biotechnol. 31, 833-838 (2013), Maeder, M. L. et al. Nat. Methods 10, 977-979 (2013), Gilbert, L. A. et al. Cell 154(2):442-451 (2013), and Nunez, J. K. et al. Cell 184(9):2503-2519 (2021).

In some aspects, provided is a DNA-targeting system comprising a fusion protein comprising a DNA-binding domain comprising a nuclease-inactive Cas protein or variant thereof, and an effector domain for reducing transcription or inducing transcriptional repression (i.e. a transcriptional repressor) when targeted to the target gene in the cell (e.g. hepatocyte). In such embodiments, the DNA-targeting system also includes one or more gRNAs, provided in combination or as a complex with the dCas protein or variant thereof, for targeting of the DNA-targeting system to the target site of the target gene. In some embodiments, the fusion protein is guided to a specific target site sequence of the target gene by the guide RNA, wherein the effector domain mediates targeted epigenetic modification to reduce or repress transcription of the target gene. In some embodiments, a combination of gRNAs guides the fusion protein to a combination of target site sequences in a combination of genes, wherein the effector domain mediates targeted epigenetic modification to reduce or repress transcription of the combination of target genes. Any of a variety of effector domains that reduce or repress transcription can be used as described further below.

i. CRISPR-based DNA-binding domains

In some aspects, the DNA-binding domain comprises a CRISPR-associated (Cas) protein or variant thereof, or is derived from a Cas protein or variant thereof. In particular embodiments here, the Cas protein is nuclease-inactive (i.e. is a dCas protein).

In some embodiments, the Cas protein is derived from a Class 1 CRISPR system (i.e. multiple Cas protein system), such as a Type I, Type III, or Type IV CRISPR system. In some embodiments, the Cas protein is derived from a Class 2 CRISPR system (i.e. single Cas protein system), such as a Type II, Type V, or Type VI CRISPR system. In some embodiments, the Cas protein is from a Type V CRISPR system. In some embodiments, the Cas protein is derived from a Cas12 protein (i.e. Cpf1) or variant thereof, for example as described in WO 2017/189308 and Zetsche, B. et al. Cell. 163(3):759-71 (2015). In some embodiments, the Cas protein is derived from a Type II CRISPR system. In some embodiments, the Cas protein is derived from a Cas9 protein or variant thereof, for example as described in WO 2013/176772, WO 2014/152432, WO 2014/093661, WO 2014/093655, Jinek, M. et al. Science 337(6096):816-21 (2012), Mali, P. et al. Science 339(6121):823-6 (2013), Cong, L. et al. Science 339(6121):819-23 (2013), Perez-Pinera, P. et al. Nat. Methods 10, 973-976 (2013), or Mali, P. et al. Nat. Biotechnol. 31, 833-838 (2013). Various CRISPR/Cas systems and associated Cas proteins for use in gene editing and regulation have been described, for example in Moon, S. B. et al. Exp. Mol. Med. 51, 1-11 (2019), Zhang, F. Q. Rev. Biophys. 52, E6 (2019), and Makarova K. S. et al. Methods Mol. Biol. 1311:47-75 (2015).

In some embodiments, the dCas9 protein can comprise a sequence derived from a naturally occurring Cas9 molecule, or variant thereof. In some embodiments, the dCas9 protein can comprise a sequence derived from a naturally occurring Cas9 molecule of S. pyogenes, S. thermophilus, S. aureus, C. jejuni, N. meningitidis, F. novicida, S. canis, S. auricularis, or variant thereof. In some embodiments, the dCas9 protein comprises a sequence derived from a naturally occurring Cas9 molecule of S. aureus. In some embodiments, the dCas9 protein comprises a sequence derived from a naturally occurring Cas9 molecule of S. pyogenes.

Non-limiting examples of Cas9 orthologs from other bacterial strains include but are not limited to: Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Candidatus Desulforudis audaxviator MP104C; Caldicellulosiruptor hydrothermalis 108; Clostridium phage c-st; Clostridium botulinum A3 str. Loch Maree; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum_thermopropionicum SI; Petrotoga mobilis SJ95; Polaromonas naphthalenivorans CJ2; Polaromonas sp. JS666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roseum DSM 43021; Synechococcus sp. PCC 7335; and Thermosipho africanus TCF52B (Chylinski et al., RNA Biol., 2013; 10(5): 726-737).

In some aspects, the Cas protein is a variant that lacks nuclease activity (i.e. is a dCas protein). In some embodiments, the Cas protein is mutated so that nuclease activity is reduced or eliminated. Such Cas proteins are referred to as deactivated Cas or dead Cas (dCas) or nuclease-inactive Cas (iCas) proteins, as referred to interchangeably herein. In some embodiments, the variant Cas protein is a variant Cas9 protein that lacks nuclease activity or that is a deactivated Cas9 (dCas9, or iCas9) protein.

In some embodiments, the Cas9 protein or a variant thereof is derived from a Staphylococcus aureus Cas9 (SaCas9) protein or a variant thereof. In some embodiments, the variant Cas9 is a Staphylococcus aureus dCas9 protein (dSaCas9) that comprises at least one amino acid mutation selected from D10A and N580A, with reference to numbering of positions of SEQ ID NO:204. In some embodiments, the variant Cas9 protein comprises the sequence set forth in SEQ ID NO:205, or an amino acid sequence that has at least 90%, 91%, 92%, 93%, 94%, 95%, 96%, 97%, 98%, or 99% sequence identity thereto.

In some embodiments, the Cas9 protein or variant thereof is derived from a Streptococcuspyogenes Cas9 (SpCas9) protein or a variant thereof. In some embodiments, the variant Cas9 is a Streptococcus pyogenes dCas9 (dSpCas9) protein that comprises at least one amino acid mutation selected from D10A and H840A, with reference to numbering of positions of SEQ ID NO:206. In some embodiments, the variant Cas9 protein comprises the sequence set forth in SEQ ID NO:207, or an amino acid sequence that has at least 90%, 91%, 92%, 93%, 94%, 95%, 96%, 97%, 98%, or 99% sequence identity thereto.

ii. Guide RNAs

In some embodiments, the Cas protein (e.g. dCas9) is provided in combination or as a complex with one or more guide RNA (gRNA). In some aspects, the gRNA is a nucleic acid that promotes the specific targeting or homing of the gRNA/Cas RNP complex to the target site of the target gene, such as any described above. In some embodiments, a target site of a gRNA may be referred to as a protospacer.

Provided herein are gRNAs, such as gRNAs that target or bind to a target gene or DNA regulatory element thereof, such as any described above in Section I.A. In some embodiments, the gRNA is capable of complexing with the Cas protein or variant thereof. In some embodiments, the gRNA comprises a gRNA spacer sequence (i.e. a spacer sequence or a guide sequence) that is capable of hybridizing to the target site, or that is complementary to the target site, such as any target site described in Section L.A or further below. In some embodiments, the gRNA comprises a scaffold sequence that complexes with or binds to the Cas protein.

In some embodiments, the gRNAs provided herein are chimeric gRNAs. In general, gRNAs can be unimolecular (i.e. composed of a single RNA molecule), or modular (comprising more than one, and typically two, separate RNA molecules). Modular gRNAs can be engineered to be unimolecular, wherein sequences from the separate modular RNA molecules are comprised in a single gRNA molecule, sometimes referred to as a chimeric gRNA, synthetic gRNA, or single gRNA. In some embodiments, the chimeric gRNA is a fusion of two non-coding RNA sequences: a crRNA sequence and a tracrRNA sequence, for example as described in WO 2013/176772, or Jinek, M. et al. Science 337(6096):816-21 (2012). In some embodiments, the chimeric gRNA mimics the naturally occurring crRNA:tracrRNA duplex involved in the Type II Effector system, wherein the naturally occurring crRNA:tracrRNA duplex acts as a guide for the Cas9 protein.

In some aspects, the spacer sequence of a gRNA is a polynucleotide sequence comprising at least a portion that has sufficient complementarity with the target gene or DNA regulatory element thereof (e.g. any described in Section I.A) to hybridize with a target site in the target gene and direct sequence-specific binding of a CRISPR complex to the sequence of the target site. Full complementarity is not necessarily required, provided there is sufficient complementarity to cause hybridization and promote formation of a CRISPR complex. In some embodiments, the gRNA comprises a spacer sequence that is complementary, e.g., at least 80%, 85%, 90%, 95%, 98%, 99%, or 100% (e.g., fully complementary), to the target site. The strand of the target nucleic acid comprising the target site sequence may be referred to as the “complementary strand” of the target nucleic acid.

In some embodiments, the gRNA spacer sequence is between about 14 nucleotides (nt) and about 26 nt, or between 16 nt and 22 nt in length. In some embodiments, the gRNA spacer sequence is 14 nt, 15 nt, 16 nt, 17 nt,18 nt, 19 nt, 20 nt, 21 nt or 22 nt, 23 nt, 24 nt, 25 nt, or 26 nt in length. In some embodiments, the gRNA spacer sequence is 18 nt, 19 nt, 20 nt, 21 nt or 22 nt in length. In some embodiments, the gRNA spacer sequence is 19 nt in length.

A target site of a gRNA may be referred to as a protospacer. In some aspects, the gRNA spacer is designed to target a protospacer with a specific protospacer-adjacent motif (PAM), i.e. a sequence immediately adjacent to the protospacer that contributes to and/or is required for Cas binding specificity. Different CRISPR/Cas systems have different PAM requirements for targeting. For example, in some embodiments, S. pyogenes Cas9 uses the PAM 5′-NGG-3′ (SEQ ID NO:202), where N is any nucleotide. S. aureus Cas9 uses the PAM 5′—NNGRRT-3′ (SEQ ID NO:203), where N is any nucleotide, and R is G or A. N. meningitidis Cas9 uses the PAM 5′-NNNNGATT-3′ (SEQ ID NO:210), where N is any nucleotide. C. jejuni Cas9 uses the PAM 5′-NNNNRYAC-3′ (SEQ ID NO:211), where N is any nucleotide, R is G or A, and Y is C or T. S. thermophilus uses the PAM 5′-NNAGAAW-3′(SEQ ID NO:212), where N is any nucleotide and W is A or T. F. Novicida Cas9 uses the PAM 5′-NGG-3′(SEQ ID NO:213), where N is any nucleotide. T. denticola Cas9 uses the PAM 5′-NAAAAC-3′(SEQ ID NO:214), where N is any nucleotide. Casl2a (also known as Cpf1) from various species, uses the PAM 5′-TTTV-3′(SEQ ID NO:215). Cas proteins may use or be engineered to use different PAMs from those listed above. For example, variant SpCas9 proteins may use a PAM selected from: 5′-NGG-3′ (SEQ ID NO:202), 5′-NGAN-3′ (SEQ ID NO:216), 5′-NGNG-3′(SEQ ID NO:217), 5′-NGAG-3′(SEQ ID NO:218), or 5′-NGCG-3′(SEQ ID NO:219). In some embodiments, the PAM sequence for complexing with S. pyogenes Cas9 or variant thereof is set forth in SEQ ID NO: 202. In some embodiments, the PAM sequence for complexing with S. aureus Cas9 or variant thereof is set forth in SEQ ID NO: 203.

A gRNA spacer sequence may be selected to reduce the degree of secondary structure within the spacer sequence. Secondary structure may be determined by any suitable polynucleotide folding algorithm.

In some embodiments, the gRNA (including the guide sequence) will comprise the base uracil (U), whereas DNA encoding the gRNA molecule will comprise the base thymine (T). While not wishing to be bound by theory, in some embodiments, it is believed that the complementarity of the guide sequence with the target sequence contributes to specificity of the interaction of the gRNA molecule/Cas molecule complex with a target nucleic acid. It is understood that in a guide sequence and target sequence pair, the uracil bases in the guide sequence will pair with the adenine bases in the target sequence. A gRNA spacer sequence herein may be defined by the DNA sequence encoding the gRNA spacer, and/or the RNA sequence of the spacer.

In some embodiments, one, more than one, or all of the nucleotides of a gRNA can have a modification, e.g., to render the gRNA less susceptible to degradation and/or improve bio-compatibility. By way of non-limiting example, the backbone of the gRNA can be modified with a phosphorothioate, or other modification(s). In some cases, a nucleotide of the gRNA can comprise a 2′ modification, e.g., a 2-acetylation, e.g., a 2′ methylation, or other modification(s).

Methods for designing gRNAs and exemplary targeting domains can include those described in, e.g., International PCT Pub. Nos. WO 2014/197748, WO 2016/130600, WO 2017/180915, WO 2021/226555, WO 2013/176772, WO 2014/152432, WO 2014/093661, WO 2014/093655, WO 2015/089427, WO 2016/049258, WO 2016/123578, WO 2021/076744, WO 2014/191128, WO 2015/161276, WO 2017/193107, and WO 2017/093969.

In some embodiments, a gRNA provided herein targets a target site in a gene in a cell (e.g. hepatocyte) or DNA regulatory element thereof, wherein the gene is selected from the list consisting of: PCSK9, LPA, MYLIP, ANGPTL3, APOC3, and APOB.

In some embodiments, the gRNA targets a target site that comprises a sequence selected from any one of SEQ ID NOS: 1-63, 306-317, 342-351, or 372-377, as shown in Table 3, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of any one of SEQ ID NOS:1-63, 306-317, 342-351, or 372-377 that is 14, 15, 16, 17, 18 or 19 nucleotides in length. In some embodiments, the target site is set forth in any one of SEQ ID NOS: 1-63, 306-317, 342-351, or 372-377. In some embodiments, the gRNA targets a target site in a PCSK9 promoter. In some embodiments, the gRNA targets a target site that includes or is set forth in SEQ ID NO:3 or a contiguous portion thereof of at least 14 nucleotides. In some embodiments, the gRNA targets a target site that includes or is set forth in SEQ ID NO:3.

In some embodiments, the gRNA comprises a spacer sequence selected from any one of SEQ ID NOS: 64-126, 318-329, 352-361, or 378-383, as shown in Table 3, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of any one of SEQ ID NOS: 64-126, 318-329, 352-361, or 378-383 that is 14, 15, 16, 17, 18 or 19 nucleotides in length. In some embodiments, the spacer sequence of the gRNA is set forth in any one of SEQ ID NOS: 64-126, 318-329, 352-361, or 378-383. In some embodiments, the spacer sequence of the gRNA includes or is set forth in SEQ ID NO:66 or a contiguous portion thereof of at least 14 nucleotides (e.g. 14, 15, 16, 17, 18 or 19 nucleotides) of SEQ ID NO:66. In some embodiments, the spacer sequence of the gRNA is set forth in SEQ ID NO:66.

In some embodiments, the gRNA further comprises a scaffold sequence. In some embodiments, the scaffold sequence comprises the sequence set forth in SEQ ID NO: 191 (GUUUAAGAGCUAUGCUGGAAACAGCAUAGCAAGUUUAAAUAAGGCUAGUCCG UUAUCAACUUGAAAAAGUGGCACCGAGUCGGUGC), or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the scaffold sequence is set forth in SEQ ID NO: 191. In some embodiments, the scaffold sequence comprises the DNA sequence set forth in SEQ ID NO: 190.

In some embodiments, a gRNA provided herein comprises a spacer sequence selected from any one of SEQ ID NOS: 64-126, 318-329, 352-361, or 378-383, as shown in Table 3. In some embodiments, the gRNA further comprises a scaffold sequence set forth in SEQ ID NO: 191. In some embodiments, the gRNA comprises the sequence selected from any one of SEQ ID NOS: 127-189, 330-341, 362-371, or 384-389, as shown in Table 4, or a sequence having at or at least 80%, 85%, 90%, 91%, 92%, 93%, 94%, 95%, 96%, 97%, 98%, 99%, 99.5%, 99.9%, or 100% sequence identity to any one of SEQ ID NO: 127-189, 330-341, 362-371, or 384-389. In some embodiments, the gRNA is set forth in any one of SEQ ID NOS: 127-189, 330-341, 362-371, or 384-389. In some embodiments, any of the provided gRNA sequences is complexed with or is provided in combination with a Cas9. In some embodiments, the Cas9 is a dCas9. In some embodiments, the dCas9 is a dSpCas9, such as a dSpCas9 set forth in SEQ ID NO: 207, or a variant and/or fusion thereof.

TABLE 3
Genes, target site sequences, and gRNA spacer sequences

| | | | | RNA |
|---|---|---|---|---|
| Target | target site | target | | spacer |
| Gene | (protospacer) sequence | SEQ ID | gRNA spacer sequence | SEQ ID |
| PCSK9 | GCGCGTAATCTGACGCTGTT | 1 | GCGCGUAAUCUGACGCUGUU | 64 |
| PCSK9 | ATCAGATAGGATCGTCCGAT | 2 | AUCAGAUAGGAUCGUCCGAU | 65 |
| PCSK9 | AGGTTTCCGCAGCGACGTCG | 3 | AGGUUUCCGCAGCGACGUCG | 66 |
| PCSK9 | GGGCGCCGCCGTTCAGTTCA | 4 | GGGCGCCGCCGUUCAGUUCA | 67 |
| PCSK9 | GGTGCTAGCCTTGCGTTCCG | 5 | GGUGCUAGCCUUGCGUUCCG | 68 |
| PCSK9 | CATTAACGGAACCCCCGGAC | 6 | CAUUAACGGAACCCCCGGAC | 69 |
| PCSK9 | AGGATCGTCCGATGGGGCTC | 7 | AGGAUCGUCCGAUGGGGCUC | 70 |
| PCSK9 | CCGTTAATGTTTAATCAGAT | 8 | CCGUUAAUGUUUAAUCAGAU | 71 |
| PCSK9 | CGTAATCTGACGCTGTTTG | 9 | CGUAAUCUGACGCUGUUUG | 72 |
| PCSK9 | GGTGTGGGTGCTTGACGCCT | 10 | GGUGUGGGUGCUUGACGCCU | 73 |
| PCSK9 | ACCCACTGCACGCTGGACAG | 11 | ACCCACUGCACGCUGGACAG | 74 |
| PCSK9 | GCACAGTAACAACCCCTGGT | 12 | GCACAGUAACAACCCCUGGU | 75 |
| PCSK9 | CCATCCATTCTTTCTCTAGG | 13 | CCAUCCAUUCUUUCUCUAGG | 76 |
| LPA | AAGGAGACATAAAGGCAATG | 14 | AAGGAGACAUAAAGGCAAUG | 77 |
| LPA | GGCAATGTGGAGCAGCTGAG | 15 | GGCAAUGUGGAGCAGCUGAG | 78 |
| LPA | GGAGCAGCTGAGGGGGGAAA | 16 | GGAGCAGCUGAGGGGGGAAA | 79 |
| LPA | TGTCAATAGATGCTGGGAAG | 17 | UGUCAAUAGAUGCUGGGAAG | 80 |
| LPA | AGTGCAATGTCAATAGATGC | 18 | AGUGCAAUGUCAAUAGAUGC | 81 |
| LPA | TTTATAAGACTCTATATTCA | 19 | UUUAUAAGACUCUAUAUUCA | 82 |
| LPA | CATGTAAGTCAACAATGTCC | 20 | CAUGUAAGUCAACAAUGUCC | 83 |
| LPA | GTCAACAATGTCCTGGGATT | 21 | GUCAACAAUGUCCUGGGAUU | 84 |
| LPA | CATATACAAGATTTTGAACT | 22 | CAUAUACAAGAUUUUGAACU | 85 |
| LPA | GCACCGTGACAGTCTTCACG | 23 | GCACCGUGACAGUCUUCACG | 86 |
| MYLIP | TTGGCGGGGACCCGAGCTGA | 24 | UUGGCGGGGACCCGAGCUGA | 87 |
| MYLIP | CTGTCGCAGCGCAGGCAGTT | 25 | CUGUCGCAGCGCAGGCAGUU | 88 |
| MYLIP | GCTGGAGTGCGGCGCCACCG | 26 | GCUGGAGUGCGGCGCCACCG | 89 |
| MYLIP | CGGCGCCACCGCGGAGGACA | 27 | CGGCGCCACCGCGGAGGACA | 90 |
| MYLIP | CAGCTCTGCGGACCCTTGTC | 28 | CAGCUCUGCGGACCCUUGUC | 91 |
| MYLIP | CCCCGCGCACACCAAAGAGA | 29 | CCCCGCGCACACCAAAGAGA | 92 |
| MYLIP | CCTCGTCACATAACACAGCA | 30 | CCUCGUCACAUAACACAGCA | 93 |
| MYLIP | ACCTCCATCAGCACCGCGTC | 31 | ACCUCCAUCAGCACCGCGUC | 94 |
| MYLIP | GGAGGCGAAAGCCAACGGCG | 32 | GGAGGCGAAAGCCAACGGCG | 95 |
| MYLIP | GTTGAGGCAGTCCTCGCCGT | 33 | GUUGAGGCAGUCCUCGCCGU | 96 |
| ANGLPTL3 | TACATTCGTGCAAGTTAACA | 34 | UACAUUCGUGCAAGUUAACA | 97 |
| ANGLPTL3 | CCTACCAACCTTACCTTTTC | 35 | CCUACCAACCUUACCUUUUC | 98 |
| ANGLPTL3 | TATATAGAGTTAAGAAGTCT | 36 | UAUAUAGAGUUAAGAAGUCU | 99 |
| ANGLPTL3 | AACGTGGAACTGTTTTCTTC | 37 | AACGUGGAACUGUUUUCUUC | 100 |
| ANGLPTL3 | ATTTTCAATTTCAAGCAACG | 38 | AUUUUCAAUUUCAAGCAACG | 101 |
| ANGLPTL3 | ATTCTGGAGGAAATAACTAG | 39 | AUUCUGGAGGAAAUAACUAG | 102 |
| ANGLPTL3 | GCAAATCTTGATTTTGGCTC | 40 | GCAAAUCUUGAUUUUGGCUC | 103 |
| ANGLPTL3 | AGCCAATGGCCTCCTTCAGT | 41 | AGCCAAUGGCCUCCUUCAGU | 104 |
| ANGLPTL3 | TAAGACCATGTCCCAACTGA | 42 | UAAGACCAUGUCCCAACUGA | 105 |
| ANGLPTL3 | AGACTTTGTCCATAAGACGA | 43 | AGACUUUGUCCAUAAGACGA | 106 |
| APOC3 | GGGGCACCCGTCCAGCTCCG | 44 | GGGGCACCCGUCCAGCUCCG | 107 |
| APOC3 | TGACCTTTGCCCAGCGCCCT | 45 | UGACCUUUGCCCAGCGCCCU | 108 |
| APOC3 | TCCAGATGCAGCAAGCGGGC | 46 | UCCAGAUGCAGCAAGCGGGC | 109 |
| APOC3 | TAGGGATGAACTGAGCAGAC | 47 | UAGGGAUGAACUGAGCAGAC | 110 |
| APOC3 | AGAAGCACTTGCTAGAGCTA | 48 | AGAAGCACUUGCUAGAGCUA | 111 |
| APOC3 | CTGCTCCAGGTAATGCCCTC | 49 | CUGCUCCAGGUAAUGCCCUC | 112 |
| APOC3 | GGGAGAGTTGGGAAATCCCT | 50 | GGGAGAGUUGGGAAAUCCCU | 113 |
| APOC3 | AGGAAGCCTCGGAGCTGGAC | 51 | AGGAAGCCUCGGAGCUGGAC | 114 |
| APOC3 | CCCTGGAGATGATATAAAA | 52 | CCCUGGAGAUGAUAUAAAAC | 115 |
| APOC3 | TCATAACCTGAAGAACATGG | 53 | UCAUAACCUGAAGAACAUGG | 116 |
| APOB | GTCCATCGCCAGCTGCGGTG | 54 | GUCCAUCGCCAGCUGCGGUG | 117 |
| APOB | GGCGCCCGCACCCCATTTAT | 55 | GGCGCCCGCACCCCAUUUAU | 118 |
| APOB | CAGAGCGGCCGCGCACTCAC | 56 | CAGAGCGGCCGCGCACUCAC | 119 |
| APOB | CTCAGCGGCAGCAACCGAGA | 57 | CUCAGCGGCAGCAACCGAGA | 120 |
| APOB | TCCCGGTGGGAATGCGCGGC | 58 | UCCCGGUGGGAAUGCGCGGC | 121 |
| APOB | GCATTCCCACCGGGACCTGC | 59 | GCAUUCCCACCGGGACCUGC | 122 |
| APOB | GCCTCGCGGCCCTGGCTGGC | 60 | GCCUCGCGGCCCUGGCUGGC | 123 |
| APOB | CCCGGCCAACCTCGTGCCGC | 61 | CCCGGCCAACCUCGUGCCGC | 124 |
| APOB | AGCGCCAGCAGCGCGGGCCT | 62 | AGCGCCAGCAGCGCGGGCCU | 125 |
| APOB | CTCCCTCTGCGCCCGCAGAG | 63 | CUCCCUCUGCGCCCGCAGAG | 126 |
| PCSK9 | GTCGAGGCGCTCATGGTTGC | 306 | GUCGAGGCGCUCAUGGUUGC | 318 |
| PCSK9 | TTCCAGCCCAGTTAGGATTT | 307 | UUCCAGCCCAGUUAGGAUUU | 319 |
| PCSK9 | TCCTAACTGGGCTGGAAGGC | 308 | UCCUAACUGGGCUGGAAGGC | 320 |
| PCSK9 | TCAGGAGCAGGGCGCGTGAA | 309 | UCAGGAGCAGGGCGCGUGAA | 321 |
| PCSK9 | CAGCGACGTCGAGGCGCTCA | 310 | CAGCGACGUCGAGGCGCUCA | 322 |
| PCSK9 | CCGTCAGCTCCAGGCGGTCC | 311 | CCGUCAGCUCCAGGCGGUCC | 323 |
| PCSK9 | AACCTGATCCTCCAGTCCGG | 312 | AACCUGAUCCUCCAGUCCGG | 324 |
| PCSK9 | TCATGGGCACCGTCAGCTCC | 313 | UCAUGGGCACCGUCAGCUCC | 325 |
| PCSK9 | CCGCCGGCGTGGACCGCGCA | 314 | CCGCCGGCGUGGACCGCGCA | 326 |
| PCSK9 | GAAGGCAGGCCGGCGCCCTA | 315 | GAAGGCAGGCCGGCGCCCUA | 327 |
| PCSK9 | GCGCCTTGAGCCTTGCGGTG | 316 | GCGCCUUGAGCCUUGCGGUG | 328 |
| PCSK9 | CCCGCACCTTGGCGCAGCGG | 317 | CCCGCACCUUGGCGCAGCGG | 329 |
| MYLIP | GCACTGCGGCGGCAGCCGGG | 342 | GCACUGCGGCGGCAGCCGGG | 352 |
| MYLIP | GGCGCCACCGCGGAGGACAG | 343 | GGCGCCACCGCGGAGGACAG | 353 |
| MYLIP | ATGCTCATAGGATGTATTCA | 344 | AUGCUCAUAGGAUGUAUUCA | 354 |
| MYLIP | CCACAATAAACACATGGTCT | 345 | CCACAAUAAACACAUGGUCU | 355 |
| MYLIP | GGGTCCCACCAGTGACAAGG | 346 | GGGUCCCACCAGUGACAAGG | 356 |
| MYLIP | GGACTGCCTCAACCAGGTGA | 347 | GGACUGCCUCAACCAGGUGA | 357 |
| MYLIP | CAGAGTCCCTGTCGCAGCGC | 348 | CAGAGUCCCUGUCGCAGCGC | 358 |
| MYLIP | CTCAGAGTGAGCGATCGCCC | 349 | CUCAGAGUGAGCGAUCGCCC | 359 |
| MYLIP | CTGAGTTTCCCTGGCCGCCC | 350 | CUGAGUUUCCCUGGCCGCCC | 360 |
| MYLIP | GTTTCCCTGGCCGCCCCGGG | 351 | GUUUCCCUGGCCGCCCCGGG | 361 |
| APOB | TGAGGGCCTCCCACTCTACA | 372 | UGAGGGCCUCCCACUCUACA | 378 |
| APOB | CCAGAGCACTGAAGACGCTT | 373 | CCAGAGCACUGAAGACGCUU | 379 |
| APOB | ACTGGAGGAAACCTAGAAGC | 374 | ACUGGAGGAAACCUAGAAGC | 380 |
| APOB | CACTGAAGACGCTTGGGGAA | 375 | CACUGAAGACGCUUGGGGAA | 381 |
| APOB | TGAAGAAGGCACCCCTGGTC | 376 | UGAAGAAGGCACCCCUGGUC | 382 |
| APOB | TGAGTGCGCGGCCGCTCTGC | 377 | UGAGUGCGCGGCCGCUCUGC | 383 |

TABLE 4
Genes and gene-targeting gRNAs

| Target gene | gRNA sequence | SEQ ID |
|---|---|---|
| PCSK9 | GCGCGUAAUCUGACGCUGUUGUUUAAGAGCUAUGCUGGAAACAGCAU | 127 |
| | AGCAAGUUUAAAUAAGGCUAGUCCGUUAUCAACUUGAAAAAGUGGCA | |
| | CCGAGUCGGUGC | |
| PCSK9 | AUCAGAUAGGAUCGUCCGAUGUUUAAGAGCUAUGCUGGAAACAGCAU | 128 |
| | AGCAAGUUUAAAUAAGGCUAGUCCGUUAUCAACUUGAAAAAGUGGCA | |
| | CCGAGUCGGUGC | |
| PCSK9 | AGGUUUCCGCAGCGACGUCGGUUUAAGAGCUAUGCUGGAAACAGCAU | 129 |
| | AGCAAGUUUAAAUAAGGCUAGUCCGUUAUCAACUUGAAAAAGUGGCA | |
| | CCGAGUCGGUGC | |
| PCSK9 | GGGCGCCGCCGUUCAGUUCAGUUUAAGAGCUAUGCUGGAAACAGCAU | 130 |
| | AGCAAGUUUAAAUAAGGCUAGUCCGUUAUCAACUUGAAAAAGUGGCA | |
| | CCGAGUCGGUGC | |
| PCSK9 | GGUGCUAGCCUUGCGUUCCGGUUUAAGAGCUAUGCUGGAAACAGCAU | 131 |
| | AGCAAGUUUAAAUAAGGCUAGUCCGUUAUCAACUUGAAAAAGUGGCA | |
| | CCGAGUCGGUGC | |
| PCSK9 | CAUUAACGGAACCCCCGGACGUUUAAGAGCUAUGCUGGAAACAGCAU | 132 |
| | AGCAAGUUUAAAUAAGGCUAGUCCGUUAUCAACUUGAAAAAGUGGCA | |
| | CCGAGUCGGUGC | |
| PCSK9 | AGGAUCGUCCGAUGGGGCUCGUUUAAGAGCUAUGCUGGAAACAGCAU | 133 |
| | AGCAAGUUUAAAUAAGGCUAGUCCGUUAUCAACUUGAAAAAGUGGCA | |
| | CCGAGUCGGUGC | |
| PCSK9 | CCGUUAAUGUUUAAUCAGAUGUUUAAGAGCUAUGCUGGAAACAGCAU | 134 |
| | AGCAAGUUUAAAUAAGGCUAGUCCGUUAUCAACUUGAAAAAGUGGCA | |
| | CCGAGUCGGUGC | |
| PCSK9 | CGUAAUCUGACGCUGUUUGGUUUAAGAGCUAUGCUGGAAACAGCAUA | 135 |
| | GCAAGUUUAAAUAAGGCUAGUCCGUUAUCAACUUGAAAAAGUGGCAC | |
| | CGAGUCGGUGC | |
| PCSK9 | GGUGUGGGUGCUUGACGCCUGUUUAAGAGCUAUGCUGGAAACAGCAU | 136 |
| | AGCAAGUUUAAAUAAGGCUAGUCCGUUAUCAACUUGAAAAAGUGGCA | |
| | CCGAGUCGGUGC | |
| PCSK9 | ACCCACUGCACGCUGGACAGGUUUAAGAGCUAUGCUGGAAACAGCAU | 137 |
| | AGCAAGUUUAAAUAAGGCUAGUCCGUUAUCAACUUGAAAAAGUGGCA | |
| | CCGAGUCGGUGC | |
| PCSK9 | GCACAGUAACAACCCCUGGUGUUUAAGAGCUAUGCUGGAAACAGCAU | 138 |
| | AGCAAGUUUAAAUAAGGCUAGUCCGUUAUCAACUUGAAAAAGUGGCA | |
| | CCGAGUCGGUGC | |
| PCSK9 | CCAUCCAUUCUUUCUCUAGGGUUUAAGAGCUAUGCUGGAAACAGCAU | 139 |
| | AGCAAGUUUAAAUAAGGCUAGUCCGUUAUCAACUUGAAAAAGUGGCA | |
| | CCGAGUCGGUGC | |
| LPA | AAGGAGACAUAAAGGCAAUGGUUUAAGAGCUAUGCUGGAAACAGCA | 140 |
| | UAGCAAGUUUAAAUAAGGCUAGUCCGUUAUCAACUUGAAAAAGUGGC | |
| | ACCGAGUCGGUGC | |
| LPA | GGCAAUGUGGAGCAGCUGAGGUUUAAGAGCUAUGCUGGAAACAGCAU | 141 |
| | AGCAAGUUUAAAUAAGGCUAGUCCGUUAUCAACUUGAAAAAGUGGCA | |
| | CCGAGUCGGUGC | |
| LPA | GGAGCAGCUGAGGGGGGAAAGUUUAAGAGCUAUGCUGGAAACAGCA | 142 |
| | UAGCAAGUUUAAAUAAGGCUAGUCCGUUAUCAACUUGAAAAAGUGGC | |
| | ACCGAGUCGGUGC | |
| LPA | UGUCAAUAGAUGCUGGGAAGGUUUAAGAGCUAUGCUGGAAACAGCA | 143 |
| | UAGCAAGUUUAAAUAAGGCUAGUCCGUUAUCAACUUGAAAAAGUGGC | |
| | ACCGAGUCGGUGC | |
| LPA | AGUGCAAUGUCAAUAGAUGCGUUUAAGAGCUAUGCUGGAAACAGCAU | 144 |
| | AGCAAGUUUAAAUAAGGCUAGUCCGUUAUCAACUUGAAAAAGUGGCA | |
| | CCGAGUCGGUGC | |
| LPA | UUUAUAAGACUCUAUAUUCAGUUUAAGAGCUAUGCUGGAAACAGCAU | 145 |
| | AGCAAGUUUAAAUAAGGCUAGUCCGUUAUCAACUUGAAAAAGUGGCA | |
| | CCGAGUCGGUGC | |
| LPA | CAUGUAAGUCAACAAUGUCCGUUUAAGAGCUAUGCUGGAAACAGCAU | 146 |
| | AGCAAGUUUAAAUAAGGCUAGUCCGUUAUCAACUUGAAAAAGUGGCA | |
| | CCGAGUCGGUGC | |
| LPA | GUCAACAAUGUCCUGGGAUUGUUUAAGAGCUAUGCUGGAAACAGCAU | 147 |
| | AGCAAGUUUAAAUAAGGCUAGUCCGUUAUCAACUUGAAAAAGUGGCA | |
| | CCGAGUCGGUGC | |
| LPA | CAUAUACAAGAUUUUGAACUGUUUAAGAGCUAUGCUGGAAACAGCAU | 148 |
| | AGCAAGUUUAAAUAAGGCUAGUCCGUUAUCAACUUGAAAAAGUGGCA | |
| | CCGAGUCGGUGC | |
| LPA | GCACCGUGACAGUCUUCACGGUUUAAGAGCUAUGCUGGAAACAGCAU | 149 |
| | AGCAAGUUUAAAUAAGGCUAGUCCGUUAUCAACUUGAAAAAGUGGCA | |
| | CCGAGUCGGUGC | |
| MYLIP | UUGGCGGGGACCCGAGCUGAGUUUAAGAGCUAUGCUGGAAACAGCAU | 150 |
| | AGCAAGUUUAAAUAAGGCUAGUCCGUUAUCAACUUGAAAAAGUGGCA | |
| | CCGAGUCGGUGC | |
| MYLIP | CUGUCGCAGCGCAGGCAGUUGUUUAAGAGCUAUGCUGGAAACAGCAU | 151 |
| | AGCAAGUUUAAAUAAGGCUAGUCCGUUAUCAACUUGAAAAAGUGGCA | |
| | CCGAGUCGGUGC | |
| MYLIP | GCUGGAGUGCGGCGCCACCGGUUUAAGAGCUAUGCUGGAAACAGCAU | 152 |
| | AGCAAGUUUAAAUAAGGCUAGUCCGUUAUCAACUUGAAAAAGUGGCA | |
| | CCGAGUCGGUGC | |
| MYLIP | CGGCGCCACCGCGGAGGACAGUUUAAGAGCUAUGCUGGAAACAGCAU | 153 |
| | AGCAAGUUUAAAUAAGGCUAGUCCGUUAUCAACUUGAAAAAGUGGCA | |
| | CCGAGUCGGUGC | |
| MYLIP | CAGCUCUGCGGACCCUUGUCGUUUAAGAGCUAUGCUGGAAACAGCAU | 154 |
| | AGCAAGUUUAAAUAAGGCUAGUCCGUUAUCAACUUGAAAAAGUGGCA | |
| | CCGAGUCGGUGC | |
| MYLIP | CCCCGCGCACACCAAAGAGAGUUUAAGAGCUAUGCUGGAAACAGCAU | 155 |
| | AGCAAGUUUAAAUAAGGCUAGUCCGUUAUCAACUUGAAAAAGUGGCA | |
| | CCGAGUCGGUGC | |
| MYLIP | CCUCGUCACAUAACACAGCAGUUUAAGAGCUAUGCUGGAAACAGCAU | 156 |
| | AGCAAGUUUAAAUAAGGCUAGUCCGUUAUCAACUUGAAAAAGUGGCA | |
| | CCGAGUCGGUGC | |
| MYLIP | ACCUCCAUCAGCACCGCGUCGUUUAAGAGCUAUGCUGGAAACAGCAU | 157 |
| | AGCAAGUUUAAAUAAGGCUAGUCCGUUAUCAACUUGAAAAAGUGGCA | |
| | CCGAGUCGGUGC | |
| MYLIP | GGAGGCGAAAGCCAACGGCGGUUUAAGAGCUAUGCUGGAAACAGCAU | 158 |
| | AGCAAGUUUAAAUAAGGCUAGUCCGUUAUCAACUUGAAAAAGUGGCA | |
| | CCGAGUCGGUGC | |
| MYLIP | GUUGAGGCAGUCCUCGCCGUGUUUAAGAGCUAUGCUGGAAACAGCAU | 159 |
| | AGCAAGUUUAAAUAAGGCUAGUCCGUUAUCAACUUGAAAAAGUGGCA | |
| | CCGAGUCGGUGC | |
| ANGPTL3 | UACAUUCGUGCAAGUUAACAGUUUAAGAGCUAUGCUGGAAACAGCAU | 160 |
| | AGCAAGUUUAAAUAAGGCUAGUCCGUUAUCAACUUGAAAAAGUGGCA | |
| | CCGAGUCGGUGC | |
| ANGPTL3 | CCUACCAACCUUACCUUUUCGUUUAAGAGCUAUGCUGGAAACAGCAU | 161 |
| | AGCAAGUUUAAAUAAGGCUAGUCCGUUAUCAACUUGAAAAAGUGGCA | |
| | CCGAGUCGGUGC | |
| ANGPTL3 | UAUAUAGAGUUAAGAAGUCUGUUUAAGAGCUAUGCUGGAAACAGCA | 162 |
| | UAGCAAGUUUAAAUAAGGCUAGUCCGUUAUCAACUUGAAAAAGUGGC | |
| | ACCGAGUCGGUGC | |
| ANGPTL3 | AACGUGGAACUGUUUUCUUCGUUUAAGAGCUAUGCUGGAAACAGCAU | 163 |
| | AGCAAGUUUAAAUAAGGCUAGUCCGUUAUCAACUUGAAAAAGUGGCA | |
| | CCGAGUCGGUGC | |
| ANGPTL3 | AUUUUCAAUUUCAAGCAACGGUUUAAGAGCUAUGCUGGAAACAGCAU | 164 |
| | AGCAAGUUUAAAUAAGGCUAGUCCGUUAUCAACUUGAAAAAGUGGCA | |
| | CCGAGUCGGUGC | |
| ANGPTL3 | AUUCUGGAGGAAAUAACUAGGUUUAAGAGCUAUGCUGGAAACAGCA | 165 |
| | UAGCAAGUUUAAAUAAGGCUAGUCCGUUAUCAACUUGAAAAAGUGGC | |
| | ACCGAGUCGGUGC | |
| ANGPTL3 | GCAAAUCUUGAUUUUGGCUCGUUUAAGAGCUAUGCUGGAAACAGCAU | 166 |
| | AGCAAGUUUAAAUAAGGCUAGUCCGUUAUCAACUUGAAAAAGUGGCA | |
| | CCGAGUCGGUGC | |
| ANGPTL3 | AGCCAAUGGCCUCCUUCAGUGUUUAAGAGCUAUGCUGGAAACAGCAU | 167 |
| | AGCAAGUUUAAAUAAGGCUAGUCCGUUAUCAACUUGAAAAAGUGGCA | |
| | CCGAGUCGGUGC | |
| ANGPTL3 | UAAGACCAUGUCCCAACUGAGUUUAAGAGCUAUGCUGGAAACAGCAU | 168 |
| | AGCAAGUUUAAAUAAGGCUAGUCCGUUAUCAACUUGAAAAAGUGGCA | |
| | CCGAGUCGGUGC | |
| ANGPTL3 | AGACUUUGUCCAUAAGACGAGUUUAAGAGCUAUGCUGGAAACAGCAU | 169 |
| | AGCAAGUUUAAAUAAGGCUAGUCCGUUAUCAACUUGAAAAAGUGGCA | |
| | CCGAGUCGGUGC | |
| APOC3 | GGGGCACCCGUCCAGCUCCGGUUUAAGAGCUAUGCUGGAAACAGCAU | 170 |
| | AGCAAGUUUAAAUAAGGCUAGUCCGUUAUCAACUUGAAAAAGUGGCA | |
| | CCGAGUCGGUGC | |
| APOC3 | UGACCUUUGCCCAGCGCCCUGUUUAAGAGCUAUGCUGGAAACAGCAU | 171 |
| | AGCAAGUUUAAAUAAGGCUAGUCCGUUAUCAACUUGAAAAAGUGGCA | |
| | CCGAGUCGGUGC | |
| APOC3 | UCCAGAUGCAGCAAGCGGGCGUUUAAGAGCUAUGCUGGAAACAGCAU | 172 |
| | AGCAAGUUUAAAUAAGGCUAGUCCGUUAUCAACUUGAAAAAGUGGCA | |
| | CCGAGUCGGUGC | |
| APOC3 | UAGGGAUGAACUGAGCAGACGUUUAAGAGCUAUGCUGGAAACAGCAU | 173 |
| | AGCAAGUUUAAAUAAGGCUAGUCCGUUAUCAACUUGAAAAAGUGGCA | |
| | CCGAGUCGGUGC | |
| APOC3 | AGAAGCACUUGCUAGAGCUAGUUUAAGAGCUAUGCUGGAAACAGCAU | 174 |
| | AGCAAGUUUAAAUAAGGCUAGUCCGUUAUCAACUUGAAAAAGUGGCA | |
| | CCGAGUCGGUGC | |
| APOC3 | CUGCUCCAGGUAAUGCCCUCGUUUAAGAGCUAUGCUGGAAACAGCAU | 175 |
| | AGCAAGUUUAAAUAAGGCUAGUCCGUUAUCAACUUGAAAAAGUGGCA | |
| | CCGAGUCGGUGC | |
| APOC3 | GGGAGAGUUGGGAAAUCCCUGUUUAAGAGCUAUGCUGGAAACAGCAU | 176 |
| | AGCAAGUUUAAAUAAGGCUAGUCCGUUAUCAACUUGAAAAAGUGGCA | |
| | CCGAGUCGGUGC | |
| APOC3 | AGGAAGCCUCGGAGCUGGACGUUUAAGAGCUAUGCUGGAAACAGCAU | 177 |
| | AGCAAGUUUAAAUAAGGCUAGUCCGUUAUCAACUUGAAAAAGUGGCA | |
| | CCGAGUCGGUGC | |
| APOC3 | CCCUGGAGAUGAUAUAAAACGUUUAAGAGCUAUGCUGGAAACAGCAU | 178 |
| | AGCAAGUUUAAAUAAGGCUAGUCCGUUAUCAACUUGAAAAAGUGGCA | |
| | CCGAGUCGGUGC | |
| APOC3 | UCAUAACCUGAAGAACAUGGGUUUAAGAGCUAUGCUGGAAACAGCAU | 179 |
| | AGCAAGUUUAAAUAAGGCUAGUCCGUUAUCAACUUGAAAAAGUGGCA | |
| | CCGAGUCGGUGC | |
| APOB | GUCCAUCGCCAGCUGCGGUGGUUUAAGAGCUAUGCUGGAAACAGCAU | 180 |
| | AGCAAGUUUAAAUAAGGCUAGUCCGUUAUCAACUUGAAAAAGUGGCA | |
| | CCGAGUCGGUGC | |
| APOB | GGCGCCCGCACCCCAUUUAUGUUUAAGAGCUAUGCUGGAAACAGCAU | 181 |
| | AGCAAGUUUAAAUAAGGCUAGUCCGUUAUCAACUUGAAAAAGUGGCA | |
| | CCGAGUCGGUGC | |
| APOB | CAGAGCGGCCGCGCACUCACGUUUAAGAGCUAUGCUGGAAACAGCAU | 182 |
| | AGCAAGUUUAAAUAAGGCUAGUCCGUUAUCAACUUGAAAAAGUGGCA | |
| | CCGAGUCGGUGC | |
| APOB | CUCAGCGGCAGCAACCGAGAGUUUAAGAGCUAUGCUGGAAACAGCAU | 183 |
| | AGCAAGUUUAAAUAAGGCUAGUCCGUUAUCAACUUGAAAAAGUGGCA | |
| | CCGAGUCGGUGC | |
| APOB | UCCCGGUGGGAAUGCGCGGCGUUUAAGAGCUAUGCUGGAAACAGCAU | 184 |
| | AGCAAGUUUAAAUAAGGCUAGUCCGUUAUCAACUUGAAAAAGUGGCA | |
| | CCGAGUCGGUGC | |
| APOB | GCAUUCCCACCGGGACCUGCGUUUAAGAGCUAUGCUGGAAACAGCAU | 185 |
| | AGCAAGUUUAAAUAAGGCUAGUCCGUUAUCAACUUGAAAAAGUGGCA | |
| | CCGAGUCGGUGC | |
| APOB | GCCUCGCGGCCCUGGCUGGCGUUUAAGAGCUAUGCUGGAAACAGCAU | 186 |
| | AGCAAGUUUAAAUAAGGCUAGUCCGUUAUCAACUUGAAAAAGUGGCA | |
| | CCGAGUCGGUGC | |
| APOB | CCCGGCCAACCUCGUGCCGCGUUUAAGAGCUAUGCUGGAAACAGCAU | 187 |
| | AGCAAGUUUAAAUAAGGCUAGUCCGUUAUCAACUUGAAAAAGUGGCA | |
| | CCGAGUCGGUGC | |
| APOB | AGCGCCAGCAGCGCGGGCCUGUUUAAGAGCUAUGCUGGAAACAGCAU | 188 |
| | AGCAAGUUUAAAUAAGGCUAGUCCGUUAUCAACUUGAAAAAGUGGCA | |
| | CCGAGUCGGUGC | |
| APOB | CUCCCUCUGCGCCCGCAGAGGUUUAAGAGCUAUGCUGGAAACAGCAU | 189 |
| | AGCAAGUUUAAAUAAGGCUAGUCCGUUAUCAACUUGAAAAAGUGGCA | |
| | CCGAGUCGGUGC | |
| PCSK9 | GUCGAGGCGCUCAUGGUUGCGUUUAAGAGCUAUGCUGGAAACAGCAU | 330 |
| | AGCAAGUUUAAAUAAGGCUAGUCCGUUAUCAACUUGAAAAAGUGGCA | |
| | CCGAGUCGGUGC | |
| PCSK9 | UUCCAGCCCAGUUAGGAUUUGUUUAAGAGCUAUGCUGGAAACAGCAU | 331 |
| | AGCAAGUUUAAAUAAGGCUAGUCCGUUAUCAACUUGAAAAAGUGGCA | |
| | CCGAGUCGGUGC | |
| PCSK9 | UCCUAACUGGGCUGGAAGGCGUUUAAGAGCUAUGCUGGAAACAGCAU | 332 |
| | AGCAAGUUUAAAUAAGGCUAGUCCGUUAUCAACUUGAAAAAGUGGCA | |
| | CCGAGUCGGUGC | |
| PCSK9 | UCAGGAGCAGGGCGCGUGAAGUUUAAGAGCUAUGCUGGAAACAGCAU | 333 |
| | AGCAAGUUUAAAUAAGGCUAGUCCGUUAUCAACUUGAAAAAGUGGCA | |
| | CCGAGUCGGUGC | |
| PCSK9 | CAGCGACGUCGAGGCGCUCAGUUUAAGAGCUAUGCUGGAAACAGCAU | 334 |
| | AGCAAGUUUAAAUAAGGCUAGUCCGUUAUCAACUUGAAAAAGUGGCA | |
| | CCGAGUCGGUGC | |
| PCSK9 | CCGUCAGCUCCAGGCGGUCCGUUUAAGAGCUAUGCUGGAAACAGCAU | 335 |
| | AGCAAGUUUAAAUAAGGCUAGUCCGUUAUCAACUUGAAAAAGUGGCA | |
| | CCGAGUCGGUGC | |
| PCSK9 | AACCUGAUCCUCCAGUCCGGGUUUAAGAGCUAUGCUGGAAACAGCAU | 336 |
| | AGCAAGUUUAAAUAAGGCUAGUCCGUUAUCAACUUGAAAAAGUGGCA | |
| | CCGAGUCGGUGC | |
| PCSK9 | UCAUGGGCACCGUCAGCUCCGUUUAAGAGCUAUGCUGGAAACAGCAU | 337 |
| | AGCAAGUUUAAAUAAGGCUAGUCCGUUAUCAACUUGAAAAAGUGGCA | |
| | CCGAGUCGGUGC | |
| PCSK9 | CCGCCGGCGUGGACCGCGCAGUUUAAGAGCUAUGCUGGAAACAGCAU | 338 |
| | AGCAAGUUUAAAUAAGGCUAGUCCGUUAUCAACUUGAAAAAGUGGCA | |
| | CCGAGUCGGUGC | |
| PCSK9 | GAAGGCAGGCCGGCGCCCUAGUUUAAGAGCUAUGCUGGAAACAGCAU | 339 |
| | AGCAAGUUUAAAUAAGGCUAGUCCGUUAUCAACUUGAAAAAGUGGCA | |
| | CCGAGUCGGUGC | |
| PCSK9 | GCGCCUUGAGCCUUGCGGUGGUUUAAGAGCUAUGCUGGAAACAGCAU | 340 |
| | AGCAAGUUUAAAUAAGGCUAGUCCGUUAUCAACUUGAAAAAGUGGCA | |
| | CCGAGUCGGUGC | |
| PCSK9 | CCCGCACCUUGGCGCAGCGGGUUUAAGAGCUAUGCUGGAAACAGCAU | 341 |
| | AGCAAGUUUAAAUAAGGCUAGUCCGUUAUCAACUUGAAAAAGUGGCA | |
| | CCGAGUCGGUGC | |
| MYLIP | GCACUGCGGCGGCAGCCGGGGUUUAAGAGCUAUGCUGGAAACAGCAU | 362 |
| | AGCAAGUUUAAAUAAGGCUAGUCCGUUAUCAACUUGAAAAAGUGGCA | |
| | CCGAGUCGGUGC | |
| MYLIP | GGCGCCACCGCGGAGGACAGGUUUAAGAGCUAUGCUGGAAACAGCAU | 363 |
| | AGCAAGUUUAAAUAAGGCUAGUCCGUUAUCAACUUGAAAAAGUGGCA | |
| | CCGAGUCGGUGC | |
| MYLIP | AUGCUCAUAGGAUGUAUUCAGUUUAAGAGCUAUGCUGGAAACAGCAU | 364 |
| | AGCAAGUUUAAAUAAGGCUAGUCCGUUAUCAACUUGAAAAAGUGGCA | |
| | CCGAGUCGGUGC | |
| MYLIP | CCACAAUAAACACAUGGUCUGUUUAAGAGCUAUGCUGGAAACAGCAU | 365 |
| | AGCAAGUUUAAAUAAGGCUAGUCCGUUAUCAACUUGAAAAAGUGGCA | |
| | CCGAGUCGGUGC | |
| MYLIP | GGGUCCCACCAGUGACAAGGGUUUAAGAGCUAUGCUGGAAACAGCAU | 366 |
| | AGCAAGUUUAAAUAAGGCUAGUCCGUUAUCAACUUGAAAAAGUGGCA | |
| | CCGAGUCGGUGC | |
| MYLIP | GGACUGCCUCAACCAGGUGAGUUUAAGAGCUAUGCUGGAAACAGCAU | 367 |
| | AGCAAGUUUAAAUAAGGCUAGUCCGUUAUCAACUUGAAAAAGUGGCA | |
| | CCGAGUCGGUGC | |
| MYLIP | CAGAGUCCCUGUCGCAGCGCGUUUAAGAGCUAUGCUGGAAACAGCAU | 368 |
| | AGCAAGUUUAAAUAAGGCUAGUCCGUUAUCAACUUGAAAAAGUGGCA | |
| | CCGAGUCGGUGC | |
| MYLIP | CUCAGAGUGAGCGAUCGCCCGUUUAAGAGCUAUGCUGGAAACAGCAU | 369 |
| | AGCAAGUUUAAAUAAGGCUAGUCCGUUAUCAACUUGAAAAAGUGGCA | |
| | CCGAGUCGGUGC | |
| MYLIP | CUGAGUUUCCCUGGCCGCCCGUUUAAGAGCUAUGCUGGAAACAGCAU | 370 |
| | AGCAAGUUUAAAUAAGGCUAGUCCGUUAUCAACUUGAAAAAGUGGCA | |
| | CCGAGUCGGUGC | |
| MYLIP | GUUUCCCUGGCCGCCCCGGGGUUUAAGAGCUAUGCUGGAAACAGCAU | 371 |
| | AGCAAGUUUAAAUAAGGCUAGUCCGUUAUCAACUUGAAAAAGUGGCA | |
| | CCGAGUCGGUGC | |
| APOB | UGAGGGCCUCCCACUCUACAGUUUAAGAGCUAUGCUGGAAACAGCAU | 384 |
| | AGCAAGUUUAAAUAAGGCUAGUCCGUUAUCAACUUGAAAAAGUGGCA | |
| | CCGAGUCGGUGC | |
| APOB | CCAGAGCACUGAAGACGCUUGUUUAAGAGCUAUGCUGGAAACAGCAU | 385 |
| | AGCAAGUUUAAAUAAGGCUAGUCCGUUAUCAACUUGAAAAAGUGGCA | |
| | CCGAGUCGGUGC | |
| APOB | ACUGGAGGAAACCUAGAAGCGUUUAAGAGCUAUGCUGGAAACAGCAU | 386 |
| | AGCAAGUUUAAAUAAGGCUAGUCCGUUAUCAACUUGAAAAAGUGGCA | |
| | CCGAGUCGGUGC | |
| APOB | CACUGAAGACGCUUGGGGAAGUUUAAGAGCUAUGCUGGAAACAGCAU | 387 |
| | AGCAAGUUUAAAUAAGGCUAGUCCGUUAUCAACUUGAAAAAGUGGCA | |
| | CCGAGUCGGUGC | |
| APOB | UGAAGAAGGCACCCCUGGUCGUUUAAGAGCUAUGCUGGAAACAGCAU | 388 |
| | AGCAAGUUUAAAUAAGGCUAGUCCGUUAUCAACUUGAAAAAGUGGCA | |
| | CCGAGUCGGUGC | |
| APOB | UGAGUGCGCGGCCGCUCUGCGUUUAAGAGCUAUGCUGGAAACAGCAU | 389 |
| | AGCAAGUUUAAAUAAGGCUAGUCCGUUAUCAACUUGAAAAAGUGGCA | |
| | CCGAGUCGGUGC | |

In some embodiments, a gRNA provided herein targets PCSK9 or a DNA regulatory element thereof. In some embodiments the gRNA targets a target site that is located within 500 bp of human genome assembly GRCh38 (hg38) genomic coordinates chr1i:55,039,548 (e.g., a target site that is +500 of 55,039,548 or −500 of 55,039,548 or positions between the foregoing). In some embodiments, the gRNA targets a target site that is within 400 bp, 300 bp, 200 bp, 100 bp, 80 bp, 60 bp, 50 bp, 40 bp, 30 bp or 20 bp of genomic coordinates chr1:55,039,548. In some embodiments the gRNA targets a target site that is located within about 80 bp of the genomic coordinate chr1:55,039,548. In some embodiments, the gRNA targets a target site in the region from −40 to +40 of the genomic coordinate chr1:55,039,548. In some embodiments the gRNA targets a target site that is located within 20 bp of the genomic coordinate chr1:55,039,548. In some embodiments, the gRNA targets a target site in the region from −10 to +10 of the genomic coordinate chr1:55,039,548. In some embodiments, any of such target sites include or span the genomic coordinate chr1:55,039,548, which is a PCSK9 transcription start site (TSS). In some embodiments, the gRNA targets a target site that is within or overlaps the coordinates chr1:55,039,538-55, 039,557. In some embodiments, the target site is or includes the coordinates chr1:55,039,538-55, 039,557. In some embodiments, a provided epigenetic-modifying DNA-targeting system for epigenetic modification of PCSK9, or a multiplexed epigenetic-modifying DNA-targeting system for epigenetic modification of at least two genes including PCSK9, includes any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 207, or a variant and/or fusion thereof.

In some embodiments, the gRNA targets a target site in PCSK9 or a DNA regulatory element thereof that comprises the sequence selected from any one of SEQ ID NO:1-13 or 306-317, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising the sequence selected from any one of SEQ ID NO:64-76 or 318-329, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:191, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:191. In some embodiments, the gRNA, including a spacer sequence and a scaffold sequence, comprises the sequence selected from any one of SEQ ID NO: 127-139 or 330-341, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting PCSK9 or a DNA regulatory element thereof, is set forth in the sequence selected from any one of SEQ ID NO:127-139 or 330-341. In some embodiments, a provided epigenetic-modifying DNA-targeting system for epigenetic modification of PCSK9, or a multiplexed epigenetic-modifying DNA-targeting system for epigenetic modification of at least two genes including PCSK9, includes any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 207, or a variant and/or fusion thereof.

In a particular embodiments, the gRNA targets a target site in the PCSK9 promoter. In some embodiments, the target site is or includes the sequence set forth in SEQ ID NO:3. In some embodiments, a gRNA provided herein comprises a spacer sequence set forth in SEQ ID NO:66. In some embodiments, the gRNA further comprises a scaffold sequence set forth in SEQ ID NO: 191. In some embodiments, the gRNA comprises the sequence set forth in SEQ ID NO:129, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 129. In some embodiments, the gRNA is set forth in SEQ ID NO: 129. In some embodiments, the gRNA is the gRNA designated PCSK9-C. In some embodiments, the gRNA targeting PCSK9 or a DNA regulatory element thereof, is set forth in the sequence selected from any one of SEQ ID NO:127-139 or 330-341. In some embodiments, a provided epigenetic-modifying DNA-targeting system for epigenetic modification of PCSK9, or a multiplexed epigenetic-modifying DNA-targeting system for epigenetic modification of at least two genes including PCSK9, includes any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 207, or a variant and/or fusion thereof.

In some embodiments, a gRNA provided herein targets LPA or a DNA regulatory element thereof. In some embodiments, the gRNA targets a target site that is located within 500 bp of the hg38 genomic coordinates chr6:160,664,275. In some embodiments, the gRNA targets a target site in LPA or a DNA regulatory element thereof that comprises the sequence selected from any one of SEQ ID NO:14-23, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising the sequence selected from any one of SEQ ID NO:77-86, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:191, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:191. In some embodiments, the gRNA, including a spacer sequence and a scaffold sequence, comprises the sequence selected from any one of SEQ ID NO: 140-149, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting LPA or a DNA regulatory element thereof, is set forth in the sequence selected from any one of SEQ ID NO: 140-149. In some embodiments, a provided epigenetic-modifying DNA-targeting system for epigenetic modification of LPA, or a multiplexed epigenetic-modifying DNA-targeting system for epigenetic modification of at least two genes including LPA, includes any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 207, or a variant and/or fusion thereof.

In some embodiments, a gRNA provided herein targets MYLIP or a DNA regulatory element thereof. In some embodiments, the gRNA targets a target site that is located within 500 bp of the hg38 genomic coordinates chr6:16,129,086. In some embodiments, the gRNA targets a target site in MYLIP or a DNA regulatory element thereof that comprises the sequence selected from any one of SEQ ID NO:24-33 or 342-351, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising the sequence selected from any one of SEQ ID NO:87-96 or 352-361, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:191, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:191. In some embodiments, the gRNA, including a spacer sequence and a scaffold sequence, comprises the sequence selected from any one of SEQ ID NO: 150-159 or 362-371, or a sequence having at or at least 80%, 85%, 90%, 91%, 92%, 93%, 94%, 95%, 96%, 97%, 98%, 99%, 99.5% 99.9%, or 100% sequence identity to all or a portion thereof. In some embodiments, the gRNA targeting MYLIP or a DNA regulatory element thereof, is set forth in the sequence selected from any one of SEQ ID NO:150-159 or 362-371. In some embodiments, a provided epigenetic-modifying DNA-targeting system for epigenetic modification of MYLIP, or a multiplexed epigenetic-modifying DNA-targeting system for epigenetic modification of at least two genes including MYLIP, includes any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 207, or a variant and/or fusion thereof.

In some embodiments, a gRNA provided herein targets ANGPTL3 or a DNA regulatory element thereof. In some embodiments, the gRNA targets a target site that is located within 500 bp of the hg38 genomic coordinates chr1:62,597,520. In some embodiments, the gRNA targets a target site in ANGPTL3 or a DNA regulatory element thereof that comprises the sequence selected from any one of SEQ ID NO:34-43, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising the sequence selected from any one of SEQ ID NO:97-106, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:191, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:191. In some embodiments, the gRNA, including a spacer sequence and a scaffold sequence, comprises the sequence selected from any one of SEQ ID NO: 160-169, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ANGPTL3 or a DNA regulatory element thereof, is set forth in the sequence selected from any one of SEQ ID NO:160-169. In some embodiments, a provided epigenetic-modifying DNA-targeting system for epigenetic modification of ANGPTL3, or a multiplexed epigenetic-modifying DNA-targeting system for epigenetic modification of at least two genes including ANGPTL3, includes any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 207, or a variant and/or fusion thereof.

In some embodiments, a gRNA provided herein targets APOC3 or a DNA regulatory element thereof. In some embodiments, the gRNA targets a target site that is located within 500 bp of the hg38 genomic coordinates chr11:116,829,907. In some embodiments, the gRNA targets a target site in APOC3 or a DNA regulatory element thereof that comprises the sequence selected from any one of SEQ ID NO:44-53, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5% 99.9% or 100% sequence identity to any of the foregoing. In some embodiments, the gRNA comprises a spacer sequence comprising the sequence selected from any one of SEQ ID NO:107-116, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:191, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:191. In some embodiments, the gRNA, including a spacer sequence and a scaffold sequence, comprises the sequence selected from any one of SEQ ID NO: 170-179, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting APOC3 or a DNA regulatory element thereof, is set forth in the sequence selected from any one of SEQ ID NO:170-179. In some embodiments, a provided epigenetic-modifying DNA-targeting system for epigenetic modification of APOC3, or a multiplexed epigenetic-modifying DNA-targeting system for epigenetic modification of at least two genes including APOC3, includes any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 207, or a variant and/or fusion thereof.

In some embodiments, a gRNA provided herein targets APOB or a DNA regulatory element thereof. In some embodiments, the gRNA targets a target site that is located within 500 bp of the hg38 genomic coordinates chr2:21,044,073. In some embodiments, the gRNA targets a target site in APOB or a DNA regulatory element thereof that comprises the sequence selected from any one of SEQ ID NO:54-63 or 372-377, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising the sequence selected from any one of SEQ ID NO:117-126 or 378-383, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO:191, or a sequence having at or at least 80%, 85%, 90%, 91%, 92%, 93%, 94%, 95%, 96%, 97%, 98%, 99%, 99.5%, 99.9%, or 100% sequence identity to SEQ ID NO:191. In some embodiments, the gRNA, including a spacer sequence and a scaffold sequence, comprises the sequence selected from any one of SEQ ID NO: 180-189 or 384-389, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting APOB or a DNA regulatory element thereof, is set forth in the sequence selected from any one of SEQ ID NO:180-189 or 384-389. In some embodiments, a provided epigenetic-modifying DNA-targeting system for epigenetic modification of APOB, or a multiplexed epigenetic-modifying DNA-targeting system for epigenetic modification of at least two genes including APOB, includes any of the aforementioned gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 207, or a variant and/or fusion thereof.

In some embodiments, provided herein is a combination of gRNAs. In some embodiments, provided herein is a multiplexed epigenetic-modifying DNA-targeting system comprising the combination of gRNAs.

In some embodiments, the combination of gRNAs comprises at least two gRNAs targeting at least two different genes. In some embodiments, the combination of gRNAs comprises a first gRNA targeted to a first gene and a second gRNA targeted to a second gene. In some embodiments, the first gRNA targets a gene selected from the list consisting of PCSK9, LPA, MYLIP, ANGPTL3, APOC3, and APOB, the second gRNA targets a gene selected from the list consisting of PCSK9, LPA, MYLIP, ANGPTL3, APOC3, and APOB, and the first and second gRNAs target different genes. In some embodiments, the first gRNA targets PCSK9 and the second gRNA targets a gene selected from the list consisting of LPA, MYLIP, ANGPTL3, APOC3, and APOB. In some embodiments, the first gRNA targets PCSK9 and the second gRNA targets LPA. In some embodiments, the first gRNA and second gRNA target a combination of two genes selected from the combinations of genes listed in Table 1. In some embodiments, the first gRNA and second gRNA are each independently selected from any of the gRNAs described herein.

In some embodiments, the combination of gRNAs comprises at least three gRNAs targeting at least three different genes. In some embodiments, the combination of gRNAs comprises a first gRNA targeted to a first gene, a second gRNA targeted to a second gene, and a third gRNA targeted to a third gene. In some embodiments, the first gRNA targets a gene selected from the list consisting of PCSK9, LPA, MYLIP, ANGPTL3, APOC3, and APOB, the second gRNA targets a gene selected from the list consisting of PCSK9, LPA, MYLIP, ANGPTL3, APOC3, and APOB, the third gRNA targets a gene selected from the list consisting of PCSK9, LPA, MYLIP, ANGPTL3, APOC3, and APOB, and the first, second, and third gRNA each target a different gene. In some embodiments, the first gRNA targets PCSK9, the second gRNA targets a gene selected from the list consisting of LPA, MYLIP, ANGPTL3, APOC3, and APOB, the third gRNA targets a gene selected from the list consisting of LPA, MYLIP, ANGPTL3, APOC3, and APOB, and the second gRNA and third gRNA target different genes. In some embodiments, the first gRNA targets PCSK9, the second gRNA targets LPA, and the third gRNA targets a gene selected from the list consisting of MYLIP, ANGPTL3, APOC3, and APOB. In some embodiments, the first gRNA, second gRNA, and third gRNA target a combination of three genes selected from the combinations of genes listed in Table 2. In some embodiments, the first gRNA, second gRNA, and third gRNA are each independently selected from any of the gRNAs described herein.

### C. Other DNA-Binding Domains

In some of any of the provided embodiments, the DNA-binding domain comprises a zinc finger protein (ZFP); a transcription activator-like effector (TALE); a meganuclease; a homing endonuclease; or an I-SceI enzyme or a variant thereof. In some embodiments, the DNA-binding domain comprises a catalytically inactive variant of any of the foregoing.

In some embodiments, a ZFP, a zinc finger DNA binding protein, or zinc finger DNA binding domain, is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial, or engineered, ZFPs, comprising ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some embodiments, zinc fingers are custom-designed (i.e. designed by the user), or obtained from a commercial source. Various methods for designing zinc finger proteins are available. For example, methods for designing zinc finger proteins to bind to a target DNA sequence of interest are described, for example in Liu, Q. et al., PNAS, 94(11):5525-30 (1997); Wright, D. A. et al., Nat. Protoc., 1(3):1637-52 (2006); Gersbach, C. A. et al., Acc. Chem. Res., 47(8):2309-18 (2014); Bhakta M. S. et al., Methods Mol. Biol., 649:3-30 (2010); and Gaj et al., Trends Biotechnol, 31(7):397-405 (2013). In addition, various web-based tools for designing zinc finger proteins to bind to a DNA target sequence of interest are publicly available. See, for example, the Zinc Finger Tools design web site from Scripps available on the world wide web at scripps.edu/barbas/zfdesign/zfdesignhome.php. Various commercial services for designing zinc finger proteins to bind to a DNA target sequence of interest are also available. See, for example, the commercially available services or kits offered by Creative Biolabs (world wide web at creative-biolabs.com/Design-and-Synthesis-of-Artificial-Zinc-Finger-Proteins.html), the Zinc Finger Consortium Modular Assembly Kit available from Addgene (world wide web at addgene.org/kits/zfc-modular-assembly/), or the CompoZr Custom ZFN Service from Sigma Aldrich (world wide web at sigmaaldrich.com/life-science/zinc-finger-nuclease-technology/custom-zfn.html).

Transcription activator-like effectors (TALEs), are proteins naturally found in Xanthomonas bacteria. TALEs comprise a plurality of repeated amino acid sequences, each repeat having binding specificity for one base in a target sequence. Each repeat comprises a pair of variable residues in position 12 and 13 (repeat variable diresidue; RVD) that determine the nucleotide specificity of the repeat. In some embodiments, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In some embodiments, RVDs can be mutated towards other amino acid residues in order to modulate their specificity towards nucleotides A, T, C and G and in particular to enhance this specificity. Binding domains with similar modular base-per-base nucleic acid binding properties can also be derived from different bacterial species. These alternative modular proteins may exhibit more sequence variability than TALE repeats.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a TALE is a fusion protein comprising a nucleic acid binding domain derived from a TALE and an effector domain.

Zinc finger and TALE DNA-binding domains can be engineered to bind to a predetermined nucleotide sequence, for example via engineering (altering one or more amino acids) of the recognition helix region of a naturally occurring zinc finger protein, by engineering of the amino acids in a TALE repeat involved in DNA binding (the repeat variable diresidue or RVD region), or by systematic ordering of modular DNA-binding domains, such as TALE repeats or ZFP domains. Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

### D. Effector Domains

In some aspects, the DNA-targeting systems provided herein further include one or more effector domains. In some embodiments, provided herein is a DNA-targeting system comprising a fusion protein comprising: (a) a DNA-binding domain capable of being targeted to a target site in a gene or regulatory DNA element thereof, such as any described above, and (b) at least one effector domain. In some aspects, the effector domain is capable of reducing transcription of the gene or combination of genes. In some aspects, the effector domain comprises a transcription repressor domain.

In some aspects, the effector domain induces, catalyzes, or leads to repressed and/or reduced transcription of a gene when ectopically recruited to the gene or DNA regulatory element thereof.

In some embodiments, the effector domain induces, catalyzes or leads to transcription repression, transcription co-repression, histone modification, histone acetylation, histone deacetylation, nucleosome remodeling, chromatin remodeling, heterochromatin formation, proteolysis, ubiquitination, deubiquitination, phosphorylation, dephosphorylation, splicing, DNA methylation, DNA demethylation, histone methylation, histone demethylation, or DNA base oxidation. In some embodiments, the effector domain induces, catalyzes, or leads to transcription repression or transcription co-repression. In some embodiments, the effector domain induces transcription repression. In some embodiments, the effector domain has one of the aforementioned activities itself (i.e. acts directly). In some embodiments, the effector domain recruits and/or interacts with a protein or polypeptide domain that has one of the aforementioned activities (i.e. acts indirectly).

Gene expression of endogenous mammalian genes, such as human genes, can be achieved by targeting a fusion protein comprising a DNA-binding domain, such as a dCas9, and an effector domain, such as a transcription repression domain, to mammalian genes or regulatory DNA elements thereof (e.g. a promoter or enhancer) via one or more gRNAs. Any of a variety of effector domains for transcriptional repression (e.g. transcription repression domains) are known and can be used in accord with the provided embodiments. Transcription repression domains, as well as transcriptional repression of target genes using Cas fusion proteins with the transcription repression domains, are described, for example, in WO 2014/197748, WO 2017/180915, WO 2021/226077, WO 2013/176772, WO 2014/152432, WO 2014/093661, Adli, M. Nat. Commun. 9, 1911 (2018), and Gilbert, L. A. et al. Cell 154(2):442-451 (2013).

In some embodiments, the effector domain may comprise a KRAB domain, ERF repressor domain, MXI1 domain, SID4X domain, MAD-SID domain, a DNMT family protein domain (e.g. DNMT3A or DNMT3B), a fusion of one or more DNMT family proteins or domains thereof (e.g. DNMT3A/L, which comprises a fusion of DNMT3A and DNMT3L domains), LSD1, EZH2, a SunTag domain, a partially or fully functional fragment or domain of any of the foregoing, or a combination of any of the foregoing. For example, the fusion protein may be dCas9-KRAB, or dCas9-KRAB-DNMT3A/L. In some embodiments, the fusion protein may be dCas9-KRAB. In some embodiments, the fusion protein may be DNMT3A/L-dCas9-KRAB. In some embodiments, the fusion protein may be KRAB-dCas9-DNMT3A/L.

In some embodiments, the effector domain comprises a transcriptional repressor domain described in WO 2021/226077.

In some embodiments, the effector domain comprises a KRAB domain, or a variant thereof. The KRAB-containing zinc finger proteins make up the largest family of transcriptional repressors in mammals. The Kruppel associated box (KRAB) domain is a transcriptional repressor domain present in many zinc finger protein-based transcription factors. The KRAB domain comprises charged amino acids and can be divided into sub-domains A and B. The KRAB domain recruits corepressors KAP1 (KRAB-associated protein-1), epigenetic readers such as heterochromatin protein 1 (HPI), and other chromatin modulators to induce transcriptional repression through heterochromatin formation. KRAB-mediated gene repression is associated with loss of histone H3-acetylation and an increase in H3 lysine 9 trimethylation (H3K9me3) at the repressed gene promoters. KRAB domains, including in dCas fusion proteins, have been described, for example, in WO 2017/180915, WO 2014/197748, US 2019/0127713, WO 2013/176772, Urrutia R. et al. Genome Biol. 4, 231 (2003), Groner A. C. et al. PLoS Genet. 6, e1000869 (2010). In some embodiments, the effector domain comprises at least one KRAB domain or a variant thereof. In some embodiments, an exemplary KRAB domain is set forth in SEQ ID NO: 193. In some embodiments, the effector domain comprises the sequence set forth in SEQ ID NO: 193, or a portion thereof, or an amino acid sequence that has at least 90%, 91%, 92%, 93%, 94%, 95%, 96%, 97%, 98%, or 99% sequence identity to SEQ ID NO:193. In some embodiments, an exemplary KRAB domain is set forth in SEQ ID NO: 290. In some embodiments, the effector domain comprises the sequence set forth in SEQ ID NO: 290, or a portion thereof, or an amino acid sequence that has at least 90%, 91%, 92%, 93%, 94%, 95%, 96%, 97%, 98%, or 99% sequence identity to SEQ ID NO:290.

In some embodiments, the effector domain comprises at least one ERF repressor domain, or a variant thereof. ERF (ETS2 repressor factor) is a strong transcriptional repressor that comprises a conserved ets-DNA-binding domain, and represses transcription via a distinct domain at the carboxyl-terminus of the protein. ERF repressor domains, including in dCas fusion proteins, have been described, for example, in WO2017180915, WO2014197748, WO2013176772, Mavrothalassitis, G., Ghysdael, J. Proteins of the ETS family with transcriptional repressor activity. Oncogene 19, 6524-6532 (2000). In some embodiments, the effector domain comprises at least one ERF repressor domain or a variant thereof. An exemplary ERF repressor domain is set forth in SEQ ID NO:220. In some embodiments, the effector domain comprises the sequence set forth in SEQ ID NO:220, or a portion thereof, or an amino acid sequence that has at least 90%, 91%, 92%, 93%, 94%, 95%, 96%, 97%, 98%, or 99% sequence identity to any of the foregoing.

In some embodiments, the effector domain comprises at least one MXI1 domain, or a variant thereof. The MXI1 domain functions by antagonizing the myc transcriptional activity by competing for binding to myc-associated factor x (MAX). MXI1 domains, including in dCas fusion proteins, have been described, for example, in WO2017180915, WO2014197748, US20190127713. In some embodiments, the effector domain comprises at least one MXI1 domain or a variant thereof. An exemplary MXI1 domain is set forth in SEQ ID NO:221. In some embodiments, the effector domain comprises the sequence set forth in SEQ ID NO:221, or a portion thereof, or an amino acid sequence that has at least 90%, 91%, 92%, 93%, 94%, 95%, 96%, 97%, 98%, or 99% sequence identity to any of the foregoing.

In some embodiments, the effector domain comprises at least one SID4X domain, or a variant thereof. The mSin3 interacting domain (SID) is present on different transcription repressor proteins. It interacts with the paired amphipathic alpha-helix 2 (PAH2) domain of mSin3, a transcriptional repressor domain that is attached to transcription repressor proteins such as the mSin3 A corepressor. A dCas9 molecule can be fused to four concatenated mSin3 interaction domains (SID4X). SID domains, including in dCas fusion proteins, have been described, for example, in WO2017180915, WO2014197748, WO2014093655. In some embodiments, the effector domain comprises at least one SID domain or a variant thereof. An exemplary SID domain is set forth in SEQ ID NO:222. In some embodiments, the effector domain comprises the sequence set forth in SEQ ID NO:222, or a portion thereof, or an amino acid sequence that has at least 90%, 91%, 92%, 93%, 94%, 95%, 96%, 97%, 98%, or 99% sequence identity to any of the foregoing.

In some embodiments, the effector domain comprises at least one MAD domain, or a variant thereof. The MAD family proteins, Mad1, Mxi1, Mad3, and Mad4, belong to the basic helix-loop-helix-zipper class and contain a conserved N terminal region (termed Sin3 interaction domain (SID)) necessary for repressional activity. MAD-SID domains, including in dCas fusion proteins, have been described, for example, in WO2017180915, WO2014197748, WO2013176772. In some embodiments, the effector domain comprises at least one MAD-SID domain or a variant thereof. An exemplary MAD-SID domain is set forth in SEQ ID NO:223. In some embodiments, the effector domain comprises the sequence set forth in SEQ ID NO:223, or a portion thereof, or an amino acid sequence that has at least 90%, 91%, 92%, 93%, 94%, 95%, 96%, 97%, 98%, or 99% sequence identity to any of the foregoing.

In some embodiments, the effector domain comprises at least one DNMT3 domain, or a variant thereof. In some embodiments, the at least one DNMT3 domain, or a variant thereof, is from a DNMT3 or is a portion or a functionally active variant thereof with DNA methyltransferase activity. The DNMT3A and DNMT3B are two DNA methyltransferases that catalyze de novo methylation, which depending on the site may be associated with transcriptional repression. DNMTs, such as DNMT3s, mediate transfer of a methyl group from the universal methyl donor, S-adenosyl-L-methionine (SAM), to the 5-position of cytosine residues. In some aspects, these DNMT3 DNA methyltransferases induce de novo methylation of a cytosine base to methylated 5-methylcytosine. DNMT3, including in dCas fusion proteins, have been described, for example, in US20190127713, Liu, X. S. et al. Cell 167, 233-247.e17 (2016), Lei, Y. et al. Nat. Commun. 8, 16026 (2017). DNMT3 proteins, such as DNMT3A and DNMT3B, contain an N-terminal part that is naturally involved in regulatory activity and targeting, and a C-terminal catalytic domain termed the MTase C5-type domain. In some embodiments, an effector domain in embodiments provided herein includes a catalytically active portion of a DNMT3A or a DNMT3B that contains a catalytically active C-terminal domain. In particular, isolated catalytic domains of DNMT3a and DNMT3b are catalytically active (see e.g. Gowher and Jeltsch (2002) J. Biol. Chem., 277:20409).

In some embodiments, the effector domain comprises at least one DNMT3 domain or a variant thereof. In some embodiments, the DNMT3 domain may be an effector domain of DNMT3A or DNMT3B that is catalytically active. In some embodiments, the effector domain may be the full-length of DNMT3A or DNMT3B or a catalytically active portion thereof. In some embodiments, the effector domain is a catalytically active portion that is less than the full-length sequence of DNMT3A or DNMT3B. In some embodiments, a catalytically active portion is a contiguous sequence of amino acids that confers DNA methyltransferase activity, such as by mediating methylation of a cytosine base to methylated 5-methylcytosine. In some embodiments, the contiguous sequence of amino acids is a contiguous C-terminal portion of a DNMT3 protein, such as DNMT3A, or DNMT3B, that is from 280 amino acids to 330 amino acids in length. In some embodiments, the contiguous portion is 280 amino acids, 290 amino acids, 300 amino acids, 310 amino acids, 320 amino acids, or 330 amino acids in length, or is a length of any value between any of the foregoing. In some embodiments, a catalytically active portion of a DNMT, such as a DNMT3, includes a SAM-dependent MTase C5-type domain. In some embodiments, the DNMT3 domain, such as a domain of DNMT3A or DNMT3B, is of human origin.

An exemplary DNMT3A domain is set forth in SEQ ID NO:195 or 285. An exemplary DNMT3B domain is set forth in SEQ ID NO:224. In some embodiments, the effector domain comprises the sequence set forth in SEQ ID NO: 195, SEQ ID NO: 285 or SEQ ID NO: 224, or a portion thereof, or an amino acid sequence that has at least 90%, 91%, 92%, 93%, 94%, 95%, 96%, 97%, 98%, or 99% sequence identity to any of the foregoing.

In some embodiments, the DNMT3A domain is set forth in SEQ ID NO: 195, or is a catalytically active portion thereof, or is an amino acid sequence that has at least 90%, 91%, 92%, 93%, 94%, 95%, 96%, 97%, 98%, or 99% sequence identity to SEQ ID NO:195 or the catalytically active portion thereof that exhibits DNA methyltransferase activity. In some embodiments, the DNMT3A domain is set forth in SEQ ID NO:195. In some embodiments, the DNMT3A domain is encoded by the nucleotide sequence set forth in SEQ ID NO: 194.

In some embodiments, the DNMT3A domain is set forth in SEQ ID NO:285, or is a catalytically active portion thereof, or is an amino acid sequence that has at least 90%, 91%, 92%, 93%, 94%, 95%, 96%, 97%, 98%, or 99% sequence identity to SEQ ID NO:285 or the catalytically active portion thereof that exhibits DNA methyltransferase activity. In some embodiments, the DNMT3A domain is set forth in SEQ ID NO:285.

In some embodiments, the effector domain is from DNMT3B or a catalytically active portion or variant thereof that exhibits DNA methyltransferase activity. An exemplary DNMT3B domain is set forth in SEQ ID NO:224, or is a catalytically active portion thereof, or is an amino acid sequence that has at least 90%, 91%, 92%, 93%, 94%, 95%, 96%, 97%, 98%, or 99% sequence identity to SEQ ID NO:224 or the catalytically active portion thereof that exhibits DNA methyltransferase activity. In some embodiments, the catalytically active portion is a contiguous portion of amino acids of SEQ ID NO:224 that includes the SAM-dependent MTase C5-type domain (e.g. corresponding to amino acids 575-853 of SEQ ID NO:224). In some embodiments, the contiguous sequence of amino acids of SEQ ID NO: 224 includes at least 250 amino acids, 275 amino acids, 300 amino acids or 325 amino acids, or any value between any of the foregoing. In some embodiments, the contiguous sequence of amino acids is a contiguous portion of SEQ ID NO:224 that includes amino acids 575-853 and is from 280 amino acids to 330 amino acids in length. In some embodiments, the contiguous portion is 280 amino acids, 290 amino acids, 300 amino acids, 310 amino acids, 320 amino acids, or 330 amino acids in length, or is a length of any value between any of the foregoing.

Any of a variety of assays are known to assess or monitor methyltransferase (MTase) activity. In some embodiments, exemplary assays to assess DNA methyltransferase activity include, but are not limited to, radio DNA MTase assays, colorimetric DNA MTase activity assays, fluorescent DNA MTase activity assays, chemiluminescent/bioluminescent DNA MTase activity assays, electrochemical DNA MTase activity assays, and elctrogenerated chemiluminescence (ECL) DNA MTase activity assays. Exemplary assays are described in Poh et al. Theranostics, 2016, 6:369-391; Li et al., Methods Appl. Fluoresc., 2017, 5:012002; Deng et al., Anal Chem., 2014, 86:2117-23; and Ma et al. J Mater Chem B., 2020, 8:3488-3501.

In some embodiments, the effector domain includes at least one DNMT3L domain, or a variant thereof. The DNMT3L domain or a variant thereof may be a DNMT3L or a portion of DNMT3L, or a variant of DNMT3L or the portion thereof. DNMT3L (DNA (cytosine-5)-methyltransferase 3-like) is a catalytically inactive regulatory factor of DNA methyltransferases that can either promote or inhibit DNA methylation depending on the context. DNMT3L is essential for the function of DNMT3A and DNMT3B; DNMT3L interacts with DNMT3A and DNMT3B and significantly enhances their catalytic activity. For instance, DNMT3L interacts with the catalytic domain of DNMT3A to form a heterodimer, demonstrating that DNMT3L has dual functions of binding an unmethylated histone tail and activating DNA methyltransferase. In some embodiments, reference to a portion or variant of a DNMT3L for purposes herein refers to a sufficient C-terminal sequence portion of DNMT3L that interacts with the catalytic domain of DNMT3A or DNMT3B and is able to stimulate or promote DNA methyltransferase activity of DNMT3A or DNMT3B (see e.g. Jia et al. Nature, 2007, 449:248-251; Gowher et al. J. Biol. Chem., 2005, 280: 13341-13348). In some embodiments, the DNMT3L or portion thereof is of animal origin. In some embodiments, the domain from DNMT3L is of murine origin. In some embodiments, the domain from DNMT3L is of human origin.

In some embodiments, the DNMT3L domain is a DNMT3L, or a C-terminal portion or variant thereof, that interacts with the catalytic domain of DNMT3A to form a heterodimer to provide for a more active DNA methyltransferase. In some embodiments, the effector domain is a fusion domain of a DNMT3A domain and the DNMT3L domain (DNMT3A/3L).

In some embodiments, the DNMT3L domain is a DNMT3L, or a C-terminal portion or variant thereof, that interacts with the catalytic domain of DNMT3B to form a heterodimer to provide for a more active DNA methyltransferase. In some embodiments, the effector domain is a fusion domain of a DNMT3B domain and the DNMT3L domain (DNMT3B/3L).

In some embodiments, the DNMT3L domain is a C-terminal portion of DNMT3L composed of a contiguous C-terminal portion of the full-length DNMT3L that does not include the N-terminal cysteine-rich ATRX-Dnmt3-Dnmt3L (ADD) domain (e.g. corresponding to residues 41-73 of SEQ ID NO: 197 or 75-207 of the sequence set forth in SEQ ID NO:289). In some embodiments, the DNMT3L domain is a contiguous C-terminal portion of DNMT3L that is less than 220 amino acids in length, such as between 100 and 215 amino acids, such as at or about 100, 110, 120, 130, 140, 150, 160, 170, 180, 190, 200, 210 or 215 amino acids in length, or a length between a value of any of the foregoing. In some embodiments, the DNMT3L domain is a contiguous C-terminal portion of DNMT3L that is 205, 206, 207, 208, 209, 210, 211, 212, 213, 214 or 215 amino acids in length.

An exemplary DNMT3L domain is set forth in SEQ ID NO:289, or is a portion thereof, or is an amino acid sequence that has at least 90%, 91%, 92%, 93%, 94%, 95%, 96%, 97%, 98%, or 99% sequence identity to SEQ ID NO:289 or the portion thereof. In some embodiments, the DNMT3L domain is a contiguous C-terminal portion of the full-length DNMT3L set forth in SEQ ID NO: 289 that does not include the N-terminal cysteine-rich ATRX-Dnmt3-Dnmt3L (ADD) domain (corresponding to residues 75-207 of the sequence set forth in SEQ ID NO:289). In some embodiments, the DNMT3L domain is a contiguous C-terminal portion of the full-length DNMT3L set forth in SEQ ID NO: 289 that is less than 220 amino acids in length, such as between 100 and 215 amino acids, such as at or about 100, 110, 120, 130, 140, 150, 160, 170, 180, 190, 200, 210 or 215 amino acids in length, or a length between a value of any of the foregoing. In some embodiments, the DNMT3L domain is a contiguous C-terminal portion of the full-length DNMT3L set forth in SEQ ID NO: 289 that is 205, 206, 207, 208, 209, 210, 211, 212, 213, 214 or 215 amino acids in length.

In some embodiments, the DNMT3L domain is set forth in SEQ ID NO:286, or is a portion thereof, or is an amino acid sequence that has at least 90%, 91%, 92%, 93%, 94%, 95%, 96%, 97%, 98%, or 99% sequence identity to SEQ ID NO:286. In some embodiments, the DNMT3L domain is set forth in SEQ ID NO:286. In some embodiments, the DNMT3L domain does not contain an N-terminal methionine, such as set forth in SEQ ID NO: 286.

In some embodiments, the DNMT3L domain is a human or humanized DNMT3L. Corresponding sequences of human DNMT3L are highly homologous to the DNMT3L derived from mouse and have a sequence identity of at least 90% with the murine sequence. It is within the level of a skilled artisan to humanize a non-human sequence of a DNMT3L domain, such as a domain of a murine DNMT3L. In some embodiments, the effector domain includes a DNMT3L domain that is a humanized variant of the murine DMT3L set forth in SEQ ID NO:289 or a portion thereof that is able to interact with DNMT3A or DNMT3A. In some embodiments, the effector domain includes a DNMT3L domain that is a humanized variant of the murine C-terminal portion of DNMT3L set forth in SEQ ID NO:286.

An exemplary DNMT3L domain of human origin is set forth in SEQ ID NO: 197, or is a portion thereof, or is an amino acid sequence that has at least 90%, 91%, 92%, 93%, 94%, 95%, 96%, 97%, 98%, or 99% sequence identity to SEQ ID NO:197 or the portion thereof. In some embodiments, the DNMT3L domain is a contiguous C-terminal portion of the full-length DNMT3L set forth in SEQ ID NO: 197 that does not include the N-terminal cysteine-rich ATRX-Dnmt3-Dnmt3L (ADD) domain (corresponding to residues 41-73 of the sequence set forth in SEQ ID NO:197). In some embodiments, the DNMT3L domain is a contiguous C-terminal portion of the full-length DNMT3L set forth in SEQ ID NO: 197 that is less than 220 amino acids in length, such as between 100 and 215 amino acids, such as at or about 100, 110, 120, 130, 140, 150, 160, 170, 180, 190, 200, 210 or 215 amino acids in length, or a length between a value of any of the foregoing. In some embodiments, the DNMT3L domain is a contiguous C-terminal portion of the full-length DNMT3L set forth in SEQ ID NO: 197 that is 205, 206, 207, 208, 209, 210, 211, 212, 213, 214 or 215 amino acids in length.

An exemplary DNMT3L domain is set forth in SEQ ID NO:197. In some embodiments, a DNMT3L domain comprises the sequence set forth in SEQ ID NO:197, or a portion thereof, or an amino acid sequence that has at least 90%, 91%, 92%, 93%, 94%, 95%, 96%, 97%, 98%, or 99% sequence identity to SEQ ID NO:197. In some embodiments, the DNMT3L domain is encoded by the nucleotide sequence set forth in SEQ ID NO: 196.

In some embodiments, the DNMT3L domain comprises the sequence set forth in SEQ ID NO:287, or is a portion thereof, or is an amino acid sequence that has at least 90%, 91%, 92%, 93%, 94%, 95%, 96%, 97%, 98%, or 99% sequence identity to SEQ ID NO:287. In some embodiments, the DNMT3L domain is set forth in SEQ ID NO:287. In some embodiments, the DNMT3L domain contains an N-terminal methionine.

In some embodiments, the effector domain comprises a fusion of DNMT3A and DNMT3L (DNMT3A/L). The fusion protein contains DNMT3A and DNMT3L domains that can be any as described above. In some embodiments, the fusion protein contains the DNMT3A domain set forth in SEQ ID NO: 195 and the DNMT3L domain set forth in SEQ ID NO: 289, arranged in any order. In some embodiments, the fusion protein contains the DNMT3A domain set forth in SEQ ID NO: 195 and the DNMT3L domain set forth in SEQ ID NO:286, arranged in any order. In some embodiments, the fusion protein contains the DNMT3A domain set forth in SEQ ID NO:195 and the DNMT3L domain set forth in SEQ ID NO:287, arranged in any order. In some embodiments, the fusion protein contains the DNMT3A domain set forth in SEQ ID NO: 285 and the DNMT3L domain set forth in SEQ ID NO: 289, arranged in any order. In some embodiments, the fusion protein contains the DNMT3A domain set forth in SEQ ID NO: 285 and the DNMT3L domain set forth in SEQ ID NO:286, arranged in any order. In some embodiments, the fusion protein contains the DNMT3A domain set forth in SEQ ID NO:285 and the DNMT3L domain set forth in SEQ ID NO:287, arranged in any order. In some embodiments, the DNMT3A and DNMT3L domains present in a provided fusion protein are separated from each other in the fusion protein by an intervening sequence, such as the DNA-binding domain, another effector domain or a linker. In some embodiments, the domains are either directly linked to each other or they are linked via a linker, such as a peptide linker. In some embodiments, the DNMT3A and DNMT3L domains are connected as a fusion domain via a linker that connects the DNMT3A domain and the DNMT3L domain. Exemplary linkers are described herein. In some embodiments, the linker is the linker set forth in SEQ ID NO: 288.

An exemplary DNMT3A/L fusion domain is set forth in SEQ ID NO: 199. In some embodiments, the effector domain comprises the sequence set forth in SEQ ID NO:199, or a portion thereof, or an amino acid sequence that has at least 90%, 91%, 92%, 93%, 94%, 95%, 96%, 97%, 98%, or 99% sequence identity to SEQ ID NO: 199. In some embodiments, the DNMT3A/L fusion domain is encoded by the nucleotide sequence set forth is SEQ ID NO: 198.

An exemplary DNMT3A/L fusion domain is set forth in SEQ ID NO:201. In some embodiments, the effector domain comprises the sequence set forth in SEQ ID NO:201, or a portion thereof, or an amino acid sequence that has at least 90%, 91%, 92%, 93%, 94%, 95%, 96%, 97%, 98%, or 99% sequence identity to SEQ ID NO: 201. In some embodiments, the DNMT3A/L fusion domain is encoded by the nucleotide sequence set forth is SEQ ID NO: 200.

In some embodiments, the effector domain may comprise a LSD1 domain. LSD1 (also known as Lysine-specific histone demethylase 1A) is a histone demethylase that can demethylate lysine residues of histone H3, thereby acting as a coactivator or a corepressor, depending on the context. LSD1, including in dCas fusion proteins, has been described, for example, in WO 2013/176772, WO 2014/152432, and Kearns, N. A. et al. Nat. Methods. 12(5):401-403 (2015). An exemplary LSD1 polypeptide is set forth in SEQ ID NO:225. In some embodiments, the effector domain comprises the sequence set forth in SEQ ID NO:225, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise an EZH2 domain. EZH2 (also known as Histone-lysine N-methyltransferase EZH2) is a Catalytic subunit of the PRC2/EED-EZH2 complex, which methylates ‘Lys-9’ (H3K9me) and ‘Lys-27’ (H3K27me) of histone H3, in some aspects leading to transcriptional repression of the affected target gene. EZH2, including in dCas fusion proteins, has been described, for example, in O'Geen, H. et al., Epigenetics Chromatin. 12(1):26 (2019). An exemplary EZH2 polypeptide is set forth in SEQ ID NO:283. In some embodiments, the effector domain comprises the sequence set forth in SEQ ID NO:283, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise a SunTag domain. SunTag is a repeating peptide array, which can recruit multiple copies of an antibody-fusion protein that binds the repeating peptide. The antibody-fusion protein may comprise an additional effector domain, such as a transcription repression domain (e.g. KRAB), to reduce transcription of the target gene. SunTag, including in dCas fusion proteins for gene modulation have been described, for example, in WO 2016/011070 and Tanenbaum, M. et al. Cell. 159(3):635-646 (2014). An exemplary SunTag effector domain includes a repeating GCN4 peptide having the amino acid sequence LLPKNYHLENEVARLKKLVGER (SEQ ID NO: 226) separated by linkers having the amino acid sequence GGSGG (SEQ ID NO: 227). In some embodiments, the effector domain comprises at least one copy of the sequence set forth in SEQ ID NO: 226, or a portion thereof, or an amino acid sequence that has at least 90%, 91%, 92%, 93%, 94%, 95%, 96%, 97%, 98%, or 99% sequence identity to any of the foregoing. In some embodiments, the SunTag effector domain recruits an antibody-fusion protein that comprises KRAB and binds the GCN4 peptide.

### E. Fusion Proteins

In some aspects, the DNA-targeting systems provided herein include fusion proteins. In some embodiments, provided herein is a DNA-targeting system that is a fusion protein comprising: (a) a DNA-binding domain capable of being targeted to a target site in a gene or regulatory DNA element thereof in which the gene encodes a gene product that regulates LDL, and (b) at least one effector domain. In some embodiments, provided herein is a DNA-targeting system comprising at least one DNA-targeting module, in which each of the at least one DNA-targeting module comprises a fusion protein comprising: (a) a DNA-binding domain capable of being targeted to a target site in a gene or regulatory DNA element thereof in which the gene encodes a gene product that regulates LDL, and (b) at least one effector domain. In some aspects, the fusion protein comprises at least one of any of the DNA-binding domains described herein, and at least one of any of the effector domains described herein. For instance, in some embodiments, the fusion protein contains a CRISPR-Cas DNA-binding domain, such as described in Section IIB, and at least one effector domain described herein. In some embodiments, the effector domain of a provided fusion protein is a transcriptional repressor domain, such as any described in Section I.D. In some aspects, the fusion protein is targeted to a target site in a gene or regulatory element thereof, and leads to reduced or repressed transcription of the gene. In some aspects, the fusion protein is targeted to target sites in a combination of genes or regulatory elements thereof, and leads to reduced or repressed transcription of each of the genes.

In some embodiments, the DNA-binding domain and effector domain of the fusion protein are heterologous, i.e. the domains are from different species, or at least one of the domains is not found in nature. In some aspects, the fusion protein is an engineered fusion protein, i.e. the fusion protein is not found in nature.

In some embodiments, the at least one effector domain is fused to the N-terminus, the C-terminus, or both the N-terminus and the C-terminus, of the DNA-binding domain or a component thereof. The at least one effector domain may be fused to the DNA-binding domain directly, or via any intervening amino acid sequence, such as a linker sequence or a nuclear localization sequence (NLS).

In some embodiments, the fusion protein of a provided DNA-binding system, or a DNA-targeting module thereof, comprises, from N- to C-terminal order: a transcriptional repressor effector domain and a DNA-binding domain. In some embodiments, the fusion protein of a provided DNA-binding system, or a DNA-targeting module thereof, comprises, from N- to C-terminal order: a DNA-binding domain and a transcriptional repressor effector domain.

In some embodiments, the at least one effector domain of the fusion protein includes more than one effector domains. In some embodiments, the fusion protein includes 2, 3 or 4 effector domains. In some embodiments, at least two of the effector domains of the fusion protein are different. In some embodiments, each of the effector domains of the fusion protein are different. In some embodiments, the at least one effector domain includes two effector domains in which the two effector domains are different. In some embodiments, the effector domains and the DNA-binding domain can be arranged in any order.

In some embodiments, the at least one effector domain of the fusion protein includes two different effector domains. The two different effector domains and the DNA-binding domain can be arranged in any order. In some embodiments, each of the effector domains are N-terminal to the DNA-binding domain in which a first effector domain is fused to the N-terminus of the second effector domain and the second effector domain is fused to the N-terminus of the DNA-binding domain. In some embodiments, the fusion protein of a provided DNA-binding system, or a DNA-targeting module thereof, comprises from N- to C-terminal order: a first transcriptional repressor effector domain, a second transcriptional repressor effector domain and the DNA binding domain. In some embodiments, each of the effector domains are C-terminal to the DNA-binding domain in which a first effector domain is fused to the C-terminus of the DNA-binding domain and the second effector domain is fused to the C-terminus of the first effector domain. In some embodiments, the fusion protein of a provided DNA-binding system, or a DNA-targeting module thereof, comprises from N—to C-terminal order: a DNA-binding domain, a first transcriptional repressor effector domain, and a second transcriptional repressor effector domain. In some embodiments, the DNA-binding domain is between the effector domains, in which one effector domain is fused to the N-terminus of the DNA-binding domain and the other effector domain is fused to the C-terminus of the DNA-binding domain. In some embodiments, the fusion protein of a provided DNA-binding system, or a DNA-targeting module thereof, comprises from N- to C-terminal order: a first transcriptional effector domain, a DNA-binding domain, and a second transcriptional repressor effector domain. In some embodiments, one or more of the components may be fused to each other directly, or via any intervening amino acid sequence, such as via a linker sequence or a nuclear localization sequence (NLS).

In some embodiments, the fusion protein comprises one or more linkers. In some embodiments, the linker is a peptide linker. In some embodiments, the one or more linkers connect the DNA-binding domain or a component thereof to the at least one effector domain. A linker may be included anywhere in the polypeptide sequence of the fusion protein, for example, between the effector domain and the DNA-binding domain or a component thereof. A linker may be of any length and designed to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A skilled artisan can readily choose an appropriate linker for the connection of two domains. In some embodiments, the linker is a flexible linker. Flexible linkers are generally composed of small, non-polar or polar residues such as glycine, serine or threonine. In some embodiments, the linker is the Gly₄Ser₍ₙ₎ linker, whereby n is an integer of 1 to 10. A linker may include sequential or tandem repeats of an amino acid sequence that is 2 to 20 amino acids in length. Linkers may be rich in amino acids glycine (G), serine (S), and/or alanine (A). Linkers may include, for example, a GS linker. An exemplary GS linker is represented by the sequence GGGGS (SEQ ID NO: 228). A linker may comprise repeats of a sequence, for example as represented by the formula (GGGGS)n, wherein n is an integer that represents the number of times the GGGGS sequence is repeated (e.g. between 1 and 10 times). The number of times a linker sequence is repeated can be adjusted to optimize the linker length and achieve appropriate separation of the functional domains. Other examples of linkers may include, for example, GGGGG (SEQ ID NO: 229), GGAGG (SEQ ID NO: 230), GGGGSSS (SEQ ID NO: 231), or GGGGAAA (SEQ ID NO: 232).

In some embodiments, artificial linker sequences can be used. In some embodiments, the linker is EASGSGRASPGIPGSTR (SEQ ID NO: 293). In some embodiments, the linker is linker is GIHGVPAA (SEQ ID NO: 294). In some embodiments, the linker is SSGNSNANSRGPSFSSGLVPLSLRGSH (SEQ ID NO: 288). In some embodiments, the linker is KRPAATKKAGQAKKKKASDAKSLTAWS (SEQ ID NO: 298).

In some embodiments, inclusion of a SunTag linker in the fusion protein leads to enhanced repression of the target gene. In some embodiments, a SunTag linker comprises the sequence set forth in SEQ ID NO:226, or a portion thereof, or an amino acid sequence that has at least 90%, 91%, 92%, 93%, 94%, 95%, 96%, 97%, 98%, or 99% sequence identity to any of the foregoing.

In some embodiments, the linker is an XTEN linker. In some aspects, an XTEN linker is a recombinant polypeptide (e.g., an unstructured recombinant peptide) lacking hydrophobic amino acid residues. Exemplary XTEN linkers are described in, for example, Schellenberger et al., Nature Biotechnology 27, 1186-1190 (2009) or WO 2021/247570. In some embodiments, inclusion of a linker in the fusion protein leads to enhanced repression of the target gene. In some embodiments, a linker comprises the sequence set forth in SEQ ID NO:233, or a portion thereof, or an amino acid sequence that has at least 90%, 91%, 92%, 93%, 94%, 95%, 96%, 97%, 98%, or 99% sequence identity to SEQ ID NO:233. In some aspects, the linker comprises the sequence set forth in SEQ ID NO:233, or a contiguous portion of SEQ ID NO:233 of at least 5, 10, 15, 20, 25, 30, 35, 40, 45, 50, 55, 60, 65, 70 or 75 amino acids. In some aspects, the linker consists of the sequence set forth in SEQ ID NO:233, or a contiguous portion of SEQ ID NO:233 of at least 5, 10, 15, 20, 25, 30, 35, 40, 45, 50, 55, 60, 65, 70 or 75 amino acids. In some embodiments, the linker comprises the sequence set forth in SEQ ID NO:233. In some embodiments, the linker consists of the sequence set forth in SEQ ID NO:233. In some embodiments, a linker comprises the sequence set forth in SEQ ID NO:299, or a portion thereof, or an amino acid sequence that has at least 90%, 91%, 92%, 93%, 94%, 95%, 96%, 97%, 98%, or 99% sequence identity to any of the foregoing. In some aspects, the linker comprises the sequence set forth in SEQ ID NO:299, or a contiguous portion of SEQ ID NO:299 of at least 5, 10, or 15 amino acids. In some aspects, the linker consists of the sequence set forth in SEQ ID NO:299, or a contiguous portion of SEQ ID NO:299 of at least 5, 10 or 15 amino acids. In some embodiments, the linker comprises the sequence set forth in SEQ ID NO:299. In some embodiments, the linker consists of the sequence set forth in SEQ ID NO:299. Appropriate linkers may be selected or designed based rational criteria known in the art, for example as described in Chen et al. Adv. Drug Deliv. Rev. 65(10):1357-1369 (2013). In some embodiments, a linker comprises a linker described in WO 2021/247570.

In some embodiments, the fusion protein of the DNA-targeting system, or a DNA-targeting module thereof, comprises one or more nuclear localization signals (NLS). In some embodiments, a fusion protein described herein comprises one or more nuclear localization sequences (NLSs), such as about or more than about 1, 2, 3, 4, 5, 6, 7, 8, 9, 10, or more NLSs. When more than one NLS is present, each may be selected independently of the others, such that a single NLS may be present in more than one copy and/or in combination with one or more other NLSs present in one or more copies. Non-limiting examples of NLSs include an NLS sequence derived from: the NLS of the SV40 virus large T-antigen, having the amino acid sequence PKKKRKV (SEQ ID NO: 234); the NLS from nucleoplasmin (e.g. the nucleoplasmin bipartite NLS with the sequence KRPAATKKAGQAKKKK (SEQ ID NO: 249)); the c-myc NLS having the amino acid sequence PAAKRVKLD (SEQ ID NO: 235) or RQRRNELKRSP (SEQ ID NO: 236); the hRNPA1 M9 NLS having the sequence NQSSNFGPMKGGNFGGRSSGPYGGGGQYFAKPRNQGGY (SEQ ID NO: 237); the sequence RMRIZFKNKGKDTAELRRRRVEVSVELRKAKKDEQILKRRNV (SEQ ID NO: 238) of the IBB domain from importin-alpha; the sequences VSRKRPRP (SEQ ID NO: 239) and PPKKARED (SEQ ID NO: 240) of the myoma T protein; the sequence PQPKKKPL (SEQ ID NO: 241) of human p53; the sequence SALIKKKKKMAP (SEQ ID NO: 242) of mouse c-abl IV; the sequences DRLRR (SEQ ID NO: 243) and PKQKKRK (SEQ ID NO: 244) of the influenza virus NS1; the sequence RKLKKKIKKL (SEQ ID NO: 245) of the Hepatitis virus delta antigen; the sequence REKKKFLKRR (SEQ ID NO: 246) of the mouse Mxl protein; the sequence KRKGDEVDGVDEVAKKKSKK (SEQ ID NO: 247) of the human poly(ADP-ribose) polymerase; and the sequence RKCLQAGMNLEARKTKK (SEQ ID NO: 248) of the steroid hormone receptors (human) glucocorticoid. The NLS may comprise a portion of any of the foregoing. In general, the one or more NLSs are of sufficient strength to drive accumulation of the fusion protein in a detectable amount in the nucleus of a eukaryotic cell. In general, strength of nuclear localization activity may derive from the number of NLSs in the fusion protein, the particular NLS(s) used, or a combination of these factors. Detection of accumulation in the nucleus may be performed by any suitable technique. For example, a detectable marker may be fused to the fusion protein, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of the fusion protein (e.g. an assay for altered gene expression activity in a cell transformed with the DNA-targeting system comprising the fusion protein), as compared to a control condition (e.g. an untransformed cell).

In some embodiments, the NLS is linked to the N-terminus or the C-terminus of the DNA-binding domain via a linker. In some embodiments, the NLS is linked to the N-terminus or the C-terminus of an effector domain via a linker. The linker may be any linker as described above. In some embodiments, the linker is GIHGVPAA (SEQ ID NO: 294). In some embodiments, the NLS and linker has the sequence PKKKRKVGIHGVPAA (SEQ ID NO: 291).

In some configurations, the N- or C-terminus of the fusion protein can be linked to a moiety for detection and/or purification. In some aspects, the moiety is or includes a Flag tag DYKDDDDK (SEQ ID NO:292), a 3xFlag tag MIDYKDHDGDYKDHDI DYKDDDDK (SEQ ID NO: 295), an HA tag YPYDVPDYA (SEQ ID NO: 296) or a His tag, such as HH11HHH (SEQ ID NO: 297).

### 1. Split Fusion Proteins

In some embodiments, the fusion protein is a split protein, i.e. comprises two or more separate polypeptide domains that interact or self-assemble to form a functional fusion protein. In some aspects, the split fusion protein comprises a dCas9 and an effector domain. In some aspects, the fusion protein comprises a split dCas9-effector domain fusion protein.

In some embodiments, the split fusion protein is assembled from separate polypeptide domains comprising trans-splicing inteins. Inteins are internal protein elements that self-excise from their host protein and catalyze ligation of flanking sequences with a peptide bond. In some embodiments, the split fusion protein is assembled from a first polypeptide comprising an N-terminal intein and a second polypeptide comprising a C-terminal intein. In some embodiments, the N terminal intein Is the N terminal Npu Intein set forth in SEQ ID NO:300. In some embodiments, the C terminal intein is the C terminal Npu intein set forth in SEQ ID NO:302. In some embodiments, the N terminal intein is the N terminal Npu Intein encoded by the nucleotide sequence set forth in SEQ ID NO:305. In some embodiments, the C terminal intein is the C terminal Npu intein encoded by the nucleotide sequence set forth in SEQ ID NO:301.

In some embodiments, the split fusion protein comprises a split dCas9-effector domain fusion protein assembled from two polypeptides. In an exemplary embodiment, the first polypeptide comprises an effector catalytic domain and an N-terminal fragment of dSpCas9, followed by an N terminal Npu Intein (effector domain-dSpCas9-573N), and the second polypeptide comprises a C terminal Npu Intein, followed by a C-terminal fragment of dSpCas9 (dSpCas9-573C). In some embodiments, the C terminal Npu Intein, followed by a C-terminal fragment of dSpCas9 (dSpCas9-573C) is set forth in SEQ ID NO:304. In some embodiments, the C terminal Npu Intein, followed by a C-terminal fragment of dSpCas9 (dSpCas9-573C) is encoded by the nucleotide sequences set forth in SEQ ID NO:303. The N- and C-terminal fragments of the fusion protein are split at position 573Glu of the dSpCas9 molecule, with reference to SEQ ID NO: 206 (corresponding to residue 572Glu of the dSpCas9 molecule set forth in SEQ ID NO: 207). In some aspects, the N-terminal Npu Intein (SEQ ID NO:300) and C-terminal Npu Intein (set forth in SEQ ID NO:302) may self-excise and ligate the two fragments, thereby forming the full-length dSpCas9-effector domain fusion protein when expressed in a cell.

In some embodiments, the polypeptides of a split protein may interact non-covalently to form a complex that recapitulates the activity of the non-split protein. For example, two domains of a Cas enzyme expressed as separate polypeptides may be recruited by a gRNA to form a ternary complex that recapitulates the activity of the full-length Cas enzyme in complex with the gRNA, for example as described in Wright et al. PNAS 112(10):2984-2989 (2015). In some embodiments, assembly of the split protein is inducible (e.g. light inducible, chemically inducible, small-molecule inducible).

In some aspects, the two polypeptides of a split fusion protein may be delivered and/or expressed from separate vectors, such as any of the vectors described herein. In some embodiments, the two polypeptides of a split fusion protein may be delivered to a cell and/or expressed from two separate AAV vectors, i.e. using a split AAV-based approach, for example as described in WO 2017/197238.

Approaches for the rationale design of split proteins and their delivery, including Cas proteins and fusions thereof, are described, for example, in WO 2016/114972, WO 2017/197238, Zetsche. et al. Nat. Biotechnol. 33(2):139-42 (2015), Wright et al. PNAS 112(10):2984-2989 (2015), Truong. et al. Nucleic Acids Res. 43, 6450-6458 (2015), and Fine et al. Sci. Rep. 5, 10777 (2015).

### 2. Exemplary Fusion Proteins

In some embodiments, fusion proteins of provided DNA-targeting systems, or DNA-targeting modules thereof, are composed of a DNA-binding domain targeting to a target site in a gene that encodes a gene product that regulates LDL or a regulatory element thereof, and at least one transcriptional repressor effector domain. The DNA-binding domain and transcriptional repressor domain include any as described above. Exemplary fusion proteins are further described below.

In some embodiments, a fusion protein named herein comprises elements of the named fusion protein in any configuration or order, unless a particular SEQ ID NO is identified or a particular order is specified. For example, a dCas9-KRAB fusion protein may comprise a KRAB domain fused to the N- or C-terminus of a dSpCas9 molecule. In another example, a dSpCas9-KRAB-DNMT3A/L fusion protein may comprise dSpCas9, KRAB, and DNMT3A/L in any order. For example, a dSpCas9-KRAB-DNMT3A/L fusion protein may comprise from N-terminal to C-terminal, DNMT3A/L, dSpCas9, and KRAB. In some embodiments, the fusion protein of the DNA-targeting system comprises in N-terminal to C-terminal order dSpCas9-KRAB-DNMT3A/L. In some embodiments, the fusion protein of the DNA-targeting system comprises in N-terminal to C-terminal order DNMT3A/L-dSpCas9-KRAB. A fusion protein named herein may comprise additional elements. For example, a dSpCas9-KRAB-DNMT3A/L fusion protein may comprise one or more linkers, NLS sequences, or other sequences in any combination or order. Similar orientations of fusion proteins are provided in which a different dCas is employed as the DNA-binding domain instead of the exemplary dSpCas9. Any dCas is contemplated included any as described herein. In some embodiments, the DNA-binding domain is a dCas. In some embodiments, the dCas is a dCas9. In some embodiments, the dCas9 is a dSpCas9.

In some aspects, exemplary linkers or NLS sequences can be any described herein. In some aspects, exemplary linkers or NLS sequences can be positioned in any order between the DNA-binding domain (e.g. dSpCas9) and one or more effector domains (e.g., KRAB and DNMT3A/L). In aspects of any of the above embodiments, the NLS can be any as described, such as set forth in Section I.E. In aspects of any of the above embodiments, the linker can be any as described, such as set forth in Section I.E.

In some embodiments, a fusion protein of a DNA-targeting system, or a DNA-targeting module thereof, provided herein comprises a DNA-binding domain and a KRAB domain. In some embodiments, the DNA-binding domain is a catalytically inactive Cas enzyme (dCas). In some embodiments, the Cas is a dCas9, such as a dSaCas9 or a dSpCas9. In some embodiments, the DNA-binding domain is dSpCas9. In some embodiments, the DNA-binding domain is a dSpCas9 set forth in SEQ ID NO: 207 and the KRAB domain is set forth in SEQ ID NO: 193. In some embodiments, the DNA-binding domain is a dSpCas9 set forth in SEQ ID NO: 207 and the KRAB domain is set forth in SEQ ID NO:290. In some embodiments, the fusion protein may include one or more linker or NLS sequences, such as at the N- or C-terminus of the fusion protein or between the Cas and the KRAB domain. The linker or NLS can be any as described herein.

In some embodiments, the fusion protein of a DNA-targeting system, or a DNA-targeting module thereof, comprises, from N- to C-terminal order: NLS and/or a linker, dSpCas9 set forth in SEQ ID NO:207, a linker and/or NLS, and a KRAB domain set forth in SEQ ID NO:193. In some embodiments, the fusion protein of a DNA-targeting system, or a DNA-targeting module thereof, comprises, from N- to C-terminal order: NLS and/or a linker, dSpCas9 set forth in SEQ ID NO:207, a linker and/or NLS, and a KRAB domain set forth in SEQ ID NO:290.

In some embodiments, a fusion protein provided herein comprises dCas9 and KRAB. In some embodiments, a fusion protein provided herein comprises dSpCas9-KRAB. In some embodiments, a fusion protein provided herein comprises the sequence set forth in SEQ ID NO: 209, or an amino acid sequence that has at least 90%, 91%, 92%, 93%, 94%, 95%, 96%, 97%, 98%, or 99% sequence identity thereto. In some embodiments, the fusion protein comprises the sequence set forth in SEQ ID NO:209.

In some embodiments, a fusion protein of a DNA-targeting system, or a DNA-targeting module thereof, provided herein comprises a DNA-binding domain, a DNMT3A domain and a DNMT3L domain. In some embodiments, the effector domain is a fusion domain of DNMT3A and DNMT3L (DNMT3A/L domain). In some embodiments, the DNA-binding domain is a catalytically inactive Cas enzyme (dCas). In some embodiments, the Cas is a dCas9, such as a dSaCas9 or a dSpCas9. In some embodiments, the DNA-binding domain is dSpCas9. In some embodiments, the DNA-binding domain is a dSpCas9 set forth in SEQ ID NO:207. In some embodiments, a linker or NLS connects one of the DNMT3A domain or the DNMT3L domain with the DNA-binding domain. In some embodiments, the DNA binding domain is a Cas, and a linker or NLS can be present between the Cas and one or both of the DNMT3A domain and DNMT3L domain. In some embodiments, a fusion protein contains a DNA-binding domain, a DNMT3A domain and a DNMT3L domain, in any order. In some embodiments, a linker connects a DNMT3A domain with a DNMT3L domain. In some embodiments, the DNMT3A domain an DNMT3L domain are connected as a fusion domain. In some embodiments, the fusion domain is the DNMT3A/L domain set forth in SEQ ID NO: 199. In some embodiments, the fusion domain is the DNMT3A/L domain set forth in SEQ ID NO: 201.

In some embodiments, the DNA-targeting system or fusion protein comprises, from N- to C-terminal order: NLS and/or a linker, dSpCas9 set forth in SEQ ID NO:207, a linker and/or NLS, and a DNMT3A/L domain set forth in SEQ ID NO: 199. In some embodiments, the DNA-targeting system or fusion protein comprises, from N- to C-terminal order: NLS and/or a linker, dSpCas9 set forth in SEQ ID NO:207, a linker and/or NLS, and a DNMT3A/L domain set forth in SEQ ID NO: 201.

In some embodiments, the DNA-targeting system or fusion protein comprises, from N- to C-terminal order: NLS and/or a linker, dSpCas9 set forth in SEQ ID NO:207, a linker and/or NLS, and a DNMT3A/L domain set forth in SEQ ID NO:199, and a linker and/or NLS. In some embodiments, the DNA-targeting system or fusion protein comprises, from N- to C-terminal order: NLS and/or a linker, dSpCas9 set forth in SEQ ID NO:207, a linker and/or NLS, and a DNMT3A/L domain set forth in SEQ ID NO:201, and a linker and/or NLS.

In some embodiments, a fusion protein of a DNA-targeting system, or a DNA-targeting module thereof, provided herein comprises a DNA-binding domain, and two effector domains in which one is a KRAB domain and the other is a DNMT3A domain and a DNMT3L domain. In some embodiments, the effector domain composed of the DNMT3A domain and the DNMT3L domain is a fusion domain of DNMT3A and DNMT3L (DNMT3A/L domain). In some embodiments, the DNA-binding domain is a catalytically inactive Cas enzyme (dCas). In some embodiments, the Cas is a dCas9, such as a dSaCas9 or a dSpCas9. In some embodiments, the DNA-binding domain is dSpCas9. In some embodiments, each of the KRAB domain, the DNMT3A domain and the DNMT3L domain are N-terminal to the DNA-binding domain. In some embodiments, each of the KRAB domain, the DNMT3A domain and the DNMT3L domain are C-terminal to the DNA-binding domain. In some embodiments, the DNA-binding domain is between the KRAB domain and one of the DNMT3A or DNMT3L domains. In some embodiments, the fusion domain is the DNMT3A/L domain set forth in SEQ ID NO: 199 or 201. In some embodiments, the KRAB domain is set forth in SEQ ID NO:193. In some embodiments, the KRAB domain is set forth in SEQ ID NO:290. In some embodiments, the DNA-binding domain is a dSpCas9 set forth in SEQ ID NO:207. In some embodiments, the fusion protein may include one or more linker or NLS, such as at the N- or C-terminus of the fusion protein or between the Cas and and the KRAB domain or DNMT3A/L domain. The linker or NLS can be any as described herein. In some embodiments, a linker or NLS can be present between the DNMT3A/L domain and the Cas. In some embodiments, a linker or NLS can be present between the Cas and the KRAB domain.

In some embodiments, the fusion protein contains the DNA-binding domain, and the KRAB domain and DNMT3A/3L fusion domain as first and second effector domains. In some embodiments, a first effector domain is fused to the N-terminus of the second effector domain and the second effector domain is fused to the N-terminus of the DNA-binding domain. In some embodiments, a fusion protein provided herein comprises in order: DNMT3A/L-KRAB-dSpCas9. In some embodiments, a fusion protein provided herein comprises in order: KRAB-DNMT3A/L-dSpCas9. In some embodiments, each of the KRAB domain and DNMT3A/3L domain are C-terminal to the DNA-binding domain in which a first effector domain is fused to the C-terminus of the DNA-binding domain and the second effector domain is fused to the C-terminus of the first effector domain. In some embodiments, a fusion protein provided herein comprises in order: dSpCas9—DNMT3A/L-KRAB. In some embodiments, a fusion protein provided herein comprises in order: dSpCas9-KRAB-DNMT3A/L. In some embodiments, the DNA-binding domain is between the KRAB domain and DNMT3A/3L domain, in which one effector domain is fused to the N-terminus of the DNA-binding domain and the other effector domain is fused to the C-terminus of the DNA-binding domain. In some embodiments, a fusion protein provided herein comprises in order: KRAB-dSpCas9-DNMT3A/L. In some embodiments, a fusion protein provided herein comprises in order: DNMT3A/L-dSpCas9-KRAB.

In some embodiments, the fusion protein of a DNA-targeting system, or a DNA-targeting module thereof, comprises, from N- to C-terminal order: a DNMT3A/L fusion domain set forth in SEQ ID NO: 199, an NLS and/or a linker, a dSpCas9 set forth in SEQ ID NO:207, a linker and/or NLS, and a KRAB domain set forth in SEQ ID NO: 193. In some embodiments, the fusion protein of a DNA-targeting system, or a DNA-targeting module thereof, comprises, from N- to C-terminal order: a DNMT3A/L fusion domain set forth in SEQ ID NO: 199, an NLS and/or a linker, a dSpCas9 set forth in SEQ ID NO:207, a linker and/or NLS, and a KRAB domain set forth in SEQ ID NO:290.

In some embodiments, the fusion protein of a DNA-targeting system, or a DNA-targeting module thereof, comprises, from N- to C-terminal order: a DNMT3A/L fusion domain set forth in SEQ ID NO: 201, an NLS and/or a linker, a dSpCas9 set forth in SEQ ID NO:207, a linker and/or NLS, and a KRAB domain set forth in SEQ ID NO: 193. In some embodiments, the fusion protein of a DNA-targeting system, or a DNA-targeting module thereof, comprises, from N- to C-terminal order: a DNMT3A/L fusion domain set forth in SEQ ID NO: 201, an NLS and/or a linker, a dSpCas9 set forth in SEQ ID NO:207, a linker and/or NLS, and a KRAB domain set forth in SEQ ID NO:290.

In some embodiments, a fusion protein provided herein comprises dCas9, KRAB, and DNMT3A/L. In some embodiments, a fusion protein provided herein comprises dSpCas9-KRAB-DNMT3A/L.

In some embodiments, a fusion protein provided herein comprises the sequence set forth in SEQ ID NO: 278, or an amino acid sequence that has at least 90%, 91%, 92%, 93%, 94%, 95%, 96%, 97%, 98%, or 99% sequence identity to SEQ ID NO:278. In some embodiments, the fusion protein comprises the sequence set forth in SEQ ID NO:278.

In some embodiments, a fusion protein provided herein comprises the sequence set forth in SEQ ID NO: 280, or an amino acid sequence that has at least 90%, 91%, 92%, 93%, 94%, 95%, 96%, 97%, 98%, or 99% sequence identity to SEQ ID NO: 280. In some embodiments, the fusion protein comprises the sequence set forth in SEQ ID NO:280.

In some embodiments, a fusion protein provided herein comprises the sequence set forth in SEQ ID NO: 282, or an amino acid sequence that has at least 90%, 91%, 92%, 93%, 94%, 95%, 96%, 97%, 98%, or 99% sequence identity thereto. In some embodiments, the fusion protein comprises the sequence set forth in SEQ ID NO:282.

### II. Polynucleotides, Vectors, and Related Methods for Delivery

In some aspects, provided are polynucleotides encoding any of the DNA-targeting systems described herein or a portion or a component of any of the foregoing. In some aspects, the polynucleotides can encode any of the components of the DNA-targeting systems, and/or any nucleic acid or proteinaceous molecule necessary to carry out aspects of the methods of the disclosure. In particular embodiments, provided are polynucleotides encoding any of the fusion proteins described herein. Also provided herein are polynucleotides encoding any of the gRNAs or combinations of gRNAs described herein.

In some embodiments, provided are polynucleotides comprising the gRNAs described herein. In some embodiments, the gRNA is transcribed from a genetic construct (i.e. vector or plasmid) in the target cell. In some embodiments, the gRNA is produced by in vitro transcription and delivered to the target cell. In some embodiments, the gRNA comprises one or more modified nucleotides for increased stability. In some embodiments, the gRNA is delivered to the target cell pre-complexed as a RNP with the fusion protein.

In some embodiments, a provided polynucleotide encodes a fusion protein as described herein that includes (a) a DNA-binding domain capable of being targeted to a target site of a target gene as described; and (b) at least one effector domain capable of reducing transcription of the gene. In some embodiments, the fusion protein includes a fusion protein of a Cas protein or variant thereof and at least one effector domain capable of reducing transcription of a gene. In a particular example, the Cas is a dCas, such as dCas9. In some embodiments, the dCas9 is a dSpCas9, such as polynucleotide encoding a dSpCas9 set forth in SEQ ID NO: 207. Examples of such domains and fusion proteins include any as described in Section I.

In some embodiments, the polynucleotide comprises a sequence encoding a dCas9-KRAB fusion protein. In some embodiments, the polynucleotide comprises the sequence set forth in SEQ ID NO:208, or a sequence having at least about 80%, 85%, 90%, 91%, 92%, 93%, 94%, 95%, 96%, 97%, 98%, 99%, or 100% sequence identity thereto. In some embodiments, the polynucleotide is set forth in SEQ ID NO:208. In some embodiments, the polynucleotide encodes an amino acid sequence comprising SEQ ID NO:209, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:209.

In some embodiments, the polynucleotide comprises a sequence encoding a dCas9-KRAB-DNMT3A/L fusion protein. In some embodiments, the polynucleotide comprises the sequence set forth in SEQ ID NO:277, or a sequence having at least about 80%, 85%, 90%, 91%, 92%, 93%, 94%, 95%, 96%, 97%, 98%, 99%, or 100% sequence identity thereto. In some embodiments, the polynucleotide is set forth in SEQ ID NO:277. In some embodiments, the polynucleotide encodes an amino acid sequence comprising SEQ ID NO:278, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:278.

In some embodiments, the polynucleotide comprises a sequence encoding a dCas9-KRAB-DNMT3A/L fusion protein, such as the sequence set forth in SEQ ID NO:279, or a sequence having at least about 80%, 85%, 90%, 91%, 92%, 93%, 94%, 95%, 96%, 97%, 98%, 99%, or 100% sequence identity thereto. In some embodiments, the polynucleotide is set forth in SEQ ID NO:279. In some embodiments, the polynucleotide encodes an amino acid sequence comprising SEQ ID NO:280, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:280.

In some embodiments, the polynucleotide comprises a sequence encoding a dCas9-KRAB-DNMT3A/L fusion protein, such as the sequence set forth in SEQ ID NO:281, or a sequence having at least about 80%, 85%, 90%, 91%, 92%, 93%, 94%, 95%, 96%, 97%, 98%, 99%, or 100% sequence identity thereto. In some embodiments, the polynucleotide is set forth in SEQ ID NO:281. In some embodiments, the polynucleotide encodes an amino acid sequence comprising SEQ ID NO:282, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:282.

In some embodiments, the polynucleotide is an mRNA molecule that comprises a sequence encoding a dCas9-KRAB-DNMT3A/L fusion protein, such as the sequence set forth in SEQ ID NO:395, or a sequence having at least about 80%, 85%, 90%, 91%, 92%, 93%, 94%, 95%, 96%, 97%, 98%, 99%, or 100% sequence identity thereto. In some embodiments, the polynucleotide is set forth in SEQ ID NO:395.

In some embodiments, the polynucleotide is RNA or DNA. In some embodiments, the polynucleotide, such as a polynucleotide encoding a provided fusion protein, is mRNA. In some embodiments, the gRNA is provided as RNA and a polynucleotide encoding the fusion protein is mRNA. The mRNA can be 5′ capped and/or 3′ polyadenylated. In another embodiment, a polynucleotide provided herein, such as a polynucleotide encoding a provided fusion protein, is DNA. The DNA can be present in a vector.

Also provided herein is a vector that contains any of the provided polynucleotides. In some embodiments, the vector comprises a genetic construct, such as a plasmid or an expression vector.

In some embodiments, the expression vector comprising the sequence encoding the fusion protein of a DNA-targeting system provided herein can further comprise a polynucleotide sequence encoding at least one gRNA. In some embodiments, the expression vector comprises a polynucleotide sequence or combination of polynucleotide sequences encoding two gRNAs. In some embodiments, the expression vector comprises a polynucleotide sequence or combination of polynucleotide sequences encoding three gRNAs. The sequence encoding the gRNA can be operably linked to at least one transcriptional control sequence for expression of the gRNA in the cell. For example, DNA encoding the gRNA can be operably linked to a promoter sequence that is recognized by RNA polymerase III (Pol III). Examples of suitable Pol III promoters include, but are not limited to, mammalian U6, U3, H1, and 7SL RNA promoters.

An expression vector (such as a DNA or RNA (e.g. mRNA) expression vector) can comprise any number of suitable transcriptional control sequences. For example, transcriptional control sequences may include enhancers, promoters, or untranslated regions (UTRs) such as 3′UTRs or 5′UTRs. In some embodiments, the UTRs are encoded by and/or present in the expression vector (e.g. a DNA vector). In some embodiments, mRNA encoding the fusion protein includes UTRs. In some aspects, different transcriptional control sequences may be selected for use in an expression vector, for example to achieve the appropriate level of expression. For example, in some embodiments, UTRs can be selected that facilitate expression in a specific tissue or cell type (e.g. liver or hepatocyte). In some embodiments, a 5′UTR of the expression vector encodes or comprises the sequence set forth in SEQ ID NO:393. In some embodiments, a 3′UTR of the expression vector encodes or comprises the sequence set forth in SEQ ID NO:394.

In some embodiments, provided is a vector containing a polynucleotide that encodes a fusion protein comprising a DNA-binding domain comprising a dCas and at least one effector domain capable of increasing transcription of a gene, and a polynucleotide or combination of polynucleotides encoding a gRNA, or combination of gRNAs, such as two gRNAs, or three gRNAs. In some embodiments, the dCas is a dCas9, such as dSpCas9. In some embodiments, the polynucleotide encodes a fusion protein that includes a dSpCas9 set forth in SEQ ID NO: 207. In some embodiments, the polynucleotide(s) encodes a gRNA or combination of gRNAs as described in Section II.B.ii. For example, the polynucleotide can encode a combination of gRNAs, each comprising a spacer sequence selected from any one of SEQ ID NOS: 64-126, 318-329, 352-361, or 378-383, or a contiguous portion thereof of at least 14 nt. In some embodiments the polynucleotide(s) encodes a combination of gRNAs that each comprise a sequence set forth in any one of SEQ ID NOS:127-189, 330-341, 362-371, or 384-389.

In some embodiments, the effector domain is KRAB. In some embodiments, the effector domain is DNMT3A/L. In some embodiments, the vector includes a polynucleotide comprising SEQ ID NO: 192, SEQ ID NO:277, SEQ ID NO:279, or SEQ ID NO:281, or a sequence having at least about 80%, 85%, 90%, 91%, 92%, 93%, 94%, 95%, 96%, 97%, 98%, 99%, or 100% sequence identity thereto, and a polynucleotide or polynucleotides that encode a gRNA or combination of gRNAs such as any described in Section I.B.ii. In some embodiments, the polynucleotide(s) encodes a combination of gRNAs, each comprising a spacer sequence selected from any one of SEQ ID NOS: 64-126, 318-329, 352-361, or 378-383, or a contiguous portion thereof of at least 14 nt. In some embodiments, each gRNA further comprises the sequence set forth in SEQ ID NO: 191. In some embodiments the polynucleotide(s) encodes a combination of gRNAs that each comprises a sequence set forth in any one of SEQ ID NOS:127-189, 330-341, 362-371, or 384-389.

In some embodiments, the polynucleotide encodes the fusion protein and the combination of gRNAs.

In some embodiments, the polynucleotide as provided herein can be codon optimized for efficient translation into protein in the eukaryotic cell or animal of interest. For example, codons can be optimized for expression in humans, mice, rats, hamsters, cows, pigs, cats, dogs, fish, amphibians, plants, yeast, insects, and so forth. Programs for codon optimization are available as freeware. Commercial codon optimization programs are also available.

In some embodiments, a polynucleotide described herein can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with the DNA-targeting systems, various promoters such as RNA polymerase III promoters, including for example U6 and HI, can be advantageous. Descriptions of and parameters for enhancing the use of such promoters are known in the art, and additional information and approaches are regularly being described; see, e.g., Ma, H. et al., Molecular Therapy Nucleic Acids 3, ei61 (2014) doi:10.1038/mtna.2014.12.

The expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, a cell type specific promoter (e.g. a hepatocyte specific promoter), etc.).

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Other vectors can be used so long as they are compatible with the host cell.

In some embodiments, the vector is a viral vector, such as an adeno-associated virus (AAV) vector, a retroviral vector, a lentiviral vector, or a gammaretroviral vector. In some embodiments. In some embodiments, the viral vector is an adeno-associated virus (AAV) vector. In some embodiments, the AAV vector is selected from among an AAV1, AAV2, AAV3, AAV4, AAV5, AAV6, AAV7, AAV8, or AAV9 vector. In some embodiments, the vector is a lentiviral vector. In some embodiments, the vector is a non-viral vector, for example a lipid nanoparticle, a liposome, an exosome, or a cell penetrating peptide. In some embodiments, the vector comprises one vector, or two or more vectors.

In some embodiments, a vector described herein is or comprises a lipid nanoparticle (LNP). Among provided embodiments, is a lipid nanoparticle that contains any of the provided polynucleotides for delivery of an epigenetic DNA-targeting system. In some embodiments, the LNP contains a polynucleotide that encodes a fusion protein as provided herein that includes (a) a DNA-binding domain capable of being targeted to a target site of a target gene as described; and (b) at least one effector domain capable of reducing transcription of the gene. In some embodiments, the DNA-binding domain is a Cas (e.g. dCas) and the LNP further includes a gRNA. In some embodiments, the polynucleotide encoding the fusion protein is an mRNA and the gRNA is provided as an RNA.

In some embodiments, any of the epigenetic-modifying DNA-targeting systems, gRNAs, Cas-gRNA combinations, polynucleotides, fusion proteins, or components thereof described herein, are incorporated in lipid nanoparticles (LNPs), such as for delivery. In some embodiments, the lipid nanoparticle is a vector for delivery. In some embodiments, the nanoparticle may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC 3-DMA, DLin-KC2-DMA and DODMA. Typically, the LNPs are composed of two or more lipids, such as 3, 4 or 5 lipids. In some embodiments, at least lipid is either ionoizable cationic or cationic.

Lipid nanoparticles can be used for the delivery of encapsulated or associated (e.g., complexed) therapeutic agents, including nucleic acids and proteins, such as those encoding and/or comprising CRISPR/Cas systems. See, e.g., U.S. Pat. Nos. 10,723,692, 10,941,395, and WO 2015/035136.

In some embodiments, the provided methods involve use of a lipid nanoparticle (LNP) comprising mRNA, such as mRNA encoding a protein component of any of the provided DNA-targeting systems, for example any of the fusion proteins provided herein. In some embodiments, the mRNA can be produced using methods known in the art such as in vitro transcription. In some embodiments of the method, the mRNA comprises a 5′ cap. In some embodiments, the 5′ cap is an altered nucleotide on the 5′ end of primary transcripts such as messenger RNA. In some aspects, the 5′ caps of the mRNA improves one or more of RNA stability and processing, mRNA metabolism, the processing and maturation of an RNA transcript in the nucleus, transport of mRNA from the nucleus to the cytoplasm, mRNA stability, and efficient translation of mRNA to protein. In some embodiments, a 5′ cap can be a naturally-occurring 5′ cap or one that differs from a naturally-occurring cap of an mRNA. A 5′ cap may be any 5′ cap known to a skilled artisan. In certain embodiments, the 5′ cap is selected from the group consisting of an Anti-Reverse Cap Analog (ARCA) cap, a 7-methyl-guanosine (7mG) cap, a CleanCap® analog, a vaccinia cap, and analogs thereof. For instance, the 5′ cap may include, without limitation, an anti-reverse cap analogs (ARCA) (U.S. Pat. No. 7,074,596), 7-methyl-guanosine, CleanCap® analogs, such as Cap 1 analogs (Trilink; San Diego, CA), or enzymatically capped using, for example, a vaccinia capping enzyme or the like. In some embodiments, the mRNA may be polyadenylated. The mRNA may contain various 5′ and 3′ untranslated sequence elements to enhance expression of the encoded protein and/or stability of the mRNA itself. Such elements can include, for example, posttranslational regulatory elements such as a woodchuck hepatitis virus post-transcriptional regulatory element (WPRE). In some embodiments, the mRNA comprises at least one nucleoside modification. The mRNA may contain modifications of naturally-occurring nucleosides to nucleoside analogs. Any nucleoside analogs known in the art are envisioned. Such nucleoside analogs can include, for example, those described in U.S. Pat. No. 8,278,036. In certain embodiments of the method, the nucleoside modification is selected from the group consisting of a modification from uridine to pseudouridine and uridine to Nl-methyl pseudouridine. In particular embodiments of the method the nucleoside modification is from uridine to pseudouridine.

In some embodiments, the LNP composition comprises a PEG-lipid (e.g., a lipid comprising a polyethylene glycol component). In some embodiments, the described LNP composition comprises two or more PEG-lipids. Exemplary PEG-lipids also include, but are not limited to, PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, the one or more PEG-lipids can comprise PEG-c-DOMG, PEG-DMG, PEG-DLPE, PEG-DMPE, PEG-DPPC, a PEG-DSPE lipid, or a combination thereof. In some embodiments, PEG moiety is an optionally substituted linear or branched polymer of ethylene glycol or ethylene oxide. In some embodiments, the PEG moiety is substituted, e.g., by one or more alkyl, alkoxy, acyl, hydroxy, or aryl groups. In some embodiments, the PEG moiety includes PEG copolymer such as PEG-polyurethane or PEG-polypropylene (see, e.g., j. Milton Harris, Poly(ethylene glycol) chemistry: biotechnical and biomedical applications (1992)). In some embodiments, a PEG-lipid is a PEG-lipid conjugate. In some embodiments, the PEG-lipid comprises from about 0.1 mol % to about 6 mol % of a total lipid content present in said nanoparticle composition. In some embodiments, a number average molecular weight of the PEG-lipid is from about 200 Da to about 5000 Da. In some embodiments, the LNP comprises and/or is conjugated to N-Acetylgalactosamine (GalNAc), an amino sugar derivative of galactose. In some aspects, GalNAc is a sugar molecule that can recognize and bind to a cell surface protein, the asialoglycoprotein receptor (ASGPR). ASGPR is abundantly expressed on liver cells (hepatocytes). In some embodiments, GalNAc conjugation to LNPs can improve delivery to hepatocytes. In some embodiments, the lipid nanoparticle comprises GalNAC-conjugated lipids (e.g. a GalNAc-PEG lipid). In some embodiments, the molar percentage of lipids that are GalNAC-conjugated in a lipid nanoparticle is between about 0% and about 2%.

In some embodiments, the molar percentage of lipids that are GalNAC-conjugated in a lipid nanoparticle is at or about 0.1%, at or about 0.2%, at or about 0.3%, at or about 0.4%, at or about 0.5%, at or about 0.6%, at or about 0.7%, at or about 0.8%, at or about 0.9%, at or about 1.0%, at or about 1.1%, at or about 1.2%, at or about 1.3%, at or about 1.4%, at or about 1.5%, at or about 1.6%, at or about 1.7%, at or about 1.8%, at or about 1.9%, at or about 2.0%, or more, or a value in between any of the foregoing.

In some embodiments, LNPs useful for in the present methods comprise a cationic lipid selected from DLin-DMA (1,2-dilinoleyloxy-3-dimethylaminopropane), DLin-MC3-DM A (dilinoleylmethyl-4-dimethylaminobutyrate), DLin-KC2-DMA (2,2-dilinoleyl-4-(2-dimethylaminoethyl)-[1,3]-dioxolane), DODMA (1,2-dioleyloxy-N,N-dimethyl-3-aminopropane), SS-OP (Bis[2-(4-{2-[4-(cis-9 octadecenoyloxy)phenylacetoxy]ethyl}piperidinyl)ethyl] disulfide), and derivatives thereof. DLin-MC3-DMA and derivatives thereof are described, for example, in WO 2010/144740. DODMA and derivatives thereof are described, for example, in U.S. Pat. No. 7,745,651 and Mok et al. (1999), Biochimica et Biophysica Acta, 1419(2): 137-150. DLin-DMA and derivatives thereof are described, for example, in U.S. Pat. No. 7,799,565. DLin-KC2-DMA and derivatives thereof are described, for example, in U.S. Pat. No. 9,139,554. SS-OP (NOF America Corporation, White Plains, NY) is described, for example, at https://www.nofamerica.com/store/index.php?dispatch=products.view&product_id=962. Additional and non-limiting examples of cationic lipids include methylpyridiyl-dialkyl acid (MPDACA), palmitoyl-oleoyl-nor-arginine (PONA), guanidino-dialkyl acid (GUADACA), 1,2-di-O-octadecenyl-3-trimethylammonium propane (DOTMA), 1,2-dioleoyl-3-trimethylammonium-propane (DOTAP), Bis{2-[N-methyl-N-(a-D-tocopherolhemisuccinatepropyl)amino]ethyl} disulfide (SS-33/3AP05), Bis{2-[4-(a-D-tocopherolhemisuccinateethyl)piperidyl] ethyl} disulfide (SS33/4PE15), Bis{2-[4-(cis-9-octadecenoateethyl)-1-piperidinyl] ethyl} disulfide (SS18/4PE16), and Bis{2-[4-(cis,cis-9,12-octadecadienoateethyl)-1-piperidinyl] ethyl} disulfide (SS18/4PE13). In further embodiments, the lipid nanoparticles also comprise one or more non-cationic lipids and a lipid conjugate.

In some embodiments, the molar concentration of the cationic lipid is from about 20% to about 80%, from about 30% to about 70%, from about 40% to about 60%, from about 45% to about 55%, or about 20%, about 25%, about 30%, about 35%, about 40%, about 45%, about 50%, about 55%, about 60%, about 65%, about 70%, about 75%, or about 80% of the total lipid molar concentration, wherein the total lipid molar concentration is the sum of the cationic lipid, the non-cationic lipid, and the lipid conjugate molar concentrations. In certain embodiments, the lipid nanoparticles comprise a molar ratio of cationic lipid to any of the polynucleotides of from about 1 to about 20, from about 2 to about 16, from about 4 to about 12, from about 6 to about 10, or about 1, about 2, about 3, about 4, about 5, about 6, about 7, about 8, about 9, about 10, about 11, about 12, about 13, about 14, about 15, about 16, about 17, about 18, about 19, or about 20.

In some embodiments, the lipid nanoparticles can comprise at least one non-cationic lipid. In particular embodiments, the molar concentration of the non-cationic lipids is from about 20% to about 80%, from about 30% to about 70%, from about 40% to about 70%, from about 40% to about 60%, from about 46% to about 50%, or about 20%, about 25%, about 30%, about 35%, about 40%, about 45%, about 48.5%, about 50%, about 55%, about 60%, about 65%, about 70%, about 75%, or about 80% of the total lipid molar concentration. Non-cationic lipids include, in some embodiments, phospholipids and steroids.

In some embodiments, phospholipids useful for the lipid nanoparticles described herein include, but are not limited to, 1,2-Distearoyl-sn-glycero-3-phosphocholine (DSPC), 1,2-Didecanoyl-sn-glycero-3-phosphocholine (DDPC), 1,2-Dierucoyl-sn-glycero-3-phosphate(Sodium Salt) (DEPA-NA), 1,2-Dierucoyl-sn-glycero-3-phosphocholine (DEPC), 1,2-Dierucoyl-sn-glycero-3-phosphoethanolamine (DEPE), 1,2-Dierucoyl-sn-glycero-3[Phospho-rac-(1-glycerol)(Sodium Salt) (DEPG-NA), 1,2-Dilinoleoyl-sn-glycero-3-phosphocholine (DLOPC), 1,2-Dilauroyl-sn-gly cero-3-phosphate(Sodium Salt) (DLPA-NA), 1,2-Dilauroyl-sn-glycero-3-phosphocholine (DLPC), 1,2-Dilauroyl-sn-glycero-3-phosphoethanolamine (DLPE), 1,2-Dilauroyl-sn-glycero-3[Phospho-rac-(1-glycerol)(Sodium Salt) (DLPG-NA), 1,2-Dilauroyl-sn-glycero-3[Phospho-rac-(1-glycerol)(Ammonium Salt) (DLPG-NH4), 1,2-Dilauroyl-sn-glycero-3-phosphoserine(Sodium Salt) (DLPS-NA), 1,2-Dimyristoyl-sn-glycero-3-phosphate(SodiumSalt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol)(Sodium Salt) (DMPG-NA), 1,2-Dimyristoyl-sn-glycero-3[Phospho-rac-(1-glycerol)(Ammonium Salt) (DMPG-NH4), 1,2-Dimyristoyl-sn-glycero-3[Phospho-rac-(1-glycerol)(Sodium/Ammonium Salt) (DMPG-NH4/NA), 1,2-Dimyristoyl-sn-glycero-3-phosphoserine(Sodium Salt) (DMPS-NA), 1,2-Dioleoyl-sn-glycero-3-phosphate(Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol)(Sodium Salt) (DOPG-NA), 1,2-Dioleoyl-sn-glycero-3-phosphoserine(Sodium Salt) (DOPS-NA), 1,2-Dipalmitoyl-sn-glycero-3-phosphate(Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol)(Sodium Salt) (DPPG-NA), 1,2-Dipalmitoyl-sn-glycero-3[Phospho-rac-(1-glycerol)(Ammonium Salt) (DPPG-NH4), 1,2-Dipalmitoyl-sn-glycero-3-phosphoserine(Sodium Salt) (DPPS-NA), 1,2-Distearoyl-sn-glycero-3-phosphate(Sodium Salt) (DSPA-NA), 1,2-Distearoyl-sn-glycero-3-phosphoethanolamine (DSPE), 1,2-Distearoyl-sn-glycero-3[Phospho-rac-(1-glycerol)(Sodium Salt) (DSPG-NA), 1,2-Distearoyl-sn-glycero-3[Phospho-rac-(1-glycerol)(Ammonium Salt) (DSPG-NH4), 1,2-Distearoyl-sn-glycero-3-phosphoserine(Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LY S OPCM YRIS TIC), 1-Palmitoyl-sn-glycero-3-phosphocholine (LYSOPCPALMITIC), 1-Stearoyl-sn-glycero-3-phosphocholine (LYSOPC STEARIC), 1-Myristoyl-2-palmitoyl-sn-glycero3-phosphocholine (MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol)](Sodium Salt) (POPG-NA), 1-Palmitoyl-2-stearoyl-sn-glycero-3-phosphocholine (PS PC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). In particular embodiments, the phospholipid is DSPC. In particular embodiments, the phospholipid is DOPE. In particular embodiments, the phospholipid is DOPC.

In some embodiments, the non-cationic lipids comprised by the lipid nanoparticles include one or more steroids. Steroids useful for the lipid nanoparticles described herein include, but are not limited to, cholestanes such as cholesterol, cholanes such as cholic acid, pregnanes such as progesterone, androstanes such as testosterone, and estranes such as estradiol. Further steroids include, but are not limited to, cholesterol (ovine), cholesterol sulfate, desmosterol-d6, cholesterol-d7, lathosterol-d7, desmosterol, stigmasterol, lanosterol, dehydrocholesterol, dihydrolanosterol, zymosterol, lathosterol, zymosterol-d5, 14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, sitosterol, lanosterol-95, Dihydro FF-MAS-d6, zymostenol-d7, zymostenol, sitostanol, campestanol, campesterol, 7-dehydrodesmosterol, pregnenolone, sitosterol-d7, Dihydro T-MAS, Delta 5-avenasterol, Brassicasterol, Dihydro FF-MAS, 24-methylene cholesterol, cholic acid derivatives, cholesteryl esters, and glycosylated sterols. In particular embodiments, the lipid nanoparticles comprise cholesterol.

In some embodiments, the lipid nanoparticles comprise a lipid conjugate. Such lipid conjugates include, but are not limited to, ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide, phosphoethanolamine PEG derivatives such as 16:0 PEG5000PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE 16:0 PEG1000 PE, 14:0 PEG1000 PE, 18:0 PEG1000 PE, 18:1 PEG 1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350, sterol PEG derivatives such as Chol-PEG600, and glycerol PEG derivatives such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. In some embodiments, the lipid conjugate is a DMG-PEG. In some particular embodiments, the lipid conjugate is DMG-PEG2000. In some particular embodiments, the lipid conjugate is DMG-PEG5000.

It is within the level of a skilled artisan to select the cationic lipids, non-cationic lipids and/or lipid conjugates which comprise the lipid nanoparticle, as well as the relative molar ratio of such lipids to each other, such as based upon the characteristics of the selected lipid(s), the nature of the delivery to the intended target cells, and the characteristics of the nucleic acids and/or proteins to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method can be prepared by various techniques which are known to a skilled artisan. Nucleic acid-lipid particles and methods of preparation are disclosed in, for example, U.S. Patent Publication Nos. 20040142025 and 20070042031.

In some embodiments, the lipid nanoparticles will have a size within the range of about 25 to about 500 nm. In some embodiments, the lipid nanoparticles have a size from about 50 nm to about 300 nm, or from about 60 nm to about 120 nm. The size of the lipid nanoparticles may be determined by quasi-electric light scattering (QELS) as described in Bloomfield, Ann. Rev. Biophys. Bioeng., 10:421A150 (1981). A variety of methods are known in the art for producing a population of lipid nanoparticles of particular size ranges, for example, sonication or homogenization. One such method is described in U.S. Pat. No. 4,737,323.

In some embodiments, the lipid nanoparticles comprise a cell targeting molecule such as, for example, a targeting ligand (e.g., antibodies, scFv proteins, DART molecules, peptides, aptamers, and the like) anchored on the surface of the lipid nanoparticle that selectively binds the lipid nanoparticles to the targeted cell, such as any cell described herein, e.g. a hepatocyte.

In some embodiments, the vector exhibits liver cell and/or hepatocyte tropism.

In some aspects, provided herein are pluralities of vectors that comprise any of the vectors described herein, and one or more additional vectors comprising one or more additional polynucleotides encoding an additional portion or an additional component of any of the DNA-targeting systems described herein, any of the gRNAs described herein, any of the fusion proteins described herein, or a portion or a component of any of the foregoing.

Provided are pluralities of vectors, that include: a first vector comprising any of the polynucleotides described herein; a second vector comprising any of the polynucleotides described herein; and optionally one or more additional vectors comprising any of the polynucleotides described herein.

In some aspects, vectors provided herein may be referred to as delivery vehicles. In some aspects, any of the DNA-targeting systems, components thereof, or polynucleotides disclosed herein can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. Direct delivery of the RNP complex, including the DNA-binding domain complexed with the sgRNA, can eliminate the need for intracellular transcription and translation and can offer a robust platform for host cells with low transcriptional and translational activity. The RNP complexes can be introduced into the host cell by any of the methods known in the art.

Nucleic acids or RNPs of the disclosure can be incorporated into a host using virus-like particles (VLP). VLPs contain normal viral vector components, such as envelope and capsids, but lack the viral genome. For instance, nucleic acids expressing the Cas and sgRNA can be fused to the viral vector components such as gag and introduced into producer cells. The resulting virus-like particles containing the sgRNA-expressing vectors can infect the host cell for efficient editing.

Introduction of the complexes, polypeptides, and nucleic acids of the disclosure can occur by protein transduction domains (PTDs). PTDs, including the human immunodeficiency virus-1 TAT, herpes simplex virus-1 VP22, Drsophila Antennapedia Antp, and the poluarginines, are peptide sequences that can cross the cell membrane, enter a host cell, and deliver the complexes, polypeptides, and nucleic acids into the cell.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like, for example as described in WO 2017/193107, WO 2016/123578, WO 2014/152432, WO 2014/093661, WO 2014/093655, or WO 2021/226555.

Various methods for the introduction of polynucleotides are well known and may be used with the provided methods and compositions. Exemplary methods include those for transfer of polynucleotides encoding the DNA targeting systems provided herein, including via viral, e.g., retroviral or lentiviral, transduction, transposons, and electroporation.

In some embodiments, polynucleotides can be cloned into a suitable vector, such as an expression vector or vectors. The expression vector can be any suitable recombinant expression vector, and can be used to transform or transfect any suitable cell. Suitable vectors include those designed for propagation and expansion or for expression or both, such as plasmids and viruses.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif). In some embodiments, animal expression vectors include pEUK-Cl, pMAM and pMAMneo (Clontech). In some embodiments, a viral vector is used, such as a lentiviral or retroviral vector. In some embodiments, the recombinant expression vectors can be prepared using standard recombinant DNA techniques. In some embodiments, vectors can contain regulatory sequences, such as transcription and translation initiation and termination codons, which are specific to the type of host into which the vector is to be introduced, as appropriate and taking into consideration whether the vector is DNA- or RNA-based. In some embodiments, the vector can contain a nonnative promoter operably linked to the nucleotide sequence encoding the recombinant receptor. In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other promoters known to a skilled artisan also are contemplated.

In some embodiments, recombinant nucleic acids are transferred into cells using recombinant infectious virus particles, such as, e.g., vectors derived from simian virus 40 (SV40), adenoviruses, or adeno-associated virus (AAV). In some embodiments, recombinant nucleic acids are transferred into cells (e.g. hepatocytes) using recombinant lentiviral vectors or retroviral vectors, such as gamma-retroviral vectors (see, e.g., Koste et al. (2014) Gene Therapy 2014 Apr 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3: 102-109.

In some embodiments, the vector is a lentiviral vector. In some embodiments, the lentiviral vector is an integrase-deficient lentiviral vector. In some embodiments, the lentiviral vector is a recombinant lentiviral vector. In some embodiments, the lentivirus is selected or engineered for a desired tropism (e.g. for liver cell or hepatocyte tropism). Methods of lentiviral production, transduction, and engineering are known, for example as described in Kasaraneni, N. et al. Sci. Rep. 8(1):10990 (2018), Ghaleh, H. E. G. et al. Biomed. Pharmacother. 128:110276 (2020), and Milone, M. C. et al. Leukemia. 32(7):1529-1541 (2018). Additional methods for lentiviral transduction are described, for example in Wang et al. (2012) J. Immunother. 35(9): 689-701; Cooper et al. (2003) Blood. 101: 1637-1644; Verhoeyen et al. (2009) Methods Mol Biol. 506: 97-114; and Cavalieri et al. (2003) Blood. 102(2): 497-505.

In some embodiments, recombinant nucleic acids are transferred into cells (e.g. hepatocytes) via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material into immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

### III. Pharmaceutical Compositions and Formulations

In some aspects, provided herein are compositions, such as pharmaceutical compositions and formulations for administration, that include any of the DNA-targeting systems described herein, or any of the polynucleotides or vectors encoding the same. In some aspects, the pharmaceutical composition contains one or more DNA-targeting systems provided herein or a component thereof. In some aspects, the pharmaceutical composition comprises one or more vectors that contain polynucleotides that encode one or more components of the DNA-targeting systems provided herein. Such compositions can be used in accord with the provided methods, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject or a cell to which the formulation would be administered.

In some embodiments, the pharmaceutical composition may further comprise a pharmaceutically acceptable excipient. The pharmaceutically acceptable excipient may be functional molecules as vehicles, adjuvants, carriers, or diluents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

In some embodiments, the transfection facilitating agent is a polyanion, polycation, including poly-L-glutamate (LGS), or lipid. In some embodiments, the transfection facilitating agent is poly-L-glutamate. In some embodiments, the transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the DNA-targeting system may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent in a solvent, such as in admixture with a suitable carrier, diluent, or excipient such as sterile water, physiological saline, glucose, dextrose, or the like. The formulations to be used for in vivo or ex vivo administration or use are generally sterile. Sterility may be readily accomplished, e.g., by filtration through sterile filtration membranes.

The pharmaceutical composition in some embodiments contains components in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the composition can be administered to a subject by any suitable means, for example, by bolus infusion or by injection, e.g., by intravenous or subcutaneous injection. In some embodiments, a given dose is administered by a single bolus administration of the composition. In some embodiments, the composition is administered by multiple bolus administrations of the composition, for example, over a period of no more than 3 days, or by continuous infusion administration of the composition. In some embodiments, the composition is administered parenterally, for example by intravenous, intramuscular, subcutaneous, or intraperitoneal administration. In some embodiments, the composition is administered to a subject using peripheral systemic delivery by intravenous, intraperitoneal, or subcutaneous injection.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

### IV. Methods of Treatment

Provided herein are methods of treatment, e.g., including administering any of the compositions, such as pharmaceutical compositions described herein. In some aspects, also provided are methods of administering any of the compositions described herein to a subject, such as a subject that has a disease or disorder. The compositions, such as pharmaceutical compositions, described herein are useful in a variety of therapeutic, diagnostic and prophylactic indications. For example, the compositions are useful in treating a variety of diseases and disorders in a subject. Such methods and uses include therapeutic methods and uses, for example, involving administration of the compositions, to a subject having a disease, condition, or disorder, such as cardiovascular disease and/or familial hyperholesterolemia. In some of any of the provided embodiments, the subject has a cardiovascular disease. In some of any of the provided embodiments, the subject has familial hypercholesterolemia. In some embodiments, the compositions are administered in an effective amount to effect treatment of the disease or disorder. Uses include uses of the compositions in such methods and treatments, and in the preparation of a medicament in order to carry out such therapeutic methods. In some embodiments, the methods are carried out by administering the compositions to the subject having or suspected of having the disease or condition. In some embodiments, the methods thereby treat the disease or condition or disorder in the subject. Also provided are therapeutic methods for administering the cells and compositions to subjects, e.g., patients.

In some embodiments, the compositions include a DNA-targeting system provided herein, or a polynucleotide or vector encoding the same, in which delivery of the composition to a subject modulates one or more activities or function of liver cells in a subject to thereby treat a disease or condition. For instance, in some embodiments, the subject has familial hypercholesterolemia, characterized by elevated levels of LDL in the blood. In some embodiments, administration or use of a composition that includes a DNA-targeting system provided herein, or a polynucleotide or vector encoding the same, decreases expression of a combination of genes in a population of cells in the liver, leading to increased capacity of the liver cells to reduce LDL in the blood. Reduced LDL in the blood reduces the risk of adverse effects of familial hypercholesterolemia, including cardiovascular disease.

In some embodiments, the methods of administering a composition containing the DNA-targeting system or a polynucleotide or vector encoding the same to a subject as provided herein are carried out in vivo (i.e. in a subject).

In some embodiments, the methods and uses for administering the DNA-targeting systems results in delivery of the DNA-targeting system to liver cells (e.g. hepatocytes). In some embodiments, the methods of administering the epigenetic-modifying DNA-targeting system (or polynucleotides or vectors for delivery of same to the liver cell(s) or compositions of any of the foregoing) to a subject comprises contacting the DNA-targeting system with a liver cell or a population of liver cells. In some embodiments, the contacting introduces the epigenome-modifying DNA-targeting system (or polynucleotides or vectors for delivery of same to the liver cell or compositions of any of the foregoing) into the liver cell, such as where it is able to translocate or localize to the nucleus of the liver cell. In some embodiments, the methods promote an enhanced LDL-reducing phenotype in the liver cell or one or more liver cells in the population. In some embodiments, the methods increase the percentage of liver cells with the phenotype in the population of liver cells.

In some embodiments, the delivery to liver cells leads to an epigenetic change in the genome of the cell in a gene or regulatory region, or a combination of genes or regulatory regions, that is targeted by the DNA-targeting system. In some embodiments, the epigenetic change comprises a change in at least one of: DNA accessibility, histone methylation, acetylation, phosphorylation, ubiquitylation, sumoylation, ribosylation, citrullination, and DNA methylation. In some embodiments, the epigenetic change is an altered DNA methylation of a target site in a target gene or a regulatory element thereof as described herein. In some embodiments, the epigenetic change is a histone modification of a target site in a target gene or a regulatory element thereof as described herein. In some embodiments, the delivery of the DNA-targeting system to a liver cell (e.g., hepatocyte) results in a reduction of LDL in the liver of the subject.

In some embodiments, provided herein is a liver cell with an epigenetic modification, i.e., a modified liver cell. In some embodiments, the modifications in the epigenome of the liver cell is by targeting a combination of genes as described herein with a provided epigenetic-modifying DNA-targeting system to change the epigenome of the liver cell. In some embodiments, the modified liver cell includes an epigenetic change in a gene selected from the list consisting of: PCSK9, LPA, MYLIP, ANGLPTL3, APOC3, and APOB. In some embodiments, the epigenetic change of any of the above target genes is a change in at least one of: DNA accessibility, histone methylation, acetylation, phosphorylation, ubiquitylation, sumoylation, ribosylation, citrullination, and DNA methylation, compared to a comparable unmodified cell (e.g. liver cell) not subjected to the method, i.e. not contacted or introduced with the DNA-targeting system described herein.

In some embodiments, the administration of the DNA-targeting system modulates expression of a gene or a combination of genes in a liver cell, such as a gene or a combination of target genes as described in Section I.A. In some embodiments, the target gene or combination of target genes, such as the target genes described in Section IA, is reduced in comparison to a comparable unmodified cell (e.g. liver) not subjected to the method, i.e. not contacted or introduced with the DNA-targeting system described herein. In some embodiments, the expression of the one or more genes is reduced by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, 5-fold, 10-fold, 20-fold, 30-fold, 40-fold, 50-fold, 100-fold, 200-fold, 300-fold, 400-fold, 500-fold, 1000-fold or more. In some embodiments, the expression is stably reduced or transiently reduced. In some embodiments, the reduced expression of the combination of genes promotes a phenotype in a liver cell that reduces LDL in a subject. In some embodiments, a liver cell in the subject has been modulated to have reduced expression of a combination of genes selected from the list consisting of. PCSK9, LPA, MYLIP, ANGLPTL3, APOC3, and APOB. In some embodiments, the expression of each gene of the combination of genes in the modified liver cell is reduced by at least 1.5-fold or more compared to the expression of the same gene in a comparable unmodified liver cell, such as reduced by at or about or greater than 2-fold, 3-fold, 4-fold, 5-fold, 6-fold, 7-fold, 8-fold, 9-fold, 10-fold or more.

In some embodiments, the administration of the DNA-targeting system is provided as a single dose infusion to the subject. In some embodiments, the subject is redosed with the same or a higher dose of the DNA-targeting system. In some embodiments, the administration is repeated a plurality of times at regular intervals.

In some embodiments, the disease, condition, or disorder to be treated is elevated levels of low-density lipoprotein in the blood, increased risk of cardiovascular disease, increased risk of early-onset cardiovascular disease, a mutation affecting cholesterol biosynthesis, a loss-of-function mutation in a low-density lipoprotein receptor (LDLR) gene, a loss-of-function mutation in APOB, a gain-of-function mutation in PCSK9, or familial hypercholesterolemia.

In some embodiments, the subject has or is suspected of having elevated levels of low-density lipoprotein in the blood, increased risk of cardiovascular disease, increased risk of early-onset cardiovascular disease, a mutation affecting cholesterol biosynthesis, a loss-of-function mutation in a low-density lipoprotein receptor (LDLR) gene, a loss-of-function mutation in APOB, a gain-of-function mutation in PCSK9, or familial hypercholesterolemia.

In some embodiments, the subject has or is suspected of having familial hypercholesterolemia.

In some embodiments, the disease, condition or disorder to be treated is a cardiovascular disease. In some embodiments, the cardiovascular disease is Familial Hypercholestorlemia (FH). In some embodiments, the cardiovascular disease is artheoscherotic cardiovascular disease (ASCVD).

Once the multiplexed epigenetic-modifying DNA-targeting system is administered to the subject (e.g., human), the biological activity of the modified cell populations in some aspects is measured by any of a number of known methods. Parameters to assess include reduced levels of circulating LDL (e.g. by analysis of blood serum using ELISA), reduced expression of target genes in the liver cells (e.g. ex vivo, by qRT-PCR), increased capacity for LDL uptake (e.g. based on uptake of fluorescently labeled LDL molecules as assessed by flow cytometry), and/or increased expression of LDL-R. In some aspects the biological activity is measured by assessing clinical outcome. Specific thresholds for the parameters can be set to determine the efficacy of the methods of therapy provided herein.

### V. Kits and Articles of Manufacture

Also provided are articles of manufacture, systems, apparatuses, and kits useful in performing the provided embodiments. In some embodiments, the provided articles of manufacture or kits contain any of the DNA-targeting systems described herein, any of the gRNAs described herein, any of the fusion proteins described herein, any of the polynucleotides described herein, any of the pluralities of polynucleotides described herein, any of the vectors described herein, any of the pluralities of vectors described herein, or a portion or a component of any of the foregoing, or any combination thereof. In some embodiments, the articles of manufacture or kits include polypeptides, polynucleotides, nucleic acids, vectors, and/or cells useful in performing the provided methods.

In some embodiments, the articles of manufacture or kits include one or more containers, typically a plurality of containers, packaging material, and a label or package insert on or associated with the container or containers and/or packaging, generally including instructions for use, e.g., instructions for introducing or administering.

Also provided are articles of manufacture, systems, apparatuses, and kits useful in administering the provided compositions, e.g., pharmaceutical compositions, e.g., for use in therapy or treatment. In some embodiments, the articles of manufacture or kits provided herein contain vectors and/or plurality of vectors, such as any vectors and/or plurality of vectors described herein. In some aspects, the articles of manufacture or kits provided herein can be used for administration of the vectors and/or plurality of vectors, and can include instructions for use.

The articles of manufacture and/or kits containing compositions for therapy, may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In some embodiments, “about” may refer to +25%, ±20%, ±15%, +10%, +5%, or 1^{%}.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, corresponding residues can be identified, for example, using conserved and identical amino acid residues as guides. In general, to identify corresponding positions, the sequences of amino acids are aligned so that the highest order match is obtained (see, e.g., Computational Molecular Biology, Lesk, A.M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

A “gene,” includes a DNA region encoding a gene product. Thus, the gene typically refers to coding and/or transcribed sequences. The sequence of a gene is typically present at a fixed chromosomal position or locus on a chromosome in the cell.

A “regulatory element” or “DNA regulatory element,” which terms are used interchangeably herein, in reference to a gene refers to DNA regions which regulate the production of a gene product, whether or not such regulatory sequences are adjacent to coding and/or transcribed sequences. Accordingly, a regulatory element includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, a “target site” or “target nucleic acid sequence” is a nucleic acid sequence that defines a portion of a nucleic acid to which a binding molecule (e.g. a DNA-binding domain disclosed herein) will bind, provided sufficient conditions for binding exist.

The term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. For instance, expression includes the transcription and/or translation of a particular nucleotide sequence drive by its promoter. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of or a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

As used herein, the term “DNA-targeting system” or “epigenetic-modifying DNA-targeting system” refers to a composition comprising a DNA-binding domain (such as any Cas, ZFN, or TALE-based DNA-binding domain described herein) that targets a target site of a target gene. In some embodiments, the DNA-targeting system is engineered to target the target site. In some embodiments, the DNA-targeting system comprises one or more effector domains that modify transcription of the target gene when recruited to the target site by the DNA-targeting system. In some embodiments, the DNA-targeting system is capable of targeting more than one target site (i.e. a plurality of target sites), such as 2, 3, 4, 5, 6, or more target sites.

As used herein, the term “DNA-targeting module” refers to any composition or portion of a DNA-targeting system described herein that targets one target site. For example, an individual DNA-targeting module may comprise a fusion protein comprising a DNA-binding domain (e.g. a ZFN or TALE DNA-binding domain) that targets a target site for a target gene, and a transcriptional repressor domain (e.g. KRAB). In other embodiments, a DNA-targeting module comprises (a) a fusion protein comprising a Cas protein and a transcriptional repressor domain, and (b) a gRNA that targets a target site of a target gene. A DNA-targeting system provided herein may comprise one or more (such as two) DNA-targeting modules.

In some embodiments, two DNA-targeting modules of a DNA-targeting system may comprise separate, (i.e. non-overlapping) components. For example, a DNA-targeting system may comprise two different fusion proteins, each fusion protein targeting and repressing a different gene. For example, the DNA-targeting system may comprise a first DNA-targeting module comprising a first fusion protein comprising a DNA-binding domain (such as a ZFN or TALE DNA-binding domain) that targets a target site for a first gene and a transcriptional repressor domain, and a second DNA-targeting module comprising a second fusion protein comprising a DNA-binding domain that targets a target site for a second gene and a transcriptional repressor domain. In another example, the DNA-targeting system may comprise a first DNA-targeting module comprising a first fusion protein comprising a DNA-binding domain that targets a target site for a first gene (such as a ZFN or TALE DNA-binding domain) and a transcriptional repressor domain, and a second DNA-targeting module comprising (a) a fusion protein comprising a Cas protein and a transcriptional repressor domain and (b) a gRNA that targets a target site for a second gene. In another example, the DNA-targeting system may comprise a first DNA-targeting module comprising a first fusion protein comprising a first Cas protein and a transcriptional repressor domain, and (b) a first gRNA that complexes with the first Cas protein and targets a target site for a first target gene, and a second DNA-targeting module comprising a second fusion protein comprising a second Cas protein that is different from the first Cas protein and a transcriptional repressor domain, and (b) a second gRNA that complexes with the second Cas protein and targets the second Cas protein to a target site for a second target gene. It will be understood that different Cas protein variants (e.g. SpCas9 and SaCas9) are compatible with different gRNA scaffold sequences and PAMs, as described herein. Thus, it is possible to engineer a single DNA-targeting system comprising multiple non-overlapping CRISPR/Cas-based DNA-targeting modules, each targeting a different target site.

In some embodiments, two DNA-targeting modules of a DNA-targeting system may comprise shared (i.e. overlapping) components. For example, a DNA-targeting system may comprise a first DNA-targeting module comprising (a) a fusion protein comprising a Cas protein and a transcriptional repressor domain, and (b) a gRNA that targets a target site for a first gene, and a second DNA-targeting module comprising (a) the fusion protein of the first DNA-targeting module, and (b) a gRNA that targets a target site for a second gene. It will be understood that providing two or more different gRNAs for a given Cas protein allows different molecules of the same Cas protein to be targeted to the target sites of the two or more gRNAs.

As used herein, the term “increased expression”, “enhanced expression” or “overexpression” means any form of expression that is additional to the expression in an original or source cell that does not contain the modification for modulating a particular gene expression by a. DNA-targeting system, for instance a wild-type expression level (which can be absence of expression or immeasurable expression as well). Reference herein to “increased expression,” “enhanced expression” or “overexpression” is taken to mean an increase in gene expression relative to the level in a cell that does not contain the modification, such as the original source cell prior to contacting with, or engineering to introduce, the DNA-targeting system into the cell, such as an unmodified cell or a wild-type cell. The increase in expression can be at least 5%, 10%, 20%, 30%, 40% or 50%, 60%, 70%, 80%, 85%, 90%, or 100% or even more. In some cases, the increase in expression can be at least 2-fold, 5-fold, 10-fold, 20-fold, 30-fold, 40-fold, 50-fold, 60-fold, 70-fold, 80-fold, 90-fold, 100-fold, 200-fold, 300-fold, 400-food, 500-fold, 1000-fold or more.

As used herein, the term “reduced expression” or “decreased expression” means any form of expression that is lower than the expression in an original or source cell that does not contain the modification for modulating a particular gene expression by a[DNA-targeting system, for instance a wild-type expression level (which can be absence of expression or immeasurable expression as well). Reference herein to “reduced expression,” or “decreased expression” is taken to mean a decrease in gene expression relative to the level in a cell that does not contain the modification, such as the original source cell prior to contacting with, or engineering to introduce, the DNA-binding system into the cell, such as an unmodified cell or a wild-type cell. The decrease in expression can be at least 5%, 10%, 20%, 30%, 40% or 50%, 60%, 70%, 80%, 85%, 90%, or 100% or even more. In some cases, the decrease in expression can be at least 2-fold, 5-fold, 10-fold, 20-fold, 30-fold, 40-fold, 50-fold, 60-fold, 70-fold, 80-fold, 90-fold, 100-fold, 200-fold or more.

As used herein, the term “reduced transcription” or “decreased transcription” refers to the level of transcription of a gene that is lower than the transcription of the gene in an original or source cell that does not contain the modification for modulating transcription by a DNA-targeting system, for instance a wild-type transcription level of a gene. Reference to reduced transcription or decreased transcription can refer to reduction in the levels of a transcribable product of a gene such as mRNA. Any of a variety of methods can be used to monitor or quantitate a level of a transcribable product such as mRNA, including but not limited to, real-time quantitative RT (reverse transcriptase)-polymerase chain reaction (qRT-PCR), Northern Blot, microarray analysis, or RNA sequencing (RNA-Seq). The reduction in transcription can be at least 5%, 10%, 20%, 30%, 40% or 50%, 60%, 70%, 80%, 85%, 90%, or 100% or even more. In some cases, the reduction in transcription can be at least 2-fold, 5-fold, 10-fold, 20-fold, 30-fold, 40-fold, 50-fold, 60-fold, 70-fold, 80-fold, 90-fold, 100-fold, 200-fold or more.

As used herein, an “epigenetic modification” refers to changes in the gene expression that are non-genetic modifications, i.e. not caused by changes in the DNA sequences, but are due to epigenetic changes such as events like DNA methylations or histone modifications. An epigenetic modification may result in a heritable change in gene activity and expression that occur without alteration in DNA sequence. For instance, epigenetic modifications include non-genetic modifications such as chernical modifications to the cytosine residues of DNA (DNA methylation) and histone proteins associated with DNA (histone modifications).

As used herein, the term “modification” or “modified” with reference to a cell refers to any change or alteration in a cell that impacts gene expression in the cell. In some embodiments, the modification is an epigenetic modification that directly changes the epigenetic state of a gene or regulatory elements thereof to alter (e.g. reduce or increase) expression of a gene product. In some embodiments, a modification described herein results in reduced expression of a target gene or selected polynucleotide sequence.

As used herein, a “fusion” molecule is a molecule in which two or more subunit molecules are linked, such as covalently. Examples of a fusion molecule include, but are not limited to, fusion proteins (for example, a fusion between a DNA-binding domain such as a ZFP, TALE DNA-binding domain or CRISPR-Cas protein and one or more effector domains, such as a transactivation domain). The fusion molecule also may be part of a system in which a polynucleotide component associates with a polypeptide component to form a functional molecule (e.g., a CRISPR/Cas system in which a single guide RNA associates with a functional domain to modulate gene expression). Fusion molecules also include fusion nucleic acids, for example, a nucleic acid encoding the fusion protein. Expression of a fusion protein in a cell can result from delivery of the fusion protein to the cell or by delivery of a polynucleotide encoding the fusion protein to a cell, where the polynucleotide is transcribed, and the transcript is translated, to generate the fusion protein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as adenoviral vectors or lentiviral vectors.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include, but are not limited to, cosmids, plasmids (e.g., naked or contained in liposomes) and viruses (e.g., lentiviruses, retroviruses, adenoviruses, and adeno-associated viruses) that incorporate the recombinant polynucleotide.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomelic “nucleotides.” The monomelic nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, in some embodiments, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

In some embodiments, “operably linked” may include the association of components, such as a DNA sequence, (e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human. In some embodiments, the subject is a patient that is known or suspected of having a disease, disorder or condition.

As used herein, the term “treating” and “treatment” includes administering to a subject an effective amount of a biological molecule, such as a therapeutic agent, so that the subject has a reduction in at least one symptom of the disease or an improvement in the disease, for example, beneficial or desired clinical results. For instance, a biological molecule may include cells (e.g. liver cells), such as cells that have been modified by a DNA-targeting system or polynucleotide(s) encoding the DNA-targeting system described herein. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a biological molecule, such as a compound or cells, that, when administered, is sufficient to prevent development of, or alleviate to some extent, one or more of the signs or symptoms of the disorder or disease being treated. The therapeutically effective amount will vary depending on the biological molecule, the disease and its severity and the age, weight, etc., of the subject to be treated.

### VII. Exemplary Embodiments

Among the provided embodiments are:
1. An epigenetic-modifying DNA-targeting system comprising a plurality of DNA-targeting modules for repressing transcription of a plurality of genes that regulate low-density lipoprotein (LDL),
wherein the plurality of DNA-targeting modules comprises a first DNA-targeting module for repressing transcription of a first gene of the plurality of genes, and a second DNA-targeting module for repressing transcription of a second gene of the plurality of genes, and
wherein each DNA-targeting module comprises a fusion protein comprising: (a) a DNA-binding domain for targeting to a target site of one of the plurality of genes, and (b) at least one transcriptional repressor domain.
2. The epigenetic-modifying DNA-targeting system of embodiment 1, wherein the DNA-targeting system does not introduce a genetic disruption or a DNA break.
3. The epigenetic-modifying DNA-targeting system of embodiment 1 or embodiment 2, wherein the fusion protein of each DNA-targeting module comprises a DNA-binding domain selected from: a Clustered Regularly Interspaced Short Palindromic Repeats associated (Cas) protein or a variant thereof, a zinc finger protein (ZFP); a transcription activator-like effector (TALE); a meganuclease; a homing endonuclease; or an I-SceI enzyme or a variant thereof, optionally wherein the DNA-binding domain comprises a catalytically inactive variant of any of the foregoing.
4. The epigenetic-modifying DNA-targeting system of any of embodiments 1-3, wherein:
the fusion protein of the first DNA-targeting module comprises a DNA-binding domain for targeting a target site of the first gene or regulatory DNA element thereof, and at least one transcriptional repressor domain; and
the fusion protein of the second DNA-targeting module comprises a DNA-binding domain for targeting a target site of the second gene or regulatory DNA element thereof, and at least one transcriptional repressor domain.
5. The epigenetic-modifying DNA-targeting system of any of embodiments 1-4, wherein any two or more of the DNA-targeting modules comprise the same fusion protein.
6. The epigenetic-modifying DNA-targeting system of any of embodiments 1-5, wherein the first and second DNA-targeting modules comprise the same fusion protein.
7. The epigenetic-modifying DNA-targeting system of any of embodiments 1-4, wherein any two or more of the DNA-targeting modules comprise different fusion proteins.
8. The epigenetic-modifying DNA-targeting system of any of embodiments 1-4 and 7, wherein the first and second DNA-targeting modules comprise different fusion proteins.
9. An epigenetic-modifying DNA-targeting system comprising a plurality of DNA-targeting modules for repressing transcription of a plurality of genes that regulate low-density lipoprotein (LDL), comprising:
   (1) a first DNA-targeting module that reduces transcription of a first gene that regulates low-density lipoprotein (LDL), wherein the first DNA-targeting module comprises a first fusion protein comprising (a) a DNA-binding domain for targeting a target site of the first gene or regulatory DNA element thereof, and (b) at least one transcriptional repressor domain; and
   (2) a second DNA-targeting module that reduces transcription of a second gene that regulates LDL, wherein the second DNA-targeting module comprises a second fusion protein comprising (a) a DNA-binding domain for targeting a target site of the second gene or regulatory DNA element thereof, and (b) at least one transcriptional repressor domain.
10. The epigenetic-modifying DNA-targeting system of embodiment 5 or 6, wherein the first DNA-targeting module comprises a first targeting polynucleotide for targeting to the target site of the first gene and the second DNA-targeting module comprises a second targeting polynucleotide for targeting to the target site of the second gene, wherein the first and second targeting polynucleotides complex with the DNA-binding domain of the fusion protein.
11. The epigenetic-modifying DNA-targeting system of embodiment 10, wherein the DNA-binding domain is a Clustered Regularly Interspaced Short Palindromic Repeats associated (Cas) protein or variant thereof and the first and second targeting polynucleotides comprise a first gRNA and a second gRNA, respectively.
12. An epigenetic-modifying DNA-targeting system comprising:
   (a) a fusion protein comprising a Clustered Regularly Interspaced Short Palindromic Repeats associated (Cas) protein or variant thereof and at least one transcriptional repressor domain; and
   (b) a plurality of guide RNAs (gRNAs) comprising at least a first gRNA and a second gRNA, wherein the first gRNA targets a target site of a first gene that regulates low-density lipoprotein (LDL) and the second gRNA targets a target site of a second gene that regulates LDL.
13. The epigenetic-modifying DNA-targeting system of any of embodiments 1-12, wherein the system further comprises a third DNA-targeting module for repressing transcription of a third gene that regulates low-density lipoprotein (LDL).
14. The epigenetic-modifying DNA-targeting system of any of embodiments 11-13, wherein the system further comprises a third gRNA that targets a target site of a third gene that regulates LDL, optionally wherein the system further comprises a fourth gRNA that targets a target site of a fourth gene that regulates LDL, optionally a fifth gRNA that targets a target site of a fifth gene that regulates LDL, and/or optionally a sixth gRNA that targets a target site of a sixth gene that regulates LDL.
15. The epigenetic-modifying DNA-targeting system of any of embodiments 1-14, wherein the first gene and the second gene are independently selected from the group consisting of PCSK9, LPA, MYLIP, ANGPTL3, APOB, and APOC3.
16. The epigenetic-modifying DNA-targeting system of any of embodiments 1-15, wherein the first gene and the second gene are different.
17. The epigenetic-modifying DNA-targeting system of any of embodiments 1-16, wherein the first gene and the second gene are: PCSK9 and LPA; PCSK9 and MYLIP; PCSK9 and ANGPTL3; PCSK9 and APOC3; PCSK9 and APOB; LPA and MYLIP; LPA and ANGPTL3; LPA and APOC3; LPA and APOB; MYLIP and ANGPTL3; MYLIP and APOC3; MYLIP and APOB; ANGPTL3 and APOC3; ANGPTL3 and APOB; or APOC3 and APOB.
18. The epigenetic-modifying DNA-targeting system of any of embodiments 1-17, wherein the first gene, the second gene, and the third gene are each independently selected from the group consisting of PCSK9, LPA, MYLIP, ANGPTL3, APOC3, and APOB.
19. The epigenetic-modifying DNA-targeting system of any of embodiments 13-18, wherein the first gene, the second gene, and the third gene are different.
20. The epigenetic-modifying DNA-targeting system combination of any of embodiments 13-19, wherein the first gene, the second gene, and the third gene are: PCSK9, LPA, and MYLIP; PCSK9, LPA, and ANGPTL3; PCSK9, LPA, and APOC3; PCSK9, LPA, and APOB; PCSK9, MYLIP, and ANGPTL3; PCSK9, MYLIP, and APOC3; PCSK9, MYLIP, and APOB; PCSK9, ANGPTL3, and APOC3; PCSK9, ANGPTL3, and APOB; PCSK9, APOC3, and APOB; LPA, MYLIP, and ANGPTL3; LPA, MYLIP, and APOC3; LPA, MYLIP, and APOB; LPA, ANGPTL3, and APOC3; LPA, ANGPTL3, and APOB; LPA, APOC3, and APOB; MYLIP, ANGPTL3, and APOC3; MYLIP, ANGPTL3, and APOB; MYLIP, APOC3, and APOB; or ANGPTL3, APOC3, and APOB.
21. The epigenetic-modifying DNA-targeting system of any of embodiments 1-20, wherein at least one gene is PCSK9.
22. The epigenetic-modifying DNA-targeting system of any of embodiments 1-21, wherein at least two genes are PCSK9 and LPA.
23. The epigenetic-modifying DNA-targeting system of any of embodiments 13-22, wherein at least three genes are PCSK9, LPA, and MYLIP.
24. The epigenetic-modifying DNA-targeting system of any of embodiments 1-23, wherein the target site of each of the plurality of genes is in the gene or a regulatory DNA element thereof.
25. The epigenetic-modifying DNA-targeting system of embodiment 24, wherein the regulatory DNA element is an enhancer or a promoter.
26. The epigenetic-modifying DNA-targeting system of any of embodiments 1-25, wherein the target site of the first gene and the second gene are selected from two different members of the group consisting of (a)-(f):
   (a) a target site for PCSK9, located within 500 bp of human genome assembly GRCh38 (hg38) genomic coordinates chr1:55,039,548;
   (b) a target site for LPA, located within 500 bp of the hg38 genomic coordinates chr6:160,664,275;
   (c) a target site for MYLIP, located within 500 bp of the hg38 genomic coordinates chr6:16,129,086;
   (d) a target site for ANGPTL3, located within 500 bp of the hg38 genomic coordinates chr1:62,597,520;
   (e) a target site for APOC3, located within 500 bp of the hg38 genomic coordinates chr11:116,829,907; and
   (f) a target site for APOB, located within 500 bp of the hg38 genomic coordinates chr2:21,044,073.
27. The epigenetic-modifying DNA-targeting system of any of embodiments 1-26, wherein the target site of the first gene and the second gene are selected from two different members of the group consisting of (a)-(f):
   (a) a target site located within 500 bp of a transcriptional start site of PCSK9;
   (b) a target site located within 500 bp of a transcriptional start site of LPA;
   (c) a target site located within 500 bp of a transcriptional start site of MYLIP;
   (d) a target site located within 500 bp of a transcriptional start site of ANGPTL3;
   (e) a target site located within 500 bp of a transcriptional start site of APOC3; and
   (f) a target site located within 500 bp of a transcriptional start site of APOB.
28. The epigenetic-modifying DNA-targeting system of any of embodiments 13-27, wherein the target site of the first gene, the second gene and the third gene are selected from three different members of the group consisting of (a)-(f):
   (a) a target site for PCSK9, located within 500 bp of human genome assembly GRCh38 (hg38) genomic coordinates chr1:55,039,548;
   (b) a target site for LPA, located within 500 bp of the hg38 genomic coordinates chr6:160,664,275;
   (c) a target site for MYLIP, located within 500 bp of the hg38 genomic coordinates chr6:16,129,086;
   (d) a target site for ANGPTL3, located within 500 bp of the hg38 genomic coordinates chr1:62,597,520;
   (e) a target site for APOC3, located within 500 bp of the hg38 genomic coordinates chr11:116,829,907; and
   (f) a target site for APOB, located within 500 bp of the hg38 genomic coordinates chr2:21,044,073.
29. The epigenetic-modifying DNA-targeting system of any of embodiments 13-28, wherein the target site of the first gene, the second gene and the third gene are selected from three different members of the group consisting of (a)-(f):
   (a) a target site located within 500 bp of a transcriptional start site of PCSK9;
   (b) a target site located within 500 bp of a transcriptional start site of LPA;
   (c) a target site located within 500 bp of a transcriptional start site of MYLIP;
   (d) a target site located within 500 bp of a transcriptional start site of ANGPTL3;
   (e) a target site located within 500 bp of a transcriptional start site of APOC3; and
   (f) a target site located within 500 bp of a transcriptional start site of APOB.
30. The epigenetic-modifying DNA-targeting system of any of embodiments 1-29, wherein the target site of the first gene and the second gene are selected from two different members of the group consisting of (a)-(f):
   (a) a target site for PCSK9 having the sequence set forth in any one of SEQ ID NOS:1-13 or 306-317, a contiguous portion thereof of at least 14 nucleotides (nt), or a complementary sequence of any of the foregoing, optionally wherein the target site is (i) set forth in SEQ ID NO:3, (ii) has a contiguous portion of SEQ ID NO:3 of at least 14 nucleotides, or (iii) is a complementary sequence of (i) or (ii);
   (b) a target site for LPA having the sequence set forth in any one of SEQ ID NOS:14-23, a contiguous portion thereof of at least 14 nucleotides (nt), or a complementary sequence of any of the foregoing;
   (c) a target site for MYLIP having the sequence set forth in any one of SEQ ID NOS:24-33 or 342-351, a contiguous portion thereof of at least 14 nucleotides (nt), or a complementary sequence of any of the foregoing;
   (d) a target site for ANGPTL3 having the sequence set forth in any one of SEQ ID NOS:34-43, a contiguous portion thereof of at least 14 nucleotides (nt), or a complementary sequence of any of the foregoing;
   (e) a target site for APOC3 having the sequence set forth in any one of SEQ ID NOS:44-53, a contiguous portion thereof of at least 14 nucleotides (nt), or a complementary sequence of any of the foregoing; and
   (f) a target site for APOB having the sequence set forth in any one of SEQ ID NOS:54-63 or 372-377, a contiguous portion thereof of at least 14 nucleotides (nt), or a complementary sequence of any of the foregoing.
31. The epigenetic-modifying DNA-targeting system combination of any of embodiments 1-30, wherein the target site of the first gene and the second gene are selected from two different members of the group consisting of (a)-(f):
   (a) a target site of PCSK9 having the sequence set forth in any one of SEQ ID NOS:1-13 or 306-317, optionally wherein the target site is set forth in SEQ ID NO:3;
   (b) a target site of LPA having the sequence set forth in any one of SEQ ID NOS:14-23;
   (c) a target site of MYLIP having the sequence set forth in any one of SEQ ID NOS:24-33 or 342-351;
   (d) a target site of ANGPTL3 having the sequence set forth in any one of SEQ ID NOS: 34-43;
   (e) a target site of APOC3 having the sequence set forth in any one of SEQ ID NOS:44-53; and
   (f) a target site of APOB having the sequence set forth in any one of SEQ ID NOS:54-63 or 372-377.
32. The epigenetic-modifying DNA-targeting system combination of any of embodiments 13-31, wherein the target site of the first gene, the second gene and the third gene are selected from three different members of the group consisting of (a)-(f):
   (a) a target site of PCSK9 having the sequence set forth in any one of SEQ ID NOS:1-13 or 306-317, a contiguous portion thereof of at least 14 nucleotides (nt), or a complementary sequence of any of the foregoing, optionally wherein the target site is (i) set forth in SEQ ID NO:3, (ii) has a contiguous portion of SEQ ID NO:3 of at least 14 nucleotides, or (iii) is a complementary sequence of (i) or (ii);
   (b) a target site of LPA having the sequence set forth in any one of SEQ ID NOS:14-23, a contiguous portion thereof of at least 14 nucleotides (nt), or a complementary sequence of any of the foregoing;
   (c) a target site of MYLIP having the sequence set forth in any one of SEQ ID NOS:24-33 or 342-351, a contiguous portion thereof of at least 14 nucleotides (nt), or a complementary sequence of any of the foregoing;
   (d) a target site of ANGPTL3 having the sequence set forth in any one of SEQ ID NOS:34-43, a contiguous portion thereof of at least 14 nucleotides (nt), or a complementary sequence of any of the foregoing;
   (e) a target site of APOC3 having the sequence set forth in any one of SEQ ID NOS:44-53, a contiguous portion thereof of at least 14 nucleotides (nt), or a complementary sequence of any of the foregoing; and
   (f) a target site of APOB having the sequence set forth in any one of SEQ ID NOS:54-63 or 372-377, a contiguous portion thereof of at least 14 nucleotides (nt), or a complementary sequence of any of the foregoing.
33. The epigenetic-modifying DNA-targeting system combination of any of embodiments 13-32, wherein the target site of the first gene, the second gene or the third gene are selected from three different members of the group consisting of (a)-(f):
   (a) a target site of PCSK9 having the sequence set forth in any one of SEQ ID NOS:1-13 or 306-317, optionally wherein the target site is set forth in SEQ ID NO:3;
   (b) a target site of LPA having the sequence set forth in any one of SEQ ID NOS: 14-23;
   (c) a target site of MYLIP having the sequence set forth in any one of SEQ ID NOS:24-33 or 342-351;
   (d) a target site of ANGPTL3 having the sequence set forth in any one of SEQ ID NOS: 34-43;
   (e) a target site of APOC3 having the sequence set forth in any one of SEQ ID NOS:44-53; and
   (f) a target site of APOB having the sequence set forth in any one of SEQ ID NOS:54-63 or 372-377.
34. The epigenetic-modifying DNA-targeting system of any of embodiments 11-33, wherein the Cas protein or variant thereof is a variant Cas protein that is a deactivated (dCas) protein.
35. The epigenetic-modifying DNA-targeting system of embodiment 34, wherein the dCas protein lacks nuclease activity.
36. The epigenetic-modifying DNA-targeting system of embodiment 34 or embodiment 35, wherein the dCas protein is a dCas9 protein.
37. The epigenetic-modifying DNA-targeting system of embodiment 34 or embodiment 35, wherein the dCas protein is a dCasl2 protein.
38. The epigenetic-modifying DNA-targeting system of embodiment 36, wherein the dCas9 protein is a Staphylococcus aureus dCas9 (dSaCas9) protein.
39. The epigenetic-modifying DNA-targeting system of embodiment 38, wherein the dSaCas9 comprises at least one amino acid mutation selected from D10A and N580A, with reference to numbering of positions of SEQ ID NO:204.
40. The epigenetic-modifying DNA-targeting system of embodiment 38 or embodiment 39, wherein the dSaCas9 protein comprises the sequence set forth in SEQ ID NO:205, or an amino acid sequence that has at least 90%, 91%, 92%, 93%, 94%, 95%, 96%, 97%, 98%, or 99% sequence identity thereto.
41. The epigenetic-modifying DNA-targeting system of any of embodiments 38-40, wherein the dSaCas9 is set forth in SEQ ID NO:205.
42. The epigenetic-modifying DNA-targeting system of embodiment 36, wherein the dCas9 protein is a Streptococcuspyogenes dCas9 (dSpCas9) protein.
43. The epigenetic-modifying DNA-targeting system of embodiment 42, wherein the dSpCas9 protein comprises at least one amino acid mutation selected from D10A and H840A, with reference to numbering of positions of SEQ ID NO:206.
44. The epigenetic-modifying DNA-targeting system of 42 or embodiment 43, wherein the dSpCas9 comprises the sequence set forth in SEQ ID NO:207, or an amino acid sequence that has at least 90%, 91%, 92%, 93%, 94%, 95%, 96%, 97%, 98%, or 99% sequence identity thereto.
45. The epigenetic-modifying DNA-targeting system of any of embodiments 42-44, wherein the dSpCas9 is set forth in SEQ ID NO:207.
46. The epigenetic-modifying DNA-targeting system of any of embodiments 11-45, wherein each gRNA comprises a gRNA spacer sequence that is complementary to the target site of the respective gene.
47. The epigenetic-modifying DNA-targeting system of any of embodiments 11-46, wherein the first gRNA and the second gRNA are selected from two different members of the group consisting of (a)-(f):
   (a) a gRNA targeting a target site of PCSK9 comprising a gRNA spacer sequence comprising the sequence set forth in any one of SEQ ID NOS:64-76 or 318-329, or a contiguous portion thereof of at least 14 nt, optionally wherein the gRNA spacer sequence comprises the sequence set forth in SEQ ID NO:66 or a contiguous portion of at least 14 nt;
   (b) a gRNA targeting a target site of LPA comprising a gRNA spacer sequence comprising the sequence set forth in any one of SEQ ID NOS:77-86, or a contiguous portion thereof of at least 14 nt;
   (c) a gRNA targeting a target site of MYLIP comprising a gRNA spacer sequence comprising the sequence set forth in any one of SEQ ID NOS:87-96 or 352-361, or a contiguous portion thereof of at least 14 nt;
   (d) a gRNA targeting a target site of ANGPTL3 comprising a gRNA spacer sequence comprising the sequence set forth in any one of SEQ ID NOS:97-106, or a contiguous portion thereof of at least 14 nt;
   (e) a gRNA targeting a target site of APOC3 comprising a gRNA spacer sequence comprising the sequence set forth in any one of SEQ ID NOS:107-116, or a contiguous portion thereof of at least 14 nt; and
   (f) a gRNA targeting a target site of APOB comprising a gRNA spacer sequence comprising the sequence set forth in any one of SEQ ID NOS:117-126 or 378-383, or a contiguous portion thereof of at least 14 nt.
48. The epigenetic-modifying DNA-targeting system of any of embodiments 13-47, wherein the first gRNA, the second gRNA, and the third gRNA are selected from three different members of the group consisting of (a)-(f):
   (a) a gRNA targeting a target site of PCSK9 comprising a gRNA spacer sequence comprising the sequence set forth in any one of SEQ ID NOS:64-76 or 318-329, or a contiguous portion thereof of at least 14 nt, optionally wherein the gRNA spacer sequence comprises the sequence set forth in SEQ ID NO:66 or a contiguous portion of at least 14 nt;
   (b) a gRNA targeting a target site of LPA comprising a gRNA spacer sequence comprising the sequence set forth in any one of SEQ ID NOS:77-86, or a contiguous portion thereof of at least 14 nt;
   (c) a gRNA targeting a target site of MYLIP comprising a gRNA spacer sequence comprising the sequence set forth in any one of SEQ ID NOS:87-96 or 352-361, or a contiguous portion thereof of at least 14 nt;
   (d) a gRNA targeting a target site of ANGPTL3 comprising a gRNA spacer sequence comprising the sequence set forth in any one of SEQ ID NOS:97-106, or a contiguous portion thereof of at least 14 nt;
   (e) a gRNA targeting a target site of APOC3 comprising a gRNA spacer sequence comprising the sequence set forth in any one of SEQ ID NOS:107-116, or a contiguous portion thereof of at least 14 nt; and
   (f) a gRNA targeting a target site of APOB comprising a gRNA spacer sequence comprising the sequence set forth in any one of SEQ ID NOS:117-126 or 378-383, or a contiguous portion thereof of at least 14 nt.
49. The epigenetic-modifying DNA-targeting system of any of embodiments 11-48, wherein each gRNA independently comprises a spacer sequence between 14 nt and 24 nt, or between 16 nt and 22 nt in length.
50. The epigenetic-modifying DNA-targeting system of any of embodiments 11-49, wherein each gRNA independently comprises a spacer sequence that is 18 nt, 19 nt, 20 nt, 21 nt, or 22 nt in length.
51. The epigenetic-modifying DNA-targeting system of any of embodiments 11-50, wherein the first gRNA and the second gRNA are selected from two different members of the group consisting of (a)-(f):
   (a) a gRNA targeting a target site of PCSK9 comprising the gRNA spacer sequence set forth in any one of SEQ ID NOS:64-76 or 318-329, optionally wherein the gRNA spacer is set forth in SEQ ID NO:66;
   (b) a gRNA targeting a target site in LPA comprising the gRNA spacer sequence set forth in any one of SEQ ID NOS:77-86;
   (c) a gRNA targeting a target site in MYLIP comprising the gRNA spacer sequence set forth in any one of SEQ ID NOS:87-96 or 352-361;
   (d) a gRNA targeting a target site in ANGPTL3 comprising the gRNA spacer sequence set forth in any one of SEQ ID NOS:97-106;
   (e) a gRNA targeting a target site in APOC3 comprising the gRNA spacer sequence set forth in any one of SEQ ID NOS:107-116; and
   (f) a gRNA targeting a target site in APOB comprising the gRNA spacer sequence set forth in any one of SEQ ID NOS:117-126 or 378-383.
52. The epigenetic-modifying DNA-targeting system of any of embodiments 43-45, wherein each gRNA further comprises a scaffold sequence set forth in SEQ ID NO:191.
53. The epigenetic-modifying DNA-targeting system of any of embodiments 15-52, wherein the first gRNA and the second gRNA are selected from two different members of the group consisting of (a)-(f):
   (a) a gRNA targeting a target site in PCSK9 comprising the sequence set forth in any one of SEQ ID NOS:127-139 or 330-341, optionally wherein the gRNA comprises the sequence set forth in SEQ ID NO:129;
   (b) a gRNA targeting a target site in LPA comprising the sequence set forth in any one of SEQ ID NOS:140-149;
   (c) a gRNA targeting a target site in MYLIP comprising the sequence set forth in any one of SEQ ID NOS:150-159 or 362-371;
   (d) a gRNA targeting a target site in ANGPTL3 comprising the sequence set forth in any one of SEQ ID NOS:160-169;
   (e) a gRNA targeting a target site in APOC3 comprising the sequence set forth in any one of SEQ ID NOS:170-179; and
   (f) a gRNA targeting a target site in APOB comprising the sequence set forth in any one of SEQ ID NOS:180-189 or 384-389.
54. The epigenetic-modifying DNA-targeting system of any of embodiments 15-53, wherein the first gRNA and the second gRNA are selected from two different members of the group consisting of (a)-(f):
   (a) a gRNA targeting a target site in PCSK9 set forth in any one of SEQ ID NOS:127-139 or 330-341, optionally wherein the gRNA is set forth in SEQ ID NO:129;
   (b) a gRNA targeting a target site in LPA set forth in any one of SEQ ID NOS:140-149;
   (c) a gRNA targeting a target site in MYLIP set forth in any one of SEQ ID NOS:150-159 or 362-371;
   (d) a gRNA targeting a target site in ANGPTL3 set forth in any one of SEQ ID NOS:160-169;
   (e) a gRNA targeting a target site in APOC3 set forth in any one of SEQ ID NOS:170-179; and
   (f) a gRNA targeting a target site in APOB set forth in any one of SEQ ID NOS:180-189 or 384-389.
55. The epigenetic-modifying DNA-targeting combination of any of embodiments 15-54, wherein the first gRNA, the second gRNA, and the third gRNA are selected from three different members of the group consisting of (a)-(f):
   (a) a gRNA targeting a target site in PCSK9 comprising the sequence set forth in any one of SEQ ID NOS:127-139 or 330-341, optionally wherein the gRNA comprises the sequence set forth in SEQ ID NO: 129;
   (b) a gRNA targeting a target site in LPA comprising the sequence set forth in any one of SEQ ID NOS:140-149;
   (c) a gRNA targeting a target site in MYLIP comprising the sequence set forth in any one of SEQ ID NOS:150-159 or 362-371;
   (d) a gRNA targeting a target site in ANGPTL3 comprising the sequence set forth in any one of SEQ ID NOS:160-169;
   (e) a gRNA targeting a target site in APOC3 comprising the sequence set forth in any one of SEQ ID NOS:170-179; and
   (f) a gRNA targeting a target site in APOB comprising the sequence set forth in any one of SEQ ID NOS:180-189 or 384-389.
56. The epigenetic-modifying DNA-targeting combination of any of embodiments 15-55, wherein the first gRNA, the second gRNA, and the third gRNA are selected from three different members of the group consisting of (a)-(f):
   (a) a gRNA targeting a target site in PCSK9 set forth in any one of SEQ ID NOS:127-139 or 330-341, optionally wherein the gRNA is set forth in SEQ ID NO:129;
   (b) a gRNA targeting a target site in LPA set forth in any one of SEQ ID NOS:140-149;
   (c) a gRNA targeting a target site in MYLIP set forth in any one of SEQ ID NOS:150-159 or 362-371;
   (d) a gRNA targeting a target site in ANGPTL3 set forth in any one of SEQ ID NOS:160-169;
   (e) a gRNA targeting a target site in APOC3 set forth in any one of SEQ ID NOS:170-179; and
   (f) a gRNA targeting a target site in APOB set forth in any one of SEQ ID NOS:180-189 or 384-389.
57. The epigenetic-modifying DNA-targeting system of any of embodiments 11-56, wherein at least one gRNA comprises modified nucleotides for increased stability.
58. The epigenetic-modifying DNA-targeting combination of any of embodiments 1-57, wherein the at least one transcriptional repressor domain is capable of reducing transcription of the gene.
59. The epigenetic-modifying DNA-targeting combination of any of embodiments 1-58, wherein the transcriptional repressor domain is selected from the group consisting of a KRAB domain, a DNMT3A domain, a DNMT3L domain, a DNMT3B domain, a DNMT3A-DNMT3L fusion protein domain, an ERF repressor domain, an Mxi1 repressor domain, a SID4X repressor domain, a Mad-SID repressor domain, an LSD1 repressor domain, an EZH2 repressor domain, a SunTag domain, or a variant or portion of any of the foregoing, or a combination of any of the foregoing.
60. The epigenetic-modifying DNA-targeting system of any of embodiments 1-59, wherein the transcriptional repressor domain is a KRAB domain, a DNMT3A domain, or a DNMT3L domain, or a combination of any of the foregoing.
61. The epigenetic-modifying DNA-targeting system of any of embodiments 1-60, wherein the at least one transcriptional repressor domain comprises a KRAB domain or a variant or portion thereof that exhibits transcriptional repressor activity.
62. The epigenetic-modifying DNA-targeting system of any of embodiments 1-60, wherein the at least one transcriptional repressor domain comprises the sequence set forth in SEQ ID NO:193, a portion thereof, or an amino acid sequence that has at least 90%, 91%, 92%, 93%, 94%, 95%, 96%, 97%, 98%, or 99% sequence identity to any of the foregoing.
63. The epigenetic-modifying DNA-targeting system of any of embodiments 1-59, wherein the at least one transcriptional repressor domain comprises a DNMT3A domain or a variant or portion thereof that exhibits transcriptional repressor activity.
64. The epigenetic-modifying DNA-targeting system of any of embodiments 1-59 and 63, wherein the at least one transcriptional repressor domain comprises the sequence set forth in SEQ ID NO:195, a portion thereof, or an amino acid sequence that has at least 90%, 91%, 92%, 93%, 94%, 95%, 96%, 97%, 98%, or 99% sequence identity to any of the foregoing.
65. The epigenetic-modifying DNA-targeting system of any of embodiments 1-59, wherein the at least one transcriptional repressor domain comprises a DNMT3L domain or a variant or portion thereof that exhibits transcriptional repressor activity.
66. The epigenetic-modifying DNA-targeting system of any of embodiments 1-59 and 65, wherein the at least one transcriptional repressor domain comprises the sequence set forth in SEQ ID NO: 197, a portion thereof, or an amino acid sequence that has at least 90%, 91%, 92%, 93%, 94%, 95%, 96%, 97%, 98%, or 99% sequence identity to any of the foregoing.
67. The epigenetic-modifying DNA-targeting system of any of embodiments 1-59, wherein the at least one transcriptional repressor domain is a DNMT3A-DNMT3L fusion protein domain or a variant thereof that exhibits transcriptional repressor activity.
68. The epigenetic-modifying DNA-targeting system of any of embodiments 1-59 and 67, wherein the at least one transcriptional repressor domain comprises the sequence set forth in SEQ ID NO:199 or SEQ ID NO:201, a portion thereof, or an amino acid sequence that has at least 90%, 91%, 92%, 93%, 94%, 95%, 96%, 97%, 98%, or 99% sequence identity to any of the foregoing.
69. The epigenetic-modifying DNA-targeting system of any of embodiments 1-59, wherein at least one transcriptional repressor domain comprises a sequence selected from any one of SEQ ID NOS:193, 195, 197, 199, 201, 220-226, and 283, or a domain thereof, a portion thereof, or an amino acid sequence that has at least 90%, 91%, 92%, 93%, 94%, 95%, 96%, 97%, 98%, or 99% sequence identity to any of the foregoing.
70. The epigenetic-modifying DNA-targeting system of any of embodiments 1-69, wherein the at least one transcriptional repressor domain is fused to the N-terminus, the C-terminus, or both the N-terminus and the C-terminus, of the DNA-binding domain.
71. The epigenetic-modifying DNA-targeting system of any of embodiments 1-70, wherein the fusion protein further comprises one or more nuclear localization signals (NLS).
72. The epigenetic-modifying DNA-targeting system of embodiment 71, wherein the fusion protein further comprises one or more linkers connecting two or more of: the DNA-binding domain, the at least one transcriptional repressor domain, and the one or more nuclear localization signals.
73. The epigenetic-modifying DNA-targeting system of any of embodiments 1-107, wherein the fusion protein comprises the sequence set forth in any one of SEQ ID NOS:209, 278, 280, or 282, or an amino acid sequence that has at least 90%, 91%, 92%, 93%, 94%, 95%, 96%, 97%, 98%, or 99% sequence identity thereto, optionally wherein the fusion protein comprises the sequence set forth in SEQ ID NO:280.
74. The epigenetic-modifying DNA-targeting system of any of embodiments 1-108, wherein each of the DNA-targeting modules reduces expression of each of the respective genes by a log 2 fold-change of at or lesser than −1.0.
75. The epigenetic-modifying DNA-targeting system of any of embodiments 1-74, wherein repressed transcription of the plurality of genes in a cell or population of cells leads to a reduction of low-density lipoprotein (LDL).
76. The epigenetic-modifying DNA-targeting system of embodiment 75, wherein the reduction of LDL is greater than the reduction of LDL resulting from comparable repressed transcription of any of the individual genes in the plurality of genes alone.
77. The epigenetic-modifying DNA-targeting system of embodiment 75 or 76, wherein the reduction of LDL occurs extracellularly.
78. The epigenetic-modifying DNA-targeting system of any of embodiments 75-77, wherein the cell or population of cells is a liver cell or comprises liver cells.
79. The epigenetic-modifying DNA-targeting system of any of embodiments 75-78, wherein the cell or population of cells is in a subject.
80. The epigenetic-modifying DNA-targeting system of embodiment 79, wherein the reduction of LDL occurs in the subject or a fluid, tissue, or organ thereof.
81. The epigenetic-modifying DNA-targeting system of any of embodiments 75-80, wherein the reduction of LDL occurs in the blood of a subject.
82. An epigenetic-modifying DNA-targeting system for repressing transcription of a gene that regulates low-density lipoprotein, comprising a fusion protein comprising: (a) a DNA-binding domain for targeting to a target site of a gene, and (b) at least one transcriptional repressor domain.
83. The epigenetic-modifying DNA-targeting system of embodiment 82, wherein the target site of the gene is in the gene or a regulatory DNA element thereof.
84. The epigenetic-modifying DNA-targeting system of embodiment 82 or 83, wherein the DNA-targeting system does not introduce a genetic disruption or a DNA break.
85. The epigenetic-modifying DNA-targeting system of any of embodiments 82-84, wherein the DNA-binding domain is selected from: a Clustered Regularly Interspaced Short Palindromic Repeats associated (Cas) protein or a variant thereof; a zinc finger protein (ZFP); a transcription activator-like effector (TALE); a meganuclease; a homing endonuclease; or an I-SceI enzyme or a variant thereof, optionally wherein the DNA-binding domain comprises a catalytically inactive variant of any of the foregoing.
86. The epigenetic-modifying DNA-targeting system of embodiment 85, wherein the DNA-binding domain is a Clustered Regularly Interspaced Short Palindromic Repeats associated (Cas) protein or variant thereof, and the system further comprises a gRNA for targeting the DNA-binding domain to the target site of the gene.
87. An epigenetic-modifying DNA-targeting system comprising: (a) a fusion protein comprising a Clustered Regularly Interspaced Short Palindromic Repeats associated (Cas) protein or variant thereof and at least one transcriptional repressor domain; and (b) a gRNA that targets a target site of a gene that regulates low-density lipoprotein (LDL).
88. The epigenetic-modifying DNA-targeting system of any of embodiments 82-87, wherein the target site of the gene is in the gene and/or a regulatory DNA element thereof.
89. The epigenetic-modifying DNA-targeting system of embodiment 88, wherein the regulatory DNA element is a promoter or enhancer.
90. The epigenetic-modifying DNA-targeting system of any of embodiments 82-89, wherein the gene is selected from the group consisting of: PCSK9, LPA, MYLIP, ANGPTL3, APOB, and APOC3.
91. The epigenetic-modifying DNA-targeting system of any of embodiments 82-90, wherein the target site is selected from:
   (a) a target site for PCSK9, located within 500 bp of human genome assembly GRCh38 (hg38) genomic coordinates chr1:55,039,548;
   (b) a target site for LPA, located within 500 bp of the hg38 genomic coordinates chr6:160,664,275;
   (c) a target site for MYLIP, located within 500 bp of the hg38 genomic coordinates chr6:16,129,086;
   (d) a target site for ANGPTL3, located within 500 bp of the hg38 genomic coordinates chr1:62,597,520;
   (e) a target site for APOC3, located within 500 bp of the hg38 genomic coordinates chr11:116,829,907; and
   (f) a target site for APOB, located within 500 bp of the hg38 genomic coordinates chr2:21,044,073.
92. The epigenetic-modifying DNA-targeting system of any of embodiments 82-91, wherein the target site is selected from:
   (a) a target site located within 500 bp of a transcriptional start site of PCSK9;
   (b) a target site located within 500 bp of a transcriptional start site of LPA;
   (c) a target site located within 500 bp of a transcriptional start site of MYLIP;
   (d) a target site located within 500 bp of a transcriptional start site of ANGPTL3;
   (e) a target site located within 500 bp of a transcriptional start site of APOC3; and
   (f) a target site located within 500 bp of a transcriptional start site of APOB.
93. The epigenetic-modifying DNA-targeting system of any of embodiments 82-92, wherein the target site is selected from:
   (a) a target site for PCSK9 having the sequence set forth in any one of SEQ ID NOS:1-13 or 306-317, a contiguous portion thereof of at least 14 nucleotides (nt), or a complementary sequence of any of the foregoing, optionally wherein the target site has (i) the sequence set forth in SEQ ID NO:3, (ii) a contiguous portion of at least 14 nucleotides set forth in SEQ ID NO:3; or (iii) a complementary sequence of (i) or (ii);
   (b) a target site for LPA having the sequence set forth in any one of SEQ ID NOS:14-23, a contiguous portion thereof of at least 14 nucleotides (nt), or a complementary sequence of any of the foregoing;
   (c) a target site for MYLIP having the sequence set forth in any one of SEQ ID NOS:24-33 or 342-351, a contiguous portion thereof of at least 14 nucleotides (nt), or a complementary sequence of any of the foregoing;
   (d) a target site for ANGPTL3 having the sequence set forth in any one of SEQ ID NOS:34-43, a contiguous portion thereof of at least 14 nucleotides (nt), or a complementary sequence of any of the foregoing;
   (e) a target site for APOC3 having the sequence set forth in any one of SEQ ID NOS:44-53, a contiguous portion thereof of at least 14 nucleotides (nt), or a complementary sequence of any of the foregoing; and
   (f) a target site for APOB having the sequence set forth in any one of SEQ ID NOS:54-63 or 372-377, a contiguous portion thereof of at least 14 nucleotides (nt), or a complementary sequence of any of the foregoing.
94. The epigenetic-modifying DNA-targeting system of any of embodiments 82-93, wherein the target site is selected from:
   (a) a target site of PCSK9 having the sequence set forth in any one of SEQ ID NOS:1-13 or 306-317, optionally wherein the target set has the sequence set forth in SEQ ID NO:3;
   (b) a target site of LPA having the sequence set forth in any one of SEQ ID NOS:14-23;
   (c) a target site of MYLIP having the sequence set forth in any one of SEQ ID NOS:24-33 or 342-351;
   (d) a target site of ANGPTL3 having the sequence set forth in any one of SEQ ID NOS: 34-43;
   (e) a target site of APOC3 having the sequence set forth in any one of SEQ ID NOS:44-53; and
   (f) a target site of APOB having the sequence set forth in any one of SEQ ID NOS:54-63 or 372-377.
95. The epigenetic-modifying DNA-targeting system of any of embodiments 86-94, wherein the Cas protein or variant thereof is a variant Cas protein that is a deactivated (dCas) protein.
96. The epigenetic-modifying DNA-targeting system of embodiment 95, wherein the dCas protein lacks nuclease activity.
97. The epigenetic-modifying DNA-targeting system of embodiment 95 or 96, wherein the dCas protein is a dCas9 protein.
98. The epigenetic-modifying DNA-targeting system of embodiment 95 or 96, wherein the dCas protein is a dCasl2 protein.
99. The epigenetic-modifying DNA-targeting system of 97, wherein the dCas9 protein is a Staphylococcus aureus dCas9 (dSaCas9) protein.
100. The epigenetic-modifying DNA-targeting system of embodiment 99, wherein the dSaCas9 comprises at least one amino acid mutation selected from D10A and N580A, with reference to numbering of positions of SEQ ID NO:204.
101. The epigenetic-modifying DNA-targeting system of embodiment 99 or embodiment 100, wherein the dSaCas9 protein comprises the sequence set forth in SEQ ID NO:205, or an amino acid sequence that has at least 90%, 91%, 92%, 93%, 94%, 95%, 96%, 97%, 98%, or 99% sequence identity thereto.
102. The epigenetic-modifying DNA-targeting system of any of embodiments 99-101, wherein the dSaCas9 is set forth in SEQ ID NO:205.
103. The epigenetic-modifying DNA-targeting system of embodiment 97, wherein the dCas9 protein is a Streptococcuspyogenes dCas9 (dSpCas9) protein.
104. The epigenetic-modifying DNA-targeting system of embodiment 103, wherein the dSpCas9 protein comprises at least one amino acid mutation selected from D10A and H840A, with reference to numbering of positions of SEQ ID NO:206.
105. The epigenetic-modifying DNA-targeting system of 35 or embodiment 104, wherein the dSpCas9 comprises the sequence set forth in SEQ ID NO:207, or an amino acid sequence that has at least 90%, 91%, 92%, 93%, 94%, 95%, 96%, 97%, 98%, or 99% sequence identity thereto.
106. The epigenetic-modifying DNA-targeting system of any of embodiments 103-105, wherein the dSpCas9 is set forth in SEQ ID NO:207.
107. The epigenetic-modifying DNA-targeting system of any of embodiments 86-106, wherein the gRNA comprises a gRNA spacer that is complementary to the target site of the gene.
108. The epigenetic-modifying DNA-targeting system of any of embodiments 86-107, wherein the gRNA is selected from:
   (a) a gRNA targeting a target site of PCSK9 comprising a gRNA spacer sequence comprising the sequence set forth in any one of SEQ ID NOS:64-76 or 318-329, or a contiguous portion thereof of at least 14 nt, optionally wherein the gRNA spacer comprises the sequence set forth in SEQ ID NO:66 or a contiguous portion thereof of at least 14 nt;
   (b) a gRNA targeting a target site of LPA comprising a gRNA spacer sequence comprising the sequence set forth in any one of SEQ ID NOS:77-86, or a contiguous portion thereof of at least 14 nt;
   (c) a gRNA targeting a target site of MYLIP comprising a gRNA spacer sequence comprising the sequence set forth in any one of SEQ ID NOS:87-96 or 352-361, or a contiguous portion thereof of at least 14 nt;
   (d) a gRNA targeting a target site of ANGPTL3 comprising a gRNA spacer sequence comprising the sequence set forth in any one of SEQ ID NOS:97-106, or a contiguous portion thereof of at least 14 nt;
   (e) a gRNA targeting a target site of APOC3 comprising a gRNA spacer sequence comprising the sequence set forth in any one of SEQ ID NOS:107-116, or a contiguous portion thereof of at least 14 nt; and
   (f) a gRNA targeting a target site of APOB comprising a gRNA spacer sequence comprising the sequence set forth in any one of SEQ ID NOS:117-126 or 378-383, or a contiguous portion thereof of at least 14 nt.
109. The epigenetic-modifying DNA-targeting system of any of embodiments 86-108, wherein the gRNA comprises a spacer sequence between 14 nt and 24 nt, or between 16 nt and 22 nt in length.
110. The epigenetic-modifying DNA-targeting system of any of embodiments 86-109, wherein the gRNA comprises a spacer sequence that is 18 nt, 19 nt, 20 nt, 21 nt, or 22 nt in length.
111. The epigenetic-modifying DNA-targeting system of any of embodiments 806-110, wherein the gRNA is selected from:
   (a) a gRNA targeting a target site of PCSK9 comprising the gRNA spacer sequence set forth in any one of SEQ ID NOS:64-76 or 318-329, optionally wherein the gRNA spacer sequence is set forth in SEQ ID NO:66;
   (b) a gRNA targeting a target site in LPA comprising the gRNA spacer sequence set forth in any one of SEQ ID NOS:77-86;
   (c) a gRNA targeting a target site in MYLIP comprising the gRNA spacer sequence set forth in any one of SEQ ID NOS:87-96 or 352-361;
   (d) a gRNA targeting a target site in ANGPTL3 comprising the gRNA spacer sequence set forth in any one of SEQ ID NOS:97-106;
   (e) a gRNA targeting a target site in APOC3 comprising the gRNA spacer sequence set forth in any one of SEQ ID NOS:107-116; and
   (f) a gRNA targeting a target site in APOB comprising the gRNA spacer sequence set forth in any one of SEQ ID NOS:117-126 or 378-383.
112. The epigenetic-modifying DNA-targeting system of any of embodiments 103-111, wherein the gRNA further comprises a scaffold sequence set forth in SEQ ID NO:191.
113. The epigenetic-modifying DNA-targeting system of any of embodiments 103-112, wherein the gRNA is selected from:
   (a) a gRNA targeting a target site in PCSK9 comprising the sequence set forth in any one of SEQ ID NOS:127-139 or 330-341, optionally wherein the gRNA comprises the sequence set forth in SEQ ID NO:129;
   (b) a gRNA targeting a target site in LPA comprising the sequence set forth in any one of SEQ ID NOS:140-149;
   (c) a gRNA targeting a target site in MYLIP comprising the sequence set forth in any one of SEQ ID NOS:150-159 or 362-371;
   (d) a gRNA targeting a target site in ANGPTL3 comprising the sequence set forth in any one of SEQ ID NOS:160-169;
   (e) a gRNA targeting a target site in APOC3 comprising the sequence set forth in any one of SEQ ID NOS:170-179; and
   (f) a gRNA targeting a target site in APOB comprising the sequence set forth in any one of SEQ ID NOS:180-189 or 384-389.
114. The epigenetic-modifying DNA-targeting system of any of embodiments 103-113, wherein the gRNA is selected from:
   (a) a gRNA targeting a target site in PCSK9 set forth in any one of SEQ ID NOS:127-139 or 330-341, optionally wherein the gRNA is set forth in SEQ ID NO:129;
   (b) a gRNA targeting a target site in LPA set forth in any one of SEQ ID NOS:140-149;
   (c) a gRNA targeting a target site in MYLIP set forth in any one of SEQ ID NOS:150-159 or 362-371;
   (d) a gRNA targeting a target site in ANGPTL3 set forth in any one of SEQ ID NOS:160-169;
   (e) a gRNA targeting a target site in APOC3 set forth in any one of SEQ ID NOS:170-179; and
   (f) a gRNA targeting a target site in APOB set forth in any one of SEQ ID NOS:180-189 or 384-389.
115. The epigenetic-modifying DNA-targeting system of any of embodiments 86-114, wherein the gRNA comprises modified nucleotides for increased stability.
116. The epigenetic-modifying DNA-targeting combination of any of embodiments 82-115, wherein the at least one transcriptional repressor domain is capable of reducing transcription of the gene.
117. The epigenetic-modifying DNA-targeting combination of any of embodiments 82-116, wherein the transcriptional repressor domain is selected from the group consisting of a KRAB domain, a DNMT3A domain, a DNMT3L domain, a DNMT3B domain, a DNMT3A-DNMT3L fusion protein domain, an ERF repressor domain, an Mxi1 repressor domain, a SID4X repressor domain, a Mad-SID repressor domain, an LSD1 repressor domain, an EZH2 repressor domain, a SunTag domain, or a variant or portion of any of the foregoing, or a combination of any of the foregoing.
118. The epigenetic-modifying DNA-targeting system of any of embodiments 82-117, wherein the transcriptional repressor domain is a KRAB domain, a DNMT3A domain, or a DNMT3L domain, or a combination of any of the foregoing.
119. The epigenetic-modifying DNA-targeting system of any of embodiments 82-118, wherein the at least one transcriptional repressor domain comprises a KRAB domain or a variant or portion thereof that exhibits transcriptional repressor activity.
120. The epigenetic-modifying DNA-targeting system of any of embodiments 82-119, wherein the at least one transcriptional repressor domain comprises the sequence set forth in SEQ ID NO:193, a portion thereof, or an amino acid sequence that has at least 90%, 91%, 92%, 93%, 94%, 95%, 96%, 97%, 98%, or 99% sequence identity to any of the foregoing.
121. The epigenetic-modifying DNA-targeting system of any of embodiments 82-118, wherein the at least one transcriptional repressor domain comprises a DNMT3A domain or a variant or portion thereof that exhibits transcriptional repressor activity.
122. The epigenetic-modifying DNA-targeting system of any of embodiments 82-118 and 121, wherein the at least one transcriptional repressor domain comprises the sequence set forth in SEQ ID NO:195, a portion thereof, or an amino acid sequence that has at least 90%, 91%, 92%, 93%, 94%, 95%, 96%, 97%, 98%, or 99% sequence identity to any of the foregoing.
123. The epigenetic-modifying DNA-targeting system of any of embodiments 82-118, wherein the at least one transcriptional repressor domain comprises a DNMT3L domain or a variant or portion thereof that exhibits transcriptional repressor activity.
124. The epigenetic-modifying DNA-targeting system of any of embodiments 82-118 and 123, wherein the at least one transcriptional repressor domain comprises the sequence set forth in SEQ ID NO:197, a portion thereof, or an amino acid sequence that has at least 90%, 91%, 92%, 93%, 94%, 95%, 96%, 97%, 98%, or 99% sequence identity to any of the foregoing.
125. The epigenetic-modifying DNA-targeting system of any of embodiments 82-118, wherein the at least one transcriptional repressor domain is a DNMT3A-DNMT3L fusion protein domain or a variant thereof that exhibits transcriptional repressor activity.
126. The epigenetic-modifying DNA-targeting system of any of embodiments 82-118 and 125, wherein the at least one transcriptional repressor domain comprises the sequence set forth in SEQ ID NO:199 or SEQ ID NO:201, a portion thereof, or an amino acid sequence that has at least 90%, 91%, 92%, 93%, 94%, 95%, 96%, 97%, 98%, or 99% sequence identity to any of the foregoing.
127. The epigenetic-modifying DNA-targeting system of any of embodiments 82-117, wherein at least one transcriptional repressor domain comprises a sequence selected from any one of SEQ ID NOS:193, 195, 197, 199, 201, 220-226, and 283, or a domain thereof, a portion thereof, or an amino acid sequence that has at least 90%, 91%, 92%, 93%, 94%, 95%, 96%, 97%, 98%, or 99% sequence identity to any of the foregoing.
128. The epigenetic-modifying DNA-targeting system of any of embodiments 82-127, wherein the at least one transcriptional repressor domain is fused to the N-terminus, the C-terminus, or both the N-terminus and the C-terminus, of the DNA-binding domain.
129. The epigenetic-modifying DNA-targeting system of any of embodiments 82-128, wherein the fusion protein further comprises one or more nuclear localization signals (NLS).
130. The epigenetic-modifying DNA-targeting system of embodiment 129, wherein the fusion protein further comprises one or more linkers connecting two or more of: the DNA-binding domain, the at least one transcriptional repressor domain, and the one or more nuclear localization signals.
131. The epigenetic-modifying DNA-targeting system of any of embodiments 82-130, wherein the fusion protein comprises the sequence set forth in any one of SEQ ID NOS:209, 278, 280, or 282, or an amino acid sequence that has at least 90%, 91%, 92%, 93%, 94%, 95%, 96%, 97%, 98%, or 99% sequence identity thereto, optionally wherein the fusion protein is set forth in SEQ ID NO:280.
132. The epigenetic-modifying DNA-targeting system of any of embodiments 86-131, wherein the fusion protein comprises the sequence set forth in SEQ ID NO:280 or a sequence of amino acids that has at least 90%, 91%, 92%, 93%, 94%, 95%, 96%, 97%, 98%, or 99% sequence identity to SEQ ID NO:280 and a gRNA that targets a target site of PCSK9 comprising the spacer sequence set forth in SEQ ID NO: 66, optionally wherein the gRNA has the sequence set forth in SEQ ID NO:129.
133. The epigenetic-modifying DNA-targeting system of any of embodiments 82-132, wherein each of the DNA-targeting modules reduces expression of each of the respective genes by a log 2 fold-change of at or lesser than −1.0.
134. The epigenetic-modifying DNA-targeting system of any of embodiments 82-133, wherein repressed transcription of the gene in a cell or population of cells leads to a reduction of low-density lipoprotein (LDL), optionally wherein the reduction of LDL occurs extracellularly.
135. The epigenetic-modifying DNA-targeting system of embodiment 133 or 134, wherein the cell or population of cells is a liver cell or comprises liver cells.
136. The epigenetic-modifying DNA-targeting system of any of embodiments 133-135, wherein the cell or population of cells is in a subject.
137. The epigenetic-modifying DNA-targeting system of embodiment 136, wherein the reduction of LDL occurs in the subject or a fluid, tissue, or organ thereof. 138. The epigenetic-modifying DNA-targeting system of any of embodiments 133-137, wherein the reduction of LDL occurs in the blood of a subject.
139. A combination of epigenetic-modifying DNA-targeting systems comprising at least two of the DNA-targeting systems of any of embodiments 82-138, wherein each DNA-targeting system represses transcription of a different gene.
140. The combination of epigenetic-modifying DNA-targeting systems of embodiment 139, wherein each DNA-targeting system represses transcription of a different gene.
141. A guide RNA (gRNA) that targets a target site of a gene that regulates low-density lipoprotein (LDL).
142. The gRNA of embodiment 141, wherein the gene is selected from the group consisting of PCSK9, LPA, MYLIP, ANGPTL3, APOB, and APOC3.
143. the gRNA of embodiment 141-142, wherein the target site of the gene is in the gene or a regulatory DNA element thereof.
144. The gRNA of embodiment 143, wherein the regulatory DNA element is an enhancer or a promoter.
145. The gRNA of any of embodiments 141-144, wherein the target site is selected from the group consisting of:
   (a) a target site for PCSK9, located within 500 bp of human genome assembly GRCh38 (hg38) genomic coordinates chr1:55,039,548;
   (b) a target site for LPA, located within 500 bp of the hg38 genomic coordinates chr6:160,664,275;
   (c) a target site for MYLIP, located within 500 bp of the hg38 genomic coordinates chr6:16,129,086;
   (d) a target site for ANGPTL3, located within 500 bp of the hg38 genomic coordinates chr1:62,597,520;
   (e) a target site for APOC3, located within 500 bp of the hg38 genomic coordinates chr11:116,829,907; and
   (f) a target site for APOB, located within 500 bp of the hg38 genomic coordinates chr2:21,044,073.
146. The gRNA of any of embodiments 141-145, wherein the target site is selected from the group consisting of:
   (a) a target site located within 500 bp of a transcriptional start site of PCSK9;
   (b) a target site located within 500 bp of a transcriptional start site of LPA;
   (c) a target site located within 500 bp of a transcriptional start site of MYLIP;
   (d) a target site located within 500 bp of a transcriptional start site of ANGPTL3;
   (e) a target site located within 500 bp of a transcriptional start site of APOC3; and
   (f) a target site located within 500 bp of a transcriptional start site of APOB.
147. The gRNA of any of embodiments 141-146, wherein the target site is selected from:
   (a) a target site for PCSK9 having the sequence set forth in any one of SEQ ID NOS:1-13 or 306-317, a contiguous portion thereof of at least 14 nucleotides (nt), or a complementary sequence of any of the foregoing, optionally wherein the target site has (i) the sequence set forth in SEQ ID NO: 3, (ii) a contiguous portion of SEQ ID NO:3 of at least 14 nt; or (iii) a complementary sequence of (i) or (ii);
   (b) a target site for LPA having the sequence set forth in any one of SEQ ID NOS:14-23, a contiguous portion thereof of at least 14 nucleotides (nt), or a complementary sequence of any of the foregoing;
   (c) a target site for MYLIP having the sequence set forth in any one of SEQ ID NOS:24-33 or 342-351, a contiguous portion thereof of at least 14 nucleotides (nt), or a complementary sequence of any of the foregoing;
   (d) a target site for ANGPTL3 having the sequence set forth in any one of SEQ ID NOS:34-43, a contiguous portion thereof of at least 14 nucleotides (nt), or a complementary sequence of any of the foregoing;
   (e) a target site for APOC3 having the sequence set forth in any one of SEQ ID NOS:44-53, a contiguous portion thereof of at least 14 nucleotides (nt), or a complementary sequence of any of the foregoing; and
   (f) a target site for APOB having the sequence set forth in any one of SEQ ID NOS:54-63 or 372-377, a contiguous portion thereof of at least 14 nucleotides (nt), or a complementary sequence of any of the foregoing.
148. The gRNA of any of embodiments 141-147, wherein the target site is selected from:
   (a) a target site of PCSK9 having the sequence set forth in any one of SEQ ID NOS:1-13 or 306-317, optionally wherein the target site has the sequence set forth in SEQ ID NO:3;
   (b) a target site of LPA having the sequence set forth in any one of SEQ ID NOS:14-23;
   (c) a target site of MYLIP having the sequence set forth in any one of SEQ ID NOS:24-33 or 342-351;
   (d) a target site of ANGPTL3 having the sequence set forth in any one of SEQ ID NOS: 34-43;
   (e) a target site of APOC3 having the sequence set forth in any one of SEQ ID NOS:44-53; and
   (f) a target site of APOB having the sequence set forth in any one of SEQ ID NOS:54-63 or 372-377.
149. The gRNA of any of embodiments 141-148, wherein the gRNA is selected from:
   (a) a gRNA targeting a target site of PCSK9 comprising a gRNA spacer sequence comprising the sequence set forth in any one of SEQ ID NOS:64-76 or 318-329, or a contiguous portion thereof of at least 14 nt, optionally wherein the gRNA spacer sequence comprises the sequence set forth in SEQ ID NO:66 or a contiguous portion thereof of at least 14 nt;
   (b) a gRNA targeting a target site of LPA comprising a gRNA spacer sequence comprising the sequence set forth in any one of SEQ ID NOS:77-86, or a contiguous portion thereof of at least 14 nt;
   (c) a gRNA targeting a target site of MYLIP comprising a gRNA spacer sequence comprising the sequence set forth in any one of SEQ ID NOS:87-96 or 352-361, or a contiguous portion thereof of at least 14 nt;
   (d) a gRNA targeting a target site of ANGPTL3 comprising a gRNA spacer sequence comprising the sequence set forth in any one of SEQ ID NOS:97-106, or a contiguous portion thereof of at least 14 nt;
   (e) a gRNA targeting a target site of APOC3 comprising a gRNA spacer sequence comprising the sequence set forth in any one of SEQ ID NOS:107-116, or a contiguous portion thereof of at least 14 nt; and
   (f) a gRNA targeting a target site of APOB comprising a gRNA spacer sequence comprising the sequence set forth in any one of SEQ ID NOS:117-126 or 378-383, or a contiguous portion thereof of at least 14 nt.
150. The gRNA of any of embodiments 141-149, wherein the gRNA comprises a spacer sequence between 14 nt and 24 nt, or between 16 nt and 22 nt in length.
151. The gRNA of any of embodiments 141-150, wherein the gRNA comprises a spacer sequence that is 18 nt, 19 nt, 20 nt, 21 nt, or 22 nt in length.
152. The gRNA of any of embodiments 141-151, wherein the gRNA is selected from:
   (a) a gRNA targeting a target site of PCSK9 comprising the gRNA spacer sequence set forth in any one of SEQ ID NOS:64-76 or 318-329, optionally wherein the gRNA spacer is set forth in SEQ ID NO: 66;
   (b) a gRNA targeting a target site in LPA comprising the gRNA spacer sequence set forth in any one of SEQ ID NOS:77-86;
   (c) a gRNA targeting a target site in MYLIP comprising the gRNA spacer sequence set forth in any one of SEQ ID NOS:87-96 or 352-361;
   (d) a gRNA targeting a target site in ANGPTL3 comprising the gRNA spacer sequence set forth in any one of SEQ ID NOS:97-106;
   (e) a gRNA targeting a target site in APOC3 comprising the gRNA spacer sequence set forth in any one of SEQ ID NOS:107-116; and
   (f) a gRNA targeting a target site in APOB comprising the gRNA spacer sequence set forth in any one of SEQ ID NOS:117-126 or 378-383.
153. The gRNA of any of embodiments 141-152, wherein the gRNA further comprises a scaffold sequence set forth in SEQ ID NO:191.
154. The gRNA of any of embodiments 141-153, wherein the gRNA is selected from:
   (a) a gRNA targeting a target site in PCSK9 comprising the sequence set forth in any one of SEQ ID NOS:127-139 or 330-341, optionally wherein the gRNA comprises the sequence set forth in SEQ ID NO:129;
   (b) a gRNA targeting a target site in LPA comprising the sequence set forth in any one of SEQ ID NOS:140-149;
   (c) a gRNA targeting a target site in MYLIP comprising the sequence set forth in any one of SEQ ID NOS:150-159 or 362-371;
   (d) a gRNA targeting a target site in ANGPTL3 comprising the sequence set forth in any one of SEQ ID NOS:160-169;
   (e) a gRNA targeting a target site in APOC3 comprising the sequence set forth in any one of SEQ ID NOS:170-179; or
   (f) a gRNA targeting a target site in APOB comprising the sequence set forth in any one of SEQ ID NOS:180-189 or 384-389.
155. The gRNA of any of embodiments 141-154, wherein the gRNA is selected from:
   (a) a gRNA targeting a target site in PCSK9 set forth in any one of SEQ ID NOS:127-139 or 330-341, optionally wherein the gRNA is set forth in SEQ ID NO: 129;
   (b) a gRNA targeting a target site in LPA set forth in any one of SEQ ID NOS:140-149;
   (c) a gRNA targeting a target site in MYLIP set forth in any one of SEQ ID NOS:150-159 or 362-371;
   (d) a gRNA targeting a target site in ANGPTL3 set forth in any one of SEQ ID NOS:160-169;
   (e) a gRNA targeting a target site in APOC3 set forth in any one of SEQ ID NOS:170-179; or
   (f) a gRNA targeting a target site in APOB set forth in any one of SEQ ID NOS:180-189 or 384-389.
156. The gRNA of any of embodiments 141-155, wherein the gRNA comprises modified nucleotides for increased stability.
157. A plurality of gRNAs comprising at least a first gRNA and a second gRNA, wherein the first gRNA targets a target site of a first gene that regulates low-density lipoprotein (LDL) and the second gRNA targets a target site of a second gene that regulates LDL.
158. The plurality of gRNAs of embodiment 157, comprising a third gRNA that targets a target site of a third gene that regulates LDL, optionally wherein the plurality of gRNAs further comprises a fourth gRNA that targets a target site of a fourth gene that regulates LDL, optionally a fifth gRNA that targets a target site of a fifth gene that regulates LDL, and/or optionally a sixth gRNA that targets a target site of a sixth gene that regulates LDL.
159. The plurality of gRNAs of embodiment 157 or 158, wherein each gRNA is selected from the gRNA of any of embodiments 141-156.
160. The plurality of gRNAs of any of embodiments 157-159, wherein the first gene and the second gene are independently selected from the group consisting of PCSK9, LPA, MYLIP, ANGPTL3, APOB, and APOC3.
161. The plurality of gRNAs of any of embodiments 157-160, wherein the first gene and the second gene are different.
162. The plurality of gRNAs of any of embodiments 157-161, wherein the first gene and the second gene are: PCSK9 and LPA; PCSK9 and MYLIP; PCSK9 and ANGPTL3; PCSK9 and APOC3; PCSK9 and APOB; LPA and MYLIP; LPA and ANGPTL3; LPA and APOC3; LPA and APOB; MYLIP and ANGPTL3; MYLIP and APOC3; MYLIP and APOB; ANGPTL3 and APOC3; ANGPTL3 and APOB; or APOC3 and APOB.
163. The plurality of gRNAs of any of embodiments 158-162, wherein the first gene, the second gene, and the third gene are each independently selected from the group consisting of PCSK9, LPA, MYLIP, ANGPTL3, APOC3, and APOB.
164. The plurality of gRNAs of any of embodiments 158-163, wherein the first gene, the second gene, and the third gene are different.
165. The plurality of gRNAs of any of embodiments 158-164, wherein the first gene, the second gene, and the third gene are: PCSK9, LPA, and MYLIP; PCSK9, LPA, and ANGPTL3; PCSK9, LPA, and APOC3; PCSK9, LPA, and APOB; PCSK9, MYLIP, and ANGPTL3; PCSK9, MYLIP, and APOC3; PCSK9, MYLIP, and APOB; PCSK9, ANGPTL3, and APOC3; PCSK9, ANGPTL3, and APOB; PCSK9, APOC3, and APOB; LPA, MYLIP, and ANGPTL3; LPA, MYLIP, and APOC3; LPA, MYLIP, and APOB; LPA, ANGPTL3, and APOC3; LPA, ANGPTL3, and APOB; LPA, APOC3, and APOB; MYLIP, ANGPTL3, and APOC3; MYLIP, ANGPTL3, and APOB; MYLIP, APOC3, and APOB; or ANGPTL3, APOC3, and APOB.
166. The plurality of gRNAs of any of embodiments 157-165, wherein at least one gene is PCSK9.
167. The plurality of gRNAs of any of embodiments 157-166, wherein at least two genes are PCSK9 and LPA.
168. The plurality of gRNAs of any of embodiments 157-167, wherein at least three genes are PCSK9, LPA, and MYLIP.
169. The plurality of gRNAs of any of embodiments 157-168, wherein the target site of each of the plurality of genes is in the gene or a regulatory DNA element thereof.
170. A Cas-guide RNA (gRNA) combination comprising:
   (a) a Clustered Regularly Interspaced Short Palindromic Repeats associated (Cas) protein or variant thereof; and
   (b) the gRNA of any of embodiments 141-156 or the plurality of gRNAs of any of embodiments 157-169.
171. The Cas-gRNA combination of embodiment 170, wherein the Cas protein or variant thereof is a variant Cas protein that is a deactivated (dCas) protein.
172. The Cas-gRNA combination of embodiment 171, wherein the dCas protein lacks nuclease activity.
173. The Cas-gRNA combination of embodiment 171 or embodiment 172, wherein the dCas protein is a dCas9 protein.
174. The Cas-gRNA combination of of embodiment 171 or embodiment 172, wherein the dCas protein is a dCasl2 protein.
175. The Cas-gRNA combination of embodiment 173, wherein the dCas9 protein is a Staphylococcus aureus dCas9 (dSaCas9) protein.
176. The Cas-gRNA combination of embodiment 175, wherein the dSaCas9 comprises at least one amino acid mutation selected from D10A and N580A, with reference to numbering of positions of SEQ ID NO:204.
177. The Cas-gRNA combination of embodiment 175 or embodiment 176, wherein the dSaCas9 protein comprises the sequence set forth in SEQ ID NO:205, or an amino acid sequence that has at least 90%, 91%, 92%, 93%, 94%, 95%, 96%, 97%, 98%, or 99% sequence identity thereto.
178. The Cas-gRNA combination of any of embodiments 175-177, wherein the dSaCas9 is set forth in SEQ ID NO:205.
179. The Cas-gRNA combination of embodiment 173, wherein the dCas9 protein is a Streptococcus pyogenes dCas9 (dSpCas9) protein.
180. The Cas-gRNA combination of embodiment 179, wherein the dSpCas9 protein comprises at least one amino acid mutation selected from D10A and H840A, with reference to numbering of positions of SEQ ID NO:206.
181. The Cas-gRNA combination of 179 or embodiment 180, wherein the dSpCas9 comprises the sequence set forth in SEQ ID NO:207, or an amino acid sequence that has at least 90%, 91%, 92%, 93%, 94%, 95%, 96%, 97%, 98%, or 99% sequence identity thereto.
182. The Cas-gRNA combination of any of embodiments 179-181, wherein the dSpCas9 is set forth in SEQ ID NO:207.
183. A polynucleotide encoding the epigenetic-modifying DNA-targeting system of any of embodiments 1-138, the combination of epigenetic-modifying DNA-targeting systems of embodiment 139 or 140, the gRNA of any of embodiments 141-156, the plurality of gRNAs of any of embodiments 157-169, the Cas-gRNA combination of any of embodiments 170-182, or a portion or a component of any of the foregoing.
184. A polynucleotide encoding the epigenetic-modifying DNA-targeting system of any of embodiments 1-138.
185. A polynucleotide encoding at least one DNA-targeting module of the epigenetic-modifying DNA-targeting system of any of embodiments 1-11 and 13-81.
186. A polynucleotide encoding the fusion protein and the at least first gRNA and second gRNA of the epigenetic-modifying DNA-targeting system of any of embodiments 11-81.
187. A polynucleotide encoding the fusion protein of the epigenetic-modifying DNA-targeting system of any of embodiments 82-138.
188. A polynucleotide encoding the fusion protein and gRNA of the epigenetic-modifying DNA-targeting system of any of embodiments 86-138.
189. A polynucleotide encoding the combination of epigenetic-modifying DNA-targeting systems of embodiment 139 or 140.
190. A polynucleotide encoding the gRNA of any of embodiments 141-156.
191. A polynucleotide encoding the plurality of gRNAs of any of embodiments 157-169.
192. A polynucleotide encoding the Cas-gRNA combination of any of embodiments 170-182.
193. A plurality of polynucleotides encoding the epigenetic-modifying DNA-targeting system of any of embodiments 1-138, the combination of epigenetic-modifying DNA-targeting systems of embodiment 139 or 140, the plurality of gRNAs of any of embodiments 157-169, the Cas-gRNA combination of any of embodiments 170-182, or a portion or a component of any of the foregoing.
194. A plurality of polynucleotides encoding the epigenetic-modifying DNA-targeting system of any of embodiments 1-138.
195. A plurality of polynucleotides encoding at least one DNA-targeting module of the epigenetic-modifying DNA-targeting system of any of embodiments 1-11 and 13-81.
196. A plurality of polynucleotides encoding the fusion protein and the at least first gRNA and second gRNA of the epigenetic-modifying DNA-targeting system of any of embodiments 11-81.
197. A plurality of polynucleotides encoding the fusion protein of the epigenetic-modifying DNA-targeting system of any of embodiments 82-138.
198. A plurality of polynucleotides encoding the fusion protein and gRNA of the epigenetic-modifying DNA-targeting system of any of embodiments 86-138.
199. A plurality of polynucleotides encoding the combination of epigenetic-modifying DNA-targeting systems of embodiment 139 or 140.
200. A plurality of polynucleotides encoding the plurality of gRNAs of any of embodiments 157-169.
201. A plurality of polynucleotides encoding the Cas-gRNA combination of any of embodiments 170-182.
202. A vector comprising the polynucleotide of any of embodiments 183-192.
203. A vector comprising the plurality of polynucleotides of any of embodiments 193-201.
204. The vector of embodiment 202 or embodiment 203, wherein the vector is a viral vector.
205. The vector of embodiment 204, wherein the vector is an adeno-associated virus (AAV) vector.
206. The vector of embodiment 205, wherein the vector is selected from among AAV1, AAV2, AAV3, AAV4, AAV5, AAV6, AAV7, AAV8, and AAV9.
207. The vector of embodiment 204, wherein the vector is a lentiviral vector.
208. The vector of embodiment 202 or embodiment 203, wherein the vector is a non-viral vector.
209. The vector of embodiment 208, wherein the non-viral vector is selected from: a lipid nanoparticle, a liposome, an exosome, or a cell penetrating peptide.
210. The vector of embodiment 208, wherein the non-viral vector is a lipid nanoparticle.
211. The vector of any of embodiments 202-210, wherein the vector exhibits hepatocyte tropism.
212. A lipid nanoparticle comprising, the polynucleotide of any of embodiments 183-192 or the plurality of polynucleotides of any of embodiments 193-201.
213. A method of decreasing transcription of at least two genes in a cell or population of cells, the method comprising administering to a cell or population of cells the epigenetic-modifying DNA-targeting system of any of embodiments 1-138, the combination of epigenetic-modifying DNA-targeting systems of embodiment 139 or 140, the gRNA of any of embodiments 141-156, the plurality of gRNAs of any of embodiments 157-169, the Cas-gRNA combination of any of embodiments 170-182, the polynucleotide of any of embodiments 183-192, the plurality of polynucleotides of any of embodiments 193-201, the vector of any of embodiments 202-211, or the lipid nanoparticle of embodiment 212, or a portion or a component of any of the foregoing.
214. The method of embodiment 213, wherein the at least two genes are epigenetically modified.
215. The method of embodiment 213 or embodiment 214, wherein the transcription of each of the at least two genes is decreased in comparison to a comparable cell or population of cells not subjected to the method.
216. The method of any of embodiments 213-215, wherein the transcription of each of the at least two genes is reduced by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.
217. The method of any of embodiments 213-216, wherein the reduced transcription of each of the at least two genes leads to a reduction of low-density lipoprotein (LDL).
218. The method of embodiment 217, wherein the reduction of LDL resulting from reduced transcription each of the at least two genes is greater than the reduction of LDL resulting from comparable reduced transcription of any individual gene of the at least two genes alone.
219. A method of reducing LDL, the method comprising introducing into a cell or population of cells the epigenetic-modifying DNA-targeting system of any of embodiments 1-138, the combination of epigenetic-modifying DNA-targeting systems of embodiment 139 or 140, the gRNA of any of embodiments 141-156, the plurality of gRNAs of any of embodiments 157-169, the Cas-gRNA combination of any of embodiments 170-182, the polynucleotide of any of embodiments 183-192, the plurality of polynucleotides of any of embodiments 193-201, the vector of any of embodiments 202-211, or the lipid nanoparticle of embodiment 212, or a portion or a component of any of the foregoing.
220. The method of any of embodiments 213-219, wherein the cell or population of cells is a liver cell or comprises liver cells.
221. The method of any of embodiments 213-220, wherein the cell or population of cells is in a subject and the method is carried out in vivo.
222. The method of embodiment 221, wherein LDL is reduced in the subject or a fluid, tissue, or organ thereof.
223. The method of any of embodiments 213-222, wherein LDL is reduced in the blood of a subject.
224. The method of any of embodiments 221-223, wherein the subject is a human.
225. The method of any of embodiments 221-224, wherein the subject has or is suspected of having a disease, condition, or disorder, optionally wherein the disease, condition or disorder is a cardiovascular disease.
226. The method of any of embodiments 221-225, wherein the subject has or is suspected of having one or more of: elevated levels of low-density lipoprotein in the blood, increased risk of cardiovascular disease, increased risk of early-onset cardiovascular disease, a mutation affecting cholesterol biosynthesis, a loss-of-function mutation in a low-density lipoprotein receptor (LDLR) gene, a loss-of-function mutation in APOB, a gain-of-function mutation in PCSK9, and familial hypercholesterolemia.
227. The method of any of embodiments 221-226, wherein the subject has is or is suspected of having familial hypercholesterolemia.
228. A pharmaceutical composition comprising the epigenetic-modifying DNA-targeting system of any of embodiments 1-138, the combination of epigenetic-modifying DNA-targeting systems of embodiment 139 or 140, the gRNA of any of embodiments 141-156, the plurality of gRNAs of any of embodiments 157-169, the Cas-gRNA combination of any of embodiments 170-182, the polynucleotide of any of embodiments 183-192, the plurality of polynucleotides of any of embodiments 193-201, the vector of any of embodiments 202-211, the lipid nanoparticle of embodiment 212, or a portion or a component of any of the foregoing.
229. The pharmaceutical composition of embodiment 228, for use in treating a disease, condition, or disorder in a subject, optionally wherein the disease, condition or disorder is a cardiovascular disease.
230. Use of the pharmaceutical composition of embodiment 228 in the manufacture of a medicament for treating a disease, condition, or disorder in a subject, optionally wherein the disease, condition or disorder is a cardiovascular disease.
231. The pharmaceutical composition of embodiment 229 or the use of embodiment 230, wherein the subject has or is suspected of having a disease, condition, or disorder, optionally wherein the disease, condition or disorder is a cardiovascular disease.
232. The pharmaceutical composition or use of any of embodiments 229-231, wherein the subject has or is suspected of having one or more of: elevated levels of low-density lipoprotein in the blood, increased risk of cardiovascular disease, increased risk of early-onset cardiovascular disease, a mutation affecting cholesterol biosynthesis, a loss-of-function mutation in a low-density lipoprotein receptor (LDLR) gene, a loss-of-function mutation in APOB, a gain-of-function mutation in PCSK9, and familial hypercholesterolemia.
233. The pharmaceutical composition or use of any of embodiments 229-232, wherein the subject has is or is suspected of having familial hypercholesterolemia.
234. The pharmaceutical composition or use of any of embodiments 229-233, wherein the pharmaceutical composition is to be administered to the subject in vivo.
235. The pharmaceutical composition or use of embodiment 234, wherein the pharmaceutical composition is targeted to, or is to be administered to the liver of the subject.
236. The pharmaceutical composition or use of embodiment 234 or 235, wherein following administration of the pharmaceutical composition, the expression of at least two genes is reduced in cells of the subject.
237. The pharmaceutical composition or use of any of embodiments 234-236, wherein following administration of the pharmaceutical composition, the expression of at least two genes is reduced in liver cells of the subject.
238. The pharmaceutical composition or use of embodiment 236 or 237, wherein the at least two genes are selected from the group consisting of. PCSK9, LPA, MYLIP, ANGPTL3, APOB, and APOC3.
239. A method for treating a disease, condition, or disorder associated with elevated low-density lipoprotein (LDL) in a subject in need thereof, comprising administering to the subject the epigenetic-modifying DNA-targeting system of any of embodiments 1-138, the combination of epigenetic-modifying DNA-targeting systems of embodiment 139 or 140, the gRNA of any of embodiments 141-156, the plurality of gRNAs of any of embodiments 157-169, the Cas-gRNA combination of any of embodiments 170-182, the polynucleotide of any of embodiments 183-192, the plurality of polynucleotides of any of embodiments 193-201, the vector of any of embodiments 202-211, the lipid nanoparticle of embodiment 212, the pharmaceutical composition of any of embodiments 228-238, or a portion or a component of any of the foregoing.
240. The method of embodiment 239, wherein the disease, condition or disorder associated with elevated LDL is a cardiovascular disease.
241. The method of embodiment 239 or embodiment 240, wherein the subject has or is suspected of having one or more of: elevated levels of low-density lipoprotein in the blood, increased risk of cardiovascular disease, increased risk of early-onset cardiovascular disease, a mutation affecting cholesterol biosynthesis, a loss-of-function mutation in a low-density lipoprotein receptor (LDLR) gene, a loss-of-function mutation in APOB, a gain-of-function mutation in PCSK9, and familial hypercholesterolemia.
242. A method for treating a familial hypercholesterolemia in a subject, comprising administering to the subject the epigenetic-modifying DNA-targeting system of any of embodiments 1-138, the combination of epigenetic-modifying DNA-targeting systems of embodiment 139 or 140, the gRNA of any of embodiments 141-156, the plurality of gRNAs of any of embodiments 157-169, the Cas-gRNA combination of any of embodiments 170-182, the polynucleotide of any of embodiments 183-192, the plurality of polynucleotides of any of embodiments 193-201, the vector of any of embodiments 202-211, the lipid nanoparticle of embodiment 212, the pharmaceutical composition of any of embodiments 228-238, or a portion or a component of any of the foregoing.
243. The method of any of embodiments 213-227 and 239-242, wherein the administration is a single dose infusion to the subject.
244. The method of any of embodiments 213-227 and 239-242, wherein the administration is repeated at least once, optionally a plurality of times at regular intervals.
250. The method of any of embodiments 218-232, 244-247 and 249, wherein the administration is a multiple dose administration comprising at least a first dose and a second dose.
251. The method of embodiment 250, wherein the first dose and the second dose are the same.
252. The method of embodiment 250, wherein the second dose is lower than the first dose, optionally wherein the second dose is 25% to 75% of the first dose (e.g., about 30%, about 40%, about 50%, about 60% or about 70%, or a percentage between any of the foregoing).
253. The method of embodiment 250, wherein the second dose is higher than the first dose, optionally wherein the second dose is 150% to 500% of the first dose (e.g., about 200%, about 300%, about 400% or about 500%, or a percentage between any of the foregoing).
254. The method of any of embodiments 218-232 and 244-253, wherein the lipid nanoparticle of embodiment 212 is administered to the subject.
255. The pharmaceutical composition or use of any of embodiments 233-243, wherein the pharmaceutical composition is for single dose infusion to the subject.
256. The pharmaceutical composition or use of any of embodiments 233-243, wherein the pharmaceutical composition is for repeated dose administration, optionally a plurality of times at regular intervals.
257. The pharmaceutical composition or use of any of embodiments 233-243 and
256, wherein the administration is a multiple dose administration comprising at least a first dose and a second dose.
258. The pharmaceutical composition or use of embodiment 257, wherein the first dose and the second dose are the same.
259. The pharmaceutical composition or use of embodiment 257, wherein the second dose is lower than the first dose, optionally wherein the second dose is 25% to 75% of the first dose (e.g., about 30%, about 40%, about 50%, about 60% or about 70%, or a percentage between any of the foregoing).
260. The pharmaceutical composition or use of embodiment 257, wherein the second dose is higher than the first dose, optionally wherein the second dose is 150% to 500% of the first dose (e.g., about 200%, about 300%, about 400% or about 500%, or a percentage between any of the foregoing).
261. The pharmaceutical composition or use of any of embodiments 233-243, and 255-260, wherein the pharmaceutical composition comprises the lipid nanoparticle of embodiment 217.

### VIII. EXAMPLES

The following examples are included for illustrative purposes only and are not intended to limit the scope of the invention.

### Example 1: Transcriptional Repression of Mouse PCSK9 Using an Epigenetic-Modifying DNA-Taretin2 System

DNA-targeting systems comprising an exemplary dCas9-effector fusion protein for transcriptional repression and guide RNAs (gRNAs) targeting the promoter of mouse PCSK9 were tested for the ability to repress PCSK9 in vitro.

gRNAs targeting the promoter of a mouse PCSK9 gene were designed. Target sites were selected according to the protospacer adjacent motif (PAM) sequence for SpCas9 (5′-NGG-3′; SEQ ID NO:202). gRNAs included a scaffold sequence for SpCas9 (SEQ ID NO:191). DNA target site (protospacer) sequences, and gRNA spacer sequences for gRNAs targeting mouse PCSK9 are shown in Table E1. The designed gRNA sequences targeting mouse PSCK9 are set forth in SEQ ID NOs: 268-276.

TABLE E1
gRNAs targeting mouse PCSK9

| | target site | | gRNA | gRNA |
|---|---|---|---|---|
| gRNA | (protospacer) | target | spacer | spacer |
| Name | sequence | SEQ ID | sequence | SEQ ID |
| mPCSK9-A | GGGAAGGGATACA | 250 | GGGAAGGGAU | 259 |
| | GGCTGGA | | ACAGGCUGGA | |
| mPCSK9-B | GTCCCGTTTGCAG | 251 | GUCCCGUUUG | 260 |
| | CCCAATT | | CAGCCCAAUU | |
| mPCSK9-C | GAGCGTCATTTGA | 252 | GAGCGUCAUU | 261 |
| | CGCTGTC | | UGACGCUGUC | |
| mPCSK9-D | GGATCTTCCGATG | 253 | GGAUCUUCCG | 262 |
| | GGGCTCG | | AUGGGGCUCG | |
| mPCSK9-E | GTGAAGGTGGAAG | 254 | GUGAAGGUGG | 263 |
| | CCTTCTG | | AAGCCUUCUG | |
| mPCSK9-F | GTGGACGCGCAGG | 255 | GUGGACGCGC | 264 |
| | CTGCCGG | | AGGCUGCCGG | |
| mPCSK9-G | GGGGCGAGGAGAG | 256 | GGGGCGAGGA | 265 |
| | GTGCGCG | | GAGGUGCGCG | |
| mPCSK9-H | AGTGGGTGCCCAT | 257 | AGUGGGUGCC | 266 |
| | CGGGGCG | | CAUCGGGGCG | |
| mPCSK9-I | CTACTGTGCCCCA | 258 | CUACUGUGCC | 267 |
| | CCGGCGC | | CCACCGGCGC | |

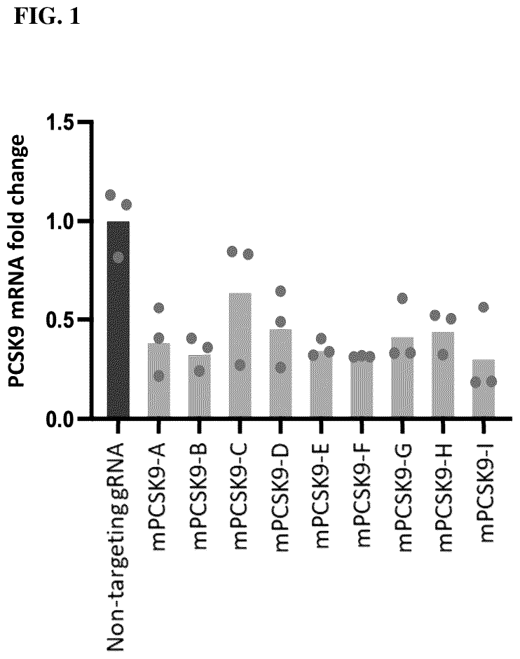

Expression plasmids were constructed, each encoding one of the designed gRNAs, GFP, and dSpCas9-KRAB-DNMT3A/L (SEQ ID NO:278), an exemplary DNA-targeting fusion protein for transcriptional repression of gRNA-targeted genes. A murine liver cell line (AML12) was transiently transfected with the plasmids encoding each gRNA, or encoding a non-targeting gRNA (negative control). One day after transfection, GFP+ cells were sorted to enrich for transfected cells. Three days after enrichment, PCKS9 mRNA levels were assessed by qRT-PCR. As shown in FIG. 1, expression of dSpCas9-KRAB-DNMT3A/L and the PCSK9-targeting gRNAs led to transcriptional repression of PCSK9.

The results showed that a DNA-targeting system comprising a gRNA targeting PCSK9 and a dCas9-effector fusion protein can facilitate transcriptional repression of PCSK9. The results also showed that a DNA-targeting system comprisinga dCas9-effector fusion with more than one effector domain can effectively mediate transcriptional repression. The results support the utility of repressing PCSK9 in vivo and for potential treatment of familial hypercholesterolemia and/or cardiovascular disease.

### Example 2: Design and Verification of gRNAs Targeting Human Genes Associated with Regulation of Low-Density Lipoprotein (LDL)

gRNAs were designed for targeting human genes associated with regulation of low-density lipoprotein (LDL), including PCSK9, LPA, MYLIP, ANGPTL3, APOC3, and APOB. The designed gRNAs were generated and assessed for the ability to mediate transcriptional repression as part of a DNA-targeting system composed of a designed gRNA and a dCas9-effector fusion protein.

Multiple gRNAs targeting the promoter region of each gene (within 500 base pairs of the transcriptional start site) were designed. Target sites were selected according to the protospacer adjacent motif (PAM) sequence for SpCas9 (5′-NGG-3′; SEQ ID NO:202). Exemplary DNA target site (protospacer) sequences are set forth in SEQ ID NOS:1-63 and gRNA spacer sequences for gRNAs targeting each site are set forth in SEQ ID NOS:64-126. These sequences are listed in Table 3 (Section LB.). Each gRNA further comprised a scaffold sequence for SpCas9, comprising the sequence: GUUIUAAGAGCUAUGCUGGAAACAGCAUAGCAAGUUIIUAAAUAAGGCUAGUCCG SUAUCAAC EUGAAAAAGUGGCACCGAGUCGGUGC (SEQ TD NO: 191). The sequences of the designed gRNAs (SEQ TD NOS: 127-189) and their respective DNA target sites (SEQ TD NOS: 1-63) are shown in Table E2 for PCSK9, Table E3 for LPA, Table E4 for MYLIP, Table E5 for ANGPTL3, Table E6 for APOC3, and Table E7 for APOB.

TABLE E2
gRNAs targeting human PCSK9

| | tar- | | | |
|---|---|---|---|---|
| | get | | | |
| | site | target | gRNA | |
| gRNA | SEQ | site | SEQ | |
| name | ID | sequence | ID | gRNA sequence |
| PCSK9- | 1 | GCGCGTAATC | 127 | GCGCGUAAUCUGACGCUGUUGUUU |
| A | | TGACGCTGTT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 2 | ATCAGATAGG | 128 | AUCAGAUAGGAUCGUCCGAUGUUU |
| B | | ATCGTCCGAT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 3 | AGGTTTCCGC | 129 | AGGUUUCCGCAGCGACGUCGGUUU |
| C | | AGCGACGTCG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 4 | GGGCGCCGCC | 130 | GGGCGCCGCCGUUCAGUUCAGUUU |
| D | | GTTCAGTTCA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 5 | GGTGCTAGCC | 131 | GGUGCUAGCCUUGCGUUCCGGUUU |
| E | | TTGCGTTCCG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 6 | CATTAACGGA | 132 | CAUUAACGGAACCCCCGGACGUUU |
| F | | ACCCCCGGAC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 7 | AGGATCGTCC | 133 | AGGAUCGUCCGAUGGGGCUCGUUU |
| G | | GATGGGGCTC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 8 | CCGTTAATGT | 134 | CCGUUAAUGUUUAAUCAGAUGUUU |
| H | | TTAATCAGAT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 9 | CGTAATCTGA | 135 | CGUAAUCUGACGCUGUUUGGUUUA |
| I | | CGCTGTTTG | | AGAGCUAUGCUGGAAACAGCAUAG |
| | | | | CAAGUUUAAAUAAGGCUAGUCCGU |
| | | | | UAUCAACUUGAAAAAGUGGCACCG |
| | | | | AGUCGGUGC |
| PCSK9- | 10 | GGTGTGGGTG | 136 | GGUGUGGGUGCUUGACGCCUGUUU |
| J | | CTTGACGCCT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 11 | ACCCACTGCA | 137 | ACCCACUGCACGCUGGACAGGUUU |
| K | | CGCTGGACAG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 12 | GCACAGTAAC | 138 | GCACAGUAACAACCCCUGGUGUUU |
| L | | AACCCCTGGT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 13 | CCATCCATTC | 139 | CCAUCCAUUCUUUCUCUAGGGUUU |
| M | | TTTCTCTAGG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |

TABLE E3
gRNAs targeting human LPA

| | tar- | | | |
|---|---|---|---|---|
| | get | | | |
| | site | target | gRNA | |
| gRNA | SEQ | site | SEQ | |
| name | ID | sequence | ID | gRNA sequence |
| LPA- | 14 | AAGGAGACAT | 140 | AAGGAGACAUAAAGGCAAUGGUUU |
| A | | AAAGGCAATG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| LPA- | 15 | GGCAATGTGG | 141 | GGCAAUGUGGAGCAGCUGAGGUUU |
| B | | AGCAGCTGAG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| LPA- | 16 | GGAGCAGCTG | 142 | GGAGCAGCUGAGGGGGGAAAGUUU |
| C | | AGGGGGGAAA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| LPA- | 17 | TGTCAATAGA | 143 | UGUCAAUAGAUGCUGGGAAGGUUU |
| D | | TGCTGGGAAG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| LPA- | 18 | AGTGCAATGT | 144 | AGUGCAAUGUCAAUAGAUGCGUUU |
| E | | CAATAGATGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| LPA- | 19 | TTTATAAGAC | 145 | UUUAUAAGACUCUAUAUUCAGUUU |
| F | | TCTATATTCA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| LPA- | 20 | CATGTAAGTC | 146 | CAUGUAAGUCAACAAUGUCCGUUU |
| G | | AACAATGTCC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| LPA- | 21 | GTCAACAATG | 147 | GUCAACAAUGUCCUGGGAUUGUUU |
| H | | TCCTGGGATT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| LPA- | 22 | CATATACAAG | 148 | CAUAUACAAGAUUUUGAACUGUUU |
| I | | ATTTTGAACT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| LPA- | 23 | GCACCGTGAC | 149 | GCACCGUGACAGUCUUCACGGUUU |
| J | | AGTCTTCACG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |

TABLE E4
gRNAs targeting human MYLIP

| | tar- | | | |
|---|---|---|---|---|
| | get | | | |
| | site | target | gRNA | |
| gRNA | SEQ | site | SEQ | |
| name | ID | sequence | ID | gRNA sequence |
| MYLIP- | 24 | TTGGCGGGGA | 150 | UUGGCGGGGACCCGAGCUGAGUUU |
| A | | CCCGAGCTGA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 25 | CTGTCGCAGC | 151 | CUGUCGCAGCGCAGGCAGUUGUUU |
| B | | GCAGGCAGTT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 26 | GCTGGAGTGC | 152 | GCUGGAGUGCGGCGCCACCGGUUU |
| C | | GGCGCCACCG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 27 | CGGCGCCACC | 153 | CGGCGCCACCGCGGAGGACAGUUU |
| D | | GCGGAGGACA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 28 | CAGCTCTGCG | 154 | CAGCUCUGCGGACCCUUGUCGUUU |
| E | | GACCCTTGTC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 29 | CCCCGCGCAC | 155 | CCCCGCGCACACCAAAGAGAGUUU |
| F | | ACCAAAGAGA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 30 | CCTCGTCACA | 156 | CCUCGUCACAUAACACAGCAGUUU |
| G | | TAACACAGCA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 31 | ACCTCCATCA | 157 | ACCUCCAUCAGCACCGCGUCGUUU |
| H | | GCACCGCGTC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 32 | GGAGGCGAAA | 158 | GGAGGCGAAAGCCAACGGCGGUUU |
| I | | GCCAACGGCG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 33 | GTTGAGGCAG | 159 | GUUGAGGCAGUCCUCGCCGUGUUU |
| J | | TCCTCGCCGT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |

TABLE E5
gRNAs targeting human ANGPTL3

| | tar- | | | |
|---|---|---|---|---|
| | get | | | |
| | site | target | gRNA | |
| gRNA | SEQ | site | SEQ | |
| name | ID | sequence | ID | gRNA sequence |
| ANGPTL3- | 34 | TACATTCG | 160 | UACAUUCGUGCAAGUUAACAGUUU |
| A | | TGCAAGTT | | AAGAGCUAUGCUGGAAACAGCAUA |
| | | AACA | | GCAAGUUUAAAUAAGGCUAGUCCG |
| | | | | UUAUCAACUUGAAAAAGUGGCACC |
| | | | | GAGUCGGUGC |
| ANGPTL3- | 35 | CCTACCAA | 161 | CCUACCAACCUUACCUUUUCGUUU |
| B | | CCTTACCT | | AAGAGCUAUGCUGGAAACAGCAUA |
| | | TTTC | | GCAAGUUUAAAUAAGGCUAGUCCG |
| | | | | UUAUCAACUUGAAAAAGUGGCACC |
| | | | | GAGUCGGUGC |
| ANGPTL3- | 36 | TATATAGA | 162 | UAUAUAGAGUUAAGAAGUCUGUUU |
| C | | GTTAAGAA | | AAGAGCUAUGCUGGAAACAGCAUA |
| | | GTCT | | GCAAGUUUAAAUAAGGCUAGUCCG |
| | | | | UUAUCAACUUGAAAAAGUGGCACC |
| | | | | GAGUCGGUGC |
| ANGPTL3- | 37 | AACGTGGA | 163 | AACGUGGAACUGUUUUCUUCGUUU |
| D | | ACTGTTTT | | AAGAGCUAUGCUGGAAACAGCAUA |
| | | CTTC | | GCAAGUUUAAAUAAGGCUAGUCCG |
| | | | | UUAUCAACUUGAAAAAGUGGCACC |
| | | | | GAGUCGGUGC |
| ANGPTL3- | 38 | ATTTTCAA | 164 | AUUUUCAAUUUCAAGCAACGGUUU |
| E | | TTTCAAGC | | AAGAGCUAUGCUGGAAACAGCAUA |
| | | AACG | | GCAAGUUUAAAUAAGGCUAGUCCG |
| | | | | UUAUCAACUUGAAAAAGUGGCACC |
| | | | | GAGUCGGUGC |
| ANGPTL3- | 39 | ATTCTGGA | 165 | AUUCUGGAGGAAAUAACUAGGUUU |
| F | | GGAAATAA | | AAGAGCUAUGCUGGAAACAGCAUA |
| | | CTAG | | GCAAGUUUAAAUAAGGCUAGUCCG |
| | | | | UUAUCAACUUGAAAAAGUGGCACC |
| | | | | GAGUCGGUGC |
| ANGPTL3- | 40 | GCAAATCT | 166 | GCAAAUCUUGAUUUUGGCUCGUUU |
| G | | TGATTTTG | | AAGAGCUAUGCUGGAAACAGCAUA |
| | | GCTC | | GCAAGUUUAAAUAAGGCUAGUCCG |
| | | | | UUAUCAACUUGAAAAAGUGGCACC |
| | | | | GAGUCGGUGC |
| ANGPTL3- | 41 | AGCCAATG | 167 | AGCCAAUGGCCUCCUUCAGUGUUU |
| H | | GCCTCCTT | | AAGAGCUAUGCUGGAAACAGCAUA |
| | | CAGT | | GCAAGUUUAAAUAAGGCUAGUCCG |
| | | | | UUAUCAACUUGAAAAAGUGGCACC |
| | | | | GAGUCGGUGC |
| ANGPTL3- | 42 | TAAGACCA | 168 | UAAGACCAUGUCCCAACUGAGUUU |
| I | | TGTCCCAA | | AAGAGCUAUGCUGGAAACAGCAUA |
| | | CTGA | | GCAAGUUUAAAUAAGGCUAGUCCG |
| | | | | UUAUCAACUUGAAAAAGUGGCACC |
| | | | | GAGUCGGUGC |
| ANGPTL3- | 43 | AGACTTTG | 169 | AGACUUUGUCCAUAAGACGAGUUU |
| J | | TCCATAAG | | AAGAGCUAUGCUGGAAACAGCAUA |
| | | ACGA | | GCAAGUUUAAAUAAGGCUAGUCCG |
| | | | | UUAUCAACUUGAAAAAGUGGCACC |
| | | | | GAGUCGGUGC |

TABLE E6
gRNAs targeting human APOC3

| | tar- | | | |
|---|---|---|---|---|
| | get | | | |
| | site | target | gRNA | |
| gRNA | SEQ | site | SEQ | |
| name | ID | sequence | ID | gRNA sequence |
| APOC3- | 44 | GGGGCACCCG | 170 | GGGGCACCCGUCCAGCUCCGGUUU |
| A | | TCCAGCTCCG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOC3- | 45 | TGACCTTTGC | 171 | UGACCUUUGCCCAGCGCCCUGUUU |
| B | | CCAGCGCCCT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOC3- | 46 | TCCAGATGCA | 172 | UCCAGAUGCAGCAAGCGGGCGUUU |
| C | | GCAAGCGGGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOC3- | 47 | TAGGGATGAA | 173 | UAGGGAUGAACUGAGCAGACGUUU |
| D | | CTGAGCAGAC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOC3- | 48 | AGAAGCACTT | 174 | AGAAGCACUUGCUAGAGCUAGUUU |
| E | | GCTAGAGCTA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOC3- | 49 | CTGCTCCAGG | 175 | CUGCUCCAGGUAAUGCCCUCGUUU |
| F | | TAATGCCCTC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOC3- | 50 | GGGAGAGTTG | 176 | GGGAGAGUUGGGAAAUCCCUGUUU |
| G | | GGAAATCCCT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOC3- | 51 | AGGAAGCCTC | 177 | AGGAAGCCUCGGAGCUGGACGUUU |
| H | | GGAGCTGGAC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOC3- | 52 | CCCTGGAGAT | 178 | CCCUGGAGAUGAUAUAAAACGUUU |
| I | | GATATAAAAC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOC3- | 53 | TCATAACCTG | 179 | UCAUAACCUGAAGAACAUGGGUUU |
| J | | AAGAACATGG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |

TABLE E7
gRNAs targeting human APOB

| | tar- | | | |
|---|---|---|---|---|
| | get | | | |
| | site | target | gRNA | |
| gRNA | SEQ | site | SEQ | |
| name | ID | sequence | ID | gRNA sequence |
| APOB- | 54 | GTCCATCGCC | 180 | GUCCAUCGCCAGCUGCGGUGGUUU |
| A | | AGCTGCGGTG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 55 | GGCGCCCGCA | 181 | GGCGCCCGCACCCCAUUUAUGUUU |
| B | | CCCCATTTAT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 56 | CAGAGCGGCC | 182 | CAGAGCGGCCGCGCACUCACGUUU |
| C | | GCGCACTCAC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 57 | CTCAGCGGCA | 183 | CUCAGCGGCAGCAACCGAGAGUUU |
| D | | GCAACCGAGA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 58 | TCCCGGTGGG | 184 | UCCCGGUGGGAAUGCGCGGCGUUU |
| E | | AATGCGCGGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 59 | GCATTCCCAC | 185 | GCAUUCCCACCGGGACCUGCGUUU |
| F | | CGGGACCTGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 60 | GCCTCGCGGC | 186 | GCCUCGCGGCCCUGGCUGGCGUUU |
| G | | CCTGGCTGGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 61 | CCCGGCCAAC | 187 | CCCGGCCAACCUCGUGCCGCGUUU |
| H | | CTCGTGCCGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 62 | AGCGCCAGCA | 188 | AGCGCCAGCAGCGCGGGCCUGUUU |
| I | | GCGCGGGCCT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 63 | CTCCCTCTGC | 189 | CUCCCUCUGCGCCCGCAGAGGUUU |
| J | | GCCCGCAGAG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |

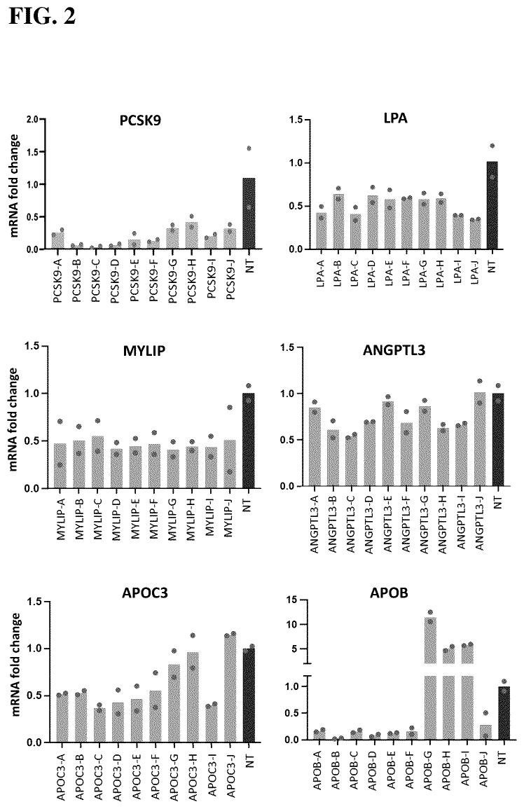

Ten gRNAs were tested for each gene. A human hepatocellular carcinoma cell line (Huh7) was transfected with dSpCas9-KRAB-DNMT3A/L in combination with each designed gRNA. mRNA was isolated from the transfected cells seven days after transfection and assessed for knockdown of the target gene by qRT-PCR. As shown in FIG. 2, co-expression of dSpCas9-KRAB-DNMT3A/L and agRNA led to transcriptional repression of the target genes, with the exception of 3 or 4 gRNAs targeting ANGPTL3, APOC3, and APOB.

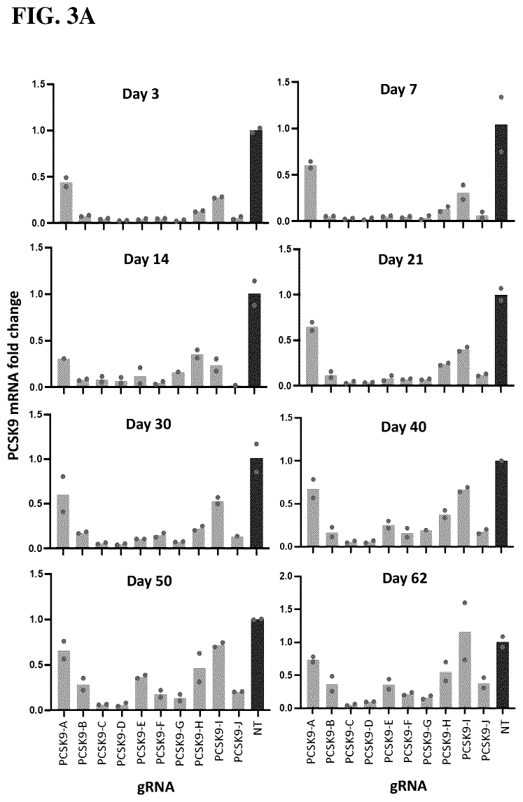

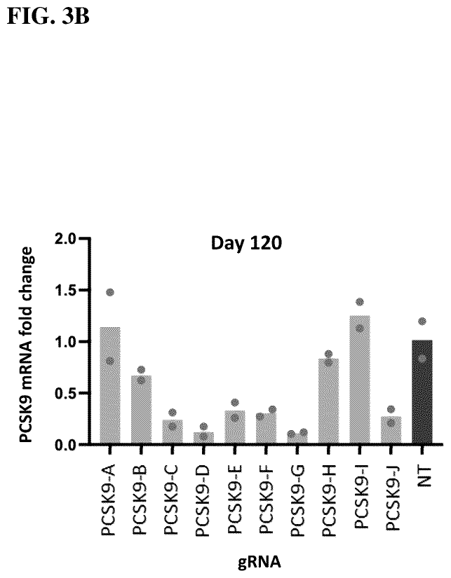

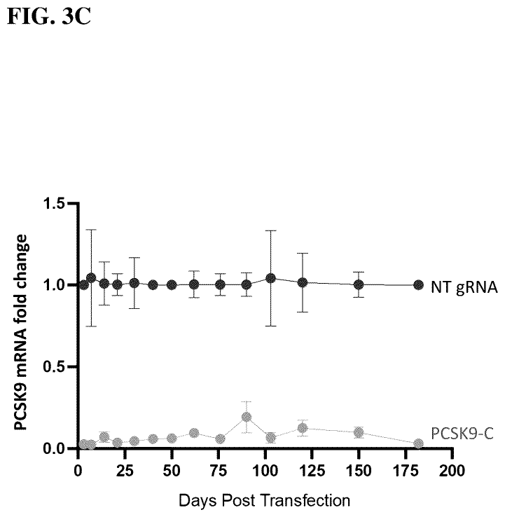

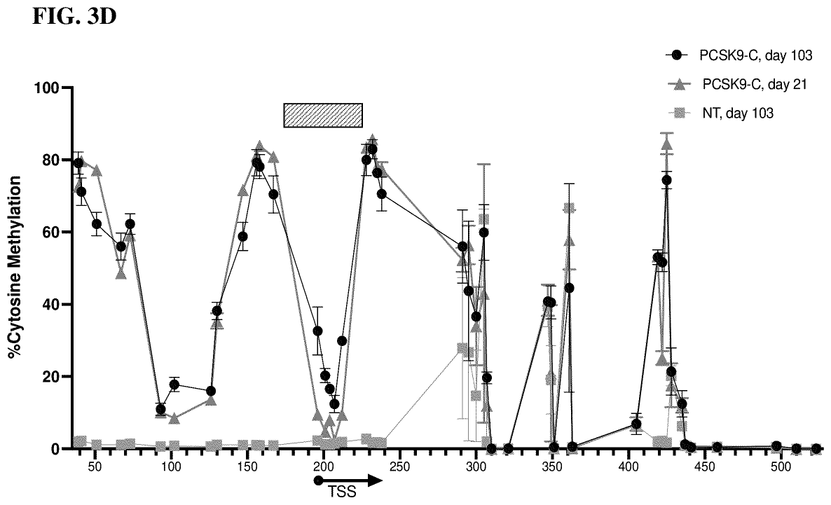

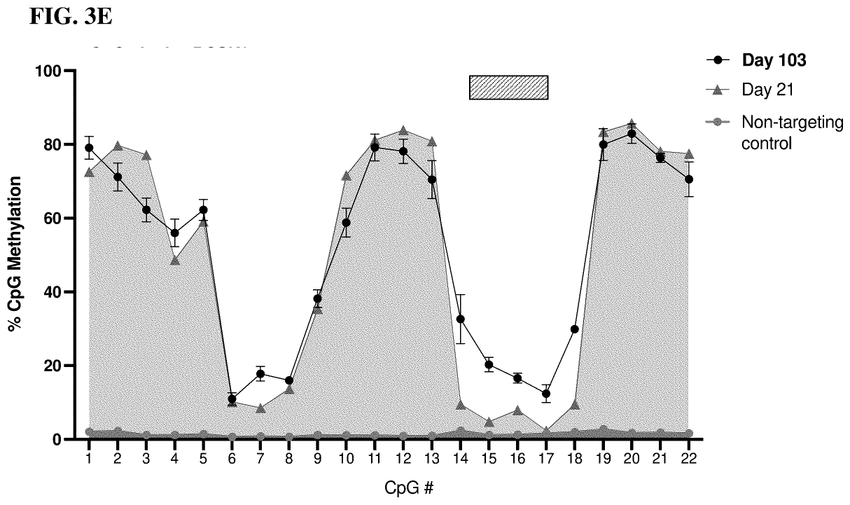

To test the durability of the repression induced by the DNA-targeting systems, PCSK9 mRNA levels were assessed at multiple time points post-transfection with the PCSK9-targeting gRNAs and dSpCas9-KRAB-DNMT3A/L (FIG. 3A). After transfection, cells were passaged approximately every 4-6 days, as necessary. As shown in FIG. 3B, sustained repression of PCSK9 mRNA post-transfection was observed 120 days post-transfection with 8/10 of the gRNAs tested in comparison to a non-targeting gRNA. Analysis at subsequent timepoints demonstrated stable repression of PCSK9 mRNA was sustained for six months after the single transient exposure of liver cells to dSpCas9-KRAB-DNMT3A/L (SEQ ID NO: 278) and gRNA PCSK9-C(SEQ ID NO:129) (FIG. 3C).

To assess if the observed repression of PCSK9 mRNA was due to increased CpG methylation at or near the PCSK9 promoter, DNA was extracted from Huh7 cells transfected with dSpCas9-KRAB-DNMT3A/L (SEQ ID NO: 278) and gRNA PCSK9-C(SEQ ID NO:129) or a non-targeting (NT) gRNA for sequencing analysis and quantification of differential CpG methylation. As shown in FIG. 3D, increased methylation of CpGs at the PCSK9 promoter was observed at day 21(triangles) and day 103 (circles) post-transfection with gRNA PCSK9-C as compared to the non-targeting control (squares),particularly in the region surrounding the PSCK9-C target site (FIG. 3E). These results are consistent with stable methylation around the target site facilitated by the epigenetic-modifying DNA-targeting system in human liver cells.

The results showed that a DNA-targeting system comprising a designed gRNA and a dCas9-effector fusion protein can facilitate durable transcriptional repression of targeted genes and increase CpG methylation around gRNA target sites in human cells. The results further support the utility of repressing PCSK9 and other genes associated with regulation of LDL in vivo for potential treatment of familial hypercholesterolemia and/or cardiovascular disease.

### Example 3: Multiplexed Targeted Transcriptional Repression for Reducing LDL

DNA-targeting systems comprising different combinations of the gRNAs identified in Example 2 are screened to identify conditions in which multiplexed targeted transcriptional repression of at least two genes promotes cellular phenotypes that could reduce LDL in vivo.

DNA-targeting systems comprising gRNAs and a dSpCas9-effector are transiently transfected into Huh7 cells. gRNAs are transfected individually, or in combinations of two gRNAs or three gRNAs, with each gRNA targeting a different gene.

In one example, a combination of two gRNAs comprises a first gRNA targeting one of PCSK9, LPA, MYLIP, ANGLPTL3, APOC3, and APOB, and a second gRNA targeting one of PCSK9, LPA, MYLIP, ANGLPTL3, APOC3, and APOB, wherein the first and second gRNA target different genes. In another example, a combination of two gRNAs comprises a first gRNA targeting PCSK9 and a second gRNA targeting one of LPA, MYLIP, ANGLPTL3, APOC3, and APOB. In another example, a combination of two gRNAs comprises a first gRNA targeting PCSK9 and a second gRNA targeting LPA.

In another example, a combination of three gRNAs comprises a first gRNA targeting one of PCSK9, LPA, MYLIP, ANGLPTL3, APOC3, and APOB, a second gRNA targeting one of PCSK9, LPA, MYLIP, ANGLPTL3, APOC3, and APOB, and a third gRNA targeting one of PCSK9, LPA, MYLIP, ANGLPTL3, APOC3, and APOB, wherein the first, second, and third gRNA each target a different gene. In another example, a combination of three gRNAs comprises a first gRNA targeting PCSK9, a second gRNA targeting one of LPA, MYLIP, ANGLPTL3, APOC3, and APOB, and a third gRNA targeting one of LPA, MYLIP, ANGLPTL3, APOC3, and APOB, wherein the second and third gRNA each target a different gene. In another example, a combination of three gRNAs comprises a first gRNA targeting PCSK9, a second gRNA targeting LPA, and a third gRNA targeting one of MYLIP, ANGLPTL3, APOC3, and APOB.

Following transfection, cells are assessed for phenotypes that could promote reduction of LDL in vivo. Specifically, cells are assessed for increased LDL uptake or increased cell-surface expression of LDL receptor (LDL-R).

To assess LDL uptake, fluorescently labeled LDL (BODIPY-LDL; ThermoFisher #L3483) is added to the cell media, and cells are incubated for 2 hours. Cells are then washed and assessed by flow cytometry for LDL uptake based on fluorescence of BODIPY-LDL. Conditions resulting in increased LDL uptake in comparison to control cells (e.g., cells not transfected with a gRNA) are identified. In addition, conditions are identified in which multiplexed targeted repression of a combination of genes (e.g., PCSK9 and LPA) leads to greater LDL uptake than targeted repression of any individual gene of the combination alone (e.g., PCSK9 alone or LPA alone).

Cells are assessed for cell-surface expression of LDL-R by flow cytometry, using a fluorescently labeled anti-LDL-R antibody (R&D Biosystems #FAB2148P. Conditions resulting in increased expression of LDL-R in comparison to control cells are identified. In addition, conditions are identified in which multiplexed targeted repression of a combination of genes (e.g., PCSK9 and LPA) leads to greater LDL-R expression than targeted repression of any individual gene of the combination alone (e.g., PCSK9 alone or LPA alone).

DNA-targeting systems with combinations of gRNAs are identified that mediate multiplexed targeted transcriptional repression of at least two genes to promote LDL reduction.

### Example 4: In Vivo Targeted Transcriptional Repression for Reducing LDL in a Humanized Mouse Model

DNA-targeting systems comprising a gRNA identified in Example 2 or combinations of gRNAs for multiplexed targeted transcriptional repression identified in Example 3, are assessed for efficacy in vivo using a liver-humanized mouse model. Liver-humanized mice are Fah/Rag2/Il2rg triple mutants (FRG KO) in which the liver is repopulated with human hepatocytes, for example as described in Azuma et al., Nat Biotechnol. 25(8):903-910 (2007).

The identified gRNA or gRNA combinations are delivered with a dSpCas9-effector fusion protein for targeted gene repression (e.g., dSpCas9-KRAB or dSpCas9-KRAB-DNMT3A/L). Lipid nanoparticles (LNPs) comprising the gRNAs and fusion protein (or nucleic acids encoding either component) are formulated for the delivery. In one example, the LNPs include the gRNAs and mRNA encoding the fusion protein. LNPs are delivered systemically via injection (e.g. into the tail vein). Levels of circulating cholesterol, including LDL, and target protein knockdown are assessed based on analysis of blood serum using enzyme-linked immunoassay (ELISA).

Decreased levels of circulating LDL support the utility of repressing genes associated with regulation of LDL in vivo, including with the multiplexed epigenetic-modifying DNA-targeting systems provided herein, for potential treatment of familial hypercholesterolemia and/or cardiovascular disease.

### Example 5: Identification of 2RNAs that Repress Target Genes Associated with Regulation of LDL Using Flow FISH

A library of gRNAs were designed for targeting human genes associated with regulation of low-density lipoprotein (LDL), including PCSK9, MYLIP, and APOB. The designed gRNAs were assessed using FlowFISHfor their ability to mediate transcriptional repression as part of a DNA-targeting system composed of a gRNA and a dCas9-effector fusion protein. FlowFISH is a flow cytometry-based fluorescent in situ hybridization technique for assessing gene expression by measuring fluorescently labeled RNA. The FlowFISH technique provides a more direct analysis of gRNA effects by staining for mRNA expression and is a useful alternative to antibody staining since the gene targets include those that encode cell-secretable proteins.

gRNAs were designed that target an area surrounding the transcriptional start site of each target gene, ˜5kb upstream and downstream for each gene, as well as open chromatin and possible enhancer regions lying outside of this 10kb region, up to 30kb in either direction. Target regions were selected based on DNase hypersensitive regions and H3k27ac ChTP called peaks in human liver tissue identified in ENCODE data. Target sites were selected according to the protospacer adjacent motif (PAM) sequence for SpCas9 (5′-NGG-3′; SEQ ID NO:202). Each gRNA further comprised a scaffold sequence for SpCas9, set forth in SEQ ID NO: 191.

Huh7 cells were transduced with lentivirus vector comprising a gRNA and transiently transfected with mRNA encoding dSpCas9-KRAB-DNMT3A/L (SEQ ID NO:278) fusion protein. Transfected cells were collected at day 28, fixed, permeabilized, and stained with fluorescently labeled probes that bind the mRNA of the target genes to enable gene expression analysis using FlowFISH.

gRNAs that repressed PCSK9, MYLIP, and APOB were identified and are listed in Table E8 (PCSK9), Table E9 (MYLIP) and Table E10 (APOB). The DNA target site (protospacer) sequences are set forth in SEQ ID NOS:3, 4, 29, 54, 57, 306-317, 342-351 and 372-377 and gRNA spacer sequences for gRNAs targeting each site are set forth in SEQ ID NOS: 66, 67, 92, 117, 120, 318-329, 352-361, and 378-383. These sequences are listed in Table 3 (Section LB.). In some cases, the identified gRNAs had the same or similar sequences to gRNAs identified in Example 2 above.

TABLE E8
gRNAs targeting human PCSK9

| | tar- | | | |
|---|---|---|---|---|
| | get | | | |
| | site | target | gRNA | |
| gRNA | SEQ | site | SEQ | |
| name | ID | sequence | ID | gRNA sequence |
| PCSK9- | 306 | GTCGAGGCGC | 330 | GUCGAGGCGCUCAUGGUUGCGUUU |
| N | | TCATGGTTGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 3 | AGGTTTCCGC | 129 | AGGUUUCCGCAGCGACGUCGGUUU |
| C | | AGCGACGTCG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 307 | TTCCAGCCCA | 331 | UUCCAGCCCAGUUAGGAUUUGUUU |
| O | | GTTAGGATTT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 308 | TCCTAACTGG | 332 | UCCUAACUGGGCUGGAAGGCGUUU |
| P | | GCTGGAAGGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 309 | TCAGGAGCAG | 333 | UCAGGAGCAGGGCGCGUGAAGUUU |
| Q | | GGCGCGTGAA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 310 | CAGCGACGTC | 334 | CAGCGACGUCGAGGCGCUCAGUUU |
| R | | GAGGCGCTCA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 311 | CCGTCAGCTC | 335 | CCGUCAGCUCCAGGCGGUCCGUUU |
| S | | CAGGCGGTCC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 312 | AACCTGATCC | 336 | AACCUGAUCCUCCAGUCCGGGUUU |
| T | | TCCAGTCCGG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 313 | TCATGGGCAC | 337 | UCAUGGGCACCGUCAGCUCCGUUU |
| U | | CGTCAGCTCC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 314 | CCGCCGGCGT | 338 | CCGCCGGCGUGGACCGCGCAGUUU |
| V | | GGACCGCGCA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 315 | GAAGGCAGGC | 339 | GAAGGCAGGCCGGCGCCCUAGUUU |
| W | | CGGCGCCCTA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 316 | GCGCCTTGAG | 340 | GCGCCUUGAGCCUUGCGGUGGUUU |
| X | | CCTTGCGGTG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 4 | GGGCGCCGCC | 130 | GGGCGCCGCCGUUCAGUUCAGUUU |
| D | | GTTCAGTTCA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| PCSK9- | 317 | CCCGCACCTT | 341 | CCCGCACCUUGGCGCAGCGGGUUU |
| Y | | GGCGCAGCGG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |

TABLE E9
gRNAs targeting human MYLIP

| | tar- | | | |
|---|---|---|---|---|
| | get | | | |
| | site | target | gRNA | |
| gRNA | SEQ | site | SEQ | |
| name | ID | sequence | ID | gRNA sequence |
| MYLIP- | 342 | GCACTGCGGC | 362 | GCACUGCGGCGGCAGCCGGGGUUU |
| K | | GGCAGCCGGG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 343 | GGCGCCACCG | 363 | GGCGCCACCGCGGAGGACAGGUUU |
| L | | CGGAGGACAG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 344 | ATGCTCATAG | 364 | AUGCUCAUAGGAUGUAUUCAGUUU |
| M | | GATGTATTCA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 345 | CCACAATAAA | 365 | CCACAAUAAACACAUGGUCUGUUU |
| N | | CACATGGTCT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 346 | GGGTCCCACC | 366 | GGGUCCCACCAGUGACAAGGGUUU |
| O | | AGTGACAAGG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 347 | GGACTGCCTC | 367 | GGACUGCCUCAACCAGGUGAGUUU |
| P | | AACCAGGTGA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 348 | CAGAGTCCCT | 368 | CAGAGUCCCUGUCGCAGCGCGUUU |
| Q | | GTCGCAGCGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 349 | CTCAGAGTGA | 369 | CUCAGAGUGAGCGAUCGCCCGUUU |
| R | | GCGATCGCCC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 29 | CCCCGCGCAC | 155 | CCCCGCGCACACCAAAGAGAGUUU |
| F | | ACCAAAGAGA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 350 | CTGAGTTTCC | 370 | CUGAGUUUCCCUGGCCGCCCGUUU |
| S | | CTGGCCGCCC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| MYLIP- | 351 | GTTTCCCTGG | 371 | GUUUCCCUGGCCGCCCCGGGGUUU |
| T | | CCGCCCCGGG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |

TABLE E10
gRNAs targeting human APOB

| | tar- | | | |
|---|---|---|---|---|
| | get | | | |
| | site | target | gRNA | |
| gRNA | SEQ | site | SEQ | |
| name | ID | sequence | ID | gRNA sequence |
| APOB- | 54 | GTCCATCGCC | 180 | GUCCAUCGCCAGCUGCGGUGGUUU |
| A | | AGCTGCGGTG | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 372 | TGAGGGCCTC | 384 | UGAGGGCCUCCCACUCUACAGUUU |
| K | | CCACTCTACA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 373 | CCAGAGCACT | 385 | CCAGAGCACUGAAGACGCUUGUUU |
| L | | GAAGACGCTT | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 374 | ACTGGAGGAA | 386 | ACUGGAGGAAACCUAGAAGCGUUU |
| M | | ACCTAGAAGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 57 | CTCAGCGGCA | 183 | CUCAGCGGCAGCAACCGAGAGUUU |
| D | | GCAACCGAGA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 375 | CACTGAAGAC | 387 | CACUGAAGACGCUUGGGGAAGUUU |
| N | | GCTTGGGGAA | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 376 | TGAAGAAGGC | 388 | UGAAGAAGGCACCCCUGGUCGUUU |
| O | | ACCCCTGGTC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |
| APOB- | 377 | TGAGTGCGCG | 389 | UGAGUGCGCGGCCGCUCUGCGUUU |
| P | | GCCGCTCTGC | | AAGAGCUAUGCUGGAAACAGCAUA |
| | | | | GCAAGUUUAAAUAAGGCUAGUCCG |
| | | | | UUAUCAACUUGAAAAAGUGGCACC |
| | | | | GAGUCGGUGC |

### Example 6: In Vivo Targeted PCSK9 Repression in a Non-Human Primate

DNA-targeting systems comprising an exemplary dCas9-effector fusion protein for transcriptional repression and a PCSK9 targeting guide RNA were assessed in vivo using a non-human primate model (NHP).

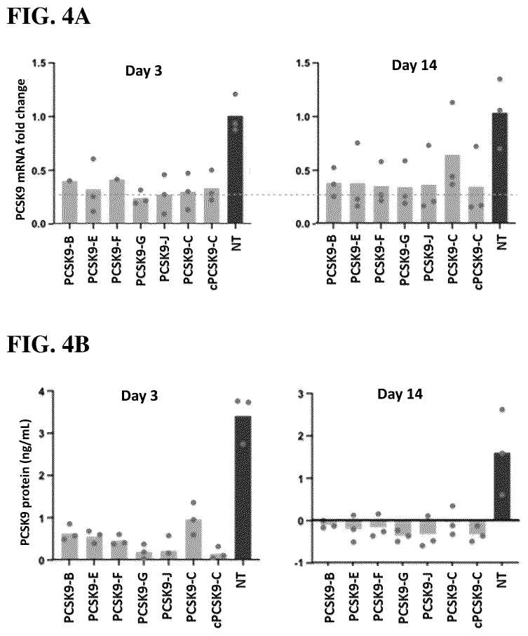

To ensure targeting of Cynomolgus macaque PCSK9, primary hepatocytes from a Cynomolgus macaque donor were transfected with a PCSK9 targeting gRNA and dSpCas9-KRAB-DNMT3A/L. mRNA was isolated from the transfected cells 3 days and 14 days after transfection and assessed for knockdown of the target gene by qRT-PCR. PCSK9 target protein knockdown was also assessed in supernatant using enzyme-linked immunoassay (ELISA). The gRNAs included those targeting target sites: PCSK9-B (SEQ ID NO:2; gRNA SEQ ID NO:128), PCSK9-E (SEQ ID NO:5; gRNA SEQ ID NO:131), PCSK9-F (SEQ ID NO:6; gRNA SEQ ID NO:132), PCSK9-G (SEQ ID NO:7; gRNA SEQ ID NO:133), PCSK9-J (SEQ ID NO:10; gRNA SEQ ID NO:136), and PCSK9-C(SEQ ID NO:3; gRNA SEQ ID NO:129), as well as a cynomolgus-specific version of PCSK9-C(cPCSK9-C) targeting the DNA target site set forth in SEQ ID NO:390. The corresponding gRNA spacer sequence is set forth in SEQ ID NO:391 and the full cPCSK9-C gRNA sequence is set forth in SEQ ID NO:392. As shown in FIGS. 4A and 4B, co-expression of dSpCas9-KRAB-DNMT3A/L and the respective PCSK9 gRNA led to transcriptional repression of PCSK9 at day 3 and 14 post-transfection, as shown by reduced levels of both mRNA (FIG. 4A) and protein (FIG. 4B). After a single administration, PCSK9 mRNA was repressed by about 93% and protein expression dropped below the limit of assay detection 14 days after transfection, which is the approximate lifespan of primary hepatocytes in culture.

To test targeted PCSK9 repression in vivo, ten cynomolgus macaques were divided into four treatment groups: a) PBS-treated control (n=2); b) High dose treatment: 3.0 mg/kg (n=3); c) Low dose treatment: 1.0 mg/kg (n=3); and d) Low dose treatment: 1.0 mg/kg (n=2) for assessing biodistribution. The treatment group for each NHP is listed in Table E11.

TABLE E11
NHP study design for PCSK9-targeting system

| Subject | Treatment Group |
|---|---|
| NHP1 | High dose (3.0 mg/kg) |
| NHP2 | High dose (3.0 mg/kg) |
| NHP3 | High dose (3.0 mg/kg) |
| NHP4 | Low dose (1.0 mg/kg) |
| NHP5 | Low dose (1.0 mg/kg) |
| NHP6 | Low dose (1.0 mg/kg) |
| NHP7 | PBS control |
| NHP8 | PBS control |
| NHP9 | Low dose (1.0 mg/kg) |
| NHP10 | Low dose (1.0 mg/kg) |

Lipid nanoparticles (LNPs) containing gRNA cPCSK9-C(SEQ ID:392) and dSpCas9-KRAB-DNMT3A/L (SEQ ID NO:280) were formulated and delivered systemically via intravenous infusion. Levels of circulating cholesterol (i.e., LDL-C), liver enzymes (i.e., alanine transaminase (ALT) and aspartate aminotransferase (AST)), and PCSK9 protein knockdown were assessed at multiple time-points post-infusion based on blood serum samples analyzed using enzyme-linked immunoassay (ELISA).

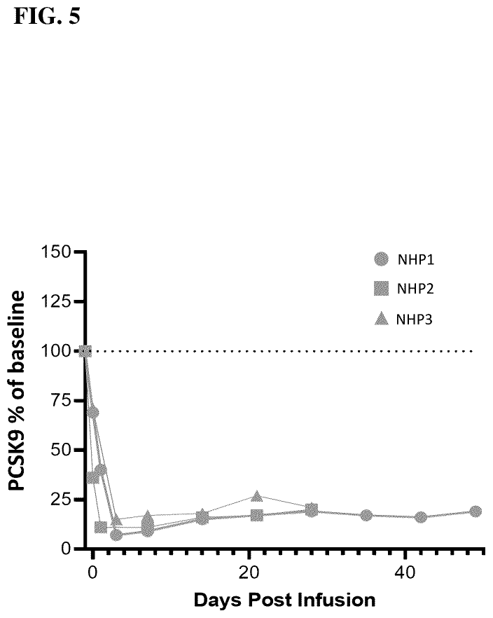

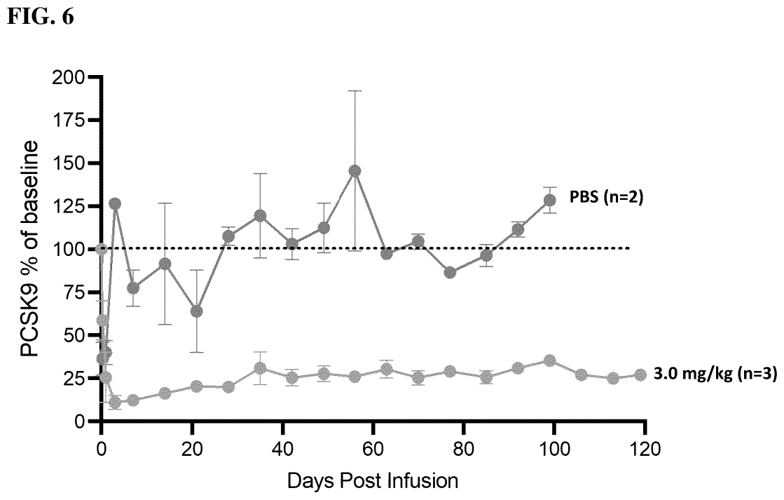

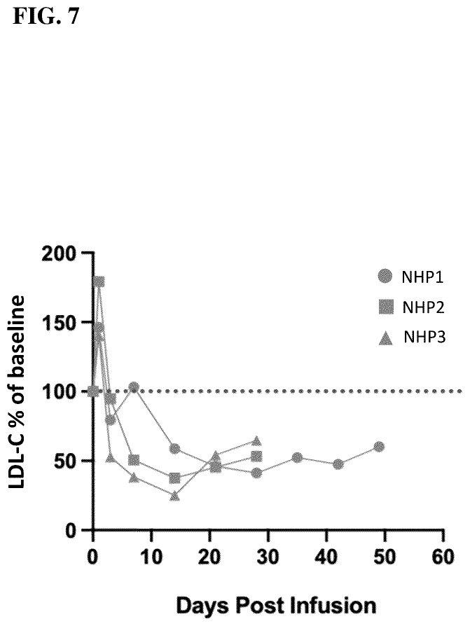

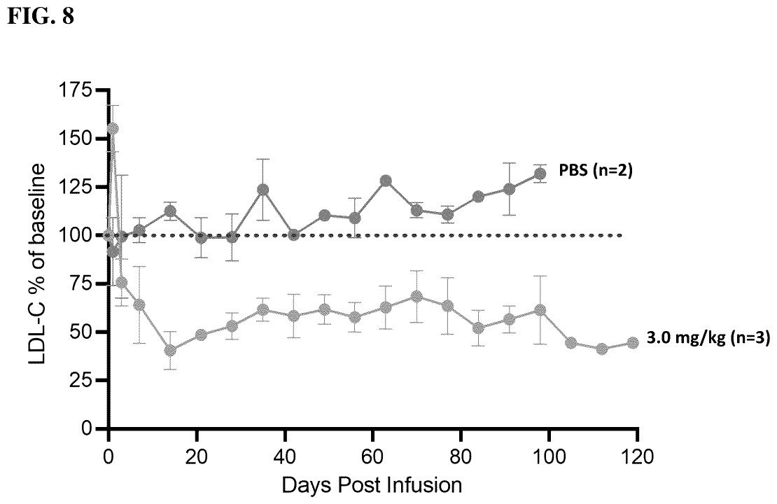

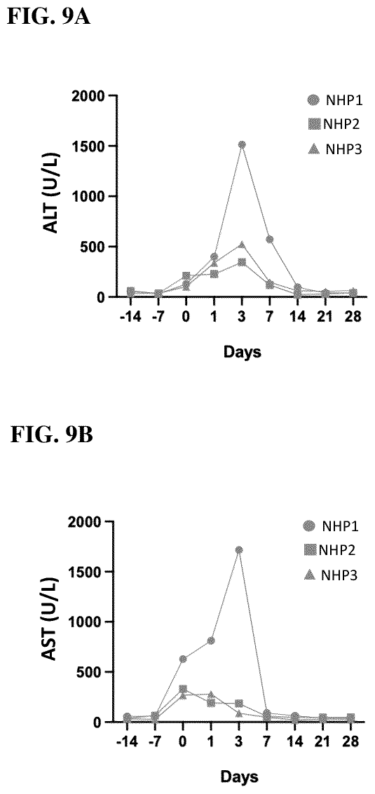

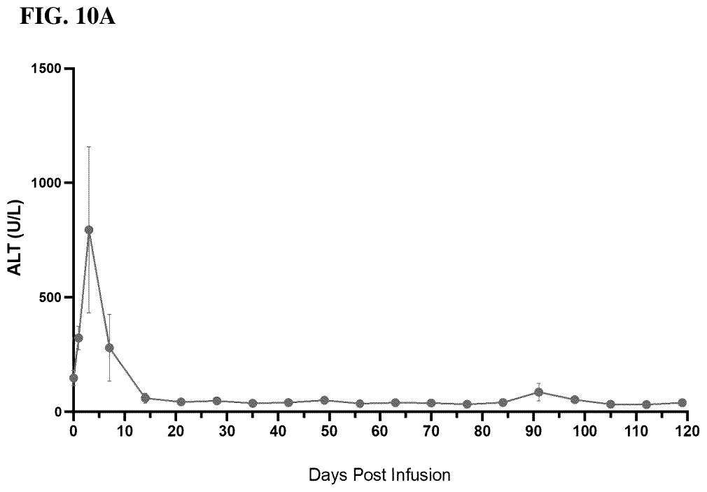

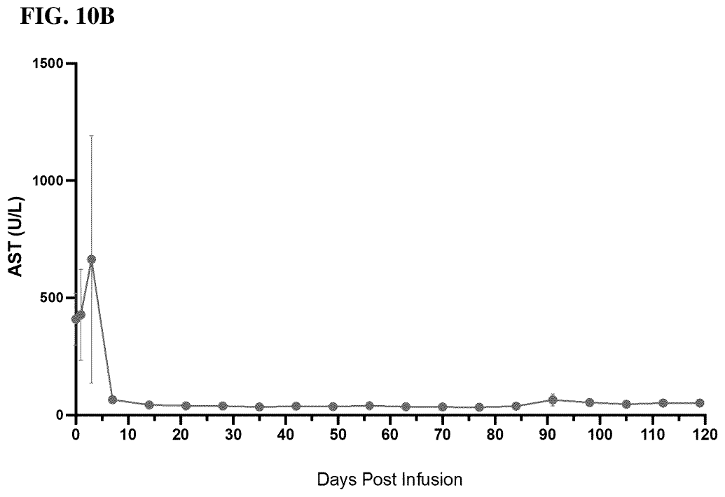

Blood PCSK9 protein levels for the high dose (3.0 mg/kg, n=3) group dropped substantially by day 3 post-infusion and the repression from the single intravenous dose was maintained throughout the measured time points. FIG. 5 shows results for data collected through day 49 post-infusion for NHP 1(circles) and through day 28 post-infusion for NHP 2 (squares) and NHP3 (triangles). At day 28 post-infusion, all three subjects exhibited a reduction of blood PCSK9 levels of ˜80%. FIG. 6 shows sustained PCSK9 repression of ˜80% out to 120 days post-infusion FIG. 7 shows that the high dose treatment also resulted in a downward trend of LDL-C levels, with a stable reduction of ˜50% for all three subjects at day 28 post-infusion. FIG. 8 shows sustained LDL-C repression of ˜50% out to 120 days post-infusion.. FIGS. 9A and 9B show a transient spike in ALT and AST liver enzyme levels at day 3 post-infusion with the high dose treatment that normalized to baseline levels within 1-2 weeks and remained at baseline through day 120 (FIGS. 10A and 10B). Otherwise, the animals were observed to be healthy and normal without any adverse clinical symptoms throughout the treatment.

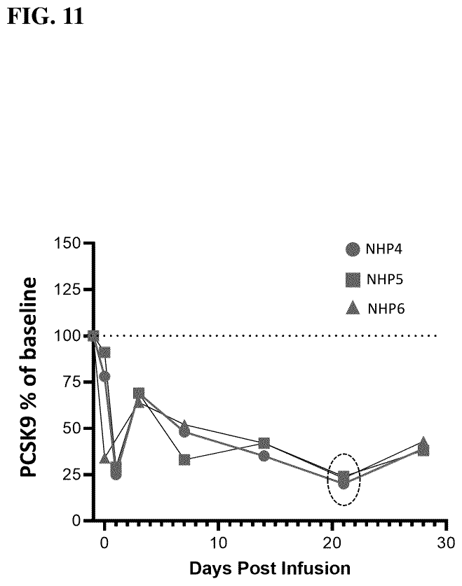

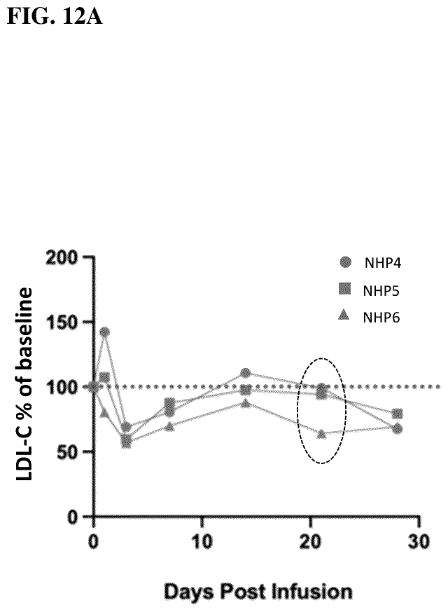

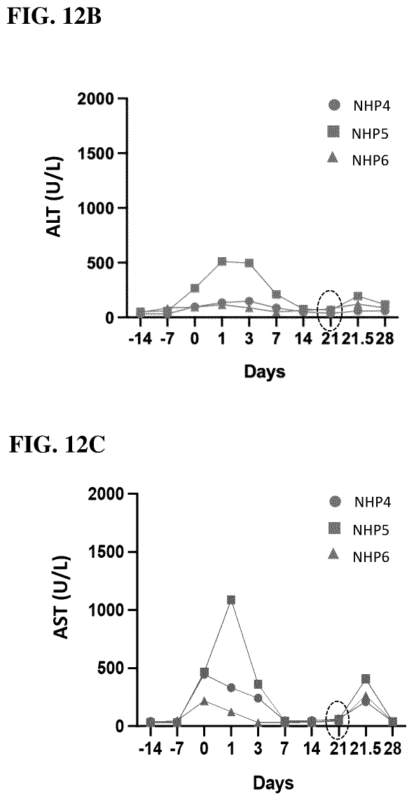

As shown in FIG. 11, blood PCSK9 protein levels for the low dose (1.0 mg/kg, n=3) group decreased ˜60% by day 7. Each NHP in the low dose group was re-dosed at 1.0 mg/kg at day 21 post-infusion and data was collected for an additional 7 days. FIG. 12A shows that LDL-C levels decreased modestly after the administration of the re-dose. FIGS. 12B and 12C show that ALT and AST liver enzyme levels spiked after the initial infusion but returned to baseline by day 7 and that a second smaller spike occurred after the re-dose at day 21 but returned to baseline by day 28.

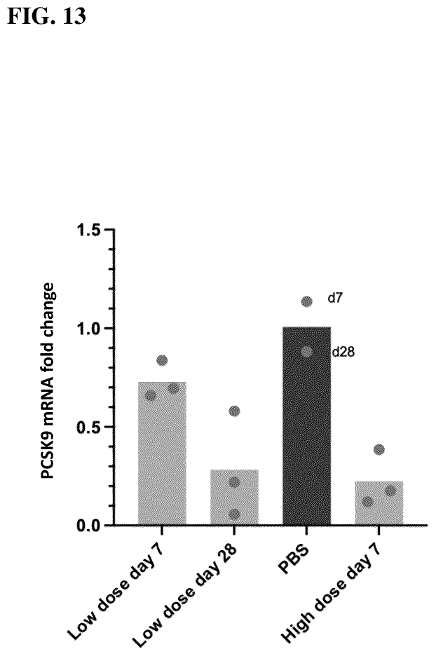

To assess the tissue-specific efficacy of the DNA-targeting system, liver biopsies were performed at day 7 for the low dose (1.0 mg/kg, n=3), high dose (3.0 mg/kg, n=3), and control (PBS, n=2) treatment groups. To determine the impact of the re-dose at day 21 post-infusion, a second liver biopsy was performed for the low dose and PBS control groups 7 days after the low dose group was re-dosed at 1.0 mg/kg. As shown in FIG. 13, the low dose group exhibited a modest reduction in PCSK9 mRNA at day 7 and a further reduction at day 28 that approached the strong reduction of PCSK9 mRNA observed in the high dose group at day 7. These results indicate that a re-dose of a dCas9-effector fusion and PCSK9 gRNA can further decrease PCSK9 mRNA levels in a tissue-specific manner.

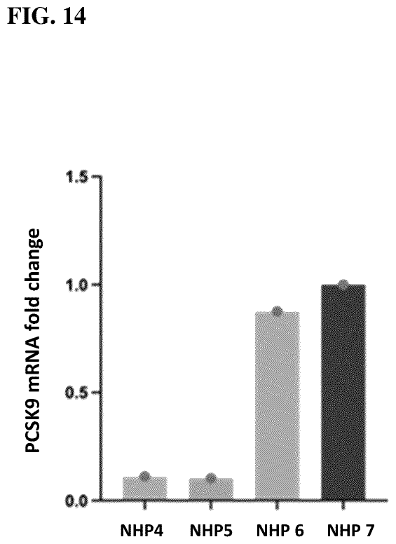

To assess the efficacy of redosing at a higher dose, 126 days after the initial infusion NHP4 and NHP5 from the low dose group were redosed at 3.0 mg/kg and NHP6 was redosed at 1.0 mg/kg. NHP5 experienced an adverse event after the redose. PCSK9 mRNA levels were assessed from liver biopsies performed at day 129 for NHP5 and day 140 for NHP4, NHP6, and NHP7 from the PBS control group. As shown in FIG. 14, the 3.0 mg/kg redose for NHP4 and NHP5 resulted in a substantial drop in PCSK9 mRNA levels compared to NHP6 which exhibited only a modest reduction compared to the PBS control (NHP7).

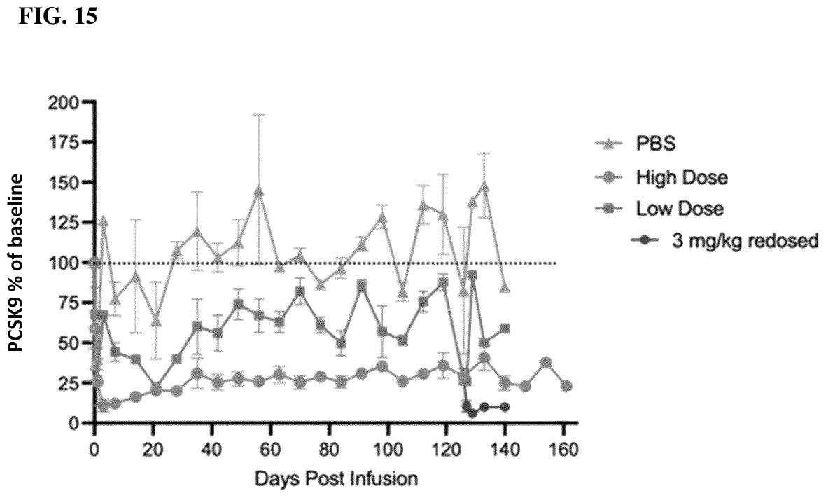

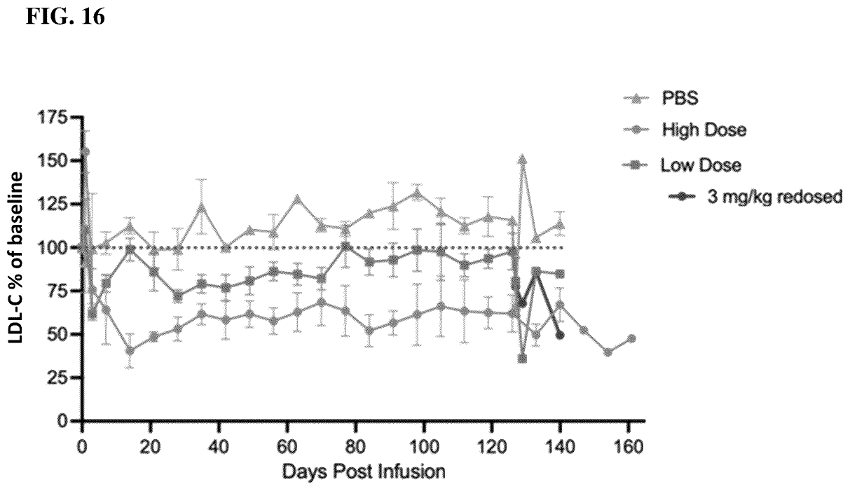

FIG. 15 shows results for blood PCSK9 protein levels through day 160 post-infusion for the high dose group (circles) and through day 140 post-infusion for the low dose group (squares) and the PBS-treated group (triangles). The high dose group continued to maintain stable repression of blood PCSK9 levels through day 160. The 3.0 mg/kg redose for NHP4 at day 126 resulted in a drop in blood PCSK9 levels below the levels observed for the high dose group that was sustained through day 140. FIG. 16 shows results for LDL-C levels through day 160 post-infusion for the high dose group (circles) and through day 140 post-infusion for the low dose group (squares) and the PBS-treated group (triangles). The high dose group continued to maintain stable repression of LDL-C levels through day 160. The 3.0 mg/kg redose for NHP4 at day 126 resulted in a downward trend of LDL-C levels that reached the levels observed for the high dose group. These results indicate that a higher re-dose of a dCas9-effector fusion and PCSK9 gRNA can further decrease PCSK9 protein and LDL-C levels in vivo.

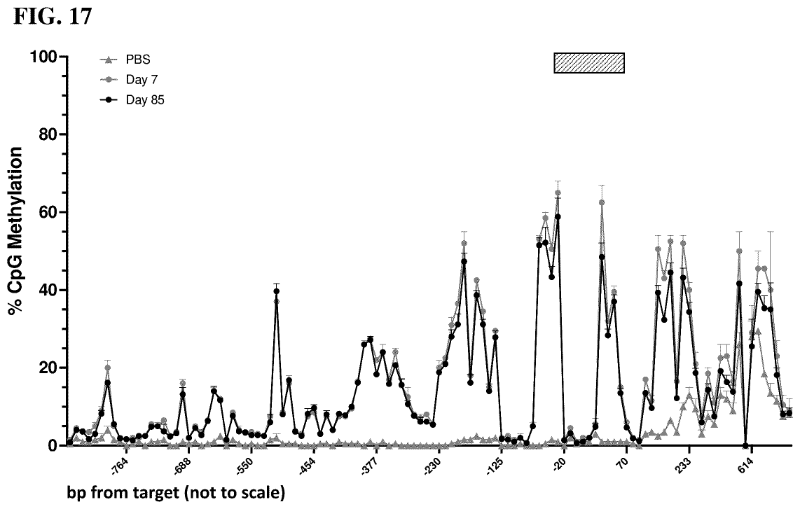

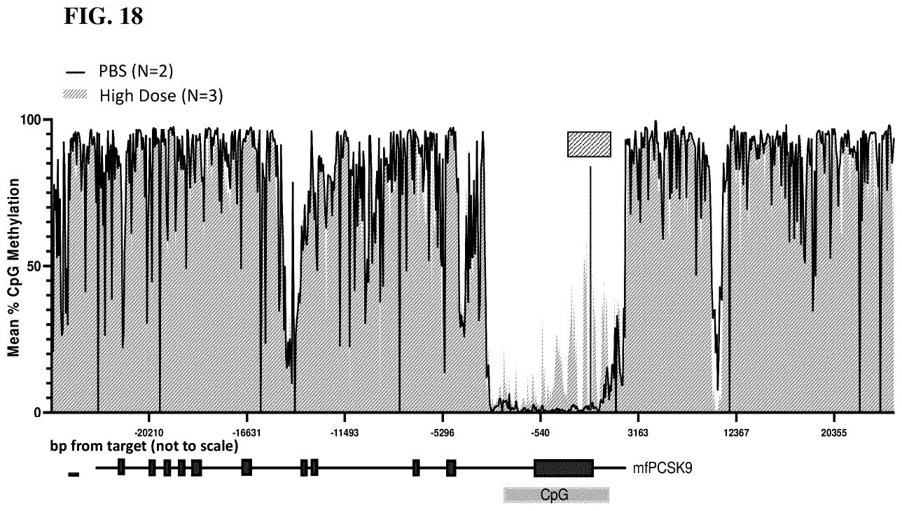

To investigate the mechanistic basis for the observed reductions in PCSK9, DNA was extracted from the high dose and control liver biopsies performed at day 7 post-infusion for targeted methylation sequencing. A 50 kb region surrounding the gRNA target site was sequenced and differential CpG methylation was quantified. As shown in FIG. 17, at day 7 (light circles) the high dose (3.0 mg/kg) subjects exhibited peak methylation in the region surrounding the gRNA target site (e.g., −500 bp), with up to 68% CpG methylation at certain residues compared to ˜1% in PBS controls (triangles). To assess the durability of the treatment, liver biopsies for the high dose and control groups were performed at day 85 post-infusion and DNA was extracted for targeted methylation sequencing. Notably, methylation levels at day 85 (dark circles) were similar to levels measured at day 7 (FIG. 17). The spread of methylation at day 85 post LNP administration was limited to a ˜2kb region surrounding the PCSK9 CpG island (FIG. 18).

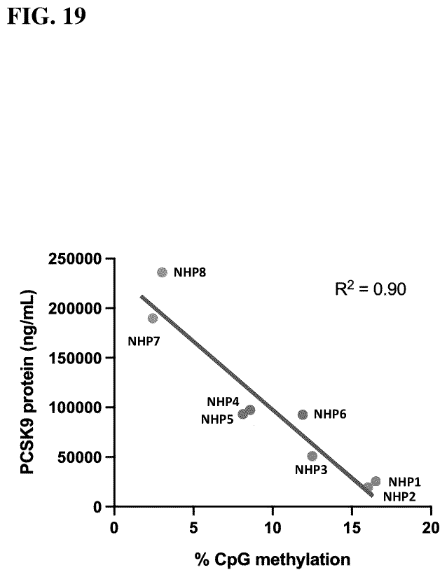

FIG. 19 shows that PCSK9 protein levels decrease as CpG methylation increases across the three treatment groups. Specifically, PCSK9 protein levels were plotted against the average % CpG methylation across the 2.2 kB PCSK9 promoter region for each individual NHP in the three treatment groups. The PBS-treated control group, NHP7 and NHP8, show high levels of PCSK9 and minimal CpG methylation. The low dose (1.0 mg/kg) group, NHP 4, NHP5, and NHP 6, exhibited ˜60% reduction in PCSK9 levels compared to the PBS control group, which corresponded to ˜7-12% average CpG methylation. The high dose group exhibited a strong reduction in PCSK9 levels (˜80%), which corresponded to ˜13-17% average CpG methylation. The calculated R² value (coefficient of determination) for these data was 0.90, indicating that 90% of the variance in PCSK9 protein levels can be predicted by % CpG methylation of the PCSK9 promoter.

The results demonstrated that a DNA-targeting system composed of a PCSK9 targeting gRNA and a dCas9-effector fusion protein can substantially and stably reduce PCSK9 blood protein and LDL-C levels in a non-human primate by increasing CpG methylation of the PCSK9 promoter. Notably, increased methylation of the PCSK9 promoter reduces PSCK9 and LDL-C levels without altering genomic DNA sequences, minimizes off-target effects that can occur from gene editing, and is more durable than RNAi. The results further support the utility of targeted repression of PCSK9 in vivo for potential treatment of familial hypercholesterolemia and/or cardiovascular disease, such as artheroscherotic cardiovascular disease (ASCVD).

### Example 7: In Vitro Targeted PCSK9 Repression in Human Hepatocytes

DNA-targeting systems comprising an exemplary dCas9-effector fusion protein for transcriptional repression and a PCSK9 targeting guide RNA were assessed in primary human hepatocytes.

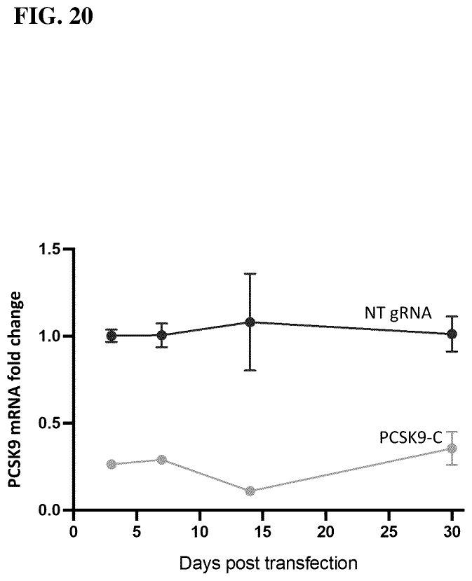

Primary human hepatocytes were transfected with PCSK9-C gRNA (SEQ ID NO: 129; target site SEQ ID NO:3) and dSpCas9-KRAB-DNMT3A/L. mRNA was isolated from the transfected cells at day 3, 7, 14 and 30 post-transfection and assessed for knockdown of the target gene by qRT-PCR. As shown in FIG. 20, co-expression of dSpCas9-KRAB-DNMT3A/L and PCSK9-C gRNA led to transcriptional repression of PCSK9 at every time point as shown by reduced levels of PCSK9 mRNA. After a single administration, there was substantial PCSK9 mRNA repression through day 30 post-transfection.

The results demonstrated that a DNA-targeting system composed of a PCSK9 targeting gRNA and a dCas9-effector fusion protein can substantially and stably reduce PCSK9 mRNA levels in primary human hepatocytes.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | | |
| NO. | Sequence | Annotation |
| 190 | GTTTAAGAGCTATGCTGGAAACAGCATAGCAAGTTTAAATAAGGCTAGTCCGTTA | SpCas9 |
| | TCAACTTGAAAAAGTGGCACCGAGTCGGTGC | gRNA |
| | | scaffold |
| | | (DNA) |
| 191 | GUUUAAGAGCUAUGCUGGAAACAGCAUAGCAAGUUUAAAUAAGGCUAGUCCGU | SpCas9 |
| | UAUCAACUUGAAAAAGUGGCACCGAGUCGGUGC | gRNA |
| | | scaffold |
| | | (RNA) |
| 192 | CGGACACTGGTGACCTTCAAGGATGTGTTTGTGGACTTCACCAGGGAGGAGTGGA | KRAB (nt) |
| | AGCTGCTGGACACTGCTCAGCAGATCCTGTACAGAAATGTGATGCTGGAGAACTA | |
| | TAAGAACCTGGTTTCCTTGGGTTATCAGCTTACTAAGCCAGATGTGATCCTCCGGT | |
| | TGGAGAAGGGAGAAGAGCCCTGGCTGGTG | |
| 193 | RTLVTFKDVFVDFTREEWKLLDTAQQILYRNVMLENYKNLVSLGYQLTKPDVILRLE | KRAB |
| | KGEEPWLV | (AA) |
| 194 | ACCTACGGGCTGCTGCGGCGGCGAGAGGACTGGCCCTCCCGGCTCCAGATGTTCT | DNMT3A |
| | TCGCTAATAACCACGACCAGGAATTTGACCCTCCAAAGGTTTACCCACCTGTCCCA | (nt) |
| | GCTGAGAAGAGGAAGCCCATCCGGGTGCTGTCTCTCTTTGATGGAATCGCTACAG | |
| | GGCTCCTGGTGCTGAAGGACTTGGGCATTCAGGTGGACCGCTACATTGCCTCGGA | |
| | GGTGTGTGAGGACTCCATCACGGTGGGCATGGTGCGGCACCAGGGGAAGATCATG | |
| | TACGTCGGGGACGTCCGCAGCGTCACACAGAAGCATATCCAGGAGTGGGGCCCAT | |
| | TCGATCTGGTGATTGGGGGCAGTCCCTGCAATGACCTCTCCATCGTCAACCCTGCT | |
| | CGCAAGGGCCTCTACGAGGGCACTGGCCGGCTCTTCTTTGAGTTCTACCGCCTCCT | |
| | GCATGATGCGCGGCCCAAGGAGGGAGATGATCGCCCCTTCTTCTGGCTCTTTGAG | |
| | AATGTGGTGGCCATGGGCGTTAGTGACAAGAGGGACATCTCGCGATTTCTCGAGT | |
| | CCAACCCTGTGATGATTGATGCCAAAGAAGTGTCAGCTGCACACAGGGCCCGCTA | |
| | CTTCTGGGGTAACCTTCCCGGTATGAACAGGCCGTTGGCATCCACTGTGAATGATA | |
| | AGCTGGAGCTGCAGGAGTGTCTGGAGCATGGCAGGATAGCCAAGTTCAGCAAAGT | |
| | GAGGACCATTACTACGAGGTCAAACTCCATAAAGCAGGGCAAAGACCAGCATTTT | |
| | CCTGTCTTCATGAATGAGAAAGAGGACATCTTATGGTGCACTGAAATGGAAAGGG | |
| | TATTTGGTTTCCCAGTCCACTATACTGACGTATCCAACATGAGCCGCTTGGCGAGG | |
| | CAGAGACTGCTGGGCCGGTCATGGAGCGTGCCAGTCATCCGCCACCTCTTCGCTCC | |
| | GCTGAAGGAGTATTTTGCGTGTGTG | |
| 195 | TYGLLRRREDWPSRLQMFFANNHDQEFDPPKVYPPVPAEKRKPIRVLSLFDGIATGLL | DNMT3A |
| | VLKDLGIQVDRYIASEVCEDSITVGMVRHQGKIMYVGDVRSVTQKHIQEWGPFDLVI | (AA) |
| | GGSPCNDLSIVNPARKGLYEGTGRLFFEFYRLLHDARPKEGDDRPFFWLFENVVAMG | |
| | VSDKRDISRFLESNPVMIDAKEVSAAHRARYFWGNLPGMNRPLASTVNDKLELQECL | |
| | EHGRIAKFSKVRTITTRSNSIKQGKDQHFPVFMNEKEDILWCTEMERVFGFPVHYTDV | |
| | SNMSRLARQRLLGRSWSVPVIRHLFAPLKEYFACV | |
| 196 | ATGGCGGCCATCCCAGCCCTGGACCCAGAGGCCGAGCCCAGCATGGACGTGATTT | DNMT3L |
| | TGGTGGGATCCAGTGAGCTCTCAAGCTCCGTTTCACCCGGGACAGGCAGAGATCT | (nt) |
| | TATTGCATATGAAGTCAAGGCTAACCAGCGAAATATAGAAGACATCTGCATCTGC | |
| | TGCGGAAGTCTCCAGGTTCACACACAGCACCCTCTGTTTGAGGGAGGGATCTGCG | |
| | CCCCATGTAAGGACAAGTTCCTGGATGCCCTCTTCCTGTACGACGATGACGGGTAC | |
| | CAATCCTACTGCTCCATCTGCTGCTCCGGAGAAACGCTGCTCATCTGCGGAAACCC | |
| | TGATTGCACCCGATGCTACTGCTTCGAGTGTGTGGATAGCCTGGTCGGCCCCGGGA | |
| | CCTCGGGGAAGGTGCACGCCATGAGCAACTGGGTGTGCTACCTGTGCCTGCCGTC | |
| | CTCCCGAAGCGGGCTGCTGCAGCGTCGGAGGAAGTGGCGCAGCCAGCTCAAGGCC | |
| | TTCTACGACCGAGAGTCGGAGAATCCCCTTGAGATGTTCGAAACCGTGCCTGTGT | |
| | GGAGGAGACAGCCAGTCCGGGTGCTGTCCCTTTTTGAAGACATCAAGAAAGAGCT | |
| | GACGAGTTTGGGCTTTTTGGAAAGTGGTTCTGACCCGGGACAACTGAAGCATGTG | |
| | GTTGATGTCACAGACACAGTGAGGAAGGATGTGGAGGAGTGGGGACCCTTCGATC | |
| | TTGTGTACGGCGCCACACCTCCCCTGGGCCACACCTGTGACCGTCCTCCCAGCTGG | |
| | TACCTGTTCCAGTTCCACCGGCTCCTGCAGTACGCACGGCCCAAGCCAGGCAGCC | |
| | CCAGGCCCTTCTTCTGGATGTTCGTGGACAATCTGGTGCTGAACAAGGAAGACCT | |
| | GGACGTCGCATCTCGCTTCCTGGAGATGGAGCCAGTCACCATCCCAGATGTCCAC | |
| | GGCGGATCCTTGCAGAATGCTGTCCGCGTGTGGAGCAACATCCCAGCCATAAGGA | |
| | GCAGGCACTGGGCTCTGGTTTCGGAAGAAGAATTGTCCCTGCTGGCCCAGAACAA | |
| | GCAGAGCTCGAAGCTCGCGGCCAAGTGGCCCACCAAGCTGGTGAAGAACTGCTTT | |
| | CTCCCCCTAAGAGAATATTTCAAGTATTTTTCAACAGAACTCACTTCCTCTTTA | |
| 197 | MAAIPALDPEAEPSMDVILVGSSELSSSVSPGTGRDLIAYEVKANQRNIEDICICCGSLQ | DNMT3L |
| | VHTQHPLFEGGICAPCKDKFLDALFLYDDDGYQSYCSICCSGETLLICGNPDCTRCYCF | (AA) |
| | ECVDSLVGPGTSGKVHAMSNWVCYLCLPSSRSGLLQRRRKWRSQLKAFYDRESENPL | |
| | EMFETVPVWRRQPVRVLSLFEDIKKELTSLGFLESGSDPGQLKHVVDVTDTVRKDVE | |
| | EWGPFDLVYGATPPLGHTCDRPPSWYLFQFHRLLQYARPKPGSPRPFFWMFVDNLVL | |
| | NKEDLDVASRFLEMEPVTIPDVHGGSLQNAVRVWSNIPAIRSRHWALVSEEELSLLAQ | |
| | NKQSSKLAAKWPTKLVKNCFLPLREYFKYFSTELTSSL | |
| 198 | AACCATGACCAGGAATTTGACCCCCCAAAGGTTTACCCACCTGTGCCAGCTGAGA | DNMT3A/ |
| | AGAGGAAGCCCATCCGCGTGCTGTCTCTCTTTGATGGGATTGCTACAGGGCTCCTG | L v1 (nt) |
| | GTGCTGAAGGACCTGGGCATCCAAGTGGACCGCTACATTGCCTCCGAGGTGTGTG | |
| | AGGACTCCATCACGGTGGGCATGGTGCGGCACCAGGGAAAGATCATGTACGTCGG | |
| | GGACGTCCGCAGCGTCACACAGAAGCATATCCAGGAGTGGGGCCCATTCGACCTG | |
| | GTGATTGGAGGCAGTCCCTGCAATGACCTCTCCATTGTCAACCCTGCCCGCAAGG | |
| | GACTTTATGAGGGTACTGGCCGCCTCTTCTTTGAGTTCTACCGCCTCCTGCATGAT | |
| | GCGCGGCCCAAGGAGGGAGATGATCGCCCCTTCTTCTGGCTCTTTGAGAATGTGG | |
| | TGGCCATGGGCGTTAGTGACAAGAGGGACATCTCGCGATTTCTTGAGTCTAACCC | |
| | CGTGATGATTGACGCCAAAGAAGTGTCTGCTGCACACAGGGCCCGTTACTTCTGG | |
| | GGTAACCTTCCTGGCATGAACAGGCCTTTGGCATCCACTGTGAATGATAAGCTGG | |
| | AGCTGCAAGAGTGTCTGGAGCACGGCAGAATAGCCAAGTTCAGCAAAGTGAGGA | |
| | CCATTACCACCAGGTCAAACTCTATAAAGCAGGGCAAAGACCAGCATTTCCCCGT | |
| | CTTCATGAACGAGAAGGAGGACATCCTGTGGTGCACTGAAATGGAAAGGGTGTTT | |
| | GGCTTCCCCGTCCACTACACAGACGTCTCCAACATGAGCCGCTTGGCGAGGCAGA | |
| | GACTGCTGGGCCGATCGTGGAGCGTGCCGGTCATCCGCCACCTCTTCGCTCCGCTG | |
| | AAGGAATATTTTGCTTGTGTGTCTAGCGGCAATAGTAACGCTAACAGCCGCGGGC | |
| | CGAGCTTCAGCAGCGGCCTGGTGCCGTTAAGCTTGCGCGGCAGCCATATGGGCCC | |
| | TATGGAGATATACAAGACAGTGTCTGCATGGAAGAGACAGCCAGTGCGGGTACTG | |
| | AGCCTCTTCAGAAACATCGACAAGGTACTAAAGAGTTTGGGCTTCTTGGAAAGCG | |
| | GTTCTGGTTCTGGGGGAGGAACGCTGAAGTACGTGGAAGATGTCACAAATGTCGT | |
| | GAGGAGAGACGTGGAGAAATGGGGCCCCTTTGACCTGGTGTACGGCTCGACGCAG | |
| | CCCCTAGGCAGCTCTTGTGATCGCTGTCCCGGCTGGTACATGTTCCAGTTCCACCG | |
| | GATCCTGCAGTATGCGCTGCCTCGCCAGGAGAGTCAGCGGCCCTTCTTCTGGATAT | |
| | TCATGGACAATCTGCTGCTGACTGAGGATGACCAAGAGACAACTACCCGCTTCCT | |
| | TCAGACAGAGGCTGTGACCCTCCAGGATGTCCGTGGCAGAGACTACCAGAATGCT | |
| | ATGCGGGTGTGGAGCAACATTCCAGGGCTGAAGAGCAAGCATGCGCCCCTGACCC | |
| | CAAAGGAAGAAGAGTATCTGCAAGCCCAAGTCAGAAGCAGGAGCAAGCTGGACG | |
| | CCCCGAAAGTTGACCTCCTGGTGAAGAACTGCCTTCTCCCGCTGAGAGAGTACTTC | |
| | AAGTATTTTTCTCAAAACTCACTTCCTCTT | |
| 199 | NHDQEFDPPKVYPPVPAEKRKPIRVLSLFDGIATGLLVLKDLGIQVDRYIASEVCEDSIT | DNMT3A/ |
| | VGMVRHQGKIMYVGDVRSVTQKHIQEWGPFDLVIGGSPCNDLSIVNPARKGLYEGTG | L v1 (AA) |
| | RLFFEFYRLLHDARPKEGDDRPFFWLFENVVAMGVSDKRDISRFLESNPVMIDAKEVS | |
| | AAHRARYFWGNLPGMNRPLASTVNDKLELQECLEHGRIAKFSKVRTITTRSNSIKQGK | |
| | DQHFPVFMNEKEDILWCTEMERVFGFPVHYTDVSNMSRLARQRLLGRSWSVPVIRHL | |
| | FAPLKEYFACVSSGNSNANSRGPSFSSGLVPLSLRGSHMGPMEIYKTVSAWKRQPVRV | |
| | LSLFRNIDKVLKSLGFLESGSGSGGGTLKYVEDVTNVVRRDVEKWGPFDLVYGSTQP | |
| | LGSSCDRCPGWYMFQFHRILQYALPRQESQRPFFWIFMDNLLLTEDDQETTTRFLQTE | |
| | AVTLQDVRGRDYQNAMRVWSNIPGLKSKHAPLTPKEEEYLQAQVRSRSKLDAPKVD | |
| | LLVKNCLLPLREYFKYFSQNSLPL | |
| 200 | AACCACGATCAGGAGTTTGACCCCCCTAAGGTGTACCCACCCGTGCCAGCCGAGA | DNMT3A/ |
| | AGAGGAAGCCCATCCGCGTGCTGTCCCTGTTCGACGGCATCGCCACAGGCCTGCT | L v2 (nt) |
| | GGTGCTGAAGGATCTGGGCATCCAGGTGGACAGATATATCGCCTCCGAGGTGTGC | |
| | GAGGATTCTATCACCGTGGGCATGGTGAGGCACCAGGGCAAGATCATGTACGTGG | |
| | GCGACGTGCGCAGCGTGACACAGAAGCACATCCAGGAGTGGGGACCCTTCGACCT | |
| | GGTCATCGGAGGCAGCCCCTGTAATGACCTGTCCATCGTGAACCCTGCAAGGAAG | |
| | GGCCTGTATGAGGGAACCGGCAGACTGTTCTTTGAGTTCTACAGGCTGCTGCACG | |
| | ACGCCCGCCCTAAGGAGGGCGATGACAGGCCATTCTTTTGGCTGTTTGAGAACGT | |
| | GGTGGCCATGGGCGTGAGCGACAAGCGGGATATCTCCAGATTCCTGGAGTCTAAT | |
| | CCCGTGATGATCGATGCAAAGGAGGTGTCTGCCGCACACAGGGCAAGGTACTTTT | |
| | GGGGAAATCTGCCTGGCATGAACCGCCCACTGGCCAGCACCGTGAACGACAAGCT | |
| | GGAGCTGCAGGAGTGCCTGGAGCACGGAAGGATCGCCAAGTTCTCCAAGGTGCG | |
| | GACAATCACCACAAGATCTAACAGCATCAAGCAGGGCAAGGATCAGCACTTCCCC | |
| | GTGTTCATGAATGAGAAGGAGGACATCCTGTGGTGTACCGAGATGGAGCGCGTGT | |
| | TCGGCTTTCCAGTGCACTATACAGACGTGAGCAATATGAGCCGGCTGGCAAGGCA | |
| | GAGACTGCTGGGCCGGTCCTGGTCTGTGCCAGTGATCAGACACCTGTTCGCCCCCC | |
| | TGAAGGAGTACTTTGCCTGCGTGTCTAGCGGCAACTCTAATGCCAACAGCAGAGG | |
| | CCCTTCCTTTTCCTCTGGCCTGGTGCCACTGTCTCTGAGGGGCAGCCACATGGGCC | |
| | CCATGGAGATCTACAAGACCGTGTCCGCCTGGAAGAGGCAGCCTGTGCGCGTGCT | |
| | GTCTCTGTTCCGCAACATCGACAAGGTGCTGAAGAGCCTGGGCTTTCTGGAGAGC | |
| | GGATCCGGATCTGGAGGAGGCACCCTGAAGTATGTGGAGGATGTGACAAATGTGG | |
| | TGCGGAGAGATGTGGAGAAGTGGGGCCCCTTCGATCTGGTGTACGGATCCACCCA | |
| | GCCACTGGGAAGCTCCTGCGATAGGTGTCCAGGATGGTATATGTTCCAGTTTCACA | |
| | GAATCCTGCAGTACGCACTGCCAAGGCAGGAGAGCCAGCGCCCTTTCTTTTGGAT | |
| | CTTTATGGACAACCTGCTGCTGACAGAGGATGACCAGGAGACAACAACCCGCTTC | |
| | CTGCAGACAGAGGCAGTGACCCTGCAGGATGTGAGGGGACGCGACTATCAGAAT | |
| | GCCATGCGGGTGTGGTCTAACATCCCTGGCCTGAAGAGCAAGCACGCCCCCCTGA | |
| | CCCCTAAGGAGGAGGAGTACCTGCAGGCCCAGGTGCGGAGCAGATCCAAGCTGG | |
| | ATGCCCCTAAGGTGGACCTGCTGGTGAAGAATTGTCTGCTGCCACTGCGGGAGTA | |
| | CTTCAAGTACTTTAGTCAGAATAGCCTGCCACTG | |
| 201 | NHDQEFDPPKVYPPVPAEKRKPIRVLSLFDGIATGLLVLKDLGIQVDRYIASEVCEDSIT | DNMT3A/ |
| | VGMVRHQGKIMYVGDVRSVTQKHIQEWGPFDLVIGGSPCNDLSIVNPARKGLYEGTG | L v2 (AA) |
| | RLFFEFYRLLHDARPKEGDDRPFFWLFENVVAMGVSDKRDISRFLESNPVMIDAKEVS | |
| | AAHRARYFWGNLPGMNRPLASTVNDKLELQECLEHGRIAKFSKVRTITTRSNSIKQGK | |
| | DQHFPVFMNEKEDILWCTEMERVFGFPVHYTDVSNMSRLARQRLLGRSWSVPVIRHL | |
| | FAPLKEYFACVSSGNSNANSRGPSFSSGLVPLSLRGSHMGPMEIYKTVSAWKRQPVRV | |
| | LSLFRNIDKVLKSLGFLESGSGSGGGTLKYVEDVTNVVRRDVEKWGPFDLVYGSTQP | |
| | LGSSCDRCPGWYMFQFHRILQYALPRQESQRPFFWIFMDNLLLTEDDQETTTRFLQTE | |
| | AVTLQDVRGRDYQNAMRVWSNIPGLKSKHAPLTPKEEEYLQAQVRSRSKLDAPKVD | |
| | LLVKNCLLPLREYFKYFSQNSLPL | |
| 202 | NGG | PAM- |
| | | SpCas9 |
| 203 | NNGRRT | PAM- |
| | | SaCas9 |
| 204 | MKRNYILGLDIGITSVGYGIIDYETRDVIDAGVRLFKEANVENNEGRRSKRGARRLKR | SaCas9 |
| | RRRHRIQRVKKLLFDYNLLTDHSELSGINPYEARVKGLSQKLSEEEFSAALLHLAKRR | (AA) |
| | GVHNVNEVEEDTGNELSTKEQISRNSKALEEKYVAELQLERLKKDGEVRGSINRFKTS | |
| | DYVKEAKQLLKVQKAYHQLDQSFIDTYIDLLETRRTYYEGPGEGSPFGWKDIKEWYE | |
| | MLMGHCTYFPEELRSVKYAYNADLYNALNDLNNLVITRDENEKLEYYEKFQIIENVF | |
| | KQKKKPTLKQIAKEILVNEEDIKGYRVTSTGKPEFTNLKVYHDIKDITARKEIIENAELL | |
| | DQIAKILTIYQSSEDIQEELTNLNSELTQEEIEQISNLKGYTGTHNLSLKAINLILDELWH | |
| | TNDNQIAIFNRLKLVPKKVDLSQQKEIPTTLVDDFILSPVVKRSFIQSIKVINAIIKKYGL | |
| | PNDIIIELAREKNSKDAQKMINEMQKRNRQTNERIEEIIRTTGKENAKYLIEKIKLHDM | |
| | QEGKCLYSLEAIPLEDLLNNPFNYEVDHIIPRSVSFDNSFNNKVLVKQEENSKKGNRTP | |
| | FQYLSSSDSKISYETFKKHILNLAKGKGRISKTKKEYLLEERDINRFSVQKDFINRNLVD | |
| | TRYATRGLMNLLRSYFRVNNLDVKVKSINGGFTSFLRRKWKFKKERNKGYKHHAED | |
| | ALIIANADFIFKEWKKLDKAKKVMENQMFEEKQAESMPEIETEQEYKEIFITPHQIKHI | |
| | KDFKDYKYSHRVDKKPNRELINDTLYSTRKDDKGNTLIVNNLNGLYDKDNDKLKKLI | |
| | NKSPEKLLMYHHDPQTYQKLKLIMEQYGDEKNPLYKYYEETGNYLTKYSKKDNGPV | |
| | IKKIKYYGNKLNAHLDITDDYPNSRNKVVKLSLKPYRFDVYLDNGVYKFVTVKNLDV | |
| | IKKENYYEVNSKCYEEAKKLKKISNQAEFIASFYNNDLIKINGELYRVIGVNNDLLNRI | |
| | EVNMIDITYREYLENMNDKRPPRIIKTIASKTQSIKKYSTDILGNLYEVKSKKHPQIIKK | |
| | G | |
| 205 | KRNYILGLAIGITSVGYGIIDYETRDVIDAGVRLFKEANVENNEGRRSKRGARRLKRRR | dSaCas9 |
| | RHRIQRVKKLLFDYNLLTDHSELSGINPYEARVKGLSQKLSEEEFSAALLHLAKRRGV | (AA) |
| | HNVNEVEEDTGNELSTKEQISRNSKALEEKYVAELQLERLKKDGEVRGSINRFKTSDY | |
| | VKEAKQLLKVQKAYHQLDQSFIDTYIDLLETRRTYYEGPGEGSPFGWKDIKEWYEML | |
| | MGHCTYFPEELRSVKYAYNADLYNALNDLNNLVITRDENEKLEYYEKFQIIENVFKQ | |
| | KKKPTLKQIAKEILVNEEDIKGYRVTSTGKPEFTNLKVYHDIKDITARKEIIENAELLDQ | |
| | IAKILTIYQSSEDIQEELTNLNSELTQEEIEQISNLKGYTGTHNLSLKAINLILDELWHTN | |
| | DNQIAIFNRLKLVPKKVDLSQQKEIPTTLVDDFILSPVVKRSFIQSIKVINAIIKKYGLPN | |
| | DIIIELAREKNSKDAQKMINEMQKRNRQTNERIEEIIRTTGKENAKYLIEKIKLHDMQE | |
| | GKCLYSLEAIPLEDLLNNPFNYEVDHIIPRSVSFDNSFNNKVLVKQEEASKKGNRTPFQ | |
| | YLSSSDSKISYETFKKHILNLAKGKGRISKTKKEYLLEERDINRFSVQKDFINRNLVDTR | |
| | YATRGLMNLLRSYFRVNNLDVKVKSINGGFTSFLRRKWKFKKERNKGYKHHAEDAL | |
| | IIANADFIFKEWKKLDKAKKVMENQMFEEKQAESMPEIETEQEYKEIFITPHQIKHIKD | |
| | FKDYKYSHRVDKKPNRELINDTLYSTRKDDKGNTLIVNNLNGLYDKDNDKLKKLINK | |
| | SPEKLLMYHHDPQTYQKLKLIMEQYGDEKNPLYKYYEETGNYLTKYSKKDNGPVIK | |
| | KIKYYGNKLNAHLDITDDYPNSRNKVVKLSLKPYRFDVYLDNGVYKFVTVKNLDVIK | |
| | KENYYEVNSKCYEEAKKLKKISNQAEFIASFYNNDLIKINGELYRVIGVNNDLLNRIEV | |
| | NMIDITYREYLENMNDKRPPRIIKTIASKTQSIKKYSTDILGNLYEVKSKKHPQIIKKG | |
| 206 | MDKKYSIGLDIGTNSVGWAVITDEYKVPSKKFKVLGNTDRHSIKKNLIGALLFDSGET | SpCas9 |
| | AEATRLKRTARRRYTRRKNRICYLQEIFSNEMAKVDDSFFHRLEESFLVEEDKKHERH | (AA) |
| | PIFGNIVDEVAYHEKYPTIYHLRKKLVDSTDKADLRLIYLALAHMIKFRGHFLIEGDLN | |
| | PDNSDVDKLFIQLVQTYNQLFEENPINASGVDAKAILSARLSKSRRLENLIAQLPGEKK | |
| | NGLFGNLIALSLGLTPNFKSNFDLAEDAKLQLSKDTYDDDLDNLLAQIGDQYADLFLA | |
| | AKNLSDAILLSDILRVNTEITKAPLSASMIKRYDEHHQDLTLLKALVRQQLPEKYKEIF | |
| | FDQSKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAWMTRKS | |
| | EETITPWNFEEVVDKGASAQSFIERMTNFDKNLPNEKVLPKHSLLYEYFTVYNELTKV | |
| | KYVTEGMRKPAFLSGEQKKAIVDLLFKTNRKVTVKQLKEDYFKKIECFDSVEISGVED | |
| | RFNASLGTYHDLLKIIKDKDFLDNEENEDILEDIVLTLTLFEDREMIEERLKTYAHLFD | |
| | DKVMKQLKRRRYTGWGRLSRKLINGIRDKQSGKTILDFLKSDGFANRNFMQLIHDDS | |
| | LTFKEDIQKAQVSGQGDSLHEHIANLAGSPAIKKGILQTVKVVDELVKVMGRHKPENI | |
| | VIEMARENQTTQKGQKNSRERMKRIEEGIKELGSQILKEHPVENTQLQNEKLYLYYLQ | |
| | NGRDMYVDQELDINRLSDYDVDHIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHV | |
| | AQILDSRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKR | |
| | VILADANLDKVLSAYNKHRDKPIREQAENIIHLFTLTNLGAPAAFKYFDTTIDRKRYTS | |
| | TKEVLDATLIHQSITGLYETRIDLSQLGGD | |
| 207 | DKKYSIGLAIGTNSVGWAVITDEYKVPSKKFKVLGNTDRHSIKKNLIGALLFDSGETA | dSpCas9 |
| | EATRLKRTARRRYTRRKNRICYLQEIFSNEMAKVDDSFFHRLEESFLVEEDKKHERHPI | (AA) |
| | FGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKN | |
| | GLFGNLIALSLGLTPNFKSNFDLAEDAKLQLSKDTYDDDLDNLLAQIGDQYADLFLAA | |
| | KNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWMTRKS | |
| | EETITPWNFEEVVDKGASAQSFIERMTNFDKNLPNEKVLPKHSLLYEYFTVYNELTKV | |
| | KYVTEGMRKPAFLSGEQKKAIVDLLFKTNRKVTVKQLKEDYFKKIECFDSVEISGVED | |
| | RFNASLGTYHDLLKIIKDKDFLDNEENEDILEDIVLTLTLFEDREMIEERLKTYAHLFD | |
| | DKVMKQLKRRRYTGWGRLSRKLINGIRDKQSGKTILDFLKSDGFANRNFMQLIHDDS | |
| | LTFKEDIQKAQVSGQGDSLHEHIANLAGSPAIKKGILQTVKVVDELVKVMGRHKPENI | |
| | VIEMARENQTTQKGQKNSRERMKRIEEGIKELGSQILKEHPVENTQLQNEKLYLYYLQ | |
| | NGRDMYVDQELDINRLSDYDVDAIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHV | |
| | AQILDSRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKR | |
| | VILADANLDKVLSAYNKHRDKPIREQAENIIHLFTLTNLGAPAAFKYFDTTIDRKRYTS | |
| | TKEVLDATLIHQSITGLYETRIDLSQLGGD | |
| 208 | CCAAAGAAGAAGCGGAAGGTCGGTATCCACGGAGTCCCAGCAGCCGACAAGAAG | dSpCas9- |
| | TACTCCATTGGGCTCGCCATCGGCACAAACAGCGTCGGCTGGGCCGTCATTACGG | KRAB (nt) |
| | ACGAGTACAAGGTGCCGAGCAAAAAATTCAAAGTTCTGGGCAATACCGATCGCCA | |
| | CAGCATAAAGAAGAACCTCATTGGCGCCCTCCTGTTCGACTCCGGGGAAACCGCC | |
| | GAAGCCACGCGGCTCAAAAGAACAGCACGGCGCAGATATACCCGCAGAAAGAAT | |
| | CGGATCTGCTACCtgcaGGAGATCTTTAGTAATGAGATGGCTAAGGTGGATGACTCT | |
| | TTCTTCCATAGGCTGGAGGAGTCCTTTTTGGTGGAGGAGGATAAAAAGCACGAGC | |
| | GCCACCCAATCTTTGGCAATATCGTGGACGAGGTGGCGTACCATGAAAAGTACCC | |
| | AACCATATATCATCTGAGGAAGAAGCTTGTAGACAGTACTGATAAGGCTGACTTG | |
| | CGGTTGATCTATCTCGCGCTGGCGCATATGATCAAATTTCGGGGACACTTCCTCAT | |
| | CGAGGGGGACCTGAACCCAGACAACAGCGATGTCGACAAACTCTTTATCCAACTG | |
| | GTTCAGACTTACAATCAGCTTTTCGAAGAGAACCCGATCAACGCATCCGGAGTTG | |
| | ACGCCAAAGCAATCCTGAGCGCTAGGCTGTCCAAATCCCGGCGGCTCGAAAACCT | |
| | CATCGCACAGCTCCCTGGGGAGAAGAAGAACGGCCTGTTTGGTAATCTTATCGCC | |
| | CTGTCACTCGGGCTGACCCCCAACTTTAAATCTAACTTCGACCTGGCCGAAGATGC | |
| | CAAGCTTCAACTGAGCAAAGACACCTACGATGATGATCTCGACAATCTGCTGGCC | |
| | CAGATCGGCGACCAGTACGCAGACCTTTTTTTGGCGGCAAAGAACCTGTCAGACG | |
| | CCATTCTGCTGAGTGATATTCTGCGAGTGAACACGGAGATCACCAAAGCTCCGCT | |
| | GAGCGCTAGTATGATCAAGCGCTATGATGAGCACCACCAAGACTTGACTTTGCTG | |
| | AAGGCCCTTGTCAGACAGCAACTGCCTGAGAAGTACAAGGAAATTTTCTTCGATC | |
| | AGTCTAAAAATGGCTACGCCGGATACATTGACGGCGGAGCAAGCCAGGAGGAAT | |
| | TTTACAAATTTATTAAGCCCATCTTGGAAAAAATGGACGGCACCGAGGAGCTGCT | |
| | GGTAAAGCTTAACAGAGAAGATCTGTTGCGCAAACAGCGCACTTTCGACAATGGA | |
| | AGCATCCCCCACCAGATTCACCTGGGCGAACTGCACGCTATCCTCAGGCGGCAAG | |
| | AGGATTTCTACCCCTTTTTGAAAGATAACAGGGAAAAGATTGAGAAAATCCTCAC | |
| | ATTTCGGATACCCTACTATGTAGGCCCCCTCGCCCGGGGAAATTCCAGATTCGCGT | |
| | GGATGACTCGCAAATCAGAAGAGACCATCACTCCCTGGAACTTCGAGGAAGTCGT | |
| | GGATAAGGGGGCCTCTGCCCAGTCCTTCATCGAAAGGATGACTAACTTTGATAAA | |
| | AATCTGCCTAACGAAAAGGTGCTTCCTAAACACTCTCTGCTGTACGAGTACTTCAC | |
| | AGTTTATAACGAGCTCACCAAGGTCAAATACGTCACAGAAGGGATGAGAAAGCC | |
| | AGCATTCCTGTCTGGAGAGCAGAAGAAAGCTATCGTGGACCTCCTCTTCAAGACG | |
| | AACCGGAAAGTTACCGTGAAACAGCTCAAAGAAGACTATTTCAAAAAGATTGAAT | |
| | GTTTCGACTCTGTTGAAATCAGCGGAGTGGAGGATCGCTTCAACGCATCCCTGGG | |
| | AACGTATCACGATCTCCTGAAAATCATTAAAGACAAGGACTTCCTGGACAATGAG | |
| | GAGAACGAGGACATTCTTGAGGACATTGTCCTCACCCTTACGTTGTTTGAAGATAG | |
| | GGAGATGATTGAAGAACGCTTGAAAACTTACGCTCATCTCTTCGACGACAAAGTC | |
| | ATGAAACAGCTCAAGAGGCGCCGATATACAGGATGGGGGCGGCTGTCAAGAAAA | |
| | CTGATCAATGGgatcCGAGACAAGCAGAGTGGAAAGACAATCCTGGATTTTCTTAA | |
| | GTCCGATGGATTTGCCAACCGGAACTTCATGCAGTTGATCCATGATGACTCTCTCA | |
| | CCTTTAAGGAGGACATCCAGAAAGCACAAGTTTCTGGCCAGGGGGACAGTCTTCA | |
| | CGAGCACATCGCTAATCTTGCAGGTAGCCCAGCTATCAAAAAGGGAATACTGCAG | |
| | ACCGTTAAGGTCGTGGATGAACTCGTCAAAGTAATGGGAAGGCATAAGCCCGAGA | |
| | ATATCGTTATCGAGATGGCCCGAGAGAACCAAACTACCCAGAAGGGACAGAAGA | |
| | ACAGTAGGGAAAGGATGAAGAGGATTGAAGAGGGTATAAAAGAACTGGGGTCCC | |
| | AAATCCTTAAGGAACACCCAGTTGAAAACACCCAGCTTCAGAATGAGAAGCTCTA | |
| | CCTGTACTACCTGCAGAACGGCAGGGACATGTACGTGGATCAGGAACTGGACATC | |
| | AATCGGCTCTCCGACTACGACGTGGATGCCATCGTGCCCCAGTCTTTTCTCAAAGA | |
| | TGATTCTATTGATAATAAAGTGTTGACAAGATCCGATAAAAATAGAGGGAAGAGT | |
| | GATAACGTCCCCTCAGAAGAAGTTGTCAAGAAAATGAAAAATTATTGGCGGCAGC | |
| | TGCTGAACGCCAAACTGATCACACAACGGAAGTTCGATAATCTGACTAAGGCTGA | |
| | ACGAGGTGGCCTGTCTGAGTTGGATAAAGCCGGCTTCATCAAAAGGCAGCTTGTT | |
| | GAGACACGCCAGATCACCAAgcacGTGGCCCAAATTCTCGATTCACGCATGAACAC | |
| | CAAGTACGATGAAAATGACAAACTGATTCGAGAGGTGAAAGTTATTACTCTGAAG | |
| | TCTAAGCTGGTCTCAGATTTCAGAAAGGACTTTCAGTTTTATAAGGTGAGAGAGAT | |
| | CAACAATTACCACCATGCGCATGATGCCTACCTGAATGCAGTGGTAGGCACTGCA | |
| | CTTATCAAAAAATATCCCAAGCTTGAATCTGAATTTGTTTACGGAGACTATAAAGT | |
| | GTACGATGTTAGGAAAATGATCGCAAAGTCTGAGCAGGAAATAGGCAAGGCCAC | |
| | CGCTAAGTACTTCTTTTACAGCAATATTATGAATTTTTTCAAGACCGAGATTACAC | |
| | TGGCCAATGGAGAGATTCGGAAGCGACCACTTATCGAAACAAACGGAGAAACAG | |
| | GAGAAATCGTGTGGGACAAGGGTAGGGATTTCGCGACAGTCCGGAAGGTCCTGTC | |
| | CATGCCGCAGGTGAACATCGTTAAAAAGACCGAAGTACAGACCGGAGGCTTCTCC | |
| | AAGGAAAGTATCCTCCCGAAAAGGAACAGCGACAAGCTGATCGCACGCAAAAAA | |
| | GATTGGGACCCCAAGAAATACGGCGGATTCGATTCTCCTACAGTCGCTTACAGTG | |
| | TACTGGTTGTGGCCAAAGTGGAGAAAGGGAAGTCTAAAAAACTCAAAAGCGTCA | |
| | AGGAACTGCTGGGCATCACAATCATGGAGCGATCAAGCTTCGAAAAAAACCCCAT | |
| | CGACTTTCTCGAGGCGAAAGGATATAAAGAGGTCAAAAAAGACCTCATCATTAAG | |
| | CTTCCCAAGTACTCTCTCTTTGAGCTTGAAAACGGCCGGAAACGAATGCTCGCTAG | |
| | TGCGGGCGAGCTGCAGAAAGGTAACGAGCTGGCACTGCCCTCTAAATACGTTAAT | |
| | TTCTTGTATCTGGCCAGCCACTATGAAAAGCTCAAAGGGTCTCCCGAAGATAATG | |
| | AGCAGAAGCAGCTGTTCGTGGAACAACACAAACACTACCTTGATGAGATCATCGA | |
| | GCAAATAAGCGAATTCTCCAAAAGAGTGATCCTCGCCGACGCTAACCTCGATAAG | |
| | GTGCTTTCTGCTTACAATAAGCACAGGGATAAGCCCATCAGGGAGCAGGCAGAAA | |
| | ACATTATCCACTTGTTTACTCTGACCAACTTGGGCGCGCCTGCAGCCTTCAAGTAC | |
| | TTCGACACCACCATAGACAGAAAGCGGTACACCTCTACAAAGGAGGTCCTGGACG | |
| | CCACACTGATTCATCAGTCAATTACGGGGCTCTATGAAACAAGAATCGACCTCTCT | |
| | CAGCTCGGTGGAGACAAAAGGCCGGCGGCCACGAAAAAGGCCGGCCAGGCAAAA | |
| | AAGAAAAAGGctagCgatgctaagtcactgactgcctggtcccggacactggtgaccttcaaggatgtgtttgtg | |
| | gacttcaccagggaggagtggaagctgctggacactgctcagcagatcctgtacagaaatgtgatgctggagaac | |
| | tataagaacctggtttccttgggttatcagcttactaagccagatgtgatcctccggttggagaagggagaagag | |
| | ccctggctggtggagagagaaattcaccaagagacccatcctgattcagagactgcatttgaaatcaaatca | |
| | tcagttCCGAAAAAGAAACGCAAAGTT | |
| 209 | PKKKRKVGIHGVPAADKKYSIGLAIGTNSVGWAVITDEYKVPSKKFKVLGNTDRHSIK | dSpCas9- |
| | KNLIGALLFDSGETAEATRLKRTARRRYTRRKNRICYLQEIFSNEMAKVDDSFFHRLE | KRAB |
| | ESFLVEEDKKHERHPIFGNIVDEVAYHEKYPTIYHLRKKLVDSTDKADLRLIYLALAH | (AA) |
| | MIKFRGHFLIEGDLNPDNSDVDKLFIQLVQTYNQLFEENPINASGVDAKAILSARLSKS | |
| | RRLENLIAQLPGEKKNGLFGNLIALSLGLTPNFKSNFDLAEDAKLQLSKDTYDDDLDN | |
| | LLAQIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKH | |
| | SLLYEYFTVYNELTKVKYVTEGMRKPAFLSGEQKKAIVDLLFKTNRKVTVKQLKEDY | |
| | FKKIECFDSVEISGVEDRFNASLGTYHDLLKIIKDKDFLDNEENEDILEDIVLTLTLFED | |
| | REMIEERLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGILQTVKV | |
| | VDELVKVMGRHKPENIVIEMARENQTTQKGQKNSRERMKRIEEGIKELGSQILKEHPV | |
| | ENTQLQNEKLYLYYLQNGRDMYVDQELDINRLSDYDVDAIVPQSFLKDDSIDNKVLT | |
| | RSDKNRGKSDNVPSEEVVKKMKNYWRQLLNAKLITQRKFDNLTKAERGGLSELDKA | |
| | GFIKRQLVETRQITKHVAQILDSRMNTKYDENDKLIREVKVITLKSKLVSDFRKDFQFY | |
| | KVREINNYHHAHDAYLNAVVGTALIKKYPKLESEFVYGDYKVYDVRKMIAKSEQEIG | |
| | KATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLV | |
| | VAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQ | |
| | HKHYLDEIIEQISEFSKRVILADANLDKVLSAYNKHRDKPIREQAENIIHLFTLTNLGAP | |
| | AAFKYFDTTIDRKRYTSTKEVLDATLIHQSITGLYETRIDLSQLGGDKRPAATKKAGQ | |
| | AKKKKASDAKSLTAWSRTLVTFKDVFVDFTREEWKLLDTAQQILYRNVMLENYKNL | |
| | VSLGYQLTKPDVILRLEKGEEPWLVEREIHQETHPDSETAFEIKSSVPKKKRKV | |
| 210 | NNNNGATT | PAM-N. |
| | | meningitidis |
| | | Cas9 |
| 211 | NNNNRYAC | PAM-C. |
| | | jejuni Cas9 |
| 212 | NNAGAAW | PAM-S. |
| | | thermophilus |
| 213 | NGG | PAM-F. |
| | | Novicida |
| 214 | NAAAAC | PAM-T. |
| | | denticola |
| 215 | TTTV | PAM- |
| | | Cas12a/Cpf1 |
| 216 | NGAN | Variant |
| | | PAM- |
| | | SpCas9 |
| | | variant |
| 217 | NGNG | Variant |
| | | PAM- |
| | | SpCas9 |
| | | variant |
| 218 | NGAG | Variant |
| | | PAM- |
| | | SpCas9 |
| | | variant |
| 219 | NGCG | Variant |
| | | PAM- |
| | | SpCas9 |
| | | variant |
| 220 | MKTPADTGFAFPDWAYKPESSPGSRQIQLWHFILELLRKEEYQGVIAWQGDYGEFVIK | ERF |
| | DPDEVARLWGVRKCKPQMNYDKLSRALRYYYNKRILHKTKGKRFTYKFNFNKLVLV | domain |
| | NYPFIDVGLAGGAVPQSAPPVPSGGSHFRFPPSTPSEVLSPTEDPRSPPACSSSSSSLFSA | (AA) |
| | VVARRLGRGSVSDCSDGTSELEEPLGEDPRARPPGPPDLGAFRGPPLARLPHDPGVFR | |
| | VYPRPRGGPEPLSPFPVSPLAGPGSLLPPQLSPALPMTPTHLAYTPSPTLSPMYPSGGGG | |
| | PSGSGGGSHFSFSPEDMKRYLQAHTQSVYNYHLSPRAFLHYPGLVVPQPQRPDKCPLP | |
| | PMAPETPPVPSSASSSSSSSSSPFKFKLQPPPLGRRQRAAGEKAVAGADKSGGSAGGLA | |
| | EGAGALAPPPPPPQIKVEPISEGESEEVEVTDISDEDEEDGEVFKTPRAPPAPPKPEPGEA | |
| | PGASQCMPLKLRFKRRWSEDCRLEGGGGPAGGFEDEGEDKKVRGEGPGEAGGPLTP | |
| | RRVSSDLQHATAQLSLEHRDS | |
| 221 | MERVKMINVQRLLEAAEFLERRERECEHGYASSFPSMPSPRLQHSKPPRRLSRAQKHS | MXI1 |
| | SGSSNTSTANRSTHNELEKNRRAHLRLCLERLKVLIPLGPDCTRHTTLGLLNKAKAHI | domain |
| | KKLEEAERKSQHQLENLEREQRFLKWRLEQLQGPQEMERIRMDSIGSTISSDRSDSERE | (AA) |
| | EIEVDVESTEFSHGEVDNISTTSISDIDDHSSLPSIGSDEGYSSASVKLSFTS | |
| 222 | ASPKKKRKVEASGSGMNIQMLLEAADYLERREREAEHGYASMLPGSGMNIQMLLEA | SID4X |
| | ADYLERREREAEHGYASMLPGSGMNIQMLLEAADYLERREREAEHGYASMLPGSGM | domain |
| | NIQMLLEAADYLERREREAEHGYASMLPSRSR | (AA) |
| 223 | MAAAVRMNIQMLLEAADYLERREREAEHGYASMLPYNNKDRDALKRRNKSKKNNS | MAD-SID |
| | SSRST | domain |
| | HNEMEKNRRAHLRLCLEKLKGLVPLGPESSRHTTLSLLTKAKLHIKKLEDCDRKAVH | (AA) |
| | QID | |
| | QLQREQRHLKRQLEKLGIERIRMDSIGSTVSSERSDSDREEIDVDVESTDYLTGDLDWS | |
| | S | |
| | SSVSDSDERGSMQSLGSDEGYSSTSIKRIKLQDSHKACLG | |
| 224 | MKGDTRHLNGEEDAGGREDSILVNGACSDQSSDSPPILEAIRTPEIRGRRSSSRLSKRE | DNMT3B |
| | VSSLLSYTQDLTGDGDGEDGDGSDTPVMPKLFRETRTRSESPAVRTRNNNSVSSRERH | (AA) |
| | RPSPRSTRGRQGRNHVDESPVEFPATRSLRRRATASAGTPWPSPPSSYLTIDLTDDTED | |
| | THGTPQSSSTPYARLAQDSQQGGMESPQVEADSGDGDSSEYQDGKEFGIGDLVWGKI | |
| | KGFSWWPAMVVSWKATSKRQAMSGMRWVQWFGDGKFSEVSADKLVALGLFSQHF | |
| | NLATFNKLVSYRKAMYHALEKARVRAGKTFPSSPGDSLEDQLKPMLEWAHGGFKPT | |
| | GIEGLKPNNTQPVVNKSKVRRAGSRKLESRKYENKTRRRTADDSATSDYCPAPKRLK | |
| | TNCYNNGKDRGDEDQSREQMASDVANNKSSLEDGCLSCGRKNPVSFHPLFEGGLCQ | |
| | TCRDRFLELFYMYDDDGYQSYCTVCCEGRELLLCSNTSCCRCFCVECLEVLVGTGTA | |
| | AEAKLQEPWSCYMCLPQRCHGVLRRRKDWNVRLQAFFTSDTGLEYEAPKLYPAIPA | |
| | ARRRPIRVLSLFDGIATGYLVLKELGIKVGKYVASEVCEESIAVGTVKHEGNIKYVND | |
| | VRNITKKNIEEWGPFDLVIGGSPCNDLSNVNPARKGLYEGTGRLFFEFYHLLNYSRPK | |
| | EGDDRPFFWMFENVVAMKVGDKRDISRFLECNPVMIDAIKVSAAHRARYFWGNLPG | |
| | MNRPVIASKNDKLELQDCLEYNRIAKLKKVQTITTKSNSIKQGKNQLFPVVMNGKED | |
| | VLWCTELERIFGFPVHYTDVSNMGRGARQKLLGRSWSVPVIRHLFAPLKDYFACE | |
| 225 | MLSGKKAAAAAAAAAAAATGTEAGPGTAGGSENGSEVAAQPAGLSGPAEVGPGAV | LSD1 (AA) |
| | GERTPRKKEPPRASPPGGLAEPPGSAGPQAGPTVVPGSATPMETGIAETPEGRRTSRRK | |
| | RAKVEYREMDESLANLSEDEYYSEEERNAKAEKEKKLPPPPPQAPPEEENESEPEEPSG | |
| | VEGAAFQSRLPHDRMTSQEAACFPDIISGPQQTQKVFLFIRNRTLQLWLDNPKIQLTFE | |
| | ATLQQLEAPYNSDTVLVHRVHSYLERHGLINFGIYKRIKPLPTKKTGKVIIIGSGVSGLA | |
| | AARQLQSFGMDVTLLEARDRVGGRVATFRKGNYVADLGAMVVTGLGGNPMAVVS | |
| | KQVNMELAKIKQKCPLYEANGQAVPKEKDEMVEQEFNRLLEATSYLSHQLDFNVLN | |
| | NKPVSLGQALEVVIQLQEKHVKDEQIEHWKKIVKTQEELKELLNKMVNLKEKIKELH | |
| | QQYKEASEVKPPRDITAEFLVKSKHRDLTALCKEYDELAETQGKLEEKLQELEANPPS | |
| | DVYLSSRDRQILDWHFANLEFANATPLSTLSLKHWDQDDDFEFTGSHLTVRNGYSCV | |
| | PVALAEGLDIKLNTAVRQVRYTASGCEVIAVNTRSTSQTFIYKCDAVLCTLPLGVLKQ | |
| | QPPAVQFVPPLPEWKTSAVQRMGFGNLNKVVLCFDRVFWDPSVNLFGHVGSTTASR | |
| | GELFLFWNLYKAPILLALVAGEAAGIMENISDDVIVGRCLAILKGIFGSSAVPQPKETV | |
| | VSRWRADPWARGSYSYVAAGSSGNDYDLMAQPITPGPSIPGAPQPIPRLFFAGEHTIR | |
| | NYPATVHGALLSGLREAGRIADQFLGAMYTLPRQATPGVPAQQSPSM | |
| 226 | LLPKNYHLENEVARLKKLVGER | SunTag |
| | | GCN4 |
| | | peptide |
| | | (AA) |
| 227 | GGSGG | GGSGG |
| | | linker (AA) |
| 228 | GGGGS | GGGGS |
| | | linker (AA) |
| 229 | GGGGG | GGGGG |
| | | linker (AA) |
| 230 | GGAGG | GGAGG |
| | | linker (AA) |
| 231 | GGGGSSS | GGGGSSS |
| | | linker (AA) |
| 232 | GGGGAAA | GGGGAAA |
| | | linker |
| | | (AA) |
| 233 | GGPSSGAPPPSGGSPAGSPTSTEEGTSESATPESGPGTSTEPSEGSAPGSPAGSPTSTEEG | XTEN80 |
| | TSTEPSEGSAPGTSTEPSE | (aa) |
| 234 | PKKKRKV | NLS- |
| | | SV40 (AA) |
| 235 | PAAKRVKLD | NLS- |
| | | cMyc (AA) |
| 236 | RQRRNELKRSP | NLS- |
| | | cMyc (AA) |
| 237 | NQSSNFGPMKGGNFGGRSSGPYGGGGQYFAKPRNQGGY | NLS- |
| | | hRNPA1 |
| | | M9 (AA) |
| 238 | RMRIZFKNKGKDTAELRRRRVEVSVELRKAKKDEQILKRRNV | NLS-IBB |
| | | domain |
| | | importin- |
| | | alpha (AA) |
| 239 | VSRKRPRP | NLS- |
| | | myoma T |
| | | protein |
| | | (AA) |
| 240 | PPKKARED | NLS- |
| | | myoma T |
| | | protein |
| | | (AA) |
| 241 | PQPKKKPL | NLS- |
| | | human p53 |
| | | (AA) |
| 242 | SALIKKKKKMAP | NLS- |
| | | mouse c- |
| | | abl IV |
| | | (AA) |
| 243 | DRLRR | NLS- |
| | | influenza |
| | | NS1 (AA) |
| 244 | PKQKKRK | NLS- |
| | | influenza |
| | | NS1 (AA) |
| 245 | RKLKKKIKKL | NLS- |
| | | hepatitis |
| | | delta |
| | | antigen |
| | | (AA) |
| 246 | REKKKFLKRR | NLS- |
| | | mouse Mx1 |
| | | (AA) |
| 247 | KRKGDEVDGVDEVAKKKSKK | NLS- |
| | | human |
| | | poly(ADP- |
| | | ribose) |
| | | polymerase |
| | | (AA) |
| 248 | RKCLQAGMNLEARKTKK | NLS- |
| | | gluco- |
| | | corticoid |
| | | (AA) |
| 249 | KRPAATKKAGQAKKKK | NLS- |
| | | nucleo- |
| | | plasmin (AA) |
| 250 | GGGAAGGGATACAGGCTGGA | gRNA- |
| | | mousePCS |
| | | K9-A |
| | | spacer |
| | | DNA |
| 251 | GTCCCGTTTGCAGCCCAATT | gRNA- |
| | | mousePCS |
| | | K9-B |
| | | spacer |
| | | DNA |
| 252 | GAGCGTCATTTGACGCTGTC | gRNA- |
| | | mousePCS |
| | | K9-C |
| | | spacer |
| | | DNA |
| 253 | GGATCTTCCGATGGGGCTCG | gRNA- |
| | | mousePCS |
| | | K9-D |
| | | spacer |
| | | DNA |
| 254 | GTGAAGGTGGAAGCCTTCTG | gRNA- |
| | | mousePCS |
| | | K9-E |
| | | spacer |
| | | DNA |
| 255 | GTGGACGCGCAGGCTGCCGG | gRNA- |
| | | mousePCS |
| | | K9-F |
| | | spacer |
| | | DNA |
| 256 | GGGGCGAGGAGAGGTGCGCG | gRNA- |
| | | mousePCS |
| | | K9-G |
| | | spacer |
| | | DNA |
| 257 | AGTGGGTGCCCATCGGGGCG | gRNA- |
| | | mousePCS |
| | | K9-H |
| | | spacer |
| | | DNA |
| 258 | CTACTGTGCCCCACCGGCGC | gRNA- |
| | | mousePCS |
| | | K9-I spacer |
| | | DNA |
| 259 | GGGAAGGGAUACAGGCUGGA | gRNA- |
| | | mousePCS |
| | | K9-A |
| | | spacer |
| | | RNA |
| 260 | GUCCCGUUUGCAGCCCAAUU | gRNA- |
| | | mousePCS |
| | | K9-B |
| | | spacer |
| | | RNA |
| 261 | GAGCGUCAUUUGACGCUGUC | gRNA- |
| | | mousePCS |
| | | K9-C |
| | | spacer |
| | | RNA |
| 262 | GGAUCUUCCGAUGGGGCUCG | gRNA- |
| | | mousePCS |
| | | K9-D |
| | | spacer |
| | | RNA |
| 263 | GUGAAGGUGGAAGCCUUCUG | gRNA- |
| | | mousePCS |
| | | K9-E |
| | | spacer |
| | | RNA |
| 264 | GUGGACGCGCAGGCUGCCGG | gRNA- |
| | | mousePCS |
| | | K9-F |
| | | spacer |
| | | RNA |
| 265 | GGGGCGAGGAGAGGUGCGCG | gRNA- |
| | | mousePCS |
| | | K9-G |
| | | spacer |
| | | RNA |
| 266 | AGUGGGUGCCCAUCGGGGCG | gRNA- |
| | | mousePCS |
| | | K9-H |
| | | spacer |
| | | RNA |
| 267 | CUACUGUGCCCCACCGGCGC | gRNA- |
| | | mousePCS |
| | | K9-I spacer |
| | | RNA |
| 268 | GGGAAGGGAUACAGGCUGGAGUUUAAGAGCUAUGCUGGAAACAGCAUAGCAAG | gRNA- |
| | UUUAAAUAAGGCUAGUCCGUUAUCAACUUGAAAAAGUGGCACCGAGUCGGUGC | mousePCS |
| | | K9-A |
| | | gRNA |
| 269 | GUCCCGUUUGCAGCCCAAUUGUUUAAGAGCUAUGCUGGAAACAGCAUAGCAAG | gRNA- |
| | UUUAAAUAAGGCUAGUCCGUUAUCAACUUGAAAAAGUGGCACCGAGUCGGUGC | mousePCS |
| | | K9-B |
| | | gRNA |
| 270 | GAGCGUCAUUUGACGCUGUCGUUUAAGAGCUAUGCUGGAAACAGCAUAGCAAG | gRNA- |
| | UUUAAAUAAGGCUAGUCCGUUAUCAACUUGAAAAAGUGGCACCGAGUCGGUGC | mousePCS |
| | | K9-C |
| | | gRNA |
| 271 | GGAUCUUCCGAUGGGGCUCGGUUUAAGAGCUAUGCUGGAAACAGCAUAGCAAG | gRNA- |
| | UUUAAAUAAGGCUAGUCCGUUAUCAACUUGAAAAAGUGGCACCGAGUCGGUGC | mousePCS |
| | | K9-D |
| | | gRNA |
| 272 | GUGAAGGUGGAAGCCUUCUGGUUUAAGAGCUAUGCUGGAAACAGCAUAGCAAG | gRNA- |
| | UUUAAAUAAGGCUAGUCCGUUAUCAACUUGAAAAAGUGGCACCGAGUCGGUGC | mousePCS |
| | | K9-E |
| | | gRNA |
| 273 | GUGGACGCGCAGGCUGCCGGGUUUAAGAGCUAUGCUGGAAACAGCAUAGCAAG | gRNA- |
| | UUUAAAUAAGGCUAGUCCGUUAUCAACUUGAAAAAGUGGCACCGAGUCGGUGC | mousePCS |
| | | K9-F |
| | | gRNA |
| 274 | GGGGCGAGGAGAGGUGCGCGGUUUAAGAGCUAUGCUGGAAACAGCAUAGCAAG | gRNA- |
| | UUUAAAUAAGGCUAGUCCGUUAUCAACUUGAAAAAGUGGCACCGAGUCGGUGC | mousePCS |
| | | K9-G |
| | | gRNA |
| 275 | AGUGGGUGCCCAUCGGGGCGGUUUAAGAGCUAUGCUGGAAACAGCAUAGCAAG | gRNA- |
| | UUUAAAUAAGGCUAGUCCGUUAUCAACUUGAAAAAGUGGCACCGAGUCGGUGC | mousePCS |
| | | K9-H |
| | | gRNA |
| 276 | CUACUGUGCCCCACCGGCGCGUUUAAGAGCUAUGCUGGAAACAGCAUAGCAAG | gRNA- |
| | UUUAAAUAAGGCUAGUCCGUUAUCAACUUGAAAAAGUGGCACCGAGUCGGUGC | mousePCS |
| | | K9-I gRNA |
| 277 | AACcacgatcaggagtttgacccccctaaggtgtacccacccgtgccagccgagaagaggaagcccatccgcgt | dSpCas9- |
| | gctgtccctgttcgacggcatcgccacaggcctgctggtgctgaaggatctgggcatccaggtggacagatata | KRAB- |
| | tcgcctccgaggtgtgcgaggattctatcaccgtgggcatggtgaggcaccagggcaagatcatgtacgtgggc | DNTM3A/ |
| | gacgtgcgcagcgtgacacagaagcacatccaggagtggggacccttcgacctggtcatcggaggcagcccctg | L (nt) |
| | taatgacctgtccatcgtgaaccctgcaaggaagggcctgtatgagggaaccggcagactgttctttgagttct | |
| | acaggctgctgcacgacgcccgccctaaggagggcgatgacaggccattcttttggctgtttgagaacgtggtg | |
| | gccatgggcgtgagcgacaagcgggatatctccagattcctggagtctaatcccgtgatgatcgatgcaaagga | |
| | ggtgtctgccgcacacagggcaaggtacttttggggaaatctgcctggcatgaaccgcccactggccagcaccg | |
| | tgaacgacaagctggagctgcaggagtgcctggagcacggaaggatcgccaagttctccaaggtgcggacaatc | |
| | accacaagatctaacagcatcaagcagggcaaggatcagcacttccccgtgttcatgaatgagaaggaggacat | |
| | cctgtggtgtaccgagatggagcgcgtgttcggctttccagtgcactatacagacgtgagcaatatgagccggc | |
| | tggcaaggcagagactgctgggccggtcctggtctgtgccagtgatcagacacctgttcgcccccctgaaggag | |
| | tactttgcctgcgtgtctagcggcaactctaatgccaacagcagaggcccttccttttcctctggcctggtgcc | |
| | actgtctctgaggggcagccacatgggccccatggagatctacaagaccgtgtccgcctggaagaggcagcctg | |
| | tgcgcgtgctgtctctgttccgcaacatcgacaaggtgctgaagagcctgggctttctggagagcggatccgga | |
| | tctggaggaggcaccctgaagtatgtggaggatgtgacaaatgtggtgcggagagatgtggagaagtggggccc | |
| | cttcgatctggtgtacggatccacccagccactgggaagctcctgcgataggtgtccaggatggtatatgttcc | |
| | agtttcacagaatcctgcagtacgcactgccaaggcaggagagccagcgccctttcttttggatctttatggac | |
| | aacctgctgctgacagaggatgaccaggagacaacaacccgcttcctgcagacagaggcagtgaccctgcagga | |
| | tgtgaggggacgcgactatcagaatgccatgcgggtgtggtctaacatccctggcctgaagagcaagcacgccc | |
| | ccctgacccctaaggaggaggagtacctgcaggcccaggtgcggagcagatccaagctggatgcccctaaggtg | |
| | gacctgctggtgaagaattgtctgctgccactgcgggagtacttcaagtactttagtcagaatagcctgccact | |
| | ggaggcaagcggatccggaagggcatctcctggaatcccaggaagcacccgcAACCCCAAGAAGAAGCGGAAGG | |
| | TGGGCATCCACGGCGTGCCCGCCGCCGACAAGAAGTACAGCATCGGCCTGGCCATCGGCACCAACAGCGTGGGC | |
| | TGGGCCGTGATCACCGACGAGTACAAGGTGCCCAGCAAGAAGTTCAAGGTGCTGGGCAACACCG | |
| | ACCGGCACAGCATCAAGAAGAACCTGATCGGCGCCCTGCTGTTCGACAGCGGCGA | |
| | GACCGCCGAGGCCACCCGGCTGAAGCGGACCGCCCGGCGGCGGTACACCCGGCG | |
| | GAAGAACCGGATCTGCTACCTGCAGGAGATCTTCAGCAACGAGATGGCCAAGGTG | |
| | GACGACAGCTTCTTCCACCGGCTGGAGGAGAGCTTCCTGGTGGAGGAGGACAAGA | |
| | AGCACGAGCGGCACCCCATCTTCGGCAACATCGTGGACGAGGTGGCCTACCACGA | |
| | GAAGTACCCCACCATCTACCACCTGCGGAAGAAGCTGGTGGACAGCACCGACAAG | |
| | GCCGACCTGCGGCTGATCTACCTGGCCCTGGCCCACATGATCAAGTTCCGGGGCC | |
| | ACTTCCTGATCGAGGGCGACCTGAACCCCGACAACAGCGACGTGGACAAGCTGTT | |
| | CATCCAGCTGGTGCAGACCTACAACCAGCTGTTCGAGGAGAACCCCATCAACGCC | |
| | AGCGGCGTGGACGCCAAGGCCATCCTGAGCGCCCGGCTGAGCAAGAGCCGGCGG | |
| | CTGGAGAACCTGATCGCCCAGCTGCCCGGCGAGAAGAAGAACGGCCTGTTCGGCA | |
| | ACCTGATCGCCCTGAGCCTGGGCCTGACCCCCAACTTCAAGAGCAACTTCGACCT | |
| | GGCCGAGGACGCCAAGCTGCAGCTGAGCAAGGACACCTACGACGACGACCTGGA | |
| | CAACCTGCTGGCCCAGATCGGCGACCAGTACGCCGACCTGTTCCTGGCCGCCAAG | |
| | AACCTGAGCGACGCCATCCTGCTGAGCGACATCCTGCGGGTGAACACCGAGATCA | |
| | CCAAGGCCCCCCTGAGCGCCAGCATGATCAAGCGGTACGACGAGCACCACCAGG | |
| | ACCTGACCCTGCTGAAGGCCCTGGTGCGGCAGCAGCTGCCCGAGAAGTACAAGGA | |
| | GATCTTCTTCGACCAGAGCAAGAACGGCTACGCCGGCTACATCGACGGCGGCGCC | |
| | AGCCAGGAGGAGTTCTACAAGTTCATCAAGCCCATCCTGGAGAAGATGGACGGCA | |
| | CCGAGGAGCTGCTGGTGAAGCTGAACCGGGAGGACCTGCTGCGGAAGCAGCGGA | |
| | CCTTCGACAACGGCAGCATCCCCCACCAGATCCACCTGGGCGAGCTGCACGCCAT | |
| | CCTGCGGCGGCAGGAGGACTTCTACCCCTTCCTGAAGGACAACCGGGAGAAGATC | |
| | GAGAAGATCCTGACCTTCCGGATCCCCTACTACGTGGGCCCCCTGGCCCGGGGCA | |
| | ACAGCCGGTTCGCCTGGATGACCCGGAAGAGCGAGGAGACCATCACCCCCTGGAA | |
| | CTTCGAGGAGGTGGTGGACAAGGGCGCCAGCGCCCAGAGCTTCATCGAGCGGATG | |
| | ACCAACTTCGACAAGAACCTGCCCAACGAGAAGGTGCTGCCCAAGCACAGCCTGC | |
| | TGTACGAGTACTTCACCGTGTACAACGAGCTGACCAAGGTGAAGTACGTGACCGA | |
| | GGGCATGCGGAAGCCCGCCTTCCTGAGCGGCGAGCAGAAGAAGGCCATCGTGGA | |
| | CCTGCTGTTCAAGACCAACCGGAAGGTGACCGTGAAGCAGCTGAAGGAGGACTAC | |
| | TTCAAGAAGATCGAGTGCTTCGACAGCGTGGAGATCAGCGGCGTGGAGGACCGGT | |
| | TCAACGCCAGCCTGGGCACCTACCACGACCTGCTGAAGATCATCAAGGACAAGGA | |
| | CTTCCTGGACAACGAGGAGAACGAGGACATCCTGGAGGACATCGTGCTGACCCTG | |
| | ACCCTGTTCGAGGACCGGGAGATGATCGAGGAGCGGCTGAAGACCTACGCCCACC | |
| | TGTTCGACGACAAGGTGATGAAGCAGCTGAAGCGGCGGCGGTACACCGGCTGGG | |
| | GCCGGCTGAGCCGGAAGCTGATCAACGGCATCCGGGACAAGCAGAGCGGCAAGA | |
| | CCATCCTGGACTTCCTGAAGAGCGACGGCTTCGCCAACCGGAACTTCATGCAGCT | |
| | GATCCACGACGACAGCCTGACCTTCAAGGAGGACATCCAGAAGGCCCAGGTGAG | |
| | CGGCCAGGGCGACAGCCTGCACGAGCACATCGCCAACCTGGCCGGCAGCCCCGCC | |
| | ATCAAGAAGGGCATCCTGCAGACCGTGAAGGTGGTGGACGAGCTGGTGAAGGTG | |
| | ATGGGCCGGCACAAGCCCGAGAACATCGTGATCGAGATGGCCCGGGAGAACCAG | |
| | ACCACCCAGAAGGGCCAGAAGAACAGCCGGGAGCGGATGAAGCGGATCGAGGAG | |
| | GGCATCAAGGAGCTGGGCAGCCAGATCCTGAAGGAGCACCCCGTGGAGAACACC | |
| | CAGCTGCAGAACGAGAAGCTGTACCTGTACTACCTGCAGAACGGCCGGGACATGT | |
| | ACGTGGACCAGGAGCTGGACATCAACCGGCTGAGCGACTACGACGTGGACGCCAT | |
| | CGTGCCCCAGAGCTTCCTGAAGGACGACAGCATCGACAACAAGGTGCTGACCCGG | |
| | AGCGACAAGAACCGGGGCAAGAGCGACAACGTGCCCAGCGAGGAGGTGGTGAAG | |
| | AAGATGAAGAACTACTGGCGGCAGCTGCTGAACGCCAAGCTGATCACCCAGCGG | |
| | AAGTTCGACAACCTGACCAAGGCCGAGCGGGGCGGCCTGAGCGAGCTGGACAAG | |
| | GCCGGCTTCATCAAGCGGCAGCTGGTGGAGACCCGGCAGATCACCAAGCACGTGG | |
| | CCCAGATCCTGGACAGCCGGATGAACACCAAGTACGACGAGAACGACAAGCTGA | |
| | TCCGGGAGGTGAAGGTGATCACCCTGAAGAGCAAGCTGGTGAGCGACTTCCGGAA | |
| | GGACTTCCAGTTCTACAAGGTGCGGGAGATCAACAACTACCACCACGCCCACGAC | |
| | GCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGC | |
| | CAAGAGCGAGCAGGAGATCGGCAAGGCCACCGCCAAGTACTTCTTCTACAGCAAC | |
| | ATCATGAACTTCTTCAAGACCGAGATCACCCTGGCCAACGGCGAGATCCGGAAGC | |
| | GGCCCCTGATCGAGACCAACGGCGAGACCGGCGAGATCGTGTGGGACAAGGGCC | |
| | GGGACTTCGCCACCGTGCGGAAGGTGCTGAGCATGCCCCAGGTGAACATCGTGAA | |
| | GAAGACCGAGGTGCAGACCGGCGGCTTCAGCAAGGAGAGCATCCTGCCCAAGCG | |
| | GAACAGCGACAAGCTGATCGCCCGGAAGAAGGACTGGGACCCCAAGAAGTACGG | |
| | CGGCTTCGACAGCCCCACCGTGGCCTACAGCGTGCTGGTGGTGGCCAAGGTGGAG | |
| | AAGGGCAAGAGCAAGAAGCTGAAGAGCGTGAAGGAGCTGCTGGGCATCACCATC | |
| | ATGGAGCGGAGCAGCTTCGAGAAGAACCCCATCGACTTCCTGGAGGCCAAGGGCT | |
| | ACAAGGAGGTGAAGAAGGACCTGATCATCAAGCTGCCCAAGTACAGCCTGTTCGA | |
| | GCTGGAGAACGGCCGGAAGCGGATGCTGGCCAGCGCCGGCGAGCTGCAGAAGGG | |
| | CAACGAGCTGGCCCTGCCCAGCAAGTACGTGAACTTCCTGTACCTGGCCAGCCAC | |
| | TACGAGAAGCTGAAGGGCAGCCCCGAGGACAACGAGCAGAAGCAGCTGTTCGTG | |
| | GAGCAGCACAAGCACTACCTGGACGAGATCATCGAGCAGATCAGCGAGTTCAGC | |
| | AAGCGGGTGATCCTGGCCGACGCCAACCTGGACAAGGTGCTGAGCGCCTACAACA | |
| | AGCACCGGGACAAGCCCATCCGGGAGCAGGCCGAGAACATCATCCACCTGTTCAC | |
| | CCTGACCAACCTGGGCGCCCCCGCCGCCTTCAAGTACTTCGACACCACCATCGACC | |
| | GGAAGCGGTACACCAGCACCAAGGAGGTGCTGGACGCCACCCTGATCCACCAGA | |
| | GCATCACCGGCCTGTACGAGACCCGGATCGACCTGAGCCAGCTGGGCGGCGACAG | |
| | CGGCGGCAAGCGGCCCGCCGCCACCAAGAAGGCCGGCCAGGCCAAGAAGAAGAA | |
| | GGctagCgatgctaagtcactgactgcctggtcccggacactggtgaccttcaaggatgtgtttgtggactt | |
| | caccagggaggagtggaagctgctggacactgctcagcagatcctgtacagaaatgtgatgctggagaacta | |
| | taagaacctggtttccttgggttatcagcttactaagccagatgtgatcctccggttggagaagggagaaga | |
| | gccctggctggtggagagagaaattcaccaagagacccatcctgattcagagactgcatttgaaatcaaatc | |
| | atcagttCCGAAAAAGAAACGCAAAGTTTAG | |
| 278 | NHDQEFDPPKVYPPVPAEKRKPIRVLSLFDGIATGLLVLKDLGIQVDRYIASEVCEDSIT | dSpCas9- |
| | VGMVRHQGKIMYVGDVRSVTQKHIQEWGPFDLVIGGSPCNDLSIVNPARKGLYEGTG | KRAB- |
| | RLFFEFYRLLHDARPKEGDDRPFFWLFENVVAMGVSDKRDISRFLESNPVMIDAKEVS | DNTM3A/ |
| | AAHRARYFWGNLPGMNRPLASTVNDKLELQECLEHGRIAKFSKVRTITTRSNSIKQGK | L (AA) |
| | DQHFPVFMNEKEDILWCTEMERVFGFPVHYTDVSNMSRLARQRLLGRSWSVPVIRHL | |
| | FAPLKEYFACVSSGNSNANSRGPSFSSGLVPLSLRGSHMGPMEIYKTVSAWKRQPVRV | |
| | LSLFRNIDKVLKSLGFLESGSGSGGGTLKYVEDVTNVVRRDVEKWGPFDLVYGSTQP | |
| | LGSSCDRCPGWYMFQFHRILQYALPRQESQRPFFWIFMDNLLLTEDDQETTTRFLQTE | |
| | AVTLQDVRGRDYQNAMRVWSNIPGLKSKHAPLTPKEEEYLQAQVRSRSKLDAPKVD | |
| | LLVKNCLLPLREYFKYFSQNSLPLEASGSGRASPGIPGSTRNPKKKRKVGIHGVPAAD | |
| | KKYSIGLAIGTNSVGWAVITDEYKVPSKKFKVLGNTDRHSIKKNLIGALLFDSGETAE | |
| | ATRLKRTARRRYTRRKNRICYLQEIFSNEMAKVDDSFFHRLEESFLVEEDKKHERHPIF | |
| | GNIVDEVAYHEKYPTIYHLRKKLVDSTDKADLRLIYLALAHMIKFRGHFLIEGDLNPD | |
| | NSDVDKLFIQLVQTYNQLFEENPINASGVDAKAILSARLSKSRRLENLIAQLPGEKKNG | |
| | LFGNLIALSLGLTPNFKSNFDLAEDAKLQLSKDTYDDDLDNLLAQIGDQYADLFLAAK | |
| | NLSDAILLSDILRVNTEITKAPLSASMIKRYDEHHQDLTLLKALVRQQLPEKYKEIFFD | |
| | QSKNGYAGYIDGGASQEEFYKFIKPILEKMDGTEELLVKLNREDLLRKQRTFDNGSIP | |
| | HQIHLGELHAILRRQEDFYPFLKDNREKIEKILTFRIPYYVGPLARGNSRFAWMTRKSE | |
| | ETITPWNFEEVVDKGASAQSFIERMTNFDKNLPNEKVLPKHSLLYEYFTVYNELTKVK | |
| | YVTEGMRKPAFLSGEQKKAIVDLLFKTNRKVTVKQLKEDYFKKIECFDSVEISGVEDR | |
| | FNASLGTYHDLLKIIKDKDFLDNEENEDILEDIVLTLTLFEDREMIEERLKTYAHLFDD | |
| | KVMKQLKRRRYTGWGRLSRKLINGIRDKQSGKTILDFLKSDGFANRNFMQLIHDDSL | |
| | TFKEDIQKAQVSGQGDSLHEHIANLAGSPAIKKGILQTVKVVDELVKVMGRHKPENIV | |
| | IEMARENQTTQKGQKNSRERMKRIEEGIKELGSQILKEHPVENTQLQNEKLYLYYLQN | |
| | GRDMYVDQELDINRLSDYDVDAIVPQSFLKDDSIDNKVLTRSDKNRGKSDNVPSEEV | |
| | VKKMKNYWRQLLNAKLITQRKFDNLTKAERGGLSELDKAGFIKRQLVETRQITKHVA | |
| | QILDSRMNTKYDENDKLIREVKVITLKSKLVSDFRKDFQFYKVREINNYHHAHDAYL | |
| | NAVVGTALIKKYPKLESEFVYGDYKVYDVRKMIAKSEQEIGKATAKYFFYSNIMNFF | |
| | KTEITLANGEIRKRPLIETNGETGEIVWDKGRDFATVRKVLSMPQVNIVKKTEVQTGG | |
| | FSKESILPKRNSDKLIARKKDWDPKKYGGFDSPTVAYSVLVVAKVEKGKSKKLKSVK | |
| | ELLGITIMERSSFEKNPIDFLEAKGYKEVKKDLIIKLPKYSLFELENGRKRMLASAGELQ | |
| | KGNELALPSKYVNFLYLASHYEKLKGSPEDNEQKQLFVEQHKHYLDEIIEQISEFSKRV | |
| | ILADANLDKVLSAYNKHRDKPIREQAENIIHLFTLTNLGAPAAFKYFDTTIDRKRYTST | |
| | KEVLDATLIHQSITGLYETRIDLSQLGGDSGGKRPAATKKAGQAKKKKASDAKSLTA | |
| | WSRTLVTFKDVFVDFTREEWKLLDTAQQILYRNVMLENYKNLVSLGYQLTKPDVILR | |
| | LEKGEEPWLVEREIHQETHPDSETAFEIKSSVPKKKRKV | |
| 279 | ATGAACCACGATCAGGAGTTTGACCCCCCTAAGGTGTACCCACCCGTGCCAGCCG | DNMT3A/ |
| | AGAAGAGGAAGCCCATCCGCGTGCTGTCCCTGTTCGACGGCATCGCCACAGGCCT | L- |
| | GCTGGTGCTGAAGGATCTGGGCATCCAGGTGGACAGATATATCGCCTCCGAGGTG | XTEN80- |
| | TGCGAGGATTCTATCACCGTGGGCATGGTGAGGCACCAGGGCAAGATCATGTACG | dSpCas9- |
| | TGGGCGACGTGCGCAGCGTGACACAGAAGCACATCCAGGAGTGGGGACCCTTCG | KRAB (nt) |
| | ACCTGGTCATCGGAGGCAGCCCCTGTAATGACCTGTCCATCGTGAACCCTGCAAG | |
| | GAAGGGCCTGTATGAGGGAACCGGCAGACTGTTCTTTGAGTTCTACAGGCTGCTG | |
| | CACGACGCCCGCCCTAAGGAGGGCGATGACAGGCCATTCTTTTGGCTGTTTGAGA | |
| | ACGTGGTGGCCATGGGCGTGAGCGACAAGCGGGATATCTCCAGATTCCTGGAGTC | |
| | TAATCCCGTGATGATCGATGCAAAGGAGGTGTCTGCCGCACACAGGGCAAGGTAC | |
| | TTTTGGGGAAATCTGCCTGGCATGAACCGCCCACTGGCCAGCACCGTGAACGACA | |
| | AGCTGGAGCTGCAGGAGTGCCTGGAGCACGGAAGGATCGCCAAGTTCTCCAAGGT | |
| | GCGGACAATCACCACAAGATCTAACAGCATCAAGCAGGGCAAGGATCAGCACTTC | |
| | CCCGTGTTCATGAATGAGAAGGAGGACATCCTGTGGTGTACCGAGATGGAGCGCG | |
| | TGTTCGGCTTTCCAGTGCACTATACAGACGTGAGCAATATGAGCCGGCTGGCAAG | |
| | GCAGAGACTGCTGGGCCGGTCCTGGTCTGTGCCAGTGATCAGACACCTGTTCGCC | |
| | CCCCTGAAGGAGTACTTTGCCTGCGTGTCTAGCGGCAACTCTAATGCCAACAGCA | |
| | GAGGCCCTTCCTTTTCCTCTGGCCTGGTGCCACTGTCTCTGAGGGGCAGCCACATG | |
| | GGCCCCATGGAGATCTACAAGACCGTGTCCGCCTGGAAGAGGCAGCCTGTGCGCG | |
| | TGCTGTCTCTGTTCCGCAACATCGACAAGGTGCTGAAGAGCCTGGGCTTTCTGGAG | |
| | AGCGGATCCGGATCTGGAGGAGGCACCCTGAAGTATGTGGAGGATGTGACAAAT | |
| | GTGGTGCGGAGAGATGTGGAGAAGTGGGGCCCCTTCGATCTGGTGTACGGATCCA | |
| | CCCAGCCACTGGGAAGCTCCTGCGATAGGTGTCCAGGATGGTATATGTTCCAGTTT | |
| | CACAGAATCCTGCAGTACGCACTGCCAAGGCAGGAGAGCCAGCGCCCTTTCTTTT | |
| | GGATCTTTATGGACAACCTGCTGCTGACAGAGGATGACCAGGAGACAACAACCCG | |
| | CTTCCTGCAGACAGAGGCAGTGACCCTGCAGGATGTGAGGGGACGCGACTATCAG | |
| | AATGCCATGCGGGTGTGGTCTAACATCCCTGGCCTGAAGAGCAAGCACGCCCCCC | |
| | TGACCCCTAAGGAGGAGGAGTACCTGCAGGCCCAGGTGCGGAGCAGATCCAAGC | |
| | TGGATGCCCCTAAGGTGGACCTGCTGGTGAAGAATTGTCTGCTGCCACTGCGGGA | |
| | GTACTTCAAGTACTTTAGTCAGAATAGCCTGCCACTGGGAGGGCCGAGCTCTGGC | |
| | GCACCCCCACCAAGTGGAGGGTCTCCTGCCGGGTCCCCAACATCTACTGAAGAAG | |
| | GCACCAGCGAATCCGCAACGCCCGAGTCAGGCCCTGGTACCTCCACAGAACCATC | |
| | TGAAGGTAGTGCGCCTGGTTCCCCAGCTGGAAGCCCTACTTCCACCGAAGAAGGC | |
| | ACGTCAACCGAACCAAGTGAAGGATCTGCCCCTGGGACCAGCACTGAACCATCTG | |
| | AGGTTAACCCCAAGAAGAAGCGGAAGGTGGGCATCCACGGCGTGCCCGCCGCCG | |
| | ACAAGAAGTACAGCATCGGCCTGGCCATCGGCACCAACAGCGTGGGCTGGGCCGT | |
| | GATCACCGACGAGTACAAGGTGCCCAGCAAGAAGTTCAAGGTGCTGGGCAACAC | |
| | CGACCGGCACAGCATCAAGAAGAACCTGATCGGCGCCCTGCTGTTCGACAGCGGC | |
| | GAGACCGCCGAGGCCACCCGGCTGAAGCGGACCGCCCGGCGGCGGTACACCCGG | |
| | CGGAAGAACCGGATCTGCTACCTGCAGGAGATCTTCAGCAACGAGATGGCCAAGG | |
| | TGGACGACAGCTTCTTCCACCGGCTGGAGGAGAGCTTCCTGGTGGAGGAGGACAA | |
| | GAAGCACGAGCGGCACCCCATCTTCGGCAACATCGTGGACGAGGTGGCCTACCAC | |
| | GAGAAGTACCCCACCATCTACCACCTGCGGAAGAAGCTGGTGGACAGCACCGACA | |
| | AGGCCGACCTGCGGCTGATCTACCTGGCCCTGGCCCACATGATCAAGTTCCGGGG | |
| | CCACTTCCTGATCGAGGGCGACCTGAACCCCGACAACAGCGACGTGGACAAGCTG | |
| | TTCATCCAGCTGGTGCAGACCTACAACCAGCTGTTCGAGGAGAACCCCATCAACG | |
| | CCAGCGGCGTGGACGCCAAGGCCATCCTGAGCGCCCGGCTGAGCAAGAGCCGGC | |
| | GGCTGGAGAACCTGATCGCCCAGCTGCCCGGCGAGAAGAAGAACGGCCTGTTCGG | |
| | CAACCTGATCGCCCTGAGCCTGGGCCTGACCCCCAACTTCAAGAGCAACTTCGAC | |
| | CTGGCCGAGGACGCCAAGCTGCAGCTGAGCAAGGACACCTACGACGACGACCTG | |
| | GACAACCTGCTGGCCCAGATCGGCGACCAGTACGCCGACCTGTTCCTGGCCGCCA | |
| | AGAACCTGAGCGACGCCATCCTGCTGAGCGACATCCTGCGGGTGAACACCGAGAT | |
| | CACCAAGGCCCCCCTGAGCGCCAGCATGATCAAGCGGTACGACGAGCACCACCAG | |
| | GACCTGACCCTGCTGAAGGCCCTGGTGCGGCAGCAGCTGCCCGAGAAGTACAAGG | |
| | AGATCTTCTTCGACCAGAGCAAGAACGGCTACGCCGGCTACATCGACGGCGGCGC | |
| | CAGCCAGGAGGAGTTCTACAAGTTCATCAAGCCCATCCTGGAGAAGATGGACGGC | |
| | ACCGAGGAGCTGCTGGTGAAGCTGAACCGGGAGGACCTGCTGCGGAAGCAGCGG | |
| | ACCTTCGACAACGGCAGCATCCCCCACCAGATCCACCTGGGCGAGCTGCACGCCA | |
| | TCCTGCGGCGGCAGGAGGACTTCTACCCCTTCCTGAAGGACAACCGGGAGAAGAT | |
| | CGAGAAGATCCTGACCTTCCGGATCCCCTACTACGTGGGCCCCCTGGCCCGGGGC | |
| | AACAGCCGGTTCGCCTGGATGACCCGGAAGAGCGAGGAGACCATCACCCCCTGGA | |
| | ACTTCGAGGAGGTGGTGGACAAGGGCGCCAGCGCCCAGAGCTTCATCGAGCGGAT | |
| | GACCAACTTCGACAAGAACCTGCCCAACGAGAAGGTGCTGCCCAAGCACAGCCTG | |
| | CTGTACGAGTACTTCACCGTGTACAACGAGCTGACCAAGGTGAAGTACGTGACCG | |
| | AGGGCATGCGGAAGCCCGCCTTCCTGAGCGGCGAGCAGAAGAAGGCCATCGTGG | |
| | ACCTGCTGTTCAAGACCAACCGGAAGGTGACCGTGAAGCAGCTGAAGGAGGACT | |
| | ACTTCAAGAAGATCGAGTGCTTCGACAGCGTGGAGATCAGCGGCGTGGAGGACCG | |
| | GTTCAACGCCAGCCTGGGCACCTACCACGACCTGCTGAAGATCATCAAGGACAAG | |
| | GACTTCCTGGACAACGAGGAGAACGAGGACATCCTGGAGGACATCGTGCTGACCC | |
| | TGACCCTGTTCGAGGACCGGGAGATGATCGAGGAGCGGCTGAAGACCTACGCCCA | |
| | CCTGTTCGACGACAAGGTGATGAAGCAGCTGAAGCGGCGGCGGTACACCGGCTGG | |
| | GGCCGGCTGAGCCGGAAGCTGATCAACGGCATCCGGGACAAGCAGAGCGGCAAG | |
| | ACCATCCTGGACTTCCTGAAGAGCGACGGCTTCGCCAACCGGAACTTCATGCAGC | |
| | TGATCCACGACGACAGCCTGACCTTCAAGGAGGACATCCAGAAGGCCCAGGTGAG | |
| | CGGCCAGGGCGACAGCCTGCACGAGCACATCGCCAACCTGGCCGGCAGCCCCGCC | |
| | ATCAAGAAGGGCATCCTGCAGACCGTGAAGGTGGTGGACGAGCTGGTGAAGGTG | |
| | ATGGGCCGGCACAAGCCCGAGAACATCGTGATCGAGATGGCCCGGGAGAACCAG | |
| | ACCACCCAGAAGGGCCAGAAGAACAGCCGGGAGCGGATGAAGCGGATCGAGGAG | |
| | GGCATCAAGGAGCTGGGCAGCCAGATCCTGAAGGAGCACCCCGTGGAGAACACC | |
| | CAGCTGCAGAACGAGAAGCTGTACCTGTACTACCTGCAGAACGGCCGGGACATGT | |
| | ACGTGGACCAGGAGCTGGACATCAACCGGCTGAGCGACTACGACGTGGACGCCAT | |
| | CGTGCCCCAGAGCTTCCTGAAGGACGACAGCATCGACAACAAGGTGCTGACCCGG | |
| | AGCGACAAGAACCGGGGCAAGAGCGACAACGTGCCCAGCGAGGAGGTGGTGAAG | |
| | AAGATGAAGAACTACTGGCGGCAGCTGCTGAACGCCAAGCTGATCACCCAGCGG | |
| | AAGTTCGACAACCTGACCAAGGCCGAGCGGGGCGGCCTGAGCGAGCTGGACAAG | |
| | GCCGGCTTCATCAAGCGGCAGCTGGTGGAGACCCGGCAGATCACCAAGCACGTGG | |
| | CCCAGATCCTGGACAGCCGGATGAACACCAAGTACGACGAGAACGACAAGCTGA | |
| | TCCGGGAGGTGAAGGTGATCACCCTGAAGAGCAAGCTGGTGAGCGACTTCCGGAA | |
| | GGACTTCCAGTTCTACAAGGTGCGGGAGATCAACAACTACCACCACGCCCACGAC | |
| | GCCTACCTGAACGCCGTGGTGGGCACCGCCCTGATCAAGAAGTACCCCAAGCTGG | |
| | AGAGCGAGTTCGTGTACGGCGACTACAAGGTGTACGACGTGCGGAAGATGATCGC | |
| | CAAGAGCGAGCAGGAGATCGGCAAGGCCACCGCCAAGTACTTCTTCTACAGCAAC | |
| | ATCATGAACTTCTTCAAGACCGAGATCACCCTGGCCAACGGCGAGATCCGGAAGC | |
| | GGCCCCTGATCGAGACCAACGGCGAGACCGGCGAGATCGTGTGGGACAAGGGCC | |
| | GGGACTTCGCCACCGTGCGGAAGGTGCTGAGCATGCCCCAGGTGAACATCGTGAA | |
| | GAAGACCGAGGTGCAGACCGGCGGCTTCAGCAAGGAGAGCATCCTGCCCAAGCG | |
| | GAACAGCGACAAGCTGATCGCCCGGAAGAAGGACTGGGACCCCAAGAAGTACGG | |
| | CGGCTTCGACAGCCCCACCGTGGCCTACAGCGTGCTGGTGGTGGCCAAGGTGGAG | |
| | AAGGGCAAGAGCAAGAAGCTGAAGAGCGTGAAGGAGCTGCTGGGCATCACCATC | |
| | ATGGAGCGGAGCAGCTTCGAGAAGAACCCCATCGACTTCCTGGAGGCCAAGGGCT | |
| | ACAAGGAGGTGAAGAAGGACCTGATCATCAAGCTGCCCAAGTACAGCCTGTTCGA | |
| | GCTGGAGAACGGCCGGAAGCGGATGCTGGCCAGCGCCGGCGAGCTGCAGAAGGG | |
| | CAACGAGCTGGCCCTGCCCAGCAAGTACGTGAACTTCCTGTACCTGGCCAGCCAC | |
| | TACGAGAAGCTGAAGGGCAGCCCCGAGGACAACGAGCAGAAGCAGCTGTTCGTG | |
| | GAGCAGCACAAGCACTACCTGGACGAGATCATCGAGCAGATCAGCGAGTTCAGC | |
| | AAGCGGGTGATCCTGGCCGACGCCAACCTGGACAAGGTGCTGAGCGCCTACAACA | |
| | AGCACCGGGACAAGCCCATCCGGGAGCAGGCCGAGAACATCATCCACCTGTTCAC | |
| | CCTGACCAACCTGGGCGCCCCCGCCGCCTTCAAGTACTTCGACACCACCATCGACC | |
| | GGAAGCGGTACACCAGCACCAAGGAGGTGCTGGACGCCACCCTGATCCACCAGA | |
| | GCATCACCGGCCTGTACGAGACCCGGATCGACCTGAGCCAGCTGGGCGGCGACAG | |
| | CGGCGGCAAGCGGCCCGCCGCCACCAAGAAGGCCGGCCAGGCCAAGAAGAAGAA | |
| | GGctagCgatgctaagtcactgactgcctggtcccggacactggtgaccttcaaggatgtgtttgtggacttc | |
| | accagggaggagtggaagctgctggacactgctcagcagatcctgtacagaaatgtgatgctggagaactata | |
| | agaacctggtttccttgggttatcagcttactaagccagatgtgatcctccggttggagaagggagaagagcc | |
| | ctggctggtggagagagaaattcaccaagagacccatcctgattcagagactgcatttgaaatcaaatcatca | |
| | gttCCGAAAAAGAAACGCAAAGTTTAG | |
| 280 | MNHDQEFDPPKVYPPVPAEKRKPIRVLSLFDGIATGLLVLKDLGIQVDRYIASEVCEDS | DNMT3A/ |
| | ITVGMVRHQGKIMYVGDVRSVTQKHIQEWGPFDLVIGGSPCNDLSIVNPARKGLYEG | L- |
| | TGRLFFEFYRLLHDARPKEGDDRPFFWLFENVVAMGVSDKRDISRFLESNPVMIDAKE | XTEN80- |
| | VSAAHRARYFWGNLPGMNRPLASTVNDKLELQECLEHGRIAKFSKVRTITTRSNSIKQ | dSpCas9- |
| | GKDQHFPVFMNEKEDILWCTEMERVFGFPVHYTDVSNMSRLARQRLLGRSWSVPVIR | KRAB |
| | HLFAPLKEYFACVSSGNSNANSRGPSFSSGLVPLSLRGSHMGPMEIYKTVSAWKRQPV | (AA) |
| | RVLSLFRNIDKVLKSLGFLESGSGSGGGTLKYVEDVTNVVRRDVEKWGPFDLVYGST | |
| | QPLGSSCDRCPGWYMFQFHRILQYALPRQESQRPFFWIFMDNLLLTEDDQETTTRFLQ | |
| | TEAVTLQDVRGRDYQNAMRVWSNIPGLKSKHAPLTPKEEEYLQAQVRSRSKLDAPK | |
| | VDLLVKNCLLPLREYFKYFSQNSLPLGGPSSGAPPPSGGSPAGSPTSTEEGTSESATPES | |
| | GPGTSTEPSEGSAPGSPAGSPTSTEEGTSTEPSEGSAPGTSTEPSEVNPKKKRKVGIHGV | |
| | PAADKKYSIGLAIGTNSVGWAVITDEYKVPSKKFKVLGNTDRHSIKKNLIGALLFDSG | |
| | ETAEATRLKRTARRRYTRRKNRICYLQEIFSNEMAKVDDSFFHRLEESFLVEEDKKHE | |
| | RHPIFGNIVDEVAYHEKYPTIYHLRKKLVDSTDKADLRLIYLALAHMIKFRGHFLIEGD | |
| | LNPDNSDVDKLFIQLVQTYNQLFEENPINASGVDAKAILSARLSKSRRLENLIAQLPGE | |
| | KKNGLFGNLIALSLGLTPNFKSNFDLAEDAKLQLSKDTYDDDLDNLLAQIGDQYADL | |
| | FLAAKNLSDAILLSDILRVNTEITKAPLSASMIKRYDEHHQDLTLLKALVRQQLPEKYK | |
| | EIFFDQSKNGYAGYIDGGASQEEFYKFIKPILEKMDGTEELLVKLNREDLLRKQRTFDN | |
| | GSIPHQIHLGELHAILRRQEDFYPFLKDNREKIEKILTFRIPYYVGPLARGNSRFAWMTR | |
| | KSEETITPWNFEEVVDKGASAQSFIERMTNFDKNLPNEKVLPKHSLLYEYFTVYNELT | |
| | KVKYVTEGMRKPAFLSGEQKKAIVDLLFKTNRKVTVKQLKEDYFKKIECFDSVEISGV | |
| | EDRFNASLGTYHDLLKIIKDKDFLDNEENEDILEDIVLTLTLFEDREMIEERLKTYAHLF | |
| | DDKVMKQLKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKVMGRHKPEN | |
| | IVIEMARENQTTQKGQKNSRERMKRIEEGIKELGSQILKEHPVENTQLQNEKLYLYYL | |
| | QNGRDMYVDQELDINRLSDYDVDAIVPQSFLKDDSIDNKVLTRSDKNRGKSDNVPSE | |
| | EVVKKMKNYWRQLLNAKLITQRKFDNLTKAERGGLSELDKAGFIKRQLVETRQITKH | |
| | VAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMN | |
| | FFKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKR | |
| | VILADANLDKVLSAYNKHRDKPIREQAENIIHLFTLTNLGAPAAFKYFDTTIDRKRYTS | |
| | TKEVLDATLIHQSITGLYETRIDLSQLGGDSGGKRPAATKKAGQAKKKKASDAKSLTA | |
| | WSRTLVTFKDVFVDFTREEWKLLDTAQQILYRNVMLENYKNLVSLGYQLTKPDVILR | |
| | LEKGEEPWLVEREIHQETHPDSETAFEIKSSVPKKKRKV | |
| 281 | AGTAACCATGACCAGGAATTTGACCCCCCAAAGGTTTACCCACCTGTGCCAGCTG | DNMT3A/ |
| | AGAAGAGGAAGCCCATCCGCGTGCTGTCTCTCTTTGATGGGATTGCTACAGGGCT | L (CRISPR |
| | CCTGGTGCTGAAGGACCTGGGCATCCAAGTGGACCGCTACATTGCCTCCGAGGTG | OFF)- |
| | TGTGAGGACTCCATCACGGTGGGCATGGTGCGGCACCAGGGAAAGATCATGTACG | XTEN80- |
| | TCGGGGACGTCCGCAGCGTCACACAGAAGCATATCCAGGAGTGGGGCCCATTCGA | dSpCas9- |
| | CCTGGTGATTGGAGGCAGTCCCTGCAATGACCTCTCCATTGTCAACCCTGCCCGCA | KRAB (nt) |
| | AGGGACTTTATGAGGGTACTGGCCGCCTCTTCTTTGAGTTCTACCGCCTCCTGCAT | |
| | GATGCGCGGCCCAAGGAGGGAGATGATCGCCCCTTCTTCTGGCTCTTTGAGAATG | |
| | TGGTGGCCATGGGCGTTAGTGACAAGAGGGACATCTCGCGATTTCTTGAGTCTAA | |
| | CCCCGTGATGATTGACGCCAAAGAAGTGTCTGCTGCACACAGGGCCCGTTACTTCT | |
| | GGGGTAACCTTCCTGGCATGAACAGGCCTTTGGCATCCACTGTGAATGATAAGCT | |
| | GGAGCTGCAAGAGTGTCTGGAGCACGGCAGAATAGCCAAGTTCAGCAAAGTGAG | |
| | GACCATTACCACCAGGTCAAACTCTATAAAGCAGGGCAAAGACCAGCATTTCCCC | |
| | GTCTTCATGAACGAGAAGGAGGACATCCTGTGGTGCACTGAAATGGAAAGGGTGT | |
| | TTGGCTTCCCCGTCCACTACACAGACGTCTCCAACATGAGCCGCTTGGCGAGGCA | |
| | GAGACTGCTGGGCCGATCGTGGAGCGTGCCGGTCATCCGCCACCTCTTCGCTCCGC | |
| | TGAAGGAATATTTTGCTTGTGTGTCTAGCGGCAATAGTAACGCTAACAGCCGCGG | |
| | GCCGAGCTTCAGCAGCGGCCTGGTGCCGTTAAGCTTGCGCGGCAGCCATATGGGC | |
| | CCTATGGAGATATACAAGACAGTGTCTGCATGGAAGAGACAGCCAGTGCGGGTAC | |
| | TGAGCCTCTTCAGAAACATCGACAAGGTACTAAAGAGTTTGGGCTTCTTGGAAAG | |
| | CGGTTCTGGTTCTGGGGGAGGAACGCTGAAGTACGTGGAAGATGTCACAAATGTC | |
| | GTGAGGAGAGACGTGGAGAAATGGGGCCCCTTTGACCTGGTGTACGGCTCGACGC | |
| | AGCCCCTAGGCAGCTCTTGTGATCGCTGTCCCGGCTGGTACATGTTCCAGTTCCAC | |
| | CGGATCCTGCAGTATGCGCTGCCTCGCCAGGAGAGTCAGCGGCCCTTCTTCTGGAT | |
| | ATTCATGGACAATCTGCTGCTGACTGAGGATGACCAAGAGACAACTACCCGCTTC | |
| | CTTCAGACAGAGGCTGTGACCCTCCAGGATGTCCGTGGCAGAGACTACCAGAATG | |
| | CTATGCGGGTGTGGAGCAACATTCCAGGGCTGAAGAGCAAGCATGCGCCCCTGAC | |
| | CCCAAAGGAAGAAGAGTATCTGCAAGCCCAAGTCAGAAGCAGGAGCAAGCTGGA | |
| | CGCCCCGAAAGTTGACCTCCTGGTGAAGAACTGCCTTCTCCCGCTGAGAGAGTAC | |
| | TTCAAGTATTTTTCTCAAAACTCACTTCCTCTTGGAGGGCCGAGCTCTGGCGCACC | |
| | CCCACCAAGTGGAGGGTCTCCTGCCGGGTCCCCAACATCTACTGAAGAAGGCACC | |
| | AGCGAATCCGCAACGCCCGAGTCAGGCCCTGGTACCTCCACAGAACCATCTGAAG | |
| | GTAGTGCGCCTGGTTCCCCAGCTGGAAGCCCTACTTCCACCGAAGAAGGCACGTC | |
| | AACCGAACCAAGTGAAGGATCTGCCCCTGGGACCAGCACTGAACCATCTGAGGTT | |
| | AACCCCAAGAAGAAGCGGAAGGTGGGCATCCACGGCGTGCCCGCCGCCGACAAG | |
| | AAGTACAGCATCGGCCTGGCCATCGGCACCAACAGCGTGGGCTGGGCCGTGATCA | |
| | CCGACGAGTACAAGGTGCCCAGCAAGAAGTTCAAGGTGCTGGGCAACACCGACC | |
| | GGCACAGCATCAAGAAGAACCTGATCGGCGCCCTGCTGTTCGACAGCGGCGAGAC | |
| | CGCCGAGGCCACCCGGCTGAAGCGGACCGCCCGGCGGCGGTACACCCGGCGGAA | |
| | GAACCGGATCTGCTACCTGCAGGAGATCTTCAGCAACGAGATGGCCAAGGTGGAC | |
| | GACAGCTTCTTCCACCGGCTGGAGGAGAGCTTCCTGGTGGAGGAGGACAAGAAGC | |
| | ACGAGCGGCACCCCATCTTCGGCAACATCGTGGACGAGGTGGCCTACCACGAGAA | |
| | GTACCCCACCATCTACCACCTGCGGAAGAAGCTGGTGGACAGCACCGACAAGGCC | |
| | GACCTGCGGCTGATCTACCTGGCCCTGGCCCACATGATCAAGTTCCGGGGCCACTT | |
| | CCTGATCGAGGGCGACCTGAACCCCGACAACAGCGACGTGGACAAGCTGTTCATC | |
| | CAGCTGGTGCAGACCTACAACCAGCTGTTCGAGGAGAACCCCATCAACGCCAGCG | |
| | GCGTGGACGCCAAGGCCATCCTGAGCGCCCGGCTGAGCAAGAGCCGGCGGCTGG | |
| | AGAACCTGATCGCCCAGCTGCCCGGCGAGAAGAAGAACGGCCTGTTCGGCAACCT | |
| | GATCGCCCTGAGCCTGGGCCTGACCCCCAACTTCAAGAGCAACTTCGACCTGGCC | |
| | GAGGACGCCAAGCTGCAGCTGAGCAAGGACACCTACGACGACGACCTGGACAAC | |
| | CTGCTGGCCCAGATCGGCGACCAGTACGCCGACCTGTTCCTGGCCGCCAAGAACC | |
| | TGAGCGACGCCATCCTGCTGAGCGACATCCTGCGGGTGAACACCGAGATCACCAA | |
| | GGCCCCCCTGAGCGCCAGCATGATCAAGCGGTACGACGAGCACCACCAGGACCTG | |
| | ACCCTGCTGAAGGCCCTGGTGCGGCAGCAGCTGCCCGAGAAGTACAAGGAGATCT | |
| | TCTTCGACCAGAGCAAGAACGGCTACGCCGGCTACATCGACGGCGGCGCCAGCCA | |
| | GGAGGAGTTCTACAAGTTCATCAAGCCCATCCTGGAGAAGATGGACGGCACCGAG | |
| | GAGCTGCTGGTGAAGCTGAACCGGGAGGACCTGCTGCGGAAGCAGCGGACCTTCG | |
| | ACAACGGCAGCATCCCCCACCAGATCCACCTGGGCGAGCTGCACGCCATCCTGCG | |
| | GCGGCAGGAGGACTTCTACCCCTTCCTGAAGGACAACCGGGAGAAGATCGAGAA | |
| | GATCCTGACCTTCCGGATCCCCTACTACGTGGGCCCCCTGGCCCGGGGCAACAGC | |
| | CGGTTCGCCTGGATGACCCGGAAGAGCGAGGAGACCATCACCCCCTGGAACTTCG | |
| | AGGAGGTGGTGGACAAGGGCGCCAGCGCCCAGAGCTTCATCGAGCGGATGACCA | |
| | ACTTCGACAAGAACCTGCCCAACGAGAAGGTGCTGCCCAAGCACAGCCTGCTGTA | |
| | CGAGTACTTCACCGTGTACAACGAGCTGACCAAGGTGAAGTACGTGACCGAGGGC | |
| | ATGCGGAAGCCCGCCTTCCTGAGCGGCGAGCAGAAGAAGGCCATCGTGGACCTGC | |
| | TGTTCAAGACCAACCGGAAGGTGACCGTGAAGCAGCTGAAGGAGGACTACTTCAA | |
| | GAAGATCGAGTGCTTCGACAGCGTGGAGATCAGCGGCGTGGAGGACCGGTTCAAC | |
| | GCCAGCCTGGGCACCTACCACGACCTGCTGAAGATCATCAAGGACAAGGACTTCC | |
| | TGGACAACGAGGAGAACGAGGACATCCTGGAGGACATCGTGCTGACCCTGACCCT | |
| | GTTCGAGGACCGGGAGATGATCGAGGAGCGGCTGAAGACCTACGCCCACCTGTTC | |
| | GACGACAAGGTGATGAAGCAGCTGAAGCGGCGGCGGTACACCGGCTGGGGCCGG | |
| | CTGAGCCGGAAGCTGATCAACGGCATCCGGGACAAGCAGAGCGGCAAGACCATC | |
| | CTGGACTTCCTGAAGAGCGACGGCTTCGCCAACCGGAACTTCATGCAGCTGATCC | |
| | ACGACGACAGCCTGACCTTCAAGGAGGACATCCAGAAGGCCCAGGTGAGCGGCC | |
| | AGGGCGACAGCCTGCACGAGCACATCGCCAACCTGGCCGGCAGCCCCGCCATCAA | |
| | GAAGGGCATCCTGCAGACCGTGAAGGTGGTGGACGAGCTGGTGAAGGTGATGGG | |
| | CCGGCACAAGCCCGAGAACATCGTGATCGAGATGGCCCGGGAGAACCAGACCAC | |
| | CCAGAAGGGCCAGAAGAACAGCCGGGAGCGGATGAAGCGGATCGAGGAGGGCAT | |
| | CAAGGAGCTGGGCAGCCAGATCCTGAAGGAGCACCCCGTGGAGAACACCCAGCT | |
| | GCAGAACGAGAAGCTGTACCTGTACTACCTGCAGAACGGCCGGGACATGTACGTG | |
| | GACCAGGAGCTGGACATCAACCGGCTGAGCGACTACGACGTGGACGCCATCGTGC | |
| | CCCAGAGCTTCCTGAAGGACGACAGCATCGACAACAAGGTGCTGACCCGGAGCG | |
| | ACAAGAACCGGGGCAAGAGCGACAACGTGCCCAGCGAGGAGGTGGTGAAGAAGA | |
| | TGAAGAACTACTGGCGGCAGCTGCTGAACGCCAAGCTGATCACCCAGCGGAAGTT | |
| | CGACAACCTGACCAAGGCCGAGCGGGGCGGCCTGAGCGAGCTGGACAAGGCCGG | |
| | CTTCATCAAGCGGCAGCTGGTGGAGACCCGGCAGATCACCAAGCACGTGGCCCAG | |
| | ATCCTGGACAGCCGGATGAACACCAAGTACGACGAGAACGACAAGCTGATCCGG | |
| | GAGGTGAAGGTGATCACCCTGAAGAGCAAGCTGGTGAGCGACTTCCGGAAGGAC | |
| | TTCCAGTTCTACAAGGTGCGGGAGATCAACAACTACCACCACGCCCACGACGCCT | |
| | ACCTGAACGCCGTGGTGGGCACCGCCCTGATCAAGAAGTACCCCAAGCTGGAGAG | |
| | CGAGTTCGTGTACGGCGACTACAAGGTGTACGACGTGCGGAAGATGATCGCCAAG | |
| | AGCGAGCAGGAGATCGGCAAGGCCACCGCCAAGTACTTCTTCTACAGCAACATCA | |
| | TGAACTTCTTCAAGACCGAGATCACCCTGGCCAACGGCGAGATCCGGAAGCGGCC | |
| | CCTGATCGAGACCAACGGCGAGACCGGCGAGATCGTGTGGGACAAGGGCCGGGA | |
| | CTTCGCCACCGTGCGGAAGGTGCTGAGCATGCCCCAGGTGAACATCGTGAAGAAG | |
| | ACCGAGGTGCAGACCGGCGGCTTCAGCAAGGAGAGCATCCTGCCCAAGCGGAAC | |
| | AGCGACAAGCTGATCGCCCGGAAGAAGGACTGGGACCCCAAGAAGTACGGCGGC | |
| | TTCGACAGCCCCACCGTGGCCTACAGCGTGCTGGTGGTGGCCAAGGTGGAGAAGG | |
| | GCAAGAGCAAGAAGCTGAAGAGCGTGAAGGAGCTGCTGGGCATCACCATCATGG | |
| | AGCGGAGCAGCTTCGAGAAGAACCCCATCGACTTCCTGGAGGCCAAGGGCTACAA | |
| | GGAGGTGAAGAAGGACCTGATCATCAAGCTGCCCAAGTACAGCCTGTTCGAGCTG | |
| | GAGAACGGCCGGAAGCGGATGCTGGCCAGCGCCGGCGAGCTGCAGAAGGGCAAC | |
| | GAGCTGGCCCTGCCCAGCAAGTACGTGAACTTCCTGTACCTGGCCAGCCACTACG | |
| | AGAAGCTGAAGGGCAGCCCCGAGGACAACGAGCAGAAGCAGCTGTTCGTGGAGC | |
| | AGCACAAGCACTACCTGGACGAGATCATCGAGCAGATCAGCGAGTTCAGCAAGC | |
| | GGGTGATCCTGGCCGACGCCAACCTGGACAAGGTGCTGAGCGCCTACAACAAGCA | |
| | CCGGGACAAGCCCATCCGGGAGCAGGCCGAGAACATCATCCACCTGTTCACCCTG | |
| | ACCAACCTGGGCGCCCCCGCCGCCTTCAAGTACTTCGACACCACCATCGACCGGA | |
| | AGCGGTACACCAGCACCAAGGAGGTGCTGGACGCCACCCTGATCCACCAGAGCAT | |
| | CACCGGCCTGTACGAGACCCGGATCGACCTGAGCCAGCTGGGCGGCGACAGCGGC | |
| | GGCAAGCGGCCCGCCGCCACCAAGAAGGCCGGCCAGGCCAAGAAGAAGAAGGcta | |
| | gCgatgctaagtcactgactgcctggtcccggacactggtgaccttcaaggatgtgtttgtggacttcacca | |
| | gggaggagtggaagctgctggacactgctcagcagatcctgtacagaaatgtgatgctggagaactataaga | |
| | acctggtttccttgggttatcagcttactaagccagatgtgatcctccggttggagaagggagaagagccct | |
| | ggctggtggagagagaaattcaccaagagacccatcctgattcagagactgcatttgaaatcaaatcatcag | |
| | ttCCGAAAAAGAAACGCAAAGTTTAG | |
| 282 | SNHDQEFDPPKVYPPVPAEKRKPIRVLSLFDGIATGLLVLKDLGIQVDRYIASEVCEDSI | DNMT3A/ |
| | TVGMVRHQGKIMYVGDVRSVTQKHIQEWGPFDLVIGGSPCNDLSIVNPARKGLYEGT | L (CRISPR |
| | GRLFFEFYRLLHDARPKEGDDRPFFWLFENVVAMGVSDKRDISRFLESNPVMIDAKEV | OFF)- |
| | SAAHRARYFWGNLPGMNRPLASTVNDKLELQECLEHGRIAKFSKVRTITTRSNSIKQG | XTEN80- |
| | KDQHFPVFMNEKEDILWCTEMERVFGFPVHYTDVSNMSRLARQRLLGRSWSVPVIRH | dSpCas9- |
| | LFAPLKEYFACVSSGNSNANSRGPSFSSGLVPLSLRGSHMGPMEIYKTVSAWKRQPVR | KRAB |
| | VLSLFRNIDKVLKSLGFLESGSGSGGGTLKYVEDVTNVVRRDVEKWGPFDLVYGSTQ | (AA) |
| | PLGSSCDRCPGWYMFQFHRILQYALPRQESQRPFFWIFMDNLLLTEDDQETTTRFLQT | |
| | EAVTLQDVRGRDYQNAMRVWSNIPGLKSKHAPLTPKEEEYLQAQVRSRSKLDAPKV | |
| | DLLVKNCLLPLREYFKYFSQNSLPLGGPSSGAPPPSGGSPAGSPTSTEEGTSESATPESG | |
| | PGTSTEPSEGSAPGSPAGSPTSTEEGTSTEPSEGSAPGTSTEPSEVNPKKKRKVGIHGVP | |
| | AADKKYSIGLAIGTNSVGWAVITDEYKVPSKKFKVLGNTDRHSIKKNLIGALLFDSGE | |
| | TAEATRLKRTARRRYTRRKNRICYLQEIFSNEMAKVDDSFFHRLEESFLVEEDKKHER | |
| | HPIFGNIVDEVAYHEKYPTIYHLRKKLVDSTDKADLRLIYLALAHMIKFRGHFLIEGDL | |
| | NPDNSDVDKLFIQLVQTYNQLFEENPINASGVDAKAILSARLSKSRRLENLIAQLPGEK | |
| | KNGLFGNLIALSLGLTPNFKSNFDLAEDAKLQLSKDTYDDDLDNLLAQIGDQYADLFL | |
| | AAKNLSDAILLSDILRVNTEITKAPLSASMIKRYDEHHQDLTLLKALVRQQLPEKYKEI | |
| | FFDQSKNGYAGYIDGGASQEEFYKFIKPILEKMDGTEELLVKLNREDLLRKQRTFDNG | |
| | SIPHQIHLGELHAILRRQEDFYPFLKDNREKIEKILTFRIPYYVGPLARGNSRFAWMTRK | |
| | SEETITPWNFEEVVDKGASAQSFIERMTNFDKNLPNEKVLPKHSLLYEYFTVYNELTK | |
| | VKYVTEGMRKPAFLSGEQKKAIVDLLFKTNRKVTVKQLKEDYFKKIECFDSVEISGVE | |
| | DRFNASLGTYHDLLKIIKDKDFLDNEENEDILEDIVLTLTLFEDREMIEERLKTYAHLF | |
| | DDKVMKQLKRRRYTGWGRLSRKLINGIRDKQSGKTILDFLKSDGFANRNFMQLIHDD | |
| | SLTFKEDIQKAQVSGQGDSLHEHIANLAGSPAIKKGILQTVKVVDELVKVMGRHKPEN | |
| | IVIEMARENQTTQKGQKNSRERMKRIEEGIKELGSQILKEHPVENTQLQNEKLYLYYL | |
| | QNGRDMYVDQELDINRLSDYDVDAIVPQSFLKDDSIDNKVLTRSDKNRGKSDNVPSE | |
| | EVVKKMKNYWRQLLNAKLITQRKFDNLTKAERGGLSELDKAGFIKRQLVETRQITKH | |
| | VAQILDSRMNTKYDENDKLIREVKVITLKSKLVSDFRKDFQFYKVREINNYHHAHDA | |
| | YLNAVVGTALIKKYPKLESEFVYGDYKVYDVRKMIAKSEQEIGKATAKYFFYSNIMN | |
| | FFKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKR | |
| | VILADANLDKVLSAYNKHRDKPIREQAENIIHLFTLTNLGAPAAFKYFDTTIDRKRYTS | |
| | TKEVLDATLIHQSITGLYETRIDLSQLGGDSGGKRPAATKKAGQAKKKKASDAKSLTA | |
| | WSRTLVTFKDVFVDFTREEWKLLDTAQQILYRNVMLENYKNLVSLGYQLTKPDVILR | |
| | LEKGEEPWLVEREIHQETHPDSETAFEIKSSVPKKKRKV | |
| 283 | MGQTGKKSEKGPVCWRKRVKSEYMRLRQLKRFRRADEVKTMFSSNRQKILERTETL | EZH2 (AA) |
| | NQEWKQRRIQPVHIMTSVSSLRGTRECSVTSDLDFPAQVIPLKTLNAVASVPIMYSWS | |
| | PLQQNFMVEDETVLHNIPYMGDEVLDQDGTFIEELIKNYDGKVHGDRECGFINDEIFV | |
| | ELVNALGQYNDDDDDDDGDDPDEREEKQKDLEDNRDDKETCPPRKFPADKIFEAISS | |
| | MFPDKGTAEELKEKYKELTEQQLPGALPPECTPNIDGPNAKSVQREQSLHSFHTLFCR | |
| | RCFKYDCFLHPFHATPNTYKRKNTETALDNKPCGPQCYQHLEGAKEFAAALTAERIK | |
| | TPPKRPGGRRRGRLPNNSSRPSTPTISVLESKDTDSDREAGTETGGENNDKEEEEKKDE | |
| | TSSSSEANSRCQTPIKMKPNIEPPENVEWSGAEASMFRVLIGTYYDNFCAIARLIGTKT | |
| | CRQVYEFRVKESSIIAPVPTEDVDTPPRKKKRKHRLWAAHCRKIQLKKDGSSNHVYN | |
| | YQPCDHPRQPCDSSCPCVIAQNFCEKFCQCSSECQNRFPGCRCKAQCNTKQCPCYLAV | |
| | RECDPDLCLTCGAADHWDSKNVSCKNCSIQRGSKKHLLLAPSDVAGWGIFIKDPVQK | |
| | NEFISEYCGEIISQDEADRRGKVYDKYMCSFLFNLNNDFVVDATRKGNKIRFANHSVN | |
| | PNCYAKVMMVNGDHRIGIFAKRAIQTGEELFFDYRYSQADALKYVGIEREMEIP | |
| 284 | MPAMPSSGPGDTSSSAAEREEDRKDGEEQEEPRGKEERQEPSTTARKVGRPG | DNMT3A |
| | RKRKHPPVESGDTPKDPAVISKSPSMAQDSGASELLPNGDLEKRSEPQPEEGS | (AA) |
| | PAGGQKGGAPAEGEGAAETLPEASRAVENGCCTPKEGRGAPAEAGKEQKET | |
| | NIESMKMEGSRGRLRGGLGWESSLRQRPMPRLTFQAGDPYYISKRKRDEWL | |
| | ARWKREAEKKAKVIAGMNAVEENQGPGESQKVEEASPPAVQQPTDPASPTV | |
| | ATTPEPVGSDAGDKNATKAGDDEPEYEDGRGFGIGELVWGKLRGFSWWPGR | |
| | IVSWWMTGRSRAAEGTRWVMWFGDGKFSVVCVEKLMPLSSFCSAFHQATY | |
| | NKQPMYRKAIYEVLQVASSRAGKLFPVCHDSDESDTAKAVEVQNKPMIEWA | |
| | LGGFQPSGPKGLEPPEEEKNPYKEVYTDMWVEPEAAAYAPPPPAKKPRKSTA | |
| | EKPKVKEIIDERTRERLVYEVRQKCRNIEDICISCGSLNVTLEHPLFVGGMCQN | |
| | CKNCFLECAYQYDDDGYQSYCTICCGGREVLMCGNNNCCRCFCVECVDLLV | |
| | GPGAAQAAIKEDPWNCYMCGHKGTYGLLRRREDWPSRLQMFFANNHDQEF | |
| | DPPKVYPPVPAEKRKPIRVLSLFDGIATGLLVLKDLGIQVDRYIASEVCEDSIT | |
| | VGMVRHQGKIMYVGDVRSVTQKHIQEWGPFDLVIGGSPCNDLSIVNPARKG | |
| | LYEGTGRLFFEFYRLLHDARPKEGDDRPFFWLFENVVAMGVSDKRDISRFLE | |
| | SNPVMIDAKEVSAAHRARYFWGNLPGMNRPLASTVNDKLELQECLEHGRIA | |
| | KFSKVRTITTRSNSIKQGKDQHFPVFMNEKEDILWCTEMERVFGFPVHYTDVS | |
| | NMSRLARQRLLGRSWSVPVIRHLFAPLKEYFA | |
| 285 | NHDQEFDPPKVYPPVPAEKRKPIRVLSLFDGIATGLLVLKDLGIQVDRYIASEV | Human |
| | CEDSITVGMVRHQGKIMYVGDVRSVTQKHIQEWGPFDLVIGGSPCNDLSIVN | DNMT3A |
| | PARKGLYEGTGRLFFEFYRLLHDARPKEGDDRPFFWLFENVVAMGVSDKRDI | |
| | SRFLESNPVMIDAKEVSAAHRARYFWGNLPGMNRPLASTVNDKLELQECLE | |
| | HGRIAKFSKVRTITTRSNSIKQGKDQHFPVFMNEKEDILWCTEMERVFGFPVH | |
| | YTDVSNMSRLARQRLLGRSWSVPVIRHLFAPLKEYFACV | |
| 286 | MGPMEIYKTVSAWKRQPVRVLSLFRNIDKVLKSLGFLESGSGSGGGTLKYVE | Murine |
| | DVTNVVRRDVEKWGPFDLVYGSTQPLGSSCDRCPGWYMFQFHRILQYALPR | DNMT3L |
| | QESQRPFFWIFMDNLLLTEDDQETTTRFLQTEAVTLQDVRGRDYQNAMRVW | |
| | SNIPGLKSKHAPLTPKEEEYLQAQVRSRSKLDAPKVDLLVKNCLLPLREYFKY | |
| | FSQNSLPL | |
| 287 | MNPLEMFETVPVWRRQPVRVLSLFEDIKKELTSLGFLESGSDPGQLKHVVDV | C-terminal |
| | TDTVRKDVEEWGPFDL | human |
| | VYGATPPLGHTCDRPPSWYLFQFHRLLQYARPKPGSPRPFFWMFVDNLVLNK | DNM3L |
| | EDLDVASR | |
| | FLEMEPVTIPDVHGGSLQNAVRVWSNIPAIRSRHWALVSEEELSLLAQNKQSS | |
| | KLAAKWP | |
| | TKLVKNCFLPLREYFKYFSTELTSSL | |
| 288 | SSGNSNANSRGPSFSSGLVPLSLRGSH | Linker |
| 289 | MGSRETPSSCSKTLETLDLETSDSSSPDADSPLEEQWLKSSPALKEDSVDVVL | Murine |
| | EDCKEPL | DNMT3L |
| | SPSSPPTGREMIRYEVKVNRRSIEDICLCCGTLQVYTRHPLFEGGLCAPCKDKF | |
| | LESLFL | |
| | YDDDGHQSYCTICCSGGTLFICESPDCTRCYCFECVDILVGPGTSERINAMAC | |
| | WVCFLCL | |
| | PFSRSGLLQRRKRWRHQLKAFHDQEGAGPMEIYKTVSAWKRQPVRVLSLFR | |
| | NIDKVLKSL | |
| | GFLESGSGSGGGTLKYVEDVTNVVRRDVEKWGPFDLVYGSTQPLGSSCDRCP | |
| | GWYMFQFH | |
| | RILQYALPRQESQRPFFWIFMDNLLLTEDDQETTTRFLQTEAVTLQDVRGRDY | |
| | QNAMRVW | |
| | SNIPGLKSKHAPLTPKEEEYLQAQVRSRSKLDAPKVDLLVKNCLLPLREYFKY | |
| | FSQNSLP | |
| | L | |
| 290 | RTLVTFKDVFVDFTREEWKLLDTAQQILYRNVMLENYKNLVSLGYQL | KRAB |
| | TKPDVILRLEKGEEPWLVEREIHQETHPDSETAFEIKSSV | |
| 291 | PKKKRKVGIHGVPAA | NLS and |
| | | linker |
| 292 | DYKDDDDK | Flag tag |
| 293 | EASGSGRASPGIPGSTR | Linker |
| 294 | GIHGVPAA | Linker |
| 295 | MDYKDHDGDYKDHDI DYKDDDDK | 3 x Flag |
| | | peptide |
| 296 | YPYDVPDYÅ | HA tag |
| 297 | HHHHHH | poly- |
| | | histidine |
| | | tag |
| 298 | KRPAATKKAGQAKKKKASDAKSLTAWS | Linker |
| 299 | SGSETPGTSESATPES | Linke |
| | | (aa) |
| 300 | CLSYETEILTVEYGLLPIGKIVEKRIECTVYSVDNNGNIYTQPVAQWHDRGEQ | N term |
| | EVFEYCLEDGSLIRATKDHKFMTVDGQMLPID | Npu Intein |
| | | (aa) |
| 301 | ATCAAGATTGCTACACGGAAATACCTGGGAAAGCAGAACGTGTACGACAT | C term |
| | CGGCGTGGAGCGGGATCACAACTTCGCCCTGAAGAATGGCTTTATCGCCA | Npu Intein |
| | GCAAT | (nt) |
| 302 | IKIATRKYLGKQNVYDIGVERDHNFALKNGFIASN | C term |
| | | Npu Intein |
| | | (aa) |
| 303 | ATGAAACGGACAGCCGACGGAAGCGAGTTCGAGTCACCAAAGAAGAAGC | dSpCas9- |
| | GGAAAGTCATCAAGATTGCTACACGGAAATACCTGGGAAAGCAGAACGT | 573-C |
| | GTACGACATCGGCGTGGAGCGGGATCACAACTTCGCCCTGAAGAATGGCT | Intein (nt) |
| | TTATCGCCAGCAATTGTTTCGACTCTGTTGAAATCAGCGGAGTGGAGGATC | |
| | GCTTCAACGCATCCCTGGGAACGTATCACGATCTCCTGAAAATCATTAAA | |
| | GACAAGGACTTCCTGGACAATGAGGAGAACGAGGACATTCTTGAGGACA | |
| | TTGTCCTCACCCTTACGTTGTTTGAAGATAGGGAGATGATTGAAGAACGCT | |
| | TGAAAACTTACGCTCATCTCTTCGACGACAAAGTCATGAAACAGCTCAAG | |
| | AGGCGCCGATATACAGGATGGGGGCGGCTGTCAAGAAAACTGATCAATG | |
| | GgatcCGAGACAAGCAGAGTGGAAAGACAATCCTGGATTTTCTTAAGTCCG | |
| | ATGGATTTGCCAACCGGAACTTCATGCAGTTGATCCATGATGACTCTCTCA | |
| | CCTTTAAGGAGGACATCCAGAAAGCACAAGTTTCTGGCCAGGGGGACAGT | |
| | CTTCACGAGCACATCGCTAATCTTGCAGGTAGCCCAGCTATCAAAAAGGG | |
| | AATACTGCAGACCGTTAAGGTCGTGGATGAACTCGTCAAAGTAATGGGAA | |
| | GGCATAAGCCCGAGAATATCGTTATCGAGATGGCCCGAGAGAACCAAACT | |
| | ACCCAGAAGGGACAGAAGAACAGTAGGGAAAGGATGAAGAGGATTGAA | |
| | GAGGGTATAAAAGAACTGGGGTCCCAAATCCTTAAGGAACACCCAGTTGA | |
| | AAACACCCAGCTTCAGAATGAGAAGCTCTACCTGTACTACCTGCAGAACG | |
| | GCAGGGACATGTACGTGGATCAGGAACTGGACATCAATCGGCTCTCCGAC | |
| | TACGACGTGGATGCCATCGTGCCCCAGTCTTTTCTCAAAGATGATTCTATT | |
| | GATAATAAAGTGTTGACAAGATCCGATAAAAATAGAGGGAAGAGTGATA | |
| | ACGTCCCCTCAGAAGAAGTTGTCAAGAAAATGAAAAATTATTGGCGGCAG | |
| | CTGCTGAACGCCAAACTGATCACACAACGGAAGTTCGATAATCTGACTAA | |
| | GGCTGAACGAGGTGGCCTGTCTGAGTTGGATAAAGCCGGCTTCATCAAAA | |
| | GGCAGCTTGTTGAGACACGCCAGATCACCAAgcacGTGGCCCAAATTCTCG | |
| | ATTCACGCATGAACACCAAGTACGATGAAAATGACAAACTGATTCGAGAG | |
| | GTGAAAGTTATTACTCTGAAGTCTAAGCTGGTCTCAGATTTCAGAAAGGA | |
| | CTTTCAGTTTTATAAGGTGAGAGAGATCAACAATTACCACCATGCGCATG | |
| | ATGCCTACCTGAATGCAGTGGTAGGCACTGCACTTATCAAAAAATATCCC | |
| | AAGCTTGAATCTGAATTTGTTTACGGAGACTATAAAGTGTACGATGTTAG | |
| | GAAAATGATCGCAAAGTCTGAGCAGGAAATAGGCAAGGCCACCGCTAAG | |
| | TACTTCTTTTACAGCAATATTATGAATTTTTTCAAGACCGAGATTACACTG | |
| | GCCAATGGAGAGATTCGGAAGCGACCACTTATCGAAACAAACGGAGAAA | |
| | CAGGAGAAATCGTGTGGGACAAGGGTAGGGATTTCGCGACAGTCCGGAA | |
| | GGTCCTGTCCATGCCGCAGGTGAACATCGTTAAAAAGACCGAAGTACAGA | |
| | CCGGAGGCTTCTCCAAGGAAAGTATCCTCCCGAAAAGGAACAGCGACAA | |
| | GCTGATCGCACGCAAAAAAGATTGGGACCCCAAGAAATACGGCGGATTC | |
| | GATTCTCCTACAGTCGCTTACAGTGTACTGGTTGTGGCCAAAGTGGAGAA | |
| | AGGGAAGTCTAAAAAACTCAAAAGCGTCAAGGAACTGCTGGGCATCACA | |
| | ATCATGGAGCGATCAAGCTTCGAAAAAAACCCCATCGACTTTCTCGAGGC | |
| | GAAAGGATATAAAGAGGTCAAAAAAGACCTCATCATTAAGCTTCCCAAGT | |
| | ACTCTCTCTTTGAGCTTGAAAACGGCCGGAAACGAATGCTCGCTAGTGCG | |
| | GGCGAGCTGCAGAAAGGTAACGAGCTGGCACTGCCCTCTAAATACGTTAA | |
| | TTTCTTGTATCTGGCCAGCCACTATGAAAAGCTCAAAGGGTCTCCCGAAG | |
| | ATAATGAGCAGAAGCAGCTGTTCGTGGAACAACACAAACACTACCTTGAT | |
| | GAGATCATCGAGCAAATAAGCGAATTCTCCAAAAGAGTGATCCTCGCCGA | |
| | CGCTAACCTCGATAAGGTGCTTTCTGCTTACAATAAGCACAGGGATAAGC | |
| | CCATCAGGGAGCAGGCAGAAAACATTATCCACTTGTTTACTCTGACCAAC | |
| | TTGGGCGCGCCTGCAGCCTTCAAGTACTTCGACACCACCATAGACAGAAA | |
| | GCGGTACACCTCTACAAAGGAGGTCCTGGACGCCACACTGATTCATCAGT | |
| | CAATTACGGGGCTCTATGAAACAAGAATCGACCTCTCTCAGCTCGGTGGA | |
| | GACAAAAGGCCGGCGGCCACGAAAAAGGCCGGCCAGGCAAAAAAGAAA | |
| | AAGTGA | |
| 304 | MKRTADGSEFESPKKKRKVIKIATRKYLGKQNVYDIGVERDHNFALKNGFIA | dSpCas9- |
| | SNCFDSVEISGVEDRFNASLGTYHDLLKIIKDKDFLDNEENEDILEDIVLTLTLF | 573-C |
| | EDREMIEERLKTYAHLFDDKVMKQLKRRRYTGWGRLSRKLINGIRDKQSGK | Intein (aa) |
| | TILDFLKSDGFANRNFMQLIHDDSLTFKEDIQKAQVSGQGDSLHEHIANLAGS | |
| | PAIKKGILQTVKVVDELVKVMGRHKPENIVIEMARENQTTQKGQKNSRERM | |
| | KRIEEGIKELGSQILKEHPVENTQLQNEKLYLYYLQNGRDMYVDQELDINRLS | |
| | DYDVDAIVPQSFLKDDSIDNKVLTRSDKNRGKSDNVPSEEVVKKMKNYWRQ | |
| | LLNAKLITQRKFDNLTKAERGGLSELDKAGFIKRQLVETRQITKHVAQILDSR | |
| | MNTKYDENDKLIREVKVITLKSKLVSDFRKDFQFYKVREINNYHHAHDAYLN | |
| | AVVGTALIKKYPKLESEFVYGDYKVYDVRKMIAKSEQEIGKATAKYFFYSNI | |
| | MNFFKTEITLANGEIRKRPLIETNGETGEIVWDKGRDFATVRKVLSMPQVNIV | |
| | KKTEVQTGGFSKESILPKRNSDKLIARKKDWDPKKYGGFDSPTVAYSVLVVA | |
| | KVEKGKSKKLKSVKELLGITIMERSSFEKNPIDFLEAKGYKEVKKDLIIKLPKY | |
| | SLFELENGRKRMLASAGELQKGNELALPSKYVNFLYLASHYEKLKGSPEDNE | |
| | QKQLFVEQHKHYLDEIIEQISEFSKRVILADANLDKVLSAYNKHRDKPIREQA | |
| | ENIIHLFTLTNLGAPAAFKYFDTTIDRKRYTSTKEVLDATLIHQSITGLYETRID | |
| | LSQLGGDKRPAATKKAGQAKKKK | |
| 305 | TGCCTGTCCTACGAGACAGAGATCCTGACAGTGGAGTATGGCCTGCTGCC | N term |
| | AATCGGCAAGATCGTGGAGAAGAGGATCGAGTGTACCGTGTACTCTGTGG | Npu Intein |
| | ATAACAATGGCAACATCTATACACAGCCCGTGGCACAGTGGCACGATAGG | (nt) |
| | GGAGAGCAGGAGGTGTTCGAGTATTGCCTGGAGGACGGCAGCCTGATCA | |
| | GGGCAACCAAGGACCACAAGTTCATGACAGTGGATGGCCAGATGCTGCCC | |
| | ATCGAC | |
| 390 | AGGTTTCCGCAGCGGCGTCG | cPCSK9- |
| | | C gRNA |
| | | target site |
| 391 | AGGUUUCCGCAGCGGCGUCG | cPCSK9- |
| | | C gRNA |
| | | spacer |
| 392 | AGGUUUCCGCAGCGGCGUCGGUUUUAGAGCUAGAAAUAGCAAGU | cPCSK9- |
| | UAAAAUAAGGCUAGUCCGUUAUCAACUUGAAAAAGUGGCACCGA | C full |
| | GUCGGUGCUUUU | length |
| | | gRNA |
| 393 | ggagcaauugaccgggaagcucagaauaaacgcucaacuuuggccggaucuucuagagccacc | 5′ UTR |
| | | (5U4) |
| 394 | uaguaagaauucaaagaaaguuucuucacauucucucgagcguacg | 3′ UTR |
| | | (3U2) |
| 395 | ggagcaauugaccgggaagcucagaauaaacgcucaacuuuggccggaucuucuagagccaccauga | dCas9- |
| | accacgaucaggaguuugaccccccuaagguguacccacccgugccagccgagaagaggaagcccauc | KRAB- |
| | cgcgugcugucccuguucgacggcaucgccacaggccugcuggugcugaaggaucugggcauccag | DNMT3 |
| | guggacagauauaucgccuccgaggugugcgaggauucuaucaccgugggcauggugaggcaccag | A/L |
| | ggcaagaucauguacgugggcgacgugcgcagcgugacacagaagcacauccaggaguggggacccu | mRNA |
| | ucgaccuggucaucggaggcagccccuguaaugaccuguccaucgugaacccugcaaggaagggccu | |
| | guaugagggaaccggcagacuguucuuugaguucuacaggcugcugcacgacgcccgcccuaaggag | |
| | ggcgaugacaggccauucuuuuggcuguuugagaacgugguggccaugggcgugagcgacaagcgg | |
| | gauaucuccagauuccuggagucuaaucccgugaugaucgaugcaaaggaggugucugccgcacaca | |
| | gggcaagguacuuuuggggaaaucugccuggcaugaaccgcccacuggccagcaccgugaacgacaa | |
| | gcuggagcugcaggagugccuggagcacggaaggaucgccaaguucuccaaggugeggacaaucacc | |
| | acaagaucuaacagcaucaagcagggcaaggaucagcacuuccccguguucaugaaugagaaggagg | |
| | acauccugugguguaccgagauggagcgcguguucggcuuuccagugcacuauacagacgugagca | |
| | auaugagccggcuggcaaggcagagacugcugggccgguccuggucugugccagugaucagacacc | |
| | uguucgccccccugaaggaguacuuugccugcgugucuagcggcaacucuaaugccaacagcagagg | |
| | cccuuccuuuuccucuggccuggugccacugucucugaggggcagccacaugggccccauggagauc | |
| | uacaagaccguguccgccuggaagaggcagccuguggcgugcugucucuguuccgcaacaucgaca | |
| | aggugcucaagagccugggcuuucuggagagcggauccggaucuggaggaggcacccugaaguaug | |
| | uggaggaugugacaaauguggugcggagagauguggagaaguggggccccuucgaucugguguacg | |
| | gauccacccagccacugggaagcuccugcgauagguguccaggaugguauauguuccaguuucacag | |
| | aauccugcaguacgcacugccaaggcaggagagccagegcccuuucuuuuggaucuuuauggacaac | |
| | cugcugcugacagaggaugaccaggagacaacaacccgcuuccugcagacagaggcagugacccugc | |
| | aggaugugaggggacgcgacuaucagaaugccaugcggguguggucuaacaucccuggccugaaaa | |
| | gcaagcacgccccccugaccccuaaggaggaggaguaccugcaggcccaggugcggagcagauccaa | |
| | gcuggaugccccuaagguggaccugcuggugaagaauugucugcugccacugcgggaguacuucaa | |
| | guacuuuagucagaauagccugccacuggaggcaagcggauccggaagggcaucuccuggaauccca | |
| | ggaagcacccgcaaccccaagaagaagcggaaggugggcauccacggcgugcccgccgccgacaagaa | |
| | guacagcaucggccuggccaucggcaccaacagcgugggcugggccgugaucaccgacgaguacaag | |
| | gugcccagcaagaaguucaaggugcugggcaacaccgaccggcacagcaucaagaagaaccugaucg | |
| | gcgcccugcuguucgacagcggcgagaccgccgaggccacccggcugaagcggaccgcccggcggcg | |
| | guacacccggcggaagaaccggaucugcuaccugcaggagaucuucagcaacgagauggccaaggug | |
| | gacgacagcuucuuccaccggcuggaggagagcuuccugguggaggaggacaagaagcacgagcggc | |
| | accccaucuucggcaacaucguggacgagguggccuaccacgagaaguaccccaccaucuaccaccug | |
| | cggaagaagcugguggacagcaccgacaaggccgaccugcggcugaucuaccuggcccuggcccaca | |
| | ugaucaaguuccggggccacuuccugaucgagggcgaccugaaccccgacaacagcgacguggacaa | |
| | gcuguucauccagcuggugcagaccuacaaccagcuguucgaggagaaccccaucaacgccagcggc | |
| | guggacgccaaggccauccugagcgcccggcugagcaagagccggcggcuggagaaccugaucgccc | |
| | agcugcccggcgagaagaagaacggccuguucggcaaccugaucgcccugagccugggccugacccc | |
| | caacuucaagagcaacuucgaccuggccgaggacgccaagcugcagcugagcaaggacaccuacgacg | |
| | acgaccuggacaaccugcuggcccagaucggcgaccaguacgccgaccuguuccuggccgccaagaac | |
| | cugagcgacgccauccugcugagcgacauccugcgggugaacaccgagaucaccaaggccccccuga | |
| | gcgccagcaugaucaagcgguacgacgagcaccaccaggaccugacccugcugaaggcccuggugcg | |
| | gcagcagcugcccgagaaguacaaggagaucuucuucgaccagagcaagaacggcuacgccggcuac | |
| | aucgacggcggcgccagccaggaggaguucuacaaguucaucaagcccauccuggagaagauggacg | |
| | gcaccgaggagcugcuggugaagcugaaccgggaggaccugcugcggaagcagcggaccuucgacaa | |
| | cggcagcaucccccaccagauccaccugggcgagcugcacgccauccugcggggcaggaggacuuc | |
| | uaccccuuccugaaggacaaccgggagaagaucgagaagauccugaccuuccggauccccuacuacg | |
| | ugggcccccuggcccggggcaacagccgguucgccuggaugacccggaaaagcgaggagaccaucac | |
| | ccccuggaacuucgaggaggugguggacaagggcgccagegcccagagcuucaucgagcggaugacc | |
| | aacuucgacaagaaccugcccaacgagaaggugcugcccaagcacagccugcuguacgaguacuucac | |
| | cguguacaacgagcugaccaaggugaaguacgugaccgagggcaugeggaagcccgccuuccugagc | |
| | ggcgagcagaagaaggccaucguggaccugcuguucaagaccaaccggaaggugaccgugaagcagc | |
| | ugaaggaggacuacuucaagaagaucgagugcuucgacagcguggagaucagcggcguggaggacc | |
| | gguucaacgccagccugggcaccuaccacgaccugcugaagaucaucaaggacaaggacuuccugga | |
| | caacgaggagaacgaggacauccuggaggacaucgugcugacccugacccuguucgaggaccgggag | |
| | augaucgaggagcggcugaagaccuacgcccaccuguucgacgacaaggugaugaagcagcugaagc | |
| | ggcggcgguacaccggcuggggccggcugagccggaagcugaucaacggcauccgggacaagcagag | |
| | cggcaagaccauccuggacuuccugaaaagcgacggcuucgccaaccggaacuucaugcagcugauc | |
| | cacgacgacagccugaccuucaaggaggacauccagaaggcccaggugagcggccagggcgacagcc | |
| | ugcacgagcacaucgccaaccuggccggcagccccgccaucaagaagggcauccugcagaccgugaag | |
| | gugguggacgagcuggugaaggugaugggccggcacaagcccgagaacaucgugaucgagauggcc | |
| | cgggagaaccagaccacccagaagggccagaagaacagccgggagcggaugaagcggaucgaggagg | |
| | gcaucaaggagcugggcagccagauccugaaggagcaccccguggagaacacccagcugcagaacga | |
| | gaagcuguaccuguacuaccugcagaacggccgggacauguacguggaccaggagcuggacaucaac | |
| | cggcugagcgacuacgacguggacgccaucgugccccagagcuuccugaaggacgacagcaucgaca | |
| | acaaggugcugacccggagcgacaagaaccggggcaagagcgacaacgugcccagcgaggagguggu | |
| | gaagaagaugaagaacuacuggcggcagcugcugaacgccaagcugaucacccageggaaguucgac | |
| | aaccugaccaaggccgagggggcggccugagcgagcuggacaaggccggcuucaucaagcggcagc | |
| | ugguggagacccggcagaucaccaagcacguggcccagauccuggacagccggaugaacaccaagua | |
| | cgacgagaacgacaagcugauccgggaggugaaggugaucacccugaaaagcaagcuggugagcgac | |
| | uuccggaaggacuuccaguucuacaaggugcgggagaucaacaacuaccaccacgcccacgacgccua | |
| | ccugaacgccguggugggcaccgcccugaucaagaaguaccccaagcuggagagcgaguucguguac | |
| | ggcgacuacaagguguacgacgugcggaagaugaucgccaagagcgagcaggagaucggcaaggcca | |
| | ccgccaaguacuucuucuacagcaacaucaugaacuucuucaagaccgagaucacccuggccaacggc | |
| | gagauccggaagcggccccugaucgagaccaacggcgagaccggcgagaucgugugggacaagggcc | |
| | gggacuucgccaccgugcggaaggugcugagcaugccccaggugaacaucgugaagaagaccgaggu | |
| | gcagaccggcggcuucagcaaggagagcauccugcccaagcggaacagcgacaagcugaucgcccgg | |
| | aagaaggacugggaccccaagaaguacggcggcuucgacagccccaccguggccuacagcgugcugg | |
| | ugguggccaagguggagaagggcaagagcaagaagcugaaaagcgugaaggagcugcugggcaucac | |
| | caucauggagcggagcagcuucgagaagaaccccaucgacuuccuggaggccaagggcuacaaggag | |
| | gugaagaaggaccugaucaucaagcugcccaaguacagccuguucgagcuggagaacggccggaagc | |
| | ggaugcuggccagcgccggcgagcugcagaagggcaacgagcuggcccugcccagcaaguacgugaa | |
| | cuuccuguaccuggccagccacuacgagaagcugaagggcagccccgaggacaacgagcagaagcagc | |
| | uguucguggagcagcacaagcacuaccuggacgagaucaucgagcagaucagcgaguucagcaagcg | |
| | ggugauccuggccgacgccaaccuggacaaggugcugagcgccuacaacaagcaccgggacaagccca | |
| | uccgggagcaggccgagaacaucauccaccuguucacccugaccaaccuggggcccccgccgccuuc | |
| | aaguacuucgacaccaccaucgaccggaagcgguacaccagcaccaaggaggugcuggacgccacccu | |
| | gauccaccagagcaucaccggccuguacgagacccggaucgaccugagccagcugggggcgacagc | |
| | ggcggcaagcggcccgccgccaccaagaaggccggccaggccaagaagaagaaggcuagcgaugcuaa | |
| | gucacugacugccuggucccggacacuggugaccuucaaggauguguuuguggacuucaccaggga | |
| | ggaguggaagcugcuggacacugcucagcagauccuguacagaaaugugaugcuggagaacuauaag | |
| | aaccugguuuccuuggguuaucagcuuacuaagccagaugugauccuccgguuggagaagggagag | |
| | gaacccuggcugguggagagagaaauucaccaagagacccauccugauucagagacugcauuugaaa | |
| | ucaaaucaucaguuccgaaaaagaaacgcaaaguuuaguaagaauucaaagaaaguuucuucacauuc | |
| | ucucgagcguacgaaaaaaaaaaaaaaaaaaaaaaaaaaaaaaaaaaaaaaaaaaaaaaaaaaaaaaaaa | |
| | aaaaaaaaaaaaaaaaaaaaaaaaaaaaaaaaaaaaaaaaaaaaaaaaaaaaaaaaaaaaaaa | |

1. A polynucleotide encoding an epigenetic-modifying DNA-targeting system, wherein the epigenetic-modifying DNA-targeting system is for repressing transcription of PCSK9 and comprises (a) a fusion protein comprising a dead Streptococcuspyogenes Clustered Regularly Interspaced Short Palindromic Repeats associated protein 9 (dSpCas9), a DNA-methyltransferase (DNMT) 3A/L domain, and a Kruppel associated box (KRAB) domain; and (b) a gRNA comprising the sequence set forth in SEQ ID NO: 66.

2. The polynucleotide of claim 1, wherein the dSpCas9 comprises at least 95% sequence identity to the amino acid sequence set forth in SEQ ID NO: 207 and comprises an alanine (A) at amino acid position 9 and an alanine at position 839 corresponding to amino acid residues in SEQ ID NO:207.

3. The polynucleotide of claim 1, wherein the KRAB domain comprises an amino acid sequence that has at least 95% sequence identity to SEQ ID NO:193.

4. The polynucleotide of claim 1, wherein the DNMT3A/L domain comprises an amino acid sequence that has at least 95% sequence to SEQ ID NO: 199.

5. The polynucleotide of claim 1, wherein the KRAB domain and DNMT3A/L domain are independently fused to the N-terminus, the C-terminus, or both the N-terminus and the C-terminus, of the dSpCas9.

6. The polynucleotide of claim 1, wherein the fusion protein further comprises one or more nuclear localization signals (NLS).

7. The polynucleotide of claim 6, wherein the one or more NLS comprise the amino acid sequence PKKKRKV (SEQ ID NO: 234) or the amino acid sequence KRPAATKKAGQAKKKK (SEQ ID NO: 249).

8. The polynucleotide of claim 1, wherein the fusion protein comprises in order from the N- to C-terminus: the DNMT 3A/L domain, the dSpCas9, and the KRAB domain.

9. The polynucleotide of claim 8, wherein the DNMT 3A/L domain comprises the amino acid sequence set forth in SEQ ID NO: 199, the dSpCas9 comprises the amino acid sequence set forth in SEQ ID NO: 207, and the KRAB domain comprises the amino acid sequence set forth in SEQ ID NO:290.

10. The polynucleotide of claim 8, wherein an NLS is between the DNMT3A/L domain and the dSpCas9 protein, and an NLS is between the SpCas9 protein and the KRAB domain.

11. The polynucleotide of claim 9, wherein an NLS is between the DNMT3A/L domain and the dSpCas9 protein, and an NLS is between the SpCas9 protein and the KRAB domain.

12. The polynucleotide of claim 10, wherein the NLS between the DNMT3A/L domain and the dSpCas9 protein has the amino acid sequence PKKKRKV (SEQ ID NO: 234).

13. The polynucleotide of claim 10, wherein the NLS between the dSpCas9 protein and the KRAB domain has the amino acid sequence KRPAATKKAGQAKKKK (SEQ ID NO: 249).

14. The polynucleotide of claim 10, further comprising an NLS at the C-terminus of the fusion protein.

15. The polynucleotide of claim 14, wherein the NLS at the C-terminus of the fusion protein has the amino acid sequence PKKKRKV (SEQ ID NO: 234).

16. A polynucleotide encoding an epigenetic-modifying DNA-targeting system, wherein the epigenetic-modifying DNA-targeting system is for repressing transcription of PCSK9 and comprises (a) a fusion protein comprising an amino acid sequence that has at least 95% sequence identity to SEQ ID NO:278; and (b) a gRNA comprising the sequence set forth in SEQ ID NO: 66.

17. The polynucleotide of claim 16, wherein the fusion protein comprises the amino acid sequence set forth in SEQ ID NO: 278.

18. The polynucleotide of claim 1, wherein the gRNA comprises the sequence set forth in SEQ ID NO:129.

19. The polynucleotide of claim 9, wherein the gRNA comprises the sequence set forth in SEQ ID NO:129.

20. The polynucleotide of claim 16, wherein the gRNA comprises the sequence set forth in SEQ ID NO:129.

21. A vector comprising the polynucleotide of claim 1.

22. A vector comprising the polynucleotide of claim 16.

23. A vector comprising the polynucleotide of claim 18.

24. A vector comprising the polynucleotide of claim 20.
